(19) United States
(12) Patent Application Publication
(10) Pub. No.: US 2024/0307453 A1
(43) Pub. Date: Sep. 19, 2024

(54) METHODS FOR CONTROLLING BLEEDING IN A SUBJECT AFFLICTED WITH HERMANSKY PUDLAK SYNDROME USING PLATELET DERIVATIVE COMPOSITIONS

(71) Applicant: Cellphire, Inc., Rockville, MD (US)

(72) Inventors: Keith Andrew MOSKOWITZ, Westfield, IN (US); William Matthew DICKERSON, Washington, DC (US); Maria Teresa ABREU-BLANCO, Rockville, MD (US); Lisa Ellen BOOTH, Gaithersburg, MD (US); Emma Caitlin LESHAN, Bethesda, MD (US); Ana Maria SWANSON, Burke, VA (US); Arjun Shyam CHERUPALLA, Silver Spring, MD (US); Benjamin J. KUHN, Norfolk, VA (US); William Allan ALEXANDER, Athens, TX (US); Glen Michael FITZPATRICK, North Potomac, MD (US)

(73) Assignee: Cellphire, Inc., Rockville, MD (US)

(21) Appl. No.: 18/653,905

(22) Filed: May 2, 2024

Related U.S. Application Data
(63) Continuation-in-part of application No. PCT/US2024/019800, filed on Mar. 13, 2024.
(60) Provisional application No. 63/499,959, filed on May 3, 2023.
(60) Provisional application No. 63/499,959, filed on May 3, 2023.
(60) Provisional application No. 63/490,186, filed on Mar. 14, 2023.

(51) Int. Cl. A61K 35/19; A61K 9/00; A61P 7/04

(52) U.S. Cl. CPC A61K 35/19; A61K 9/0019; A61P 7/04

(57) ABSTRACT

Provided herein are methods for administering platelet derivatives, such as freeze-dried platelet derivatives (FDPDs) to a subject, comprising administering an effective dose of the platelet derivatives in a platelet derivative composition to the subject. Such effective dose in illustrative embodiments, includes multiple individual doses, or a continuous dose administered within a time period, for example a time period of less than 4 hours. In certain aspects herein the subject is a subject having Hermansky Pudlak Syndrome (HPS). The platelet derivatives can have numerous characteristics provided herein that make them well suited to restore hemostatic functions in the subject. In some embodiments, the platelet derivatives are from a pool of donors, and are HLA Class 1-characterized platelet derivatives (e.g., FDPDs), which in certain illustrative embodiments are HLA Class 1-matched FDPDs. Furthermore, provided herein are platelet derivatives, and methods using the same, that have numerous beneficial properties, as provided.

### CROSS-REFERENCE TO RELATED APPLICATIONS

This application claims priority to U.S. Provisional Application Ser. No. 63/499,959, filed on May 3, 2023, and is a continuation-in-part of International Application No. PCT/US2024/019800, filed on Mar. 13, 2024. International Application No. PCT/US2024/019800 claims priority to U.S. Provisional Application Ser. No. 63/490,186, filed on Mar. 14, 2023, and to U.S. Provisional Application Ser. No. 63/499,959, filed on May 3, 2023. Each of the aforementioned applications is incorporated herein by reference in its entirety.

### STATEMENT OF GOVERNMENT INTEREST

This invention was made with government support under Contract No. HHSO100201300022 awarded by the Biomedical Advanced Research and Development Authority (BARDA) of the U.S. Department of Health and Human Services. The government has certain rights in the invention.

### TECHNICAL FIELD

The present disclosure generally relates to blood products, including those containing platelet derivatives, methods of producing such blood products, and methods of treating a subject using such blood products.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Inactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Platelets are essential to achieving hemostasis and limiting blood loss. Studies have shown that early transfusion of whole blood or red blood cells and plasma can potentially reduce mortality and advance the standard of care for preventable deaths from hemorrhage (Holcomb, J. B. et al. The Prospective, Observational, Multicenter, Major Trauma Transfusion (PROMMTT) Study: Comparative Effectiveness of a Time-Varying Treatment With Competing Risks. JAMA Surg 148, 127 (2013)). However, platelet transfusions have drawbacks. Platelets stored at room temperature (RT) require special chambers with agitation and have a short shelf-life (5 to 7 days). Cryopreserved platelets require ultra-low freezers (−80° C.), time to thaw and prepare, and have reduced circulation time. The RT and cryopreserved platelet products are not suitable to support casualties in austere environments and are difficult to stockpile for mass casualty and mass transfusion events. Platelet transfusion is not available to all patients and hospitals. Platelet supplies are inadequate for current clinical demand. The American Red Cross cannot routinely supply platelets to 33% of the hospitals to which they provide other blood components (Young, P. P., Ehrhart, J. & Mair, D. Rural hospitals and access to platelets for treating active bleeding: Is the heartland bleeding? Transfusion 60, 2474-2475 (2020)). Thus, there is a need for a safe and efficacious hemostatic product with a long shelf life for benefiting patients with platelet deficiency or dysfunction, or patients afflicted with an indication that affects the functions of platelets.

Hermansky-Pudlak syndrome (HPS) is a rare autosomal recessive disorder characterized by abnormal biogenesis of lysosome-related organelles which manifests with oculocutaneous albinism and excessive bleeding of variable severity. HPS was initially described by Frantisek Hermansky and Paulus Pudlak in 1959 (Hermansky, F, and Pudlak, P. “Albinism associated with hemorrhagic diathesis and unusual pigmented reticular cells in the bone marrow: report of two cases with histochemical studies.” Blood vol. 14, 2 (1959): 162-9). These authors reported two patients with oculocutaneous albinism, bleeding diathesis due to platelet dysfunction, and reticular cells with pigment deposits in the bone marrow. Since the initial report, 10 genetic types of HPS have been identified and each is associated with a defect in biogenesis of lysosome-related organelles complex (BLOC)-1 (HPS-7, HPS-8 and HPS-9), BLOC-2 (HPS-3, HPS-5 and HPS-6), BLOC-3 (HPS-1 and HPS-4) or adapter protein (AP)-3 complex (HPS-2 and HPS-10).

Mutations observed in HPS are known to cause impairment of specialized secretory cells, including melanocytes, platelets, and lung alveolar type II epithelial cells. These patients demonstrate prolonged bleeding after surgical procedures and easy bruising (Pierson, Diane M., et al. “Pulmonary fibrosis in Hermansky-Pudlak syndrome.” Respiration 73.3 (2006): 382-395.). Platelet transfusion is one of the treatment options for subjects with HPS. However, transfusion with platelets is associated with risks including transfusion-associated acute lung injury, allergic reactions, clinically relevant immunomodulation, post-transfusion purpura, infectious risk, and alloimmunization with subsequent ineffectiveness of platelet transfusion. Further, since conventional platelets cannot be frozen, they have the highest risk of bacterial sepsis of any blood product (Corash, Laurence. “Bacterial contamination of platelet components: potential solutions to prevent transfusion-related sepsis.” Expert review of hematology 4.5 (2011): 509-525). Accordingly, there is a need for an improved platelet product for effectively managing or treating the symptoms of a subject afflicted with a platelet dysfunction, such as HPS.

More specifically with respect to platelet transfusions and HLA antibody production, multiple transfusions often lead to alloimmunization and platelet refractoriness. Once patients exhibit platelet refractoriness, the treatment options become more limited (El-Chemaly et al., “Clinical management and outcomes of patients with Hermansky-Pudlak syndrome pulmonary fibrosis evaluated for lung transplantation,” PLoS One 2018; 13(3): e0194193). Platelet-transfusion refractoriness caused by HLA alloimmunization has resulted in an increased incidence of death and complications in patients who require frequent transfusions in other diseases, such as those with acute leukemia or myelodysplastic syndromes (MDS) (Kerkhoffs J-L H et al., “The clinical impact of platelet refractoriness: correlation with bleeding and survival,” Transfusion 2008; 48:1959-1965). Thus, there is a need for an improved HLA-characterized hemostatic product for the treatment of HPS patients, that is less likely to induce or exacerbate HLA alloantibody formation.

### SUMMARY

To overcome the above-mentioned and additional problems in the art, the present disclosure provides, at least in part, blood products, such as, freeze-dried platelets, freeze-dried platelet derivatives (FDPDs) or HLA-characterized FDPDs, or freeze-dried platelet-derived hemostat (FPH) or HLA-characterized FPH for use in controlling bleeding in a subject, for example a subject having a platelet dysfunction, which in illustrative embodiments are subjects afflicted with HPS. Also provided herein, are freeze-dried platelets, FDPDs or HLA-characterized FDPDs, or FPH or HLA-characterized FPH for use in administering, for example, to control bleeding in a subject having a platelet dysfunction, for example, HPS. Furthermore, provided herein, are methods for administering the freeze-dried platelets, freeze-dried platelet derivatives (FDPDs) or HLA-characterized FDPDs, or FPH or HLA-characterized FPH in a subject having a platelet dysfunction, for example, HPS.

Accordingly, provided herein in one aspect is a method for controlling bleeding in a subject with a platelet dysfunction, for example, HPS, by administering freeze-dried platelet derivatives (FDPDs) or FPH to the subject, comprising administering an effective dose of the freeze-dried platelet derivatives in a platelet derivative composition to the subject. The FDPDs or FPH can have numerous characteristics provided herein that make them well suited to restore hemostatic functions in the subject. In some embodiments, the FDPDs or FPH are from a pool of donors and are HLA Class 1-characterized FDPDs or HLA-characterized FPH, which in certain illustrative embodiments are HLA-compatible FPH, HLA Class 1-matched FDPDs or HLA Class 1-matched FPH.

In an aspect, provided herein is a composition comprising an effective dose of platelet derivatives for use for controlling bleeding in a subject, wherein said composition is administered to the subject:
i) as a continuous infusion procedure; or
ii) at a frequency of at least one dose every 6 hours, 5 hours, 4 hours, 3 hours, 2 hours, or more frequently, in illustrative embodiments, every 1 hour, 45 minutes, 30 minutes, 15 minutes, or more frequently, wherein the administration comprises administering a total of 2 to 24 doses, in illustrative embodiments 2 to 10 doses, in a 24-hour period,
until bleeding of the subject is reduced as compared to the bleeding before the administration, or in illustrative embodiments until bleeding of the subject is stopped. In illustrative embodiments, the platelet derivatives:
a) have the ability to generate thrombin in vitro in the presence of tissue factor and phospholipids;
b) have the ability to occlude a collagen-coated microchannel in vitro; or
c) both a) and b).

In another aspect, provided herein is a composition comprising an effective dose of platelet derivatives for use for controlling bleeding in a subject,
wherein said composition is administered to the subject multiple times by administering 2 to 24 doses, 2 to 20 doses, 2 to 10 doses, 3 to 10 doses, 4 to 10 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, until bleeding of the subject is reduced as compared to the bleeding before the administering, and
wherein the platelet derivatives have the ability to generate thrombin in vitro in the presence of tissue factor and phospholipids.

In another aspect, provided herein is a method for administering freeze-dried platelet derivatives to a subject having Hermansky Pudlak Syndrome (HPS), comprising:
administering an effective dose of the freeze-dried platelet derivatives in a platelet derivative composition to the subject,
wherein the platelet derivative composition comprises a population of freeze-dried platelet derivatives (FDPDs) comprising CD 41-positive platelet derivatives, wherein less than 5% of the CD 41-positive platelet derivatives are microparticles,
and wherein the FDPDs
   a) have the ability to generate thrombin in vitro in the presence of tissue factor and phospholipids;
   b) have the ability to occlude a collagen-coated microchannel in vitro; or
   c) both a) and b).

In some embodiments, the administering increases the levels of at least one platelet biomarker selected from CD62P, PAC-1, and CD63 for endogenous platelets of the subject (recipient) as compared to before the administering. In illustrative embodiments, levels of both CD62P and PAC-1 are increased in the subject after the administering.

In some embodiments, the method further comprises before the administering, rehydrating the freeze-dried platelet derivatives to form a rehydrated platelet derivative composition, and wherein the administering is administering an effective dose of the rehydrated platelets from the rehydrated platelet derivative composition to the subject.

Further details regarding aspects and embodiments of the present disclosure are provided throughout this patent application. The preceding paragraphs in this Summary section is not an exhaustive list of aspects and embodiments disclosed herein. Sections and section headers are for ease of reading and are not intended to limit combinations of disclosure, such as methods, compositions, and kits or functional elements therein across sections.

### DESCRIPTION OF THE DRAWINGS

FIG. 1A shows flow cytometry MFI values for freeze-dried platelet-derived hemostatic product (FPH) and apheresis platelets (APU) for adhesion proteins and receptors: anti-GPIa/CD49b and anti-GPVI, anti-thrombospondin, anti-GPlbα, anti-vWF.

FIG. 1B shows flow cytometry MFI values for freeze-dried platelet-derived hemostatic product (FPH) and apheresis platelets (APU) for activation markers: anti-P-selectin/CD62 and anti-LAMP3/CD63 and PS/lactadherin.

FIG. 1C shows flow cytometry MFI values for freeze-dried platelet-derived hemostatic product (FPH) and apheresis platelets (APU) for aggregation proteins and receptors: anti-GPIIbIIIa/CD61 or anti-fibrinogen.

FIG. 2 shows aggregation of FPH (2.5×10⁵/μl) or day 4 apheresis platelets that were stimulated with 0, 2, 5, or 10 mM MgCl₂ and incubated at 37° C. with stirring.

FIG. 3A shows aggregation of FPH in representative histograms of flow cytometry results for FPH stained for active conformation GPIIbIIIa (PAC-1) with 10 mM MgCl₂ and without MgCl₂.

FIG. 3B shows aggregation of FPH in representative histograms of flow cytometry results for FPH stained for GPIIbIIIa presence (CD41) with 10 mM MgCl₂ and without MgCl₂.

FIG. 4 shows aggregation of FPH in the samples, FPH (2.5×10⁵/μl) untreated, stimulated with 5 mM MgCl₂, or stimulated with 5 mM MgCl₂ in the presence of Tirofiban and incubated at 37° C. with stirring.

FIG. 5 shows aggregation of FPH in the samples, FPH (2.5×10⁵/μl) was stimulated with 0, 2, 5, or 10 mM MgCl₂ after 29.2 nkat/ml plasmin treatment and incubated at 37° C. with stirring.

FIG. 6 shows aggregation of FPH in the samples, WPS (3×10⁶/μl) and washed FPH (3×10⁶/μl) were stimulated with 10 μg/ml collagen, 10 U/ml thrombin, 10 μg/ml arachidonic acid, or 25 μg/ml TRAP-6 in HMTA buffer. AcTcounts were taken of all samples following incubation at 37° C. with stirring.

FIG. 7 shows aggregation and interactions of washed platelets and washed FPH. Surface markers fibrinogen, GPIIIa (CD61), and complexed GPIIb/IIIa (CD41) were detected by flow cytometry for WPS (1×10⁶/μl) and washed FPH (1×10⁶/μl) treated with and without EDTA.

FIG. 8 shows aggregation and interactions of washed platelets and washed FPH. WPS (3×10⁶/μl) and washed FPH (3×10⁶/μl) mixed in a 1:1 ratio with or without 1 mM RGDS were incubated at 37° C. with stirring.

FIG. 9 shows aggregation of FPH in samples where FPH was washed and resuspended with different concentrations of plasmin and incubated for 1 hour. FPH samples were stained with anti-fibrinogen (9F9), anti-CD61, and anti-vWF antibodies. Flow cytometry MFI values normalized to the untreated condition are presented.

FIG. 10 shows aggregation and interactions of washed platelets and washed FPH. Washed FPH treated with plasmin mixed with WPS and incubated. Counts were taken on the AcTDiff 2 before and after incubation.

FIG. 11 shows aggregation and interactions of washed platelets and washed FPH. WPS were treated with 100 μM aspirin and 1 μg/ml ticagrelor. Treated and untreated WPS (3×10⁶/μl) alone and mixed 1:1 with washed FPH (3×10⁶/μl) were stimulated with 10 μg/ml collagen, or 10 μg/ml arachidonic acid in HMTA on the PAP-8E. HMTA alone served as an unstimulated control.

FIG. 12A shows thrombin generation in FPH and apheresis platelets. Peak thrombin generation for FPH (n=5) and apheresis platelets (n=5),

FIG. 12B shows thrombin generation in FPH and apheresis platelets. Time to maximum rate of thrombin generation for FPH and apheresis platelets in the 1:64 diluted samples.

FIG. 13A shows the linear dose-response of the thrombin generation of FPH with escalating concentrations of lactadherin to block PS expression.

FIG. 13B shows the time to maximum thrombin generation in FPH at the 1:64 dilution with increasing concentrations of lactadherin.

FIG. 14A shows the dose-response of the thrombin generation of FPH to anti-FV antibody.

FIG. 14B shows the time to maximum thrombin generation in FPH samples at the 1:64 dilution with increasing concentrations of anti-FV antibody.

FIG. 15A shows the linear dose-response of the thrombin generation of FPH with escalating concentrations of Rivaroxaban to inhibit FXa.

FIG. 15B shows the time to maximum thrombin generation by FPH at the 1:64 dilution with increasing concentrations of rivaroxaban.

FIG. 16A shows thrombin generation in FPH and apheresis platelets. Fresh platelets washed and resuspended in either 5 mM EDTA, 5 mM Ca2++5 mM Mg2++5 mM GPRP+20 μM TRAP-6 (“Activated”), or 5 mM Ca2++5 mM Mg2++5 mM GPRP+20 μM TRAP-6+200 μg/ml lactadherin (“Activated+Lactadherin”).

FIG. 16B shows thrombin generation in FPH and apheresis platelets. Fresh platelets and FPH washed and treated as in I) were each incubated with 500 nMFVa-FITC, for 90 minutes, then measured for binding by flow cytometry MFI.

FIG. 16C shows thrombin generation in FPH and apheresis platelets. Fresh platelets and FPH washed and treated as in I) were each incubated with 1000 nMFXa-APC, for 90 minutes, then measured for binding by flow cytometry MFI.

FIG. 16D shows flow cytometry MFI values for FXIII in a control sample (WPS or FPH), activated WPS or FPH, and activated WPS or FPH.

FIG. 17 shows flow cytometry MFI values for PAI1 in FPH, and apheresis platelets.

FIG. 18A shows the optical density versus time of the clot for both APU and FPH.

FIG. 18B shows the shows the clot lysis time for APU and FPH.

FIG. 19A shows the T-TAS signal curves for FPH and APU at 8×10⁴ cells/μl.

FIG. 19B shows the area under the curve (AUC) measurements for APU and FPH at 8×10⁴ cells/μl and 2×10⁴ cells/μl.

FIG. 20 shows the T-TAS signal curves for FPH tested at 8×10⁴ cells/μl with 0, 100, and 200 μg/mL of lactadherin pretreatment.

FIG. 21 shows the percentage recovery of FPH and apheresis platelets across a timepoint.

FIG. 22 shows the time for cessation of bleeding for the saline, APU, and different dose of FPH.

FIG. 23A shows a reduced lag time of thrombin production with the addition of freeze-dried platelet derivatives (FDPDs) to the platelet-rich plasma (PRP) obtained from the blood of an Hermansky Pudlak Syndrome (HPS) patient.

FIG. 23B shows a reduced time of peak of thrombin production with the addition of FDPDs to the PRP obtained from the blood of an HPS patient.

FIG. 24 shows that the addition of FDPDs to HPS patient blood restores clot amplitude to near normal amplitude in an ex vivo experiment.

FIG. 25A shows an improvement of clot amplitude in HPS patients with the addition of FDPDs to the blood of HPS patient in comparison to the addition of apheresis platelets (APU) in an ex vivo experiment.

FIG. 25B shows increased reduction of k time with the addition of FDPDs to the blood of HPS patient in comparison to the addition of apheresis platelets (APU) in an ex vivo experiment.

FIG. 26A shows that the addition of FDPDs to the blood of HPS patients (HPS-8, HPS-9, and HPS-11) restores the percentage of positivity of endogenous platelets that are PAC-1 positive back to normal levels in an ex vivo experiment.

FIG. 26B shows the data from FIG. 26A as the mean and standard error of the mean of the blood of HPS patients' data.

FIG. 26C shows the mean fluorescent intensity (MFI) representation of the data from FIG. 26B.

FIG. 27A shows that the addition of FDPDs to the blood of HPS patients (HPS-8, HPS-9, and HPS-11) restores the percentage of positivity of endogenous platelets that are CD62P back to normal levels in an ex vivo experiment.

FIG. 27B shows the data of FIG. 27A as the mean and standard error of the mean of the HPS patients' data.

FIG. 27C shows the MFI representation of the same data as FIG. 27B.

FIG. 28A shows that the addition of FDPDs to HPS blood increases the number of cells that are positive for CD63 in an ex vivo experiment.

FIG. 28B shows the MFI representation of the same data as FIG. 28A.

FIG. 29 shows the detection of the level of anti-platelets IgG antibodies in different serums.

FIG. 30 shows that addition of FDPDs to PRP from HPS patients improves the time to occlude.

FIG. 31A shows the histogram of a whole blood sample without FPH.

FIG. 31B shows the scattergram of a whole blood sample without FPH.

FIG. 31C shows the histogram of a whole blood sample with added FPH.

FIG. 31D shows the scattergram of a whole blood sample with added FPH.

FIG. 32A shows the linearity of FPH counts across the entire range of concentrations of FPH in the whole blood.

FIG. 32B shows the scattergram of a whole blood sample with the addition of FPH.

FIG. 33 shows that the FPH counts remain accurate with a delayed fixation.

FIG. 34A shows the histogram representation of a blood sample from a non-human primate at a pre-infusion time (pre-FPH infusion).

FIG. 34B shows the scattergram representation of a blood sample from a non-human primate at a pre-infusion time (pre-FPH infusion).

FIG. 34C shows the histogram representation of a blood sample from a non-human primate at an end-of-infusion time (EOI) (EOI of FPH).

FIG. 34D shows the scattergram representation of a blood sample from a non-human primate at an end-of-infusion time (EOI) (EOI of FPH).

FIG. 35 shows the measured FPH counts in samples from the non-human primate blood samples.

### DEFINITIONS

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

As used herein, freeze-dried platelet derivative hemostat or freeze-dried platelet-derived hemostat (“FPH”), which can also be referred to as freeze-dried platelet derivative hemostatics, freeze-dried platelet derivative hemostatic agents, freeze-dried platelet hemostatics, and freeze-dried platelet hemostatic agents. is a composition comprising freeze-dried platelet derivatives, that has hemostatic properties. Such hemostatic properties include the ability to generate thrombin in an in vitro assay and/or the ability to occlude a collagen-coated microchannel in an in vitro assay. Detailed conditions for performing such in vitro assays are provided herein.

As used herein, “hemostatic properties” include the following properties: (a) the ability to generate thrombin in a thrombin formation assay, for example in the presence of tissue factor and phospholipids; (b) the ability to occlude a collagen-coated microchannel in vitro, for example under conditions in which fresh platelets can occlude a collagen-coated microchannel in vitro; (c) the capability of thrombin-induced trapping in the presence of thrombin. As demonstrated in Examples herein, platelet derivatives, such as a freeze-dried platelet derivatives (e.g., thrombosomes) herein, in illustrative embodiments are hemostats, and thus have one, two, or all of the aforementioned hemostatic properties.

As used herein, “Platelets” may include, for example, platelets in whole blood, platelets in plasma, platelets in buffer optionally supplemented with select plasma proteins, cold stored platelets. Platelets may be from a mammal(s), such as of humans, or such as non-human mammals.

As used herein, “thrombosomes” (sometimes also called Tsomes) or “thrombosomes platelet derivatives” are platelet derivatives that have been treated with a preparation agent (e.g., any of the preparation agents described herein) and lyopreserved (typically freeze-dried). Thus, thrombosomes are typically freeze-dried platelet derivatives (FDPDs). Furthermore, thrombosomes are FDPDs that have hemostatic properties, and thus can be referred to as FPH. In some cases, thrombosomes platelet derivatives can be prepared from pooled platelets. Thrombosomes platelet derivatives can have a shelf life of 2-3 years in dry form at ambient temperature and can be rehydrated with sterile water within minutes for immediate infusion. One example of thrombosomes freeze-dried platelet derivatives are THROMBOSOMES® freeze-dried platelet derivatives (Cellphire Inc., Rockville, MD), which are in clinical trials. In non-limiting illustrative embodiments, thrombosome compositions, or illustrative freeze-dried platelet-derivative compositions herein, such as those prepared according to Example 1 of U.S. Pat. No. 11,529,587 B2, incorporated by reference herein in its entirety, and Example 1 of PCT app no. PCT/US2022/079280, incorporated by reference herein in its entirety, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein in illustrative embodiments the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, at least 80%, 85%, 90% or 95% of the platelet derivatives are at least 0.5 μm in diameter, and in some embodiments at least 80%, 85%, 90% or 95% of the platelet derivatives are 0.5 to 2.5 μm in diameter.

It is to be understood that the terminology used herein is for the purpose of describing particular aspects and embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication. Any Headings herein are for convenience only and are not intended to be limiting, and it will be understood that the disclosure including aspects and embodiments provided within one section herein can be combined with the disclosure includes aspects and embodiments provided within any other section herein.

### DETAILED DESCRIPTION

To overcome the above-mentioned and additional problems in the art, the present disclosure provides, at least in part, blood products, such as, freeze-dried platelets, freeze-dried platelet derivatives (FDPDs), HLA-characterized FDPDs, FPH, or HLA-characterized FPH for use in controlling bleeding in a subject, that relate at least in part, to the surprising finding disclosed in the Examples section herein that platelet derivatives, FDPDs, or FPH provided herein have a short, less than 4 minute, circulation half-life, i.e. have low circulation persistence in animal models, for example, NOD-SCID mice, and cynomolgus macaques. Not to be limited by theory, such relatively short circulation half-life is believed to provide the advantage that FDPDs provided herein, have sufficient time to reach a site(s) of bleeding, and less time to reach off target sites.

Accordingly, provided herein is a composition comprising an effective dose of platelet derivatives for use for controlling bleeding in a subject, or a method for controlling bleeding in a subject, by administrating multiple doses of platelet derivatives within 5, 10, 15, 20, 30, 45, minutes, 1, 2, 3, 4, 5, or 6 hours until bleeding is reduced, stopped, or otherwise a beneficial effect is observed. Furthermore, provided herein is a composition comprising an effective dose of platelet derivatives for use for controlling bleeding in a subject, or provided herein is a method for controlling bleeding in a subject, comprising administering multiple times, in illustrative embodiments each dose of at least 1×10⁸/kg, or 1×10⁹/kg of the subject, by administering 2, 3, 4, 5 or more doses of platelet derivatives every 15 minutes, 30 minutes, 45 minutes, 1 hour, or more frequently until bleeding of the subject is reduced.

In some aspects, and some embodiments of the above aspects related to multiple dosing, compositions and methods provided herein, are used to control bleeding and/or increase platelet biomarkers in a subject having a platelet dysfunction or a platelet disorder such as, for example, Hermansky Pudlak Syndrome (HPS). Accordingly, provided herein is a method for administering freeze-dried platelet derivatives (FDPDs) to a subject having HPS, comprising administering an effective dose of the freeze-dried platelet derivatives in a platelet derivative composition to the subject. The FDPDs can have numerous characteristics provided herein, that make them well suited to restore hemostatic functions in a recipient.

In some embodiments, the administering increases the levels of at least one platelet biomarker selected from CD62P, PAC-1, and CD63 for endogenous platelets of the subject (recipient) as compared to before the administering. In illustrative embodiments, levels of both CD62P and PAC-1 are increased in the subject after the administering.

### Platelet Derivatives for Controlling Bleeding

In some aspects, a platelet derivative composition, in illustrative embodiment a freeze-dried platelet derivative composition, FDPDs or HLA-characterized FDPDs, FPH or HLA-characterized FPH, including, but not limited to, those of any of the aspects or embodiments herein, or the platelet derivative composition prepared by any of the processes disclosed herein, can be administered, or delivered to a subject to control bleeding. Thus, such administering can be performed by administering an effective dose of the platelet derivative composition. Such effective dose in illustrative embodiments, includes multiple individual doses, or a continuous dose. In certain aspects herein the subject is a subject having Hermansky Pudlak Syndrome (HPS). In illustrative embodiments, the subject has an indication and thus is afflicted with a disorder or disease that could benefit from delivery of such platelet derivative compositions, during treatment for such disorder or disease, during a surgical procedure, or during a transplantation procedure. In some embodiments, platelet derivatives as disclosed herein have short circulation half-life, for example, less than 20 minutes, 15 minutes, 10 minutes, 8 minutes, 6 minutes, 5 minutes, 4 minutes, or 3 minutes, when administered to a subject, such as a mammal, in illustrative embodiments, a human. Therefore, in order to have a desirable effect of the platelet derivatives in the subject, methods herein can comprise administering more than 1, 2, 3, 4, 5, or more doses or therapeutically effective doses of the platelet derivatives herein to the subject in a given span of time. It will be understood that in order to maintain a certain population of platelet derivatives in the subject for observing the beneficial effect of the platelet derivatives in spite of short half-life circulation, methods herein can include administering multiple doses of the platelet derivatives to the subject in a span of time or at a frequency of time until bleeding is reduced, stopped, or a beneficial effect is observed. In some embodiments, a method herein can include administering the platelet derivatives to the subject as a continuous infusion over a span of time until bleeding is reduced, stopped, or a beneficial effect is observed. Such span of time for coninuous infusion can include, in some embodiments, a span of time equal to any time frame provided herein for multiple doses. In some embodiments, continuous infusion is performed for 5, 10, 15, 30, or 45 minutes, or for 1, 2, 3, 4, 6, 8, 12, or 18 hours, or for 1, 2, 3, 4, 5, 6, or 7 days. In some cases, administering can be performed by administering the platelet derivatives as a continuous infusion and also administering more than one dose the platelet derivatives in an intermittent regime until bleeding or bleeding potential of the subject is reduced, stopped, or otherwise any beneficial effects of administering are observed.

A person of skill in the art can contemplate treating a subject, such as, controlling bleeding, reducing bleeding potential of the subject, or addressing a disorder or a condition occurred by any of the indications disclosed herein using platelet derivatives as described herein as a medicament in several doses in a span of time for treating the subject. Alternatively, or in combination, in some embodiments, administering of platelet derivatives as described herein can be performed as a continuous infusion procedure. In some embodiments, administering of platelet derivatives, FDPDs, HLA-characterized FDPDs, HLA compatible FDPDs, FPH, HLA-Characterized FPH, or HLA compatible FPH, as disclosed herein, is performed for at least, or at a maximum of 2, 3, 4, 5, 6, 7, 8, 9, or 10 doses in a 4, 8, 12, 24, 48 or 72-hour period of treatment. For example, a specific dose of platelet derivatives can be decided as per the requirement of a subject, for example based on the weight of the subject, and the specific dose can be provided to the subject as a continuous infusion procedure with or without an interval between continuous infusion doses. In some embodiments, administering can be performed as a continuous infusion procedure until the bleeding and/or bleeding potential of the subject is reduced as compared to the bleeding or bleeding potential before the administering. In some embodiments, administering can be performed as a continuous infusion until the bleeding in the subject is stopped. In some embodiments, administering of platelet derivatives as described herein can be performed at regular intervals. For example, a single, double, or more doses of platelet derivatives as described herein and as per the requirement of a subject, can be administered to a subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours until a desired outcome, for example reduced or termination of bleeding, is achieved. For example, between 2 in the lower end and 7, 8, 10, 12, 15, 20 doses in the higher end, or between 3 in the lower end and 7, 8, 10, 12, 15, 20 doses in the higher end of platelet derivatives, can be administered to the recipient subject within 1 hour, 30 minutes, 20 minutes, 15 minutes, 10 minutes or 5 minutes, and such administration can be guided by the effect of the dosing on bleeding of the subject, for example at one or more specific sites of bleeding. For example, the dosing can be performed until the bleeding at one or more sites decreases and/or stops. Such bleeding determination can be made, for example, by visual inspection. The decrease can be for example, a decrease that is observable by visual inspection, a decrease that is detected by a measurement, a decrease that is no longer considered life threatening, and/or a decrease such that the bleeding is considered minor. In some embodiments, total number of doses administered to the subject can be in the range of 2-100, 2-80, 2-60, 2-50, 2-40, 2-30, 2-20, 2-15, 2-10, 2-8, 3-100, 3-80, 3-60, 3-50, 3-40, 3-30, 3-20, 3-15, 3-10, 3-8, 4-100, 4-80, 4-60, 4-50, 4-40, 4-30, 4-20, 4-15, 4-10, 4-8, 5-20, 5-15, 5-10, 5-8, 10-100, 20-100, 30-100, 40-100, 50-100, 60-100, 70-100, 80-100, 10-90, 10-80, 10-70, 10-60, 10-50, 10-40, 10-30, 10-20, or 10-15, in illustrative embodiments, within a given span of time as disclosed herein, for example, in a span of time from administering a first dose of platelet derivatives until the bleeding is controlled, stopped, bleeding potential is reduced, or any other beneficial parameter according to a therapeutic indication of the subject. In some embodiments, each of the doses administered according to any of the embodiments or aspects herein can have the same dosage of platelet derivatives/kg of the subject, or can have various dosage of platelet derivatives/kg of the subject, such that one particular dose (platelet derivatives/kg) can be different than the other doses succeeding or preceding number the particular dose. In some embodiments, the dosages in any number of doses being administered to a subject herein can include any therapeutic effective dosage (platelet derivatives/kg) as disclosed herein. In some embodiments, the specific dose for any dose of a multi-dose regimen can be influenced, guided, and/or changed, based on the outcome that is detected or observed, in illustrative embodiments, a bleeding related outcome. Thus, for example if a first dose is administered to a patient and no change in bleeding is observed, one or more additional doses can be administered within any of the timeframes provided herein, that is higher than the first dose. Furthermore, in some embodiments the desired outcome is to maintain the reduced or cessation of bleeding, when prior doses of platelet derivatives (e.g., FDPDs) successfully reduced or stopped the bleeding.

In some embodiments, the doses can be administered at a regular interval for 36 hours, 48 hours, or 72 hours from the start of the first dose. In some embodiments, administering of platelet derivatives as described herein can be performed as a mixed procedure in which the continuous infusion can be interrupted with a specific dose of platelet derivatives followed by a specific interval as per the requirement. In some embodiments, administering of platelet derivatives as described herein can be performed in a maximum of 2, 3, 4, 5, 6, 7, 8, 9, or 10 doses in a 24-hour period. In some embodiments, administering of platelet derivatives as described herein is performed in a maximum of 2, 3, 4, 5, 6, 7, 8, 9, or 10 doses in a 72-hour period of treatment. In some embodiments, methods herein comprise administering 2-50, 2-45, 2-40, 2-35, 2-30, 2-25, 2-20, 2-15, 2-10, 2-8, 2-6, 5-50, 10-50, 15-50, 20-50, 25-50, 3-15, 3-12, 3-10, 3-9, 3-8, 4-25, 4-20, 4-18, 4-15, 5-25, 5-20, 5-15, or 5-10 doses of platelet derivatives to a subject within 1, 2, 3, 4, or 8 hours, for example, after administering a first dose of platelet derivatives. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-25, 4-20, 4-18, 4-15, 4-12, 4-10, 4-8, 5-25, 5-20, 5-15, 5-10, or 5-8 doses of platelet derivatives to a subject within 2 hours. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-15, 4-12, 4-10, 4-8, 5-25, 5-20, 5-15, or 5-10 doses of platelet derivatives to a subject within 5 hours. In some embodiments, methods herein comprise administering 2-50, 2-45, 2-40, 2-35, 2-30, 2-25, 2-20, 2-15, 2-10, 2-8, 2-6, 3-15, 3-12, 3-10, 3-9, 3-8, 4-25, 4-20, 4-18, 4-15, 4-12, 4-10, 4-8, 5-25, 5-20, 5-15, 5-10, or 5-8 doses of platelet derivatives to a subject within 12 hours. In some embodiments, methods herein comprise administering 2-80, 2-60, 2-50, 2-45, 2-40, 2-35, 2-30, 2-25, 2-20, 2-15, 2-10, 2-8, 3-50, 3-45, 3-30, 3-20, 3-15, 3-12, 3-9, 4-20, 4-15, 4-12, 4-10, 4-8, 5-50, 5-45, 5-40, 5-35, 5-30, 5-25, 5-20, 5-15, 5-10, 15-80, 15-75, 15-70, 15-60, 15-45, 15-40, 15-35, 15-30, 15-25, or 15-20 doses of platelet derivatives to a subject within 24 hours.

In some situations that benefit from controlling of bleeding in a subject in a short span of time, for example, during a surgery or a transplantation procedure, methods herein can include administering more than 2, 4, or 10 doses of platelet derivatives, for example 2-20, 2-15, 2-12, 2-10, 2-8, 3-20, 3-18, 3-15, 3-12, 3-10, 4-20, 4-15, 4-10, 5-20, 5-15, or 5-10 doses every 2, 5, 10, 15, 20, 25, 30, 45 minutes, or more frequently, at the same or at variable frequencies within a timeframe until bleeding is controlled or reduced, bleeding potential is reduced, beneficial effect is observed in a subject, or until the surgery is completed. Thus, it will be understood that a frequency of a certain number of doses within a time period or more frequency, can be dosing at the same frequency or at a variable frequency within the given timeframe. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 5 minutes, or more frequently, in illustrative embodiments, every 5 minutes until bleeding is controlled or reduced, bleeding potential is reduced, beneficial effect is observed in a subject, or until the surgery is completed. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 10 minutes, or more frequently, in illustrative embodiments, every 10 minutes until bleeding is controlled or reduced, bleeding potential is reduced, beneficial effect is observed in a subject, or until the surgery is completed. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 15-45 minutes until bleeding is controlled or reduced, bleeding potential is reduced, beneficial effect is observed in a subject, or until the surgery is completed. For example, administering can include 2-20 doses every 15-20 minutes, 15-30 minutes, or 15-45 minutes from the time of a first dose. In some embodiments, methods herein include administering 2-50, 2-45, 2-40, 2-35, 2-30, 2-25, 2-20, 2-15, 2-10, 2-8, 2-6, 3-20, 3-18, 3-15, 3-12, 3-10, 4-20, 4-15, 4-10, 5-50, 5-45, 5-40, 5-35, 5-30, 5-25, 5-20, 5-15, or 5-10 doses every 15 minutes to 1 hour until bleeding is controlled or reduced, bleeding potential is reduced, beneficial effect is observed in a subject, or until the surgery is completed. For example, administering can include 2-20 doses every 15-20 minutes, 15-30 minutes, 15-45 minutes, 20 minutes-1 hour, 30 minutes-1 hour, or 45 minutes-1 hour from the time of a first dose.

In some embodiments, methods herein include administering, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 2 minutes, for example, from the time of a first dose, until bleeding is controlled or reduced, bleeding potential is reduced, beneficial effect is observed in a subject, or until the surgery is completed. In some embodiments, methods herein include administering multiple times 2-20, 2-18, 2-15, 2-12, 2-10, 2-8, 2-6, 2-4, 3-20, 3-18, 3-15, 3-12, 3-9, 4-20, 4-15, 4-12, 4-10, 4-8, 5-20, 5-15, or 5-10 doses of platelet derivatives to a subject within 5 minutes, for example, from the time of a first dose until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 10 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 15 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 30 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 45 minutes until bleeding is controlled or reduced, bleeding potential is reduced, beneficial effect is observed in a subject, or until the surgery is completed. In some embodiments, methods herein include administering, in illustrative embodiments, multiple times 2-20, 2-18, 2-15, 2-12, 2-10, 2-8, 2-6, 2-4, 3-20, 3-18, 3-15, 3-12, 3-9, 5-20, 5-15, or 5-10 doses of platelet derivatives to a subject within 1 hour until bleeding is controlled or reduced, bleeding potential is reduced, beneficial effect is observed in a subject, or until the surgery is completed. In some embodiments, methods herein include administering, in illustrative embodiments, multiple times 2-50, 2-45, 2-40, 2-35, 2-30, 2-25, 2-20, 2-15, 2-10, 2-8, 2-6, 5-50, 5-45, 5-40, 5-35, 5-30, 5-25, 5-20, 5-15, or 5-10 doses of platelet derivatives to a subject within 20 minutes to 45 minutes until bleeding is controlled or reduced, bleeding potential is reduced, beneficial effect is observed in a subject, or until the surgery is completed. A skilled artisan can understand that administering multiple times includes administering more than one time, for example, 2 times, 3 times, 4 times, 5 times, 6 times or more doses as disclosed herein. For example, administering multiple times includes 2-10 times, 2-9 times, 2-8 times, 2-6 times, or 2-4 times as disclosed herein. In some embodiments, administering of platelet derivatives can be performed at a frequency of at least one dose every 15 minutes or more frequently. For example, administering can be performed at a frequency of at least one dose every 15 minutes or more frequently starting from the first dose until the bleeding potential of the subject is reduced as compared to the bleeding potential before the administering. In some embodiments, the administering of any number of doses as disclosed herein can be performed until the bleeding stops. In some embodiments, the administering of any number of doses as disclosed herein can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently. It will be understood that the starting point for measuring dosing intervals between two doses is the time point at the moment after the entire first dose of the two doses is administered to the subject. Since it may take some time to administer each dose to a subject, a time-frame can be calculated after the end of infusion or other administration route, of a first dose or a previous dose. In some embodiments, administering each dose of platelet derivatives, FDPDs, FPH, or HLA-characterized FPH can take at least 30 seconds, 1 minute, 1.5 minutes, 2 minutes, or 3 minutes. In some embodiments, administering each dose can take 10 seconds to 10 minutes, 10 seconds to 5 minutes, 10 seconds to 3 minutes, 20 seconds to 3 minutes, 30 seconds to 3 minutes, or 1 minute to 3 minutes. Further, in some embodiments, the subject involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion defined as two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

In some embodiments of any of the aspects or embodiments herein that include a composition for use in controlling bleeding, or a method for controlling bleeding, a dose (which can be referred to herein as a single dose or an individual dose) can be administered to a subject from one vial or by combining the contents of more than one vial each containing platelet derivatives, FDPDs or HLA-characterized FDPDs, FPH or HLA-characterized FPH. In some embodiments, a single dose can be administered to a subject by combining the contents of 2 to 6, 2 to 5, or 2 to 4 vials, each vial containing platelet derivatives, FDPDs or HLA-characterized FDPDs, FPH or HLA-characterized FPH. In some embodiments, a single dose, in illustrative embodiments of at least 1×10⁷/kg, 1×10⁸/kg, 1×10⁹/kg, 1.2×10⁹/kg, 1.4×10⁹/kg, or 1.6×10⁹/kg, can be administered to a subject by combining the contents of 2 vials, 3 vials, or 4 vials, each containing platelet derivatives, FDPDs or HLA-characterized FDPDs, FPH or HLA-characterized FPH. In some embodiments, each vial containing platelet derivatives, FDPDs or HLA-characterized FDPDs, FPH or HLA-characterized FPH can have a volume in the range of 5-100 ml, 10-90 ml, 25-75 ml, or 5-40 ml, in illustrative embodiments, have a volume of 20 ml, 25 ml, 30 ml, 35 ml, or 40 ml.

A person of skill in the art can contemplate the effective dose of platelet derivatives that can be effective to treat a subject in need thereof. The need may differ based on the condition of the subject. The effective dosage can be categorized into a) low dosage; b) medium dosage; and c) high dosage. In some embodiments, a medicament or a method of treating a subject can have the effective dose as low, medium, or high dosage of platelet derivatives that can broadly range from 1.0×10⁷ on the low end of the range to 1.0×10¹⁰/kg, 1.0×10¹¹/kg or 1.0×10¹²/kg of the subject on the high end of the range. In some embodiments, a dose, an effective dose, or a therapeutically effective dose of platelet derivatives, FDPDs, FPH, as disclosed herein can be administered to a subject or a recipient as a part of a surgical procedure. In other words, platelet derivatives, FDPDs, or FPH can be provided to a subject or a recipient during a surgery, before a surgery, or as a follow-up after the surgery. For example, any dose of platelet derivatives, FDPDs, or FPH as disclosed herein can be administered during a surgery, for example, due to an increased risk of bleeding, or if bleeding, or unusually heavy bleeding is observed.

In some embodiments of any of the aspects or embodiments herein that include a composition for use for controlling bleeding of a subject, a method for controlling bleeding in a subject, or administering platelet derivatives to a subject herein, a dose or single dose of platelet derivatives, in illustrative embodiments for multiple administration can be in the range of 1.0×10⁷ to 1.0×10¹²/kg, 1.0×10⁸ to 1.0×10¹²/kg, 1.0×10⁹ to 1.0×10¹²/kg, 1.0×10⁷ to 1.0×10¹¹/kg, 1.0×10⁷ to 1.0×10¹⁰/kg, 1.0×10⁸ to 1.0×10¹¹/kg, or 1.0×10⁹ to 1.0×10¹¹/kg of the subject. In illustrative embodiments, a dose or a single dose of platelet derivatives can be in the range of 1.0×10⁹ to 1.0×10¹¹/kg, 1.2×10⁹ to 1.0×10¹¹/kg, 1.4×10⁹ to 1.0×10¹¹/kg, 1.0×10⁹ to 8.0×10¹⁰/kg, 1.0×10⁹ to 6.0×10¹⁰/kg, 1.0×10⁹ to 5.0×10¹⁰/kg, 1.0×10⁹ to 4.0×10¹⁰/kg, 1.0×10⁹ to 3.0×10¹⁰/kg, or 1.0×10⁹ to 2.0×10¹⁰/kg. In some embodiments, an effective dose can depend on the number of times a dose herein is to be administered to the subject until the bleeding is reduced, stopped, or otherwise a beneficial outcome is reached. For example, a dose or a single dose can be in the range of 1.0×10⁸ to 1.0×10¹²/kg. However, such single dose may not be effective at reducing or stopping bleeding. Thus, a second dose in such non-limiting example, can be administered and if bleeding is reduced or stopped, depending on the desired or recited outcome, then the combined two doses provide the effective dose. As such, the effective dose in this non-limiting example, is in the range of 2.0×10⁸ to 2.0×10¹²/kg. And such two doses provide a total dose in the range of 2.0×10⁸ to 2.0×10¹²/kg. In another non-limiting example, if 10 doses are administered to reduce bleeding, and that was the desired or recited outcome, and a dose or a single dose administered is in the range of 1.0×10⁹ to 1.0×10¹²/kg, then the effective dose is in the range of 1.0×10⁹ to 1.0×10¹³/kg of the subject. And the total dose, regardless of whether it was therapeutically effective, is in the range of 1.0×10⁹ to 1.0×10¹³/kg of the subject. Accordingly, in some embodiments, a total dose and/or an effective dose administered to a subject is in the range of 2.0×10⁷ to 1.0×10¹³/kg, 1.0×10⁸ to 1.0×10¹³/kg, 1.0×10⁹ to 1.0×10¹³/kg, 2.0×10⁷ to 1.0×10¹¹/kg, 2.0×10⁷ to 1.0×10¹⁰/kg, 1.0×10⁸ to 1.0×10¹¹/kg, or 1.0×10⁹ to 1.0×10¹¹/kg of the subject.

In some embodiments, 2-20, 3-20, 4-20, 5-20 doses of platelet derivatives can be administered to a subject within 15 minutes, 30 minutes, 45 minutes, 1 hour, 2 hours, 3 hours, 4 hours, 5 hours, or 6 hours, or more frequently wherein each dose can be of at least 1×10⁸/kg, 5×10⁸/kg, 1×10⁹ kg, 1.2×10⁹/kg, 1.4×10⁹/kg, or 1.6×10⁹/kg of the subject. In some embodiments, administering can be done by administering at least a single dose of at least 1×10⁸/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least a single dose of at least 5×10⁸/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least a single dose of at least 1×10⁹/kg, in illustrative embodiments, at least 1.5×10⁹/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can include administering 3, 4, 5, 6 or more doses, for example, each dose of at least 1×10⁸/kg of the subject within 15 minutes, 30 minutes, 45 minutes, 1 hours, 2, 3, 4, 5, or 6 hours, or more frequently. In some embodiments, administering can be done by administering at least 2 doses of 1×10⁸/kg to 1×10¹⁰/kg of the subject multiple times at a frequency of every 2 minutes to 15 minutes, 10 minutes to 30 minutes, 30 minutes to 45 minutes, 15 minutes to 45 minutes, or 45 minutes to 1 hour starting from a first dose. In some embodiments, administering can be done by administering at least 2 doses of 1×10⁸/kg to 1×10¹⁰/kg of the subject multiple times at a frequency of every 5 minutes, 15 minutes, 30 minutes, 45 minutes, 1 hour, 2, 3, 4, 5, 6 hours, or more frequently starting from a first dose, in illustrative embodiments, a total number of doses administered can be in the range of 2-30, 2-25, 2-20, 2-15, 2-10, 2-8, 3-30, 3-25, 3-20, 2-18, 3-15, 3-12, 3-9, 4-30, 4-25, 4-20, 4-15, 4-12, 4-8, 5-30, 5-25, 5-20, 5-15, or 5-10 within a period of 24 hours from a first dose. It will be understood a total dose and/or effective dose administered in aspects herein, can be any range that equals a range of doses and amounts per dose, provided herein.

In some embodiments of any of embodiments or aspects herein, that include administering platelet derivatives, FDPDs, FPH, or HLA-characterized FPH to a subject, administering comprises topical administration, parenteral administration, or a combination of a topical and parenteral administration. Administration via parenteral route can comprise intravenous (IV), intraperitoneal (IP), subcutaneous (SC), intramuscular (IM), or intradermal (ID). In some embodiments, parenteral administration comprises intravenous administration. In some embodiments, parenteral administration comprises subcutaneous, intraperitoneal, intramuscular, or intradermal. Topical administration route can comprise a topical application of platelet derivatives, FDPD, FPH, or HLA-characterized FPH directly at the site of bleeding. In some embodiments, platelet derivatives, FDPD, FPH, or HLA-characterized FPH can be prepared in various forms including, but not limited to, particulate, powder, solution, gel, and matrix. In some embodiments, administering herein can comprise a combination of parenteral administration, in illustrative embodiments, intravenous administration, and topical administration, in illustrative embodiments, administering topically at a site of bleeding of the subject. In some cases, the site of the parenteral administration can vary depending upon the type and extent of bleeding of the subject. For example, parenteral administration can be done near the site of bleeding. In some cases, during the surgery, or after surgery, parenteral administration can be done near the site of incision to control the bleeding. In some embodiments, administering comprises a combination of topical and parenteral administration, in illustrative embodiments, intravenous administration at the site of injury, incision, or otherwise bleeding. For example, in some cases, administering can be done in a manner such that topical administration and the parenteral administration, in illustrative embodiments, intravenous administration is alternately done. The dose ranges of platelet derivatives, FDPD, FPH, or HLA-characterized FPH for topical administration can be any of the ranges as disclosed herein. In some embodiments, the doses of parenteral administration can be the same or different as compared to the doses of topical administration at the site of injury, incision, or otherwise bleeding.

In some embodiments, a platelet derivative composition as described herein can be administered or delivered to a subject, such as a subject afflicted with any one or combination of indications as described herein, and the dose of a platelet derivative composition can be in the range of 1.0×10⁷ to 1.0×10¹² particles/kg of the subject. For example, in some embodiments, a dose, or a single dose of a composition comprising platelets, platelet derivatives (e.g., FDPDs), HLA-characterized FDPDs, FPH or HLA-characterized FPH can include between about or exactly 1.0×10⁷ on the low end of the range to 1.0×10¹² particles (e.g. FDPDs)/kg of a subject on the high end of the range, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹¹ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, 5.0×10⁷ to 1.0×10¹¹ particles (e.g. FDPDs)/kg of a subject, 5.0×10⁹ to 1.0×10¹⁰ particles (e.g. FDPDs)/kg of a subject), or 1.0×10¹⁰ to 1.0×10¹¹ or 1.0×10¹² particles (e.g. FDPDs)/kg of a subject on the high end of the range. In some embodiments, the dose can be in the range of 250 and 5000 TGPU per kg of the subject.

In some embodiments, a platelet derivative composition, such as that provided in any aspect or embodiment herein, can be administered or delivered to a subject afflicted with any one or combination of indications/diseases as disclosed herein, and the dose, or a single dose of a platelet derivative composition comprising FDPDs, HLA-characterized FDPDs, or FPH or HLA-characterized FPH can be in the range of 1.0×10⁷ on the low end of the range to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁷ to 5.0×10⁹ particles (e.g. FDPDs)/kg of a subject, or 5.0×10⁹ to 1.0×10¹⁰ particles (e.g. FDPDs)/kg of a subject). In some embodiments, a dose of a composition comprising platelets or platelet derivatives (e.g., FDPDs) can be a range of between about or exactly 1.0×10⁸, 5.0×10⁸, 1.0×10⁹, 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 5.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1.5×10⁹, 1.6×10⁹, 1.7×10⁹, 1.8×10⁹, 1.9×10⁹, 2.0×10⁹, 3.0×10⁹, 4.0×10⁹, or 5.0×10⁹ on the low end of the range to 1.0×10¹⁰ particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, or 1.2×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end of the range.

In some embodiments of any aspect or embodiment herein a therapeutically effective dose or effective dose or amount of the platelet derivatives in a platelet derivative composition is in the range of 1.5×10⁷ to 5.0×10¹⁰/kg, 2.0×10⁷ to 1.0×10¹⁰/kg, 2.5×10⁷ to 5.0×10⁹/kg, 2.75×10⁷ to 3.0×10⁹/kg, 2.8×10⁷ to 4.0×10⁹/kg, 3.0×10⁷ to 4.0×10⁹/kg, 5×10⁷ to 4.0×10⁹/kg, 6×10⁷ to 3.0×10⁹/kg, 9×10⁷ to 3.0×10⁹/kg, 1.0×10⁸ to 2.0×10⁹/kg, or 1.3×10⁸ to 1.8×10⁹/kg of the subject. In some embodiments, a therapeutically effective dose or effective dose or amount of the platelet derivatives in a platelet derivative composition is in the range of 5.0×10⁷ to 1.0×10⁹/kg, 1.0×10⁸ to 5.0×10⁸/kg, 1.2×10⁸ to 2.5×10⁸/kg, 1.6×10⁸ to 2.2×10⁸/kg, or 1.7×10⁸ to 2.0×10⁸/kg of the subject. In some embodiments, the platelet derivatives in a platelet derivative composition is 1.1×10⁸/kg, 1.2×10⁸/kg, 1.3×10⁸/kg, 1.4×10⁸/kg, 1.5×10⁸/kg, 1.6×10⁸/kg, 1.7×10⁸/kg, 1.8×10⁸/kg, 1.9×10⁸/kg, 2.0×10⁸/kg, 2.1×10⁸/kg, 2.2×10⁸/kg. 2.3×10⁸/kg, 2.4×10⁸/kg, or 2.5×10⁸/kg of the subject.

In some embodiments of any aspect or embodiment herein a therapeutically effective dose or effective dose or amount of the platelet derivatives in a platelet derivative composition is in the range of 5.1×10⁸ to 9.9×10⁸/kg, 5.5×10⁸ to 9.5×10⁸/kg, 5.8×10⁸ to 9.3×10⁸/kg, 6.1×10⁸ to 9.0×10⁸/kg, 6.5×10⁸ to 8.8×10⁸/kg, 6.8×10⁸ to 8.5×10⁸/kg, 7.0×10⁸ to 8.4×10⁸/kg, 7.5×10⁸ to 8.3×10⁸/kg, 7.8×10⁸ to 8.2×10⁸/kg, or 7.9×10⁸ to 8.1×10⁸/kg of the subject. In some embodiments, a therapeutically effective dose or effective dose or amount of the platelet derivatives in a platelet derivative composition is 7.5×10⁸/kg, 7.6×10⁸/kg, 7.7×10⁸/kg, 7.8×10⁸/kg, 7.9×10⁸/kg, 8.0×10⁸/kg, 8.1×10⁸/kg, 8.2×10⁸/kg, 8.3×10⁸/kg, 8.4×10⁸/kg, or 8.5×10⁸/kg of the subject.

In some embodiments of any aspect or embodiment herein a dose, a therapeutically effective dose, or effective dose or amount of the platelet derivatives in a platelet derivative composition is in the range of 1.0×10⁹ to 1.0×10¹⁰/kg, 1.1×10⁹ to 8.0×10⁹/kg, 1.2×10⁹ to 7.0×10⁹/kg, 1.2×10⁹ to 6.0×10⁹/kg, 1.2×10⁹ to 5.0×10⁹/kg, 1.3×10⁹ to 4.0×10⁹/kg, 1.3×10⁹ to 3.0×10⁹/kg, 1.3×10⁹ to 2.5×10⁹/kg, 1.4×10⁹ to 1.9×10⁹/kg, 1.50×10⁹ to 1.75×10⁹/kg, or 1.55×10⁹ to 1.70×10⁹/kg of the subject. In some embodiments, a dose, a therapeutically effective dose or effective dose or amount of the platelet derivatives in a platelet derivative composition is 1.1×10⁹/kg, 1.2×10⁹/kg, 1.3×10⁹/kg, 1.4×10⁹/kg 1.5×10⁹/kg, 1.55×10⁹/kg, 1.56×10⁹/kg, 1.57×10⁹/kg, 1.58×10⁹/kg, 1.59×10⁹/kg, 1.6×10⁹/kg, 1.61×10⁹/kg, 1.62×10⁹/kg, 1.63×10⁹/kg, 1.64×10⁹/kg, 1.65×10⁹/kg, 1.66×10⁹/kg, 1.7×10⁹/kg, 1.8×10⁹/kg, 1.9×10⁹/kg, or 2.0×10⁹/kg of the subject.

In some embodiments of any aspect, or embodiment herein a therapeutically effective dose or effective dose or amount of the platelet derivatives in a platelet derivative composition is in the range of 1.0×10⁷ to 1.0×10¹⁴ particles/kg of the subject, 1.6×10⁷ to 1.0×10¹⁴ particles (e.g. FDPDs/kg of subject, 1.6×10⁷ to 8×10¹³ particles (e.g. FDPDs/kg of a subject), 1.6×10⁷ to 5.1×10¹³ particles (e.g. FDPDs/kg of a subject), 1.6×10⁷ to 3.0×10¹³ particles (e.g. FDPDs)/kg of a subject, 1.6×10⁷ to 1.0×10¹³ particles (e.g. FDPDs)/kg of a subject, 1.6×10⁷ to 8.0×10¹² particles (e.g. FDPDs)/kg of a subject, 1.6×10⁷ to 5.0×10¹² particles (e.g. FDPDs)/kg of a subject, 1.6×10⁷ to 3.0×10¹² particles (e.g. FDPDs)/kg of a subject, 1.6×10⁷ to 1.0×10¹² particles (e.g. FDPDs)/kg of a subject, 1.6×10⁷ to 8.0×10¹¹ particles (e.g. FDPDs)/kg of a subject, 1.6×10⁷ to 5.0×10¹¹ particles (e.g. FDPDs)/kg of a subject, 1.6×10⁷ to 3.0×10¹¹ particles (e.g. FDPDs)/kg of a subject, 1.6×10⁷ to 1.0×10¹¹ particles (e.g. FDPDs)/kg of a subject, 1.6×10⁷ to 8.0×10¹⁰ particles (e.g. FDPDs)/kg of a subject, 1.6×10⁷ to 5.0×10¹⁰ particles (e.g. FDPDs)/kg of a subject, 1.6×10⁷ to 3.0×10¹⁰ particles (e.g. FDPDs)/kg of a subject, 1.6×10⁷ to 8.0×10¹⁰ particles (e.g. FDPDs)/kg of a subject, 1.6×10⁷ to 5.0×10¹⁰ particles (e.g. FDPDs)/kg of a subject, 1.6×10⁷ to 3.0×¹⁰1° particles (e.g. FDPDs)/kg of a subject, 1.6×10⁷ to 8.0×10¹⁹ particles (e.g. FDPDs)/kg of a subject, 5.0×10⁷ to 1.0×10¹⁴ particles (e.g. FDPDs)/kg of a subject, 8.0×10⁷ to 1.0×10¹⁴ particles (e.g. FDPDs)/kg of a subject, 1.0×10⁸ to 1.0×10¹⁴ particles (e.g. FDPDs)/kg of a subject, 3.0×10⁸ to 1.0×10¹⁴ particles (e.g. FDPDs)/kg of a subject, 5.0×10⁸ to 1.0×10¹⁴ particles (e.g. FDPDs)/kg of a subject), 8.0×10⁸ to 1.0×10¹⁴ particles (e.g. FDPDs)/kg of a subject), 1.0×10⁹ to 1.0×10¹⁴ particles (e.g. FDPDs)/kg of a subject), 3.0×10⁹ to 1.0×10¹⁴ particles (e.g. FDPDs)/kg of a subject), 5.0×10⁹ to 1.0×10¹⁴ particles (e.g. FDPDs)/kg of a subject), 8.0×10⁹ to 1.0×10¹⁴ particles (e.g. FDPDs)/kg of a subject), 1.0×10¹⁰ to 1.0×10¹⁴ particles (e.g. FDPDs)/kg of a subject), 3.0×10¹⁰ to 1.0×10¹⁴ particles (e.g. FDPDs)/kg of a subject), 5.0×10¹⁰ to 1.0×10¹⁴ particles (e.g. FDPDs)/kg of a subject), 8.0×10¹⁰ to 1.0×10¹⁴ particles (e.g. FDPDs)/kg of a subject), 1.0×10¹¹ to 1.0×10¹⁴ particles (e.g. FDPDs)/kg of a subject), 5.0×10¹¹ to 1.0×10¹⁴ particles (e.g. FDPDs)/kg of a subject), 8.0×10¹¹ to 1.0×10¹⁴ particles (e.g. FDPDs)/kg of a subject), 1.0×10¹² to 1.0×10¹⁴ particles (e.g. FDPDs)/kg of a subject), 3.0×10¹² to 1.0×10¹⁴ particles (e.g. FDPDs)/kg of a subject), 5.0×10¹² to 1.0×10¹⁴ particles (e.g. FDPDs)/kg of a subject), or 8.0×10¹² to 1.0×10¹⁴ particles (e.g. FDPDs)/kg of a subject).

In some embodiments, a medicament or a method of treating a subject can have a low, medium, or high dosage of platelet derivatives that has a potency in the range of 250 to 5000 TGPU per kg of the subject.

In some embodiments of any aspect or embodiment herein a therapeutically effective dose or an effective dose or amount of the platelet derivatives is an amount that has a potency in the range of 250 to 5000 TGPU per kg, 270 to 4500 TGPU per kg, 280 to 4300 TGPU per kg, 290 to 4100 TGPU per kg, 300 to 3800 TGPU per kg, 310 to 3500 TGPU per kg, or 320 to 3000 TGPU per kg of the subject. In some embodiments, a therapeutically effective dose or effective dose or amount of the platelet derivatives is an amount that has a potency in the range of 275 to 500 TGPU per kg, 280 to 450 TGPU per kg, 290 to 400 TGPU per kg, 300 to 375 TGPU per kg, 310 to 350 TGPU per kg, or 320 to 340 TGPU per kg of the subject. In some embodiments of any aspect or embodiment herein a therapeutically effective dose or effective dose or amount of the platelet derivatives is an amount that has a potency of 270 TGPU per kg, 280 TGPU per kg, 290 TGPU per kg, 300 TGPU per kg, 310 TGPU per kg, 320 TGPU per kg, 330 TGPU per kg, 340 TGPU per kg, or 350 TGPU per kg of the subject.

In some embodiments of any aspect or embodiment herein a therapeutically effective dose or effective dose or amount of the platelet derivatives is an amount that has a potency in the range of 1001 to 2000 TGPU per kg, 1200 to 2000 TGPU per kg, 1300 to 1950 TGPU per kg, 1400 to 1900 TGPU per kg, 1500 to 1900 TGPU per kg, 1600 to 1900 TGPU per kg, 1700 to 1900 TGPU per kg, 1750 to 1875 TGPU per kg, or 1800 to 1850 TGPU per kg of the subject. In some embodiments, a therapeutically effective dose or effective dose or amount of the platelet derivatives is an amount that has a potency of 1780 TGPU per kg, 1790 TGPU per kg, 1800 TGPU per kg, 1810 TGPU per kg, 1820 TGPU per kg, 1830 TGPU per kg, 1840 TGPU per kg, or 1850 TGPU per kg of the subject.

In some embodiments of any aspect or embodiment herein a therapeutically effective dose or effective dose or amount of the platelet derivatives is an amount that has a potency in the range of 2001 to 3500 TGPU per kg, 2300 to 3300 TGPU per kg, 2500 to 3100 TGPU per kg, 2600 to 3100 TGPU per kg, 2700 to 3100 TGPU per kg, 2800 to 3100 TGPU per kg, 2850 to 3050 TGPU per kg, 2900 to 3000 TGPU per kg, or 2940 to 2990 TGPU per kg of the subject. In some embodiments, a therapeutically effective dose or effective dose or amount of the platelet derivatives is an amount that has a potency of 2910 TGPU per kg, 2920 TGPU per kg, 2930 TGPU per kg, 2940 TGPU per kg, 2950 TGPU per kg, 2960 TGPU per kg, 2970 TGPU per kg, 2980 TGPU per kg, 2990 TGPU per kg, 3000 TGPU per kg, or 3100 TGPU per kg of the subject.

In certain embodiments, any of the dose ranges provided herein, and in illustrative embodiments those that include less than 1×10¹¹ particles/kg, or any of the ranges provided herein, for example those provided in the paragraph immediately above or any aspect or embodiment that includes an “administering” step, can be administered more than 1 time to a subject. For example, a dose range of between 1.0×10⁷ particles to about 1.0×10¹⁰ particles, can be administered between 2 and 10 times, or between 2 and 8 times, or between 2 and 6 times, or between 3 and 8 times, or between 3 and 6 times, or between 4 and 6 times in a timeframe between within 1, 2, 3, 4, 5, or 7 days from the first dose. And in some embodiments a single (e.g., 1×10¹⁰), double (e.g., 2×10¹⁰), triple (e.g., 3×10¹⁰), or higher dose can be administered.

In a method of treating a subject with platelet derivatives as described herein, there can be several endpoints that determine if the subject is treated. A method of treating can have one or more primary endpoints. A method can additionally have one or more secondary endpoints. In some embodiments, in a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In illustrative embodiments, a method or a medicament as described herein leads to cessation or decrease in bleeding at bleeding sites other than primary bleeding site at 24 hours after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition. In some embodiments, the bleeding in a subject is a non-compressible bleeding or a non-compressible hemorrhage. A non-compressible hemorrhage is a type of hemorrhage that is inaccessible to a tourniquet or pressure dressing. In some embodiments, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject. In some illustrative embodiments, the increase can be at least 5000 platelets/μl.

In some embodiments of any aspects or embodiments herein that include administering platelet derivatives, such as, as non-limiting as examples, a methods for controlling bleeding, or a method for selecting HLA-compatible FDPDs, or a method for administering FDPDs, or HLA-characterized FDPDs, wherein administering FDPDs, for example, HLA-characterized FDPDs, in illustrative embodiments, HLA-compatible FDPDs, for example, HLA-matched FDPDs, including HLA Class 1 antigen-matched FDPDs, HLA Class 1 antigens falling within the same cross reactive groups (CREGs), or HLA Class 1 epitope-based matched FDPDs leads to an improvement in at least one of the parameters used to assess the outcome, for example, successful outcome of administering the FDPDs herein. In some embodiments, a parameter, in illustrative embodiments, primary outcome of administering FDPDs herein is one or more of a clinical cessation of bleeding, stable HLA antibody status (no new generation of antibodies), clinician observation of positive outcomes of the subject, for example, vital signs), platelet count increment (PCI), longer transfusion free intervals, corrected count increment (CCI), reduced requirement of transfusions caused by bleeding events. In some embodiments where the parameter is PCI, the assessment can be for example, in terms of PCI at 1-hour post administration of FDPDs, where PCI at 1-hour post administration is the difference between the platelet count in the recipient at 1 hour after administration of FDPDs and the platelet count taken before the administration, for example, taken no more than 24 hours before the administration. In such embodiments, a successful outcome is a 1-hour platelet increment of at least 10×10⁹/L of blood. In some embodiments, a parameter can be an assessment of alloantibodies generated against HLA antigens of the HLA-characterized FDPDs, such as HLA-compatible FDPDs, for example, HLA-matched FDPDs to a recipient. In such embodiments, if there are no alloantibodies generated in the recipient who has received the FDPDs, then a successful outcome is determined. In some embodiments, a parameter can be an assessment of daily bleeding. For example, a recipient after being administered FDPDs completes a daily bleeding assessment form for at least 2, 3, 4, or 5 days after being administered. In some embodiments, the daily bleeding assessment can include details of bleeding grade assigned according to WHO. In some embodiments, such daily bleeding assessment indicates reduced bleeding events after administration of the FDPDs compared to within 3, 2, or 1 day before administration, and/or such daily bleeding assessment can indicate no bleeding events during the time period covered by the assessment.

### Platelet Derivatives for Treating Disorders

In some embodiments, a platelet derivative composition, in illustrative embodiment a freeze-dried platelet derivative, including, but not limited to, those of any of the aspects or embodiments herein, or the platelet derivative composition prepared by any of the process described in the aspects or embodiments herein, can be administered, or delivered in one, or in illustrative embodiments multiple doses and at dosing amounts, according to any of the dosing regimens and amounts disclosed herein, to a subject having an indication and thus afflicted with a disorder or disease that could benefit from delivery of such platelet derivative compositions. In some embodiments, such indication can be any one or a combination of Von Willebrand disease, immune thrombocytopenia (ITP), intracranial hemorrhage (ICH), traumatic brain injury (TBI), Hermansky Pudlak Syndrome (HPS), chemotherapy induced thrombocytopenia (CIT), Scott syndrome, Evans syndrome, hematopoietic stem cell transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich syndrome, venous thromboembolism, MYH9 related disease, acute lymphoblastic lymphoma (ALL), acute coronary syndrome, chronic lymphocytic leukemia (CLL), acute promyelocytic leukemia, cerebral venous sinus thrombosis (CVST), liver cirrhosis, factor v deficiency (Owren Parahemophilia), thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's disease (Factor VII Deficiency), hemophilia C (Factor XI Deficiency), familial hemophagocytic lymphohistiocytosis, acute lung injury, hemolytic uremic syndrome, menorrhagia, chronic myeloid leukemia, May-Hegglin Anomaly, Sebastion Syndrome, Fechter Syndrome, Epstein's Syndrome, Congenital Amegakaryocytic Thrombocytopenia (CAMT), Platelet Storage Pool Deficiency, ANKRD26-related Thrombocytopenia, RUNX1 Germline Mutations Platelet Disorder to include Autosomal-Dominant Familial Platelet Disorder (FPD), Periventricular Nodular Heterotopia (also known as Subependymal Grey Matter Heterotopia), GATA-1 Mutations Blood Disorders to include Dyserythropoietic Anemia, Diamond-Blackfan Anemia, Acute Megakaryoblastic Leukemia, and Transient Myeloproliferative Disorder. In illustrative embodiments, a platelet derivative composition of any of the aspects or embodiments herein, or the platelet derivative composition prepared by any of the process described in the aspects or embodiments herein can be administered, or delivered to a subject afflicted by Immune thrombocytopenia. In certain illustrative embodiments, a platelet derivative composition of any of the aspects or embodiments herein, or the platelet derivative composition prepared by any of the process described in the aspects or embodiments herein can be administered, or delivered to a subject afflicted by Von Willebrand disease. In some embodiments, a method of treating of any of the aspects or embodiments herein, can include a method of treating a subject afflicted with any of the indications as described herein. In any of the methods herein wherein platelet derivatives are administered to a subject having any of the listed indications/disorders, the subject can have an anti-coagulant or antiplatelet agent in their body, such as in their blood, and can be, or have been within 1 month, 1 week, 5 days, 4 days, 3, days, 2 days, 1 day, 12 hours, 8 hours, or 4 hours, taking or administered an anti-coagulant and/or an anti-platelet agent.

In some embodiments, a platelet derivative composition of any of the aspects or embodiments herein, or the platelet derivative composition prepared by any of the process described in the aspects or embodiments can be used to treat a subject having any one or a combination of any of the indications as described herein. In certain embodiments, a platelet derivative composition as described herein can be used, for example as a medicament or in the manufacture of a kit, for treating a subject having any one or a combination of indication as disclosed herein.

In some embodiments, an indication or indications can include those type of indications which require a much higher dose of the platelet derivatives herein, or would require an unsafe dose of, or cannot be treated with, unmodified, cold stored, naturally-occurring, endogenous, autologous, allogeneic, or normal platelets, platelets having the characteristics of in-vivo platelets, or conventional platelets (e.g. platelets collected by a conventional method for collecting platelets such as, for example, a platelet-rich plasma-method, a buff coat-method, or by apheresis), but are treatable using a platelet derivative composition of any of the aspects or embodiments herein, or the platelet derivative composition prepared by any of the processes disclosed herein, in illustrative embodiments freeze-dried platelet derivatives. In some embodiments, such an indication can be an indication that is associated with defective platelet production in a subject. In some embodiments, such an indication can be an indication that is associated with a defective platelet activity in a subject. In some embodiments, such an indication can be any of the indication as described herein. In some embodiments, indications that are typically cannot be treated with conventional platelets, but are treatable with a platelet derivative composition as disclosed herein are Von Willebrand disease, immune thrombocytopenia (ITP), intracranial hemorrhage (ICH), traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, or combinations thereof. In some embodiments, such an indication can be Von Willebrand disease. In some embodiments, such an indication can be Immune thrombocytopenia. In some embodiments, such an indication can be Chemotherapy induced thrombocytopenia (CIT). In some embodiments, such an indication can be fetal and neonatal alloimmune thrombocytopenia.

In some embodiments, an indication or indications can include those type of indications which typically, can be well-suited for treatment using a platelet derivative composition as described herein. In some embodiments, such an indication or indications can include Von Willebrand disease, immune thrombocytopenia, intracranial hemorrhage (ICH), traumatic brain injury (TBI), Hermansky Pudlak Syndrome (HPS), Scott syndrome, Evans syndrome, Bernard Soulier syndrome, Glanzmann thrombasthenia, coronary thrombosis (myocardial infarction), arterial thromboembolism, Wiskott Aldrich syndrome, venous thromboembolism, and acute coronary syndrome.

In some embodiments, platelet derivatives as described herein can have several applications in terms of treating a subject suffering with a disorder selected from the group consisting of alopecia areata, Von Willebrand Disease, hemophilia, thrombasthenia, thrombocytopenia, thrombocytopenic purpura, trauma, or a combination thereof. In some embodiments, the platelet derivatives can be used to treat clotting-related disorders. The platelet derivatives as disclosed herein can be used both as a topical application and systemic administration. In some embodiments, there is provided a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, a platelet derivative composition is passed through a filter of 18 μm before administering to the subject. A skilled artisan would be able to appreciate that the platelet derivative composition in the form of a powder which would be commercialized in vials would be rehydrated with an appropriate amount of a solution before administering to a subject. In some embodiments, such a rehydrated platelet derivative composition is passed through a filter of 18 μm before administering to the subject. In some embodiments, the platelet derivative composition as disclosed herein can be used in treating a coagulopathy in a subject that has been administered or is being administered an antiplatelet agent. In some embodiments, the platelet derivative composition of any of the aspects or embodiments herein is provided for use an anti-platelet reversal agent. In some embodiments, the platelet derivative composition of any of the aspects or embodiments herein can be used in treating a coagulopathy in a subject that has been administered or is being administered an anticoagulant agent.

In some embodiments, the platelet derivatives disclosed herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject and/or a wound of the subject.

In some embodiments, the administering can include administering topically. Administering can include administering parenterally. Administering can include administering intravenously. Administering can include administering intramuscularly. Administering can include administering intrathecally. Administering can include administering subcutaneously. Administering can include administering intraperitoneally.

The Platelet derivative compositions comprising platelet derivatives as described herein can be used as a medicament for treating a subject. Further, there is also provided herein, methods of treating a subject by administering to a subject a therapeutically effective amount or dose of a platelet derivative composition comprising platelet derivatives as described herein. In some embodiments, the subject is suffering from a condition, or a disease selected from the group consisting of thrombocytopenia, hematologic malignancy, bone marrow aplasia, myeloproliferative disorders, myelodysplastic syndromes, and platelet refractoriness. In some embodiments, the subject is suffering from one or more diseases or condition as described herein. In some embodiments, a therapeutically effective dose of platelet derivatives is based on units of thrombin generation activity administered per kilogram of body weight of the subject and/or the number of platelet derivatives delivered to the subject. In some embodiments of any aspect or embodiment herein the effective dose is based on the weight of the subject. It can be contemplated by a person of skill in the art that the effective dose can be based on any criteria that suits the requirement of the medical intervention of the subject.

In some embodiments, a method or a medicament leads to changes, or in other embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In illustrative embodiments, a method or a medicament leads to changes, or in other embodiments, does not lead to changes, in one or more markers of endothelial cell injury in the subject from a pre-administration time through baseline to 72 hours after administering the platelet derivative composition. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments, a method or a medicament leads to acceptable measures of coagulation in the subject at between 12 hours to 35 days, 24 hours to 32 days, 24 hours to 30 days, or 24 hours to 28 days after administering the platelet derivative composition. In illustrative embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments, administering confers an improved survival at 10, 15, 20, 25, 30, 35, 40, 45, or 50 days after administering the platelet derivatives. In some embodiments, administering leads to a decrease in administration of secondary blood products, platelets, or platelet derivatives to the subject for the first 5, 6, 7, 8, 9, or 10 days after the administering of the platelet derivatives.

### Circulation Persistence of Platelet Derivatives

Platelet derivatives herein, in some embodiments, exhibit less circulation persistence in a mammal when infused or administered to the mammal as compared to platelets, for example, fresh human platelets or apheresis human platelets. In some embodiments, the mammal is a human. In some embodiments, the mammal is a non-human mammal. In some embodiments, the mammal is an animal, for example a non-human mammal, for example, a mouse. In illustrative embodiments, the mammal is a non-human primate, for example, a cynomolgus macaque. In illustrative embodiments, the mammal is a human. The animal can be an animal model, for example for any disease/indication disclosed herein. Circulation persistence herein can be calculated in terms of platelet derivatives recovered from the animal after specific time points after the infusion or administration of platelet derivatives. For example, a NOD-SCID mice can be used to measure the differences in circulation persistence of platelet derivatives, for example, FDPDs or platelet derivative hemostats, and platelets, such as apheresis platelets. In some embodiments, less than 50%, 40%, 35%, 30%, 25%, 20%, 15%, or 10% of the infused platelet derivatives remain in circulation in the mammal (e.g. human or animal), for example, mouse, after 2 minutes of the administration. In some embodiments, 5-50%, 5-40%, 5-30%, 5-20%, 5-15%, or 5-10% of the infused platelet derivatives remain in circulation in the mammal, human, or animal (e.g. mouse) after 2 minutes of the administration. In some embodiments, less than 20%, 15%, 10%, 7.5%, 5%, 4%, 3%, 2%, or 1% of the infused platelet derivatives remain in circulation in the mammal, human, or animal (e.g. mouse) after 10 minutes of the administration. In some embodiments, 0.1-20%, 0.1-15%, 0.1-10%, 0.1-7.5%, 0.1-5%, 0.1-2.5%, 0.5-2.5%, 0.5-1.5%, 0.5-5%, 0.5-7.5%, 0.5-10%, or 0.5-20% of the infused platelet derivatives remain in circulation after 10 minutes of the administration to the mammal. Therefore, in some embodiments, platelet derivatives herein, for example, platelet derivative hemostat can have shorter circulation half-life in a mammal, such as a human or an animal, for example, a mouse as compared to platelets such as apheresis platelets. For example, after 2 minutes of infusion in a mammal, such as a human or an animal (e.g., a mouse), platelet derivatives herein can exhibit a recovery that is at least 3 folds, 4 fold, 5 fold, 6 fold, 7 fold, 8 fold, 9 fold, 10 fold, 12 fold, or 15 fold less as compared to apheresis platelets. In some embodiments, platelet derivatives can exhibit a recovery in the range of about or exactly 3-15 fold, 4-15 fold, 5-15 fold, 3-12 fold, 3-10 fold, 4-12 fold, 4-10 fold, 5-15 fold, 6-8 fold, or 5-10 fold less as compared to platelets, for example, apheresis platelets. For example, after 10 minutes of infusion to a mammal, such as a human or an animal (e.g., a mouse), platelet derivatives herein can exhibit a recovery that is at least 40 fold, 45 fold, 50 fold, 55 fold, 60 fold, or 65 fold less as compared to apheresis platelets. In some embodiments, platelet derivatives can exhibit a recovery in the range of 40-65 fold, 45-65 fold, 50-65 fold, or 40-60 fold less as compared to platelets, for example, apheresis platelets. Not to be limited by theory, it can be understood that differences in platelet circulation have been correlated with platelet activation and damage induced by different platelet conditions. Platelet derivatives herein, in some embodiments, present a highly activated phenotype which in turn can be cleared faster by the liver as compared to apheresis platelets. Accordingly, in some embodiments, the circulation half-life of platelet derivatives herein, can be less than 5 minutes, 4 minutes, 3 minutes, in illustrative embodiments, less than 2 minutes, or less than 1 minute after administration to a mammalian subject, for example a mouse. Accordingly, in some embodiments, the circulation half-life of platelet derivatives herein, can be less than 7 minutes, 6 minutes, 5 minutes, in illustrative embodiments, 4 minutes after administration to a mammalian subject, for example a human, or a non-human primate, such as cynomolgus macaque.

### Platelet Derivatives Affect Platelet Biomarkers of Endogenous Platelets

Methods herein, or compositions for use in methods herein, include administering in one, or in multiple doses, according to any dosing regimens and amounts provided herein, an effective amount of platelet derivatives, FDPDs, HLA-characterized FDPDs, HLA-matched FDPDs, HLA compatible FDPDs or freeze-dried platelet derivatives in a platelet derivative composition to a subject in need thereof. In illustrative embodiments, the subject in need thereof, has Hermansky Pudlak Syndrome (HPS). In illustrative embodiments, the platelet derivative composition comprises a population having a reduced propensity to aggregate such that no more than 10%, 8%, 7%, or 5% of the platelet derivatives in the population aggregate under aggregation conditions comprising an agonist but no platelets. In some embodiments, administering can include administering the platelet derivatives, or freeze-dried platelet derivatives for treating a subject. The treating, for example, can include a partial, or a complete restoration of platelet functions in the subject.

For example, administering platelet derivatives, or freeze-dried platelet derivatives (FDPDs) herein to a subject, patient, and/or recipient can have certain effects on the levels of at least one platelet biomarker of the endogenous platelets in the subject, patient, and/or recipient of the FDPs. A skilled artisan will understand that the endogenous platelets are the platelets of a subject, and does not include platelets, or platelet derivatives that are administered to the subject. The platelet biomarkers that can be affected upon administering, in some embodiments can include any platelet biomarker that is associated with the activation of the platelets, or endogenous platelets in a subject.

In some embodiments, the platelet biomarkers whose levels are affected by administration of FDPDs can be any one of PAC-1, CD62P, CD63, or combinations thereof. In some embodiments, the platelet biomarkers can be at least two, or all the three of the platelet biomarkers PAC-1, CD62P, CD63. In illustrative embodiments, the platelet biomarker can be PAC-1, CD62P, or both. In some embodiments, the administering can lead to restoring the levels of at least one platelet biomarkers of the endogenous platelets in the subject to levels similar to the corresponding platelet biomarkers of platelets in a healthy subject. Similar levels as used herein, can cover a range within lower values and higher values of the level of a corresponding platelet biomarker from a healthy subject. In other words, similar levels can include levels higher or lower than a control level or a normal level of the corresponding platelet biomarker in a healthy subject. For example, the administering herein, can restore the levels to between 30% higher to 30% lower, between 25% higher to 25% lower, between 20% higher to 20% lower, between 15% higher to 15% lower, between 10% higher to 10% lower, or between 5% higher to 5% lower levels of the endogenous platelets in the subject as compared to normal levels for these biomarkers. For example, the administering herein, can restore the levels within at least 5%, 10%, 15%, 20%, 25%, or 30% levels of the endogenous platelets in the subject as compared to the normal levels. In some embodiments, the administering herein can lead to improving, increasing, increasing platelets testing positive for the at least one platelet biomarker, affecting activation of at least one platelet biomarker, or restoring the at least one platelet biomarker as discussed herein. For example, the administering can lead to an increase or an improvement in the levels of at least one platelet biomarker of platelets, in illustrative embodiments, endogenous platelets, such that the levels are increased by at least 5%, 10%, 15%, 20%, 25%, 30%, 35%, 40%, 45%, 50%, 55%, 60%, or more as compared to the levels in the subject before administering the platelet derivatives, or freeze-dried platelet derivatives. In some embodiments, the administering can lead to an increase or an improvement in the levels of at least one platelet biomarker in the range of 2 to 70%, 2 to 65%, 2 to 60%, 2 to 55%, 2 to 50%, 2 to 45%, 2 to 40%, 2 to 35%, 2 to 30%, 5 to 70%, 10 to 70%, 15 to 70%, 20 to 70%, 25 to 70%, 30 to 70%, or 35 to 75%, as compared to the levels in the subject before administering the platelet derivatives, or freeze-dried platelet derivatives.

A subject having HPS can have HPS-related biomarker abnormalities. Thus, the subject can be treated by administering the platelet derivatives, or freeze-dried platelet derivatives to the subject, such that at least one HPS-related biomarker abnormality observed in the subject is improved in the subject after the administering as compared to before the administering step. In some embodiments, administering can lead to restoring the levels of at least one HPS-related biomarker to normal levels after the administering as compared to before the administering step. In some embodiments, the FDPD administration can improve, control, and/or restore the levels of biomarkers and/or hemostasis in the recipient subject, such that the subject stops taking another therapeutic for treating HPS and/or an HPS-related biomarker abnormality.

In some embodiments, administering platelet derivatives, or freeze-dried platelet derivatives can lead to an increase in the levels of at least one platelet biomarker of endogenous platelets in a recipient subject/patient compared to the levels of a corresponding platelet biomarker in a subject having HPS, but not administered with the platelet derivatives, or freeze-dried platelet derivatives. In some embodiments, administering herein, can lead to an increase in, or restore, the levels of a platelet biomarker of endogenous platelets in a recipient subject/patient as compared to a subject having HPS but administered normal platelets, in illustrative embodiments, normal apheresis platelets, and not administered, or before administering the platelet derivatives, or the freeze-dried platelet derivatives as disclosed herein.

Delivery to a Subject being Treated with an Anti-Platelet and/or Anti-Coagulant Agent

In some aspects and embodiments, platelet derivatives provided herein can be used to treat a coagulopathy in a subject that has been administered or is being administered an antiplatelet agent and/or an anticoagulant agent and/or aspirin. In some embodiments of any of the aspects or embodiments herein, platelet derivatives herein can be administered multiple times at a frequency disclosed herein or administered as a continuous infusion to a subject who has been, or is being administered an antiplatelet agent, an anticoagulant agent, or a combination thereof. For example, the subject in some embodiments has been, or is being administered, an antiplatelet agent, aspirin, and an anticoagulant. Accordingly, in related aspects and embodiments platelet derivatives as provided herein can be used as an anti-platelet reversal agent, or an anti-coagulant reversal agent. In some embodiments, methods herein comprise administering 2-50, 2-45, 2-40, 2-35, 2-30, 2-25, 2-20, 2-15, 2-10, 2-8, 2-6, 5-50, 10-50, 15-50, 20-50, 25-50, 3-15, 3-12, 3-9, 4-25, 4-20, 4-18, 4-15, 5-25, 5-20, 5-15, or 5-10 doses of platelet derivatives to a subject within 1 hour, for example, after administering a first dose of platelet derivatives, wherein the subject has been administered or is being administered one or more of an antiplatelet agent, an anticoagulant, and aspirin. For example, in a subject who has been administered or is being administered an antiplatelet agent, an anticoagulant, or aspirin, between 2 in the lower end and 7, 8, 10, 12, 15, 20 doses in the higher end of platelet derivatives, or between 3 in the lower end and 7, 8, 10, 12, 15, 20 doses in the higher end of platelet derivatives, in illustrative embodiments each dose in the range of 1.0×10⁸ to 1.0×10¹²/kg of the subject can be administered to the recipient subject within 1 hour, 30 minutes, 20 minutes, 15 minutes, 10 minutes or 5 minutes, and such administration can be guided by the effect of the dosing on bleeding of the subject, for example at one or more specific sites of bleeding. The antiplatelet class of drugs, which an illustrative class of antiplatelet agents, is widely used to prevent unwanted clotting episodes that lead to heart failure, stroke, and the like. In many cases, an antiplatelet drug may need to be reversed or stopped. In the case of advanced notice, as in a pre-planned surgery situation, the antiplatelet drug dose can sometimes be stopped before the surgery, preventing unwanted bleeding during surgery. In the case where an antiplatelet agent needs reversing quickly, reversal agents are typically not readily available, are expensive, or carry significant risk to the patient. In the case of need for rapid antiplatelet reversal, a platelet transfusion is typically administered, though the response to this is often only partial reversal. The caveat of this course of reversal is that the newly-infused platelets themselves are susceptible to circulating drug antiplatelet activity whereas, in some embodiments, compositions as described herein (e.g., including thrombosomes) are not. In some embodiments, compositions as described herein (e.g., including thrombosomes) are an active reversal agent. In some embodiments, the hemostatic activity of compositions as described herein (e.g., including thrombosomes) does not succumb to antiplatelet drugs. In some embodiments, an antiplatelet agent can be selected from the group consisting of aspirin, cangrelor, ticagrelor, clopidogrel, prasugrel, eptifibatide, tirofiban, abciximab, and combinations thereof. In some embodiments of any of the methods herein, a subject has been administered an antiplatelet agent or an anticoagulant, or is being administered an antiplatelet agent or an anticoagulant, in any appropriate time frame. For example, in some cases, a subject has been administered an antiplatelet agent or an anticoagulant before the effect of the antiplatelet agent or the anticoagulant wears off. For example, in some cases, a subject is being administered an antiplatelet agent and the effect of the antiplatelet agent has not worn off. As another example, in some cases, a subject has been administered an antiplatelet agent (e.g., the most recent dose) within about 1 week, about 5 days, about 3 days, about 36 hours, about 24 hours, about 18 hours, about 12 hours, about 8 hours, about 6 hours, about 4 hours, about 2 hours, or about 1 hour. As another example, in some cases, a subject is being administered an antiplatelet agent and the last dose (e.g., the most recent dose as prescribed by a medical professional or self-administered by the subject) was within about 1 week, about 5 days, about 3 days, about 36 hours, about 24 hours, about 18 hours, about 12 hours, about 8 hours, about 6 hours, about 4 hours, about 2 hours, or about 1 hour. As another example, determining that the subject has been administered an antiplatelet agent contrary to medical instruction can be within about 1 week, about 5 days, about 3 days, about 36 hours, about 24 hours, about 18 hours, about 12 hours, about 8 hours, about 6 hours, about 4 hours, about 2 hours, or about 1 hour of the administering a composition provided herein or a composition produced by a method described herein. For example, in some cases, a subject is being administered an anticoagulant and the effect of the anticoagulant has not worn off. As another example, in some cases, a subject has been administered an anticoagulant (e.g., the most recent dose) within about 1 week, about 5 days, about 3 days, about 36 hours, about 24 hours, about 18 hours, about 12 hours, about 8 hours, about 6 hours, about 4 hours, about 2 hours, or about 1 hour. As another example, in some cases, a subject is being administered an anticoagulant and the last dose (e.g., the most recent dose as prescribed by a medical professional or self-administered by the subject) was within about 1 week, about 5 days, about 3 days, about 36 hours, about 24 hours, about 18 hours, about 12 hours, about 8 hours, about 6 hours, about 4 hours, about 2 hours, or about 1 hour. As another example, determining that the subject has been administered an anticoagulant agent contrary to medical instruction can be within about 1 week, about 5 days, about 3 days, about 36 hours, about 24 hours, about 18 hours, about 12 hours, about 8 hours, about 6 hours, about 4 hours, about 2 hours, or about 1 hour of the administering a composition provided herein or a composition produced by a method described herein.

In some embodiments of any of the methods for treating or methods of administering aspects herein, the subject is being treated or was treated with an anti-coagulant. In certain embodiments, the anticoagulant is dabigatran, argatroban, hirudin, rivaroxaban, apixaban, edoxaban, fondaparinux, warfarin, heparin, a low molecular weight heparin, a supplement, or a combination thereof. In some embodiments of any of the methods for administering or for treating aspects herein, wherein the subject is being treated with an anticoagulant, the anticoagulant is dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a health and wellness supplement with anti-coagulant properties, or a combination thereof.

In some embodiments, there is provided a method of administering to a subject, or a method of treating coagulopathy in a subject, wherein the subject has been treated or is being treated with an antiplatelet agent or an anti-coagulant, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of restoring normal hemostasis in a subject, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of preparing a subject for surgery, the method comprising administering to the subject in need thereof an effective amount of a composition comprising platelet derivative composition as described herein and an incubating agent comprising one or more salts, a buffer, optionally a cryoprotectant, and optionally an organic solvent.

In some embodiments, the subject that has been administered or is being administered an antiplatelet agent or an anticoagulant agent in a subject having an indication and thus afflicted with a disorder or disease, that is any one or a combination of Von Willebrand disease, immune thrombocytopenia, intracranial hemorrhage (ICH), traumatic brain injury (TBI), Hermansky Pudlak Syndrome (HPS), chemotherapy induced thrombocytopenia (CIT), Scott syndrome, Evans syndrome, Hematopoietic Stem Cell Transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich Syndrome, venous thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's Disease (Factor VII deficiency), hemophilia c (factor xi deficiency), familial hemophagocytic lymphohistiocytosis, acute lung injury, hemolytic uremic syndrome, menorrhagia, chronic myeloid leukemia.

Provided herein in one aspect is a method of treating a coagulopathy in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition comprises platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of preparing a subject for surgery, the method including administering to the subject in need thereof an effective amount of a composition including platelets, or in illustrative embodiments platelet derivatives, and in further illustrative embodiments FDPDs. Various properties of exemplary embodiments of such FDPDs are provided herein, thereby treating the coagulopathy. In illustrative embodiments, the composition comprising the platelet derivatives is administered such that the bleeding potential of the subject is reduced, and in illustrative embodiments such that normal hemostasis is restored in the subject. Implementations can include one or more of the following features. The surgery can be an emergency surgery. The surgery can be a scheduled surgery.

In one aspect, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject. Various properties of exemplary embodiments of such FDPDs are provided herein. Implementations can include one or more of the following features. The surgery can be an emergency surgery. The surgery can be a scheduled surgery.

In one aspect, provided herein is a method of ameliorating the effects of an antiplatelet agent in a subject, the method including administering to the subject in need thereof an effective amount of a composition platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of ameliorating the effects of an antiplatelet agent in a subject, the method including administering to the subject in need thereof an effective amount of a composition prepared by a process including incubating platelets with an incubating agent including one or more salts, a buffer, optionally a cryoprotectant, and optionally an organic solvent, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, or of restoring hemostasis in a subject, or of reducing bleeding potential of a subject that is being administered, or has been administered, an antiplatelet agent, the method comprising: administering to the subject in need thereof an effective amount of a composition comprising platelet derivatives, thereby treating the coagulopathy. In illustrative embodiments, the platelet derivatives are freeze-dried platelet derivatives (FDPDs). In further illustrative embodiments, the composition comprising the platelet derivatives is administered such that the bleeding potential of the subject is reduced, and in illustrative embodiments such that normal hemostasis is restored in the subject.

In another aspect, provided herein is a method of treating a coagulopathy in a subject, or of restoring hemostasis in a subject, or of reducing bleeding potential of a subject, wherein the subject is being administered, or has been administered, an antiplatelet agent, the method comprising administering to the subject in need thereof an effective amount of the composition comprising FDPDs, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy. In further illustrative embodiments, the composition comprising the FDPDs is administered such that the bleeding potential of the subject is reduced, and in illustrative embodiments such that normal hemostasis is restored in the subject.

In another aspect, provided herein is a method of preventing or mitigating the potential for a coagulopathy in a subject, the method comprises: (a) determining that information regarding whether the subject was administered an antiplatelet agent is unavailable; and (b) administering to the subject an effective amount of a composition comprising freeze-dried platelet derivatives (FDPDs). In some embodiments of such a method, information regarding whether the subject was administered an antiplatelet agent is unavailable for a reason comprising that the subject cannot be identified. In some embodiments of the method, information regarding whether the subject was administered an antiplatelet agent is unavailable for a reason comprising that the medical history of the subject is unavailable. In further embodiments information regarding whether the subject was administered an antiplatelet agent is unavailable for a reason comprising that the subject is in need of emergency treatment.

In another aspect, provided herein is a method of treating a coagulopathy in a subject or of reducing the bleeding potential of a subject, or of restoring hemostasis in a subject, wherein the method comprises: administering to the subject in need thereof an effective amount of a composition comprising platelet derivatives, in illustrative embodiments, FDPDs, wherein the subject before the administering the composition comprising platelet derivatives, was administered an antiplatelet agent and a second agent that decreases platelet function, thereby treating the coagulopathy. In further illustrative embodiments, the composition comprising the platelet derivatives is administered such that the bleeding potential of the subject is reduced, and in illustrative embodiments such that normal hemostasis is restored in the subject. In illustrative embodiments, before the administering of the composition comprising FDPDs the subject was in need thereof because of an increased risk of bleeding due to, or as a result of being administered the anti-platelet agent and the second agent.

In another aspect, provided herein is a composition comprising freeze-dried platelet derivatives (FDPDs) for treating a coagulopathy in a subject, wherein the treating comprises: administering to the subject in need thereof, an effective amount of the composition comprising FDPDs such that the bleeding potential, or risk of bleeding of the subject is reduced, wherein the subject was administered an antiplatelet agent and a second agent that decreases platelet function, and wherein the subject is in need thereof because of an increased potential for, or risk of bleeding due to, or as a result of being administered the antiplatelet agent and the second agent, thereby treating the coagulopathy.

In another aspect, provided herein is a composition comprising freeze-dried platelet derivatives (FDPDs) for treating a coagulopathy in a subject having an increased potential for, or risk of bleeding as a result of being administered or having been administered an anticoagulant, wherein the treating comprises: administering to the subject having the increased potential for, or risk of bleeding, an effective amount of the composition comprising FDPDs such that the bleeding potential or risk of bleeding of the subject is reduced, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy.

In some embodiments of any of the method or use embodiments herein, a dose, and in illustrative embodiments an effective amount of a composition comprising platelets or platelet derivatives (e.g., FDPDs) administered to a subject or patient, can be in a range of between about or exactly 1.0×10⁸, 5.0×10⁸, 1.0×10⁹, 3.0×10⁹, 4.0×10⁹, 5.0×10⁹, 1.0×10¹⁰, or 5.0×10¹⁰ to 1.0×10¹² particles (e.g. platelet derivatives or FDPDs)/kg of a subject, or in a range of between about or exactly 1.0×10⁸, 5.0×10⁸, 1.0×10⁹, 3.0×10⁹, 4.0×10⁹, 5.0×10⁹, 1.0×10¹⁰, 5.0×10¹⁰, 1.0×10¹¹, 5.0×10¹¹, 1.0×10¹², 5.0×10¹², or 1.0×10¹³ to 1.0×10¹⁴ particles (e.g. platelet derivatives or FDPDs)/kg. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 5.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 1.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, or 5.0×10⁹ to 1.0×10¹⁰ particles (e.g. FDPDs)/kg of a subject. In one illustrative embodiment, and in illustrative embodiments wherein a subject has blood comprising i) an anti-platelet agent and a second agent that decreases platelet function; ii) two anti-platelet agents; and/or iii) has been administered dual anti-platelet therapy, a dose or an effective amount of a composition comprising FDPDs is between 5.0×10¹⁰ to 1.0×10¹²/kg of the subject, 5.0×10¹⁰ to 5.0×10¹¹/kg of the subject, 5.0×10¹⁰ to 1.0×10¹¹/kg of the subject, 5.0×10⁹ to 1.0×10¹¹/kg of the subject, 5.0×10⁹ to 5.0×10¹⁰/kg of the subject, or 5.0×10⁹ to 1.0×10¹⁰/kg of the subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg oft subject on the high end; or 1.1×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end.

In some embodiments, for example of aspects wherein a subject was administered the antiplatelet agent and the second agent that decreases platelet function, such a method further comprises before the administering the composition comprising FDPDs, determining that the subject was administered the antiplatelet agent and the second agent that decreases platelet function. In some embodiments, the antiplatelet agent is a first antiplatelet agent and the second agent is a second antiplatelet agent. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In some embodiments, the first antiplatelet agent and the second anti-platelet agent have different mechanisms of action. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate.

In some embodiments of any of the aspects herein, before, immediately before, at the moment before, at the moment of, and/or at an initial time of, the administering of the composition comprising platelet derivatives, for example FDPDs, the subject was or is at an increased risk of bleeding due to being administered or having been administered the anti-platelet agent. Furthermore, the subject can be at an increased risk of bleeding at 7, 6, 5, 4, 3, 2, or 1 day, 12 hours, 8 hours, 4 hours, 2 hours, 1 hour or 45, 30, 15, 10, 5, 4, 3, 2, or 1 minute before the administering of the composition comprising the platelet derivatives. In some optional embodiments, this is confirmed by laboratory testing. However, in some embodiments no laboratory testing of bleeding risk or any clotting parameter is performed 7, 6, 5, 4, 3, 2, or 1 day or sooner before and/or after the administering of the composition comprising the platelet derivatives. Bleeding risk is typically decreased after administration of an effective dose of the composition comprising platelet derivatives, in illustrative embodiments FDPDs. Furthermore, the subject may remain at an increased risk of bleeding even after the administering of the composition comprising platelet derivatives (e.g. FDPDs), for example for 1, 2, 3, 4, 5, 10, 15, 20, 30, or 45 minutes, or 1, 2, 3, 4, 5, or 8 hours, or longer after the administering, depending on how long it takes for the FDPDs to decrease the risk in the subject after they are administered. Furthermore, in some embodiments, the administration of the composition comprising the platelet derivatives (e.g. FDPDs) decreases but does not completely resolve the increased risk of bleeding in the subject.

In some embodiments, for example of aspects wherein a subject was administered the antiplatelet agent and the second agent that decreases platelet function, administration of the second agent is stopped, for example before administrating the composition comprising the platelet derivatives. In other embodiments of such aspects, administration of the second agent is continued, for example after administering the composition comprising the platelet derivatives.

In certain embodiments of any of the aspects provided herein, the method further comprises before administering the composition comprising platelet derivatives, determining in a pre-administering evaluation, that the subject has an abnormal value for one or more clotting parameters. The pre-administration evaluation, in illustrative embodiments, is an in vitro laboratory test.

In certain embodiments of any of the aspects provided herein, the antiplatelet agent is selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In other embodiments, the antiplatelet agent is selected from cangrelor, ticagrelor, abciximab, terutroban, picotamide, elinogrel, ibuprofen, vorapaxar, atopaxar, cilostazol, prostaglandin E1, epoprostenol, dipyridamole, treprostinil sodium, and sarpogrelate.

### Platelet Derivatives Affect Thrombin Generation and Clotting Ability

Administering platelet derivatives, freeze-dried platelet derivatives (FDPDs), HLA-characterized FDPDs, HLA-matched FDPDs, HLA compatible FDPDs, freeze-dried platelet derivative hemostat (FPH), HLA-characterized FPH, HLA-matched FPH, or HLA compatible FPH herein to a subject (i.e. recipient), in illustrative embodiments, to a subject (i.e. recipient) having HPS can affect at least one HPS-related hemostatic abnormality. In some embodiments, the administering can be used to treat abnormalities caused by HPS in the subject. The treatment herein can be a partial treatment or a complete treatment. For example, administering herein can lead to an improvement in one, two, three, at least one, all but one, or all HPS-related hemostatic abnormality. Furthermore, administration of the FDPDs can maintain the normal levels of hemostasis in the subject. For example, after administration of the FDPDs to the subject, the levels of hemostasis can be maintained such that the subject stops taking another therapeutic that the subject is taking for treating the HPS-related hemostatic irregularity(s). In some embodiments, administering of FDPDs herein can lead to an improvement in thrombin generation in the subject as compared to the subject before the administering. For example, administering herein can increase or improve thrombin generation in the subject in vivo, and/or in an ex vivo assay using the subject's blood, by at least 5%, 10%, 15%, 20%, 25%, 30%, 35%, 40%, 45%, 50%, 55%, 60%, or more. In other embodiments, administering the FDPDs herein, can increase thrombin generation in the subject in vivo, and/or in an ex vivo assay using the subject's blood, in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%.

A skilled artisan can use any known test(s) to assess thrombin generation in a subject. For example, thrombin generation can be assessed by a thrombin generation assay, and the assay can be performed by semi-automated methods for example using a calibrated automated thrombogram, or using fully automated systems. Thrombin generation assay is a type of coagulation test and is based on the potential of a plasma to generate thrombin over time, following addition of activators like phospholipids, tissue factor, and calcium. The results of the assay can typically be calculated as a thrombogram, or thrombin generation curve using computer software after calculation of thrombogram parameters.

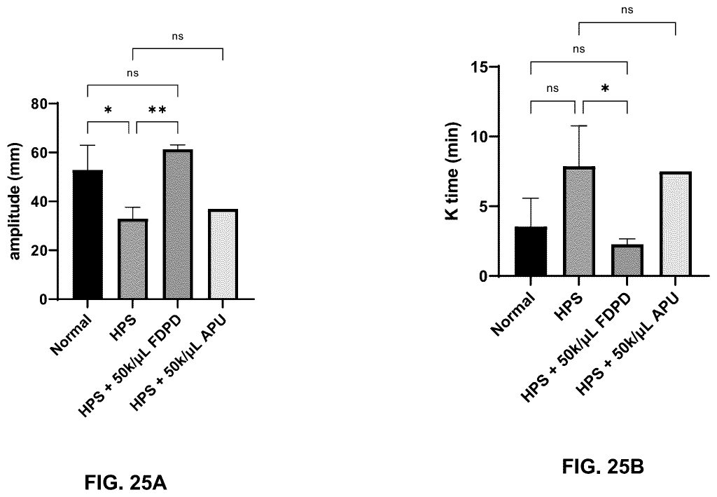

In some embodiments, administering FDPDs, HLA-characterized FDPDs, HLA-matched FDPDs, HLA compatible FDPDs, freeze-dried platelet derivative hemostat (FPH), HLA-characterized FPH, HLA-matched FPH, or HLA compatible FPH herein can lead to an improvement in clot formation in the subject as compared to the subject before the administration of FDPDs. For example, administering FDPDs herein can increase or improve clot formation in the subject by at least 5%, 10%, 15%, 20%, 25%, 30%, 35%, 40%, 45%, 50%, 55%, 60%, or more. In other embodiments, administering herein, can increase clot formation in the subject in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%. Clot formation can be monitored by known techniques, for example, a thromboelastography method (TEG). As shown in Example 25 (FIG. 25A and FIG. 25B) it was surprisingly observed that providing FDPDs ex vivo HPS patient blood restores clot amplitude better than the same dose of apheresis platelets. Clot formation parameters are measured including time to clot initiation, the rate of clot formation, the angle of clot, maximum amplitude of the size of clot, and the percent platelet activation normalized to a known sample. TEG can also be used to analyze platelet function, and fibrinolysis along with clot formation. In some embodiments, administering can lead to an improvement or increase in both thrombin generation and clot formation. In some embodiments, the administering leads to an improvement or increase in thrombin generation and/or clot formation in the subject as compared to the subject after being administered apheresis platelets, but before the administering of the platelet derivatives, or the freeze-dried platelet derivatives.

In some embodiments, platelet derivatives, HLA-characterized FDPDs, HLA-matched FDPDs, HLA compatible FDPDs, freeze-dried platelet derivative hemostat (FPH), HLA-characterized FPH, HLA-matched FPH, or HLA compatible FPH herein can exhibit a delayed clot lysis time as compared to platelets, for example, fresh platelet, or apheresis platelets. In some embodiments, clot lysis can be analyzed by calculating the time between 50% max O.D. from clot formation due to platelets or platelet derivatives herein to clot lysis. In some embodiments, the clot lysis can be analyzed at a specific concentration of platelets or platelet derivatives, for example, 20×10³ particles/μl, 40×10³ particles/μl, or 100×10³ particles/μl. For example, at a concentration of about 20×10³ particles/μl, platelet derivatives herein exhibit a clot formation that has a delayed lysis time as compared to platelets, such as fresh platelets or apheresis platelets. In some embodiments, clots formed by platelet derivatives at a concentration of about 20×10³ particles/μl exhibit a delay of at least 5 minutes, 10 minutes, 15 minutes or more as compared to a same or similar concentration of platelets, such as, fresh platelets or apheresis platelets. Not bound by any theory, it is understood that the delayed clot lysis can be in part due to the surface presence of PAI-1, and bound plasma factor XIII.

### Collections of HLA Characterized FDPDs, and Methods for Making the Same

Many of the methods, processes, collections, and compositions herein include or produce HLA-characterized (e.g., matched), in illustrative embodiments HLA Class 1-characterized (e.g., matched) platelet derivatives. Such HLA-characterized (e.g., matched) platelet derivatives can be dried or rehydrated, HLA-characterized (e.g., matched) platelet derivatives, and in illustrative embodiments are freeze-dried, HLA Class 1-characterized (e.g., matched) platelet derivatives. Such HLA-characterized (e.g., matched) platelet derivatives can be prepared in methods provided herein, using a plurality (e.g. pool) of HLA-characterized platelets, for example using methods for making a collection of HLA Class 1-characterized, in some embodiments HLA Class 1-matched, FDPDs. Such HLA Class 1-characterized (e.g., matched) platelet derivatives (e.g., FDPDs), and batches and collections of the same, can be used in methods herein that include administering FDPDs to a subject having HPS.

Accordingly, in one aspect, provided herein is a method for making a collection of HLA Class 1-characterized, freeze-dried platelet derivatives (FDPDs). In a non-limiting example, the HLA Class 1 type of a plurality of platelet donors can be determined for a plurality of platelet samples (e.g., donor apheresis platelets). Such determination can be performed for example at a blood collection center, a facility storing collected blood, or at a site at which FDPDs will be prepared from at least some of the donor platelets. Methods are known in the art, some of which are provided herein, for determining the HLA Class 1 characteristics, typically the HLA Class 1 type and/or antigens of platelets, using platelets or blood or a blood fraction from which the platelets were isolated, or from another tissue of a donor. Thus, in certain non-limiting embodiments the platelets from individual donors considered for pooling are HLA-typed (e.g. HLA Class 1 antigen types are determined) to identify the HLAs present on the surface. In some embodiments, the entity performing the method of making the collection does not actual perform the HLA Class 1 determination, but rather receives this information, for example via a computer network, such as for example the Internet.

Next, the HLA Class 1 characteristics (e.g., type and/or antigen information) regarding the plurality of platelet samples (e.g., apheresis platelets) from a plurality of platelet donors is used to select platelets from a subset of the plurality of platelet donors to include in a pool of platelets. HLA Class 1 characteristics (e.g., types and/or antigens) of the plurality of donors can be used in various ways to select platelet donors whose platelets are combined to form the pool as discussed herein. The process is carried out such that Platelets from subsets of donors that are selected based on their HLA Class 1 characteristics are pooled to form a plurality of pools of HLA-characterized platelets. The HLA Class 1 characteristics (e.g., HLA Class 1 types or antigens) for some, most, or in illustrative embodiments all of the pools of the plurality of pools are different from each other.

In some illustrative embodiments of any of the methods herein that include a pooling step, such as, but not limited to the illustrative method, or that include pooled platelets, platelets are pooled from platelet donors having HLA matched, in illustrative embodiments HLA Class 1-matched platelets using any HLA matching strategies and/or criteria known in the art, as non-limiting examples, any of the matching strategies and/or criteria provided herein. For example, platelets can be pooled from donors having cross-reactive antigens falling within the same cross-reactive group. Alternatively, platelets can be pooled from platelet donors that have matched platelets to a grade “A”, “B1U”, “B1X”, “B2U”, “B2UX”, “B2X”, “C”, or “D” match as discussed in more detail herein. The HLA Class 1 matching can be done based on HLA-A, HLA-B, and HLA-C types and/or antigens. Alternatively, the matching can be done based on the HLA-A and HLA-B types and/or antigens. In other embodiments, the matching can be done based on epitope matching of HLA Class 1 antigens between different donors. Further disclosure regarding any of these matching strategies, criteria, and grades are discussed further herein and can be used to identify donors whose platelets can be pooled.

Next, the pooled HLA characterized platelets, are used to prepare freeze-dried platelet derivatives (FDPDs). Details are provided herein for preparing FDPDs from platelets. Furthermore, U.S. Pat. No. 11,529,587 and PCT app no. PCT/US2022/079280 provide methods for preparing FDPDs/thrombosomes from platelets, including methods that use tangential flow filtration (TFF) that can be used in methods herein. Furthermore, methods for making (i.e., processes for preparing) FDPDs/thrombosomes) such as those that utilize centrifugation from WO 2006/020773, incorporated by reference herein in its entirety, can be used. A single batch of HLA-matched FDPDs is typically the contents of a single freeze-drying process, which can be distributed in a single container, or a plurality of containers. It is noteworthy that there may not be more than 1 donor that has a certain HLA type, and thus in some cases, platelets from an individual donor can used to make a batch of FDPDs for some HLA types. Optionally, the HLA Class-1 type of FDPDs in the batches can be confirmed after preparing the batches of HLA-characterized FDPDs for HLA-characterized platelet pools.

As indicated above, a plurality of pools are typically prepared as part of the process. Thus, the step of selecting or identifying platelets to pool can be repeated to create a batch of FDPDs, such that a batch of FDPDs can be prepared from each pool of platelets, and making using different platelet donors of the plurality of platelet donors to make a plurality of pools and a plurality of batches, to produce a collection of at least 3 batches of HLA Class 1-characterized FDPDs, wherein the set of HLA Class 1 antigens for each batch is different from the set of HLA Class 1 antigens for any other batch of the collection. A plurality of batches of FDPDs can then be stored in an accessible and identifiable manner, to form a collection of FDPDs, or one or more batches of FDPDs can be added to an existing collection of batches of FDPDs.

Accordingly, compositions herein, or process herein can include a collection of vessels containing platelet derivative compositions in the form of a powder, comprising: a first vessel comprising a first population of HLA Class 1-characterized platelet derivatives having a biomolecule profile indicative of more than 1 platelet donor; and a second vessel comprising a second population of HLA Class 1-characterized platelet derivatives having a biomolecule profile indicative of more than 1 platelet donor, wherein the HLA Class 1 characteristics of the first population is different than the HLA Class 1 characteristics of the second population. In some embodiments, a first vessel comprising a first population of HLA Class 1-characterized platelet derivatives obtained from more than 1 donor; and a second vessel comprising a second population of HLA Class 1-characterized platelet derivatives obtained from more than 1 donor. In some embodiments, a collection of vessels comprises a third vessel comprising a third population of HLA Class 1-characterized platelet derivatives having a biomolecule profile indicative of more than 1 platelet donor, or obtained from more than 1 donor, wherein the HLA Class 1 characteristics are different for each of the first population, the second population, and the third population. A collection of vessels as disclosed herein can comprise a plurality of 3, 4, 5, 6, 7, 8, 10, 20, 50, 100, 500, 1000 or more batches of vessels, each vessel of a batch comprising a population of HLA Class 1-characterized platelet derivatives, and the HLA Class 1 characteristics for each batch of vessels is different from the HLA Class 1 characteristics for any other batch of vessels in the collection. In some embodiments, a collection of vessels can comprise 2-5000, 2-2000, 2-1000, 2-750, 2-500, 2-400, 2-300, 2-250, 100-5000, 500-5000, 1000-5000, or 2000-5000 batches of vessels. In illustrative embodiments, a collection of vessels can comprise 2-50, 2-45, 2-40, 2-35, 2-30, 2-25, 2-20, 2-15, or 2-10 batches of vessels. In some embodiments, a collection of vessels herein can comprise 2, 3, 5, 10, 20, 20, 100, or more vessels in each batch. There can be 2-1000, 2-750, 2-500, 2-400, 2-300, 2-200, 2-100, 100-1000, 200-1000, 500-1000, or 700-1000 vessels in each batch. Thus, a large number of readily available HLA Class 1-characterized platelet derivatives can be produced and stored. Since, the freeze-dried platelet derivatives herein can have a prolonged shelf-life, many batches encompassing tens, hundreds, thousands, or more different HLA characteristics can be prepared and stored well in advance, thus, being readily available for use for example in methods of administering provided herein. Whenever a requirement of HLA-matched platelets arises, a desired batch of HLA Class 1-characterized freeze-dried platelet derivatives from a collection herein can be selected based, for example, on HLA Class 1 typing of the recipient and can be provided much more readily than is currently available. Accordingly, in a collection herein, a method herein that includes a donor and a recipient, or a method of making a collection herein having a plurality of vessels having HLA-characterized platelet derivatives or FDPDs, HLA Class-1 matched platelet derivatives can comprise FDPDs that are HLA Class 1 antigen-matched platelet derivatives, for example, the FDPDs in the vessel have HLA Class 1 antigens matched to a grade A, B1U, B1X, B2U, B2UX, B2X, C, or D match. Such matching can relate to HLA type of platelet donors used to make a pool and/or the HLA type of a donor(s) compared to the HLA type of a recipient. In some embodiments, HLA Class-1 matched platelet derivatives in a collection can comprise FDPDs that are matched based on epitope-based matching of HLA Class 1 antigens. In some embodiments, HLA Class-1 matched platelet derivatives in a collection can comprise FDPDs that have either no mismatch of HLA Class-1 antigens or no more than one mismatch of HLA Class-1 antigens. In some embodiments, HLA Class-1 matched platelet derivatives in a collection can comprise FDPDs that have HLA Class 1 cross-reactive antigens falling within the same cross-reactive group.

In illustrative embodiments, each batch of vessels comprises HLA Class 1-characterized freeze-dried platelet derivatives having a defined HLA characteristics obtained from a common pooled platelet composition comprising HLA Class 1-characterized platelets with the same or similar defined HLA characteristics. In some embodiments, the HLA characteristics of the HLA Class 1-characterized freeze-dried platelet derivatives of a batch of vessels remain identical amongst all the vessels from one batch. In some embodiments, the HLA characteristics of HLA Class 1-characterized freeze-dried platelet derivatives of one batch of vessels differs from that of another batch. It is envisioned that different batches/lots of HLA Class 1-characterized freeze-dried platelet derivatives can be prepared, and each batch has a defined HLA characteristic that is different from one another. For example, in some embodiments 5 or less, 4 or less, 3 or less, 2 or less, 1 or 0 antigens, HLA antigens, for example HLA Class 1 antigens, found in one batch are found in another batch, or 1 on the low end, to 10, 15 or 20 antigens, for example HLA Class 1 antigens, can be found in one batch that are not found in another batch. The HLA characteristics of all such batches can be categorically maintained, for example, in a database such that when a request for HLA-matched platelets is received, the database can be searched against the desired HLA characteristics matching with the recipient, and upon finding the specific batch, required number or the effective number of containers/vials of the batch can be shipped to the healthcare facility. In some embodiments, the number of batches can depend upon the most common type of HLA Class-1 antigens expressed on the platelets in the population of a geographical location. In other embodiments, the number of batches can also depend upon the HLA Class-1 antigens that are rare amongst the population of a geographical location, such that, for making a repository/bank of HLA-matched platelets expressing rare Class-1 antigens.

In some embodiments, a biomolecule profile indicative of more than 1 platelet donor can be detected by techniques not limiting to, PCR, typically, quantitative reverse transcription polymerase chain reaction (qRT-PCR). qRT-PCR is considered as one of the techniques available for quantifying RNA, such as mRNA in a sample. The RNA to be identified can be RNA specific for an individual donating the platelets, or can be used to identify platelets donated by a single donor. In illustrative embodiments, such an RNA molecule can be detected and/or quantified from a platelet sample for establishing that platelets have been donated by more than 1 individual.

The shelf-life of the freeze-dried platelet composition comprising freeze-dried platelet derivatives as disclosed herein can have a shelf-life of at least 2, 3, or 4 weeks, or 1, 2, 3, 4, 5, 6, 7, 8, 9, 10, 11, 12 months, or longer, or up to 6, 5, 4, 3, 2, or 1 year. In some embodiments, the shelf-life of the freeze-dried composition comprising freeze-dried platelet derivatives can be in the range of at least 2 weeks and up to 6 months to 4 years, 6 months to 3 years, or 6 months to 2 years, in illustrative embodiments when stored at an ambient temperature. For example, the ambient temperature can be in the range of 10-30° C., 15-30° C., or 20-30° C. The shelf-life of a freeze-dried platelet composition can be determined by numerous parameters that include in a non-limiting manner, particle counts, positivity of CD41, Annexin V, CD41, and CD62P, aggregation, and any measure of an activity of platelets. For example, shelf-life can be determined by measuring thrombin generating activity, for example by determining thrombin peak height, occlusion times using a total thrombus-formation analysis system (T-TAS), and/or thrombin generation potency units (TGPU). Example 6 of U.S. Pat. No. 11,529,587B2, incorporated herein by reference in its entirety, provides non-limiting methodologies and results obtained for the expression of surface markers CD41, AV, and CD62P on FDPDs, and thrombin peak height in FDPDs prepared as per Example 1 of the same Patent. Example 16 of U.S. Pat. No. 11,529,587B2, incorporated herein by reference in its entirety, provides a non-limiting methodology and results obtained for data regarding occlusion times obtained with the FDPDs prepared as per Example 1 of the same Patent. These same parameters and methods for determining stability can also be used to determine the stability of FDPDs after rehydration. As such, FDPDs herein can be stable for up to 8, 7, 6, 5, 4, 3, 2, or 1 month after rehydration.

The platelet derivative composition, in illustrative embodiments, HLA Class 1-characterized freeze-dried platelet derivative composition comprising HLA Class 1-characterized freeze-dried platelet derivatives as described herein are prepared from a starting material that is obtained from a plurality of donors (pooled platelet composition) having HLA Class 1-characterized platelets. As discussed hereinabove, one or more HLA characteristics (e.g. HLA Class 1 type) can be determined, identified, and/or defined before donor platelets are optionally pooled. As illustrated herein, it was surprisingly discovered that FDPDs prepared using methods provided herein, have at least some of the same HLA characteristics (e.g. HLA Class 1 type) as the platelets used to make the FDPDs. In other words, the HLA characteristics, for example, HLA Class 1 characteristics of FDPDs do not change significantly when compared to the HLA characteristics of the platelet composition or the platelet pool from which the FDPDs were prepared. Accordingly, in illustrative embodiments, FDPDs prepared from a starting material of single-donor or pooled-donor platelets, can have one or more, two or more, some, most, or all of the same HLA characteristics (e.g. HLA Class 1 antigen type) as the HLA Class 1-characterized platelets of the starting material. Accordingly, in such illustrative embodiments, the HLA characteristic of the HLA Class 1-characterized platelets of the starting material is maintained in the freeze-dried platelet derivatives. For example, a starting material comprising pooled platelets that have a defined HLA characteristic, in a hypothetical non-limiting example, such as, A1, and A3 of HLA-A, and B8, and B27 of HLA-B, used to prepare a freeze-dried platelet derivative composition herein, results in FDPDs in an FDPD composition having the same HLA characteristic(s) (e.g., HLA Class 1 type) of the starting material. Accordingly, in some embodiments, types and/or characteristics of HLA Class 1 antigens of HLA-characterized FDPDs of a batch is the sum of some, and in illustrative embodiments all the types and/or characteristics of HLA Class 1 antigens of platelets in a pool of platelets used to prepare the batch of HLA-characterized FDPDs.

The platelet derivative composition as described herein can be prepared in vessels/containers/vials that are the dried product of platelet compositions subjected to lyophilization (i.e., freeze-drying). Processes for preparing FDPDs/thrombosomes such as those that utilize TFF (e.g., U.S. Pat. No. 11,529,587 and PCT app no. PCT/US2022/079280) and/or centrifugation (e.g., WO 2006/020773, incorporated herein in its entirety) can be used. Such batch of HLA-matched FDPDs contained in vessels/containers/vials in illustrative embodiments, is labeled and stored to form a collection of HLA-matched (e.g., HLA Class 1-matched) FDPDs. Accordingly, the vessels that contain a batch of FDPDs are identifiable, for example by a label on the vessel. The location of such vial can be stored for example in a computer database that can be searched and retrieved at a later time, for example when a potential recipient in need of a hemostatic agent is identified.

In some embodiments, HLA Class 1 characteristic(s) other than HLA class 1 matching are used to select donor platelets to include in a pool of platelets. In some of these embodiments, differences between the HLA Class 1 composition of different pools can be used to construct the pools. For example, in some embodiments no HLA Class 1 antigen in any set is found in any other set. In some embodiments, the collection comprises at least 5, 6, 7, 8, or more batches. For example, in case where one of the pools used to make one of the batches comprises at least 6 platelet donors, then the selecting is performed such that the one of the pools has no more than 10, 9, 8, 7, and typically 6 HLA-A antigens and no more than 10, 9, 8, 7, and typically 6 HLA-B antigens therein. In some embodiments, one of the pools used to make one of the batches comprises at least 8 platelet donors, then the selecting is performed such that the one of the pools has no more than 14, 12, 10, and typically 8 HLA-A antigens and no more than 14, 12, 10, and typically 8 HLA-B antigens therein. In some embodiments, one of the pools used to make one of the batches comprises at least 10 platelet donors, then the selecting is performed such that the one of the pools has no more than 18, 14, 16, and typically 10 HLA-A antigens and no more than 18, 14, 16, and typically 10 HLA-B antigens therein. In some embodiments, one of the pools used to make one of the batches comprises at least 15 platelet donors, then the selecting is performed such that the one of the pools has no more than 25, 20, 18, and typically 15 HLA-A antigens and no more than 25, 20, 18, and typically 15 HLA-B antigens therein. In some embodiments, at least one of the pools used to make one of the batches can have at least 2, 3, 4, 5, 6, 7, 8, 9, or 10 platelet donors. For example, at least one of the pools used to make one of the batches can have 2-100, 2-75, 2-50, 2-25, 2-20, 2-15, 2-10, 10-100, 25-200, 50-200, 75-200, 100-200, 125-200, 150-200, or 175-200 platelet donors. In some embodiments, the methods further comprise determining the HLA Class 1 antigens of a batch of HLA Class 1-characterized FDPDs. Non-limiting methods for determining the HLA Class 1 antigens of the FDPDs or the HLA Class 1 antigens for a plurality of platelet donors include but not limited to immunological or serological assays, and nucleic acid assay, such as, molecular typing methods. In some embodiments, platelets from some of the platelet donors are not selected for any pool because of their HLA class 1 antigen characteristics. For example, platelets from some of the platelet donors are not selected for any pool because they have at least one common HLA class 1 antigen. In some embodiments, platelet donors are selected for a pool based on the frequency of their HLA Class 1 alleles in a population. For example, at least one platelet donor is selected for a pool because it has at least 1 HLA Class 1 antigen expressed from a rare allele. In other embodiments, no HLA Class 1 antigen in any set is found in any other set. In some embodiments, a pool can be made from at least 2 platelet donors, both having at least one rare allele of HLA Class 1 antigen. In some embodiments, FDPDs or platelet derivatives prepared from a pool having a rare allele of HLA Class 1 antigen can be provided to a subject in need thereof, having one of the rare alleles. In some embodiments, a pool can be prepared of at least 2, 3, 4, 5, 6, 7, 8, 9, or 10 platelet donors where each of the platelet donor has at least one rare allele of HLA Class 1 antigen. For example, the platelet donors in a pool having rare alleles can be 2-100, 2-75, 2-50, 2-25, 2-20, 2-15, 2-10, 10-100, 25-200, 50-200, 75-200, 100-200, 125-200, 150-200, or 175-200. Accordingly, there can a batch of HLA-characterized platelet derivatives or FDPDs, wherein each vessel or vial in the batch has HLA-characterized platelet derivatives or FDPDs having at least one rare allele. In illustrative embodiments, each vessel or vial in the batch has HLA-characterized platelet derivatives or FDPDs having at least one rare allele, and having a biomolecule profile indicative of more than 1 platelet donor. For example, there can be a batch of HLA-characterized FDPDs for the purpose of providing to a recipient with at least one rare HLA Class-1 allele. In such cases, the desired vessel or vial can be selected based on the methods disclosed elsewhere in the specification, in illustrative embodiments comparing to such batches of HLA-characterized platelet derivatives having at least one rare allele.

Collections or methods of making a collection herein in some embodiments include HLA-characterized platelet derivatives, or HLA-characterized FDPDs, for example, HLA Class 1-characterized FDPDs prepared from platelets of a single donor. Accordingly, in some embodiments, there can be a batch comprising 1 or more than 1 vial or vessel, each having HLA-characterized FDPDs prepared from a single platelet donor. In some embodiments, there can be more than 1 batch, such that each batch has vial(s) or vessel(s) having HLA-characterized FDPDs prepared from a single platelet donor, such that no two batches have HLA-characterized FDPDs prepared from the same platelet donor. In some embodiments, upon receiving the identity or upon characterizing the HLA type, for example, HLA Class 1 type of platelets of a donor, in illustrative embodiments, it is determined that the donor has at least one rare allele. In such embodiments, the platelets of the donor with at least one rare allele are not pooled with the platelets of other donors who do not have at least one rare allele, and is processed separately such that a set or a batch is formed with the platelets only from the single donor with a rare allele to form HLA-characterized FDPDs with at least one rare allele of a single platelet donor. In some embodiments, the rare alleles of HLA Class 1 can be a non-frequent allele in a specific population, typically, based on at least one of geographical location, ethnicity, race, or other known factors that can affect frequency of the allele. In some embodiments, the rare alleles can be polymorphic alleles with <1%, <0.75%, <0.5%, or <0.1% frequency in a given population. In some embodiments, HLA Class 1-characterized FDPDs with at least one rare allele can be used to administer a recipient, or a subject in need thereof, and having at least one rare allele. In other embodiments, upon receiving the identity or upon characterizing the HLA type, for example, HLA Class 1 type of platelets of a donor, it is determined that the platelets from the donor is to be processed separately for reasons other than the presence of a rare allele. In such embodiments, the platelets of the donor are processed separately to form HLA-characterized FDPDs from a single platelet donor.

Methods for Selecting FDPDs for a Recipient with Platelet Alloantibodies

A collection of HLA-characterized (e.g., matched) platelet derivatives for example as prepared by methods herein, which in illustrative embodiments is a collection of FDPDs, can be used herein in methods of selecting, administering, and treating disclosed herein, in illustrative embodiments that include methods of administering and/or methods for identifying HLA alloantibodies, of a recipient subject that has HPS. For example, a collection of HLA-characterized FDPDs can be used in methods for selecting HLA Class 1 compatible FDPDs for a recipient in need thereof, in illustrative embodiments who is afflicted with HPS. It is noteworthy that a “recipient”, “subject” or “patient” can be used interchangeably herein. A recipient is typically a mammalian subject, and in illustrative embodiments is a human subject. Such recipient in illustrative embodiments is in need thereof because they have anti-HLA alloantibodies and/or because they are platelet transfusion refractory. In some embodiments the recipient is platelet refractory but they do not have detectable anti-HLA alloantibodies. In some embodiments, the recipient is not refractory to platelet transfusion. Typically, alloantibodies are antibodies that recognize foreign antigens in a subject, and anti-HLA alloantibodies are antibodies that recognize HLA of platelets of a foreign subject. In some embodiments, HLA Class 1 compatible platelet derivatives or FDPDs herein are HLA characterized platelet derivatives or FDPDs that can be compatible to a recipient or a subject. For example, HLA Class 1 compatible FDPDs can be HLA Class 1 antigen-matched FDPDs to the platelets of the recipient of the subject. In some cases, HLA Class 1 compatible FDPDs can be HLA Class 1 antigen epitope-based matched to the platelets of the recipient or the subject. In some embodiments, such epitope-based matched FDPDs can be HLA Class 1 eplet-matched FDPDs, or HLA Class 1 eplet-based mismatch acceptable FDPDs. In some cases, HLA Class 1 compatible FDPDs can be matched based on the HLA Class 1 antigens of FDPDs falling within the same cross-reactive groups (CREGs) as the HLA Class 1 antigens of the platelets of the recipient or the subject. In some cases, HLA Class 1 compatible FDPDs can be HLA-characterized FDPDs against which there is no cross reactivity or least cross reactivity in a cross-match assay between an antibody-containing sample of a recipient and the HLA-characterized FDPDs. It is noteworthy in methods herein that do not explicitly recite a freeze-drying step, either FDPDs or platelet derivatives can be used. In illustrative embodiments, the platelet derivatives are not cryopreserved platelets. Cryopreserved platelets are not FDPDs. In some embodiments, methods herein include after selecting a desired batch, administering an effective dose of the HLA compatible FDPD as a single dose, or a multiple dose, for example, at least 2, 3, 4, 5, 6, 7, 8, 9, 10 dose over a span of 12 hours, 24 hours, 36 hours, 48 hours, 60 hours, 72 hours, 84 hours, 96 hours, 5 days, 6, days, 7 days, 8 days, 9 days, 10 days, or more. For example, 2-200, 2-150, 2-100, 1-75, 2-50, 2-25, 10-200, 25-200, 50-200, 100-200, or 150-200 doses. Once a desired batch of FDPD is selected a single or multiple doses as per requirement can be given from different vials or vessels of the same batch.

Such methods for selecting can utilize HLA characterization information about batches of FDPDs to select a batch of FDPDs for a recipient using HLA Class 1 virtual cross-matching. In other aspects, such methods for selecting HLA Class 1 compatible FDPDs for a recipient in need thereof can use HLA matching, which in illustrative embodiments, HLA match grades are used for such matching. In other embodiments, epitope matching is used to select a batch of FDPDs to administer to a recipient in need thereof. Furthermore, some aspects herein for selecting a batch of FDPDs to administer to a subject do not utilize previously known HLA characteristic information about a donor FDPD batch, but rather utilize an immunological physical cross-matching reaction, as discussed further herein.

Methods for selecting FDPDs herein, can include virtual cross-matching, also known as antigen-restricted method for selecting the FDPDs. For performing a virtual cross-matching of the recipient and the batches of HLA Class 1-characterized FDPDs, the information regarding the type of antibodies (for example, alloantibodies) produced in a recipient can be obtained by known techniques including, but not limited to Luminex-based phenotypic beads such as, beads coated with specific Class 1 or Class 2 HLA antigens. In some embodiments, the Luminex-based beads having only single type of antigen, such as, single-antigen bead assay (SAB) can also be used. In SAB, Luminex beads are coated with a single HLA on their surface and incubated with the serum of the recipient. If the recipient has antibodies against the specific HLA that is coated on the beads, they will bind to the bead that is coated with the respective antigen. The beads are then washed and incubated with phycoerythrin (PE)-labeled anti-human IgG antibodies. This antibody will bind to the Fc region of antibodies bound to beads. The mixture is then washed and analyzed using a Luminex instrument. Each bead is identified through a unique combination of two fluorescent dyes (red and infrared) impregnated into the bead. The anti-human IgG complex will emit fluorescence upon exposure to laser light. This fluorescence is detected on a Luminex platform and antibody reactivity is recorded as mean fluorescence intensity.

Another non-limiting example of a method for analyzing a recipient's alloantibodies includes generating a calculated panel-reactive antibody (cPRA or PRA) percentage. A cPRA can be generated by a commonly known test that employs Luminex-based phenotypic beads to screen for HLA antibodies, typically, HLAClass-1 antibodies. A cPRA provides a percentage of antibody in a test serum, typically from the recipient that are reactive against a panel of known HLA antigens. Therefore, higher cPRA percentage denotes higher number of antibodies in the serum of the recipient that are reactive against a known panel of HLA Class-1 antigens. Accordingly, in some embodiments, cPRA can be used to guide the selection and/or administration of FDPDs herein to a subject either independently or in combination with the HLA-matching between the FDPDs and the platelets of the recipient, or with the virtual cross-matching assay as disclosed herein. Accordingly, subjects, patients, or recipients in methods herein can have a PRA score of greater than 10, 15, 20, 25, 30, 40, 50, 60, 70, 75 80, 85, or 90%, or between 15% on the low end and 50, 60, 70, 75 80, 85, or 90% on the high end. In some embodiments, the subject, patient or recipient is refractory to platelet transfusions. In one illustrative embodiment, the subject, patient, or recipient has a PRA score of greater than 15% and less than 70%. In embodiment, the subject, patient, or recipient has a PRA score of greater than 70% and platelets of the subject, patient, or recipient are matched to platelet derivatives, for example FDPDs, that are administered to the subject. In embodiment, the subject, patient, or recipient has a PRA score of greater than 70% and platelets of the subject, patient, or recipient are not matched to platelet derivatives, for example FDPDs, that are administered to the subject. Once the list of HLA antigens against which the recipient produces alloantibodies is determined, the recipient can be provided the FDPDs which have at most 0, 1, 2, 3, 4, or 5 of the antigens against which the recipient produces alloantibodies. For example, among HLA Class 1 antigens, the type of HLA-A, and HLA-B against which the recipient produces the alloantibodies is determined, then the batches of HLA Class 1-characterized FDPDs can be screened for selecting a desired batch of FDPDs based on the absence of 1, 2, 3, 4 or 5 antigens recognized by alloantibodies of the recipient. In illustrative embodiments, the desired batch of FDPDs has none of the one or more allogeneic HLA Class 1 antigens recognized by the alloantibodies of the recipient.

Methods for selecting FDPDs herein, can include cross-matching the alloantibodies produced in a recipient directly with the platelet derivatives, such as FDPDs, to select cross-match compatible platelet derivatives or FDPDs. Typically, performing a cross-matching assay does not require or include identifying the specific HLA against which the recipient produces alloantibodies. In a cross-matching assay, HLA Class 1-characterized FDPDs from each of at least 2, 3, 4, 5, or more batches of a collection herein, are contacted with an antibody-containing sample of the recipient, wherein the recipient is in need of a platelet transfusion; and a desired batch of HLA Class 1-characterized FDPDs from the collection is selected based on a batch from the collection having HLA Class-1 characterized FDPDs against which there is the lowest, in illustrative embodiments, no observed cross-reaction in the cross-match assay. In some embodiments, FDPDs from each batch, wherein the number of batches range from 2-20, 2-15, or 2-10 are contacted in the cross-match assay. Cross-matching can be performed by known techniques, including, but not limited to solid-phase red cell adherence assay (SPRCA), modified antigen capture ELISA, and flow cytometry. In the SPRCA method, platelets or rehydrated FDPDs are bound to the bottom of a microtiter plate. Recipient's serum is added to each well, incubated, and washed before the addition of the AHG-coated RBCs for detecting the antigen and antibody cross-reaction.

Methods herein for selecting FDPDs herein, can include a method for matching HLA of the recipient with the batches of FDPDs. Such methods can include a step of selecting a composition of HLA-matched FDPDs, such as in a batch of HLA-matched FDPDs, that matches the HLA type of a potential recipient subject in need of a hemostatic agent, such as for example, platelets. Thus, in order to identify and select a batch of FDPDs from the collection that are an HLA match for the potential recipient (e.g., the subject in need of a hemostatic agent), HLA characteristics (e.g., HLA Class 1 antigen type) typically are, or have been determined for the potential recipient subject. For example, the HLA Class 1 type of the potential recipient can be determined.

In these embodiments, this information regarding the HLA class of the potential recipient, is transmitted or otherwise communicated to and received by an FDPD provider, manufacturer, and/or distribution center that has, stores and/or has access to information regarding the physical collection of HLA-matched FDPDs and information regarding the identity and location of vessel(s) that contain a batch of FDPDs in the collection. For example, such vessels can be stored frozen, or in illustrative embodiments, at ambient temperature, for example in a warehouse or other storage facility that meets government regulatory (e.g. FDA) requirements for such storage. Information regarding the HLA type of the potential recipient is then used to search similar information regarding batches in the collection. One or more batches with a matching HLA characteristic (e.g. HLA Class 1 type) is identified (dashed circle), if present in the collection, and selected, for example for delivery to the site of administering to the potential recipient. Thus, the selecting can be part of a method that includes shipping vessel(s) containing FDPDs of the selected batch, as part of a commercialization process to fulfill an order for such platelet derivative composition. In some embodiments, matching HLA characteristic can be matching HLA Class 1 type comprising HLA-A, HLA-B, and HLA-C antigens. In illustrative embodiments, matching HLA characteristic can be matching Class 1 type comprising HLA-A, and HLA-B antigens. A batch of platelet derivatives (e.g., FDPDs) herein can have 2, 3, 4, 5, 6, 7, 8, 9, or 10, or between 2 and 10, 9, 8, 7, 6, 5, 4, 3, or 2 HLA Class 1 antigens. In illustrative embodiments, platelet derivatives (e.g. FDPDs) FDPDs have between 2 and 4 HLA Class 1 antigens.

It is envisioned that there can be different grades of matching between the platelets of a plurality of donors forming a pooled platelet composition, or between the FDPDs herein and the platelets of a recipient. As per a standard definition of HLA-matched platelets (Mittal K K et al. Matching of histocompatibility (HL-A) antigens for platelet transfusion. Blood. 1976; 47:31-41, incorporated herein by reference in its entirety; Schmidt, Amy E et al. “HLA-Mediated Platelet Refractoriness.” American journal of clinical pathology vol. 151, 4 (2019): 353-363, incorporated herein by reference in its entirety), HLA matching can be determined for HLA-A and HLA-B antigens, and different match grades can be assigned. The HLA genes are co-dominantly expressed in an individual, therefore, an individual heterozygous for HLA genes can inherit a maximum of two alleles for each locus. Therefore, considering HLA matching of platelets for HLA-A and HLA-B genes, an individual can typically express a maximum of 4 different antigens (2 each for HLA-A and HLA-B). For example, grade “A” is assigned where there is a “4-antigen match” between the donor (or FDPDs) and recipient/subject (or other donor in case of plurality of donors for forming a pooled platelet composition). Grade “B” is assigned when there are no mismatched antigens. Grade “B” has different specific grades that can be assigned based on further classification. For example, grade “B1U” is assigned when only 3 antigens are detected in a donor (homozygous at 1 HLA allele), and all 3 antigens are identical to the recipient, grade “B1X” is assigned when out of the 3 antigens, 2 antigens are identical to the recipient and 1 antigen is cross-reactive. Grade “B2U” is assigned when only 2 antigens are detected in a donor (homozygous at 2 HLA alleles), and both the detected antigens are identical to the recipient. Grade “B2UX” is assigned when only 3 antigens are detected in the donor (homozygous at 1 HLA allele), 2 antigens are identical with the recipient, and 1 antigen is cross-reactive. Grade “B2X” is assigned when 2 antigens are identical with the recipient and 2 antigens are cross-reactive. Grade “C” is assigned when 1 antigen of donor is not present in the recipient and are not cross-reactive with the recipient. Grade “D” is assigned when 2 antigens of donor are not present in the recipient and are not cross-reactive with the recipient. Grade “R” is assigned when there is a random donor. In general, platelets with a higher degree of match have shown improved survival after platelet transfusion. For example, grades “A”, “B1U”, and “B2U” matches have been shown to provide the relative best increases in platelet counts post-transfusion (Schmidt, Amy E et al. “HLA-Mediated Platelet Refractoriness.” American journal of clinical pathology vol. 151, 4 (2019): 353-363). Platelets from donors having HLA antigens from the same cross-reactive group as the recipient's platelets have also been shown to produce better results post-transfusion (Duquesnay, R J et al. “Transfusion therapy of refractory thrombocytopenic patients with platelets from donors selectively mismatched for cross-reactive HLA antigens.” Transplantation proceedings vol. 9, 1 Suppl 1 (1977): 221-4) which can be partially due to the inability of the recipient's immune system to recognize the cross-reactive groups as foreign antigens (for example, grades “B2UX”, “B2X”). Cross-reactive groups (CREGs) have been identified in the art, for example, in the publications Rodey, G E et al. “Epitope specificity of HLA class I alloantibodies. I. Frequency analysis of antibodies to private versus public specificities in potential transplant recipients.” Human immunology vol. 39, 4 (1994): 272-80; and Wade J A, et al. “HLA mismatching within or outside of cross-reactive groups (CREGs) is associated with similar outcomes after unrelated hematopoietic stem cell transplantation”. Blood. 2007 May 1; 109(9):4064-70. Further, HLA-matched platelets with grades “B2X”, “C”, and “D” have been shown to provide platelet responses similar to transfusion of platelets from random donors.

CREGs are generally defined by sharing of a public epitope, therefore, HLA Class 1 matching can be based on cross-reactive HLA Class 1 antigens rather than matching exactly the same HLA Class 1 antigens. Accordingly, in some embodiments, platelets, or FDPDs herein can be matched by identifying the CREGs between the platelets in a platelet composition obtained from a plurality of donors, or between the freeze-dried platelet derivatives herein and the platelets of the recipient. A skilled artisan would understand that identifying CREGs can be a step that can be considered alternative to matching HLA antigens on the surface of different platelets, or freeze-dried platelet derivatives, in case there is no suitable match found between the antigens using exact matching or more stringent matching grades not based on CREG matching. Alternatively, identifying CREGs can be an independent step to assess the HLA characteristic for pooling platelets from a plurality of donors, or for matching the freeze-dried platelet derivatives to a recipient in need thereof. Accordingly, HLA-matched platelets herein can be HLA-matched because they include cross-reactive HLA antigens, typically, Class-1 antigens on the surface of platelet derivatives that fall within the same group as that of the platelets of the recipient or subject. In some embodiments, HLA-matched platelets herein can be HLA-matched for purposes of matching a recipient to FDPDs to be administered to the recipient, or for identifying donors whose platelets to include in a pool for FDPD production, because they include cross-reactive HLA Class-1 antigens, that fall within the same cross-reactive group.

In some embodiments, instead of matching entire HLA antigens between platelets of the recipient and FDPDs to be administered to the recipient, or between platelets to pool before using them to make FDPDs, HLA matching is performed, or FDPDs are HLA matched FDPDs, based on epitope matching (see e.g., Marsh et al. “An epitope-based approach of HLA-matched platelets for transfusion: a noninferiority crossover randomized trial.” Blood 2021; 137 (3): 310-32). HLA epitopes can be described or identified in the form of eplets. An eplet is a polymorphic amino acid tridimensional configuration within the epitope that is recognized by alloantibodies. In case of an HLA mismatch, a recipient can develop alloantibodies against the HLA. However, in order to produce the alloantibodies, the HLA has to be accessible at the protein level in its quaternary structure. Eplet information can provide details regarding the quaternary structure of the epitope that is recognized by alloantibodies of the recipient. Accordingly, in some embodiments, HLA typing of FDPDs from a collection herein can be compared with the HLA typing of the recipient to assess the epitope and/or eplet matching and the information can then be used to select the desired batch of FDPDs having higher number of epitope and/or eplet matching with the HLA type of the recipient. In some embodiments, the identity of HLA Class 1 antigens against which the recipient is capable of generating alloantibodies can also be used to decide the acceptability of epitope and/or eplet mismatch between FDPDs and the HLA type of the recipient.

Accordingly, in certain embodiments methods for selecting FDPDs herein include receiving an HLA Class 1 type of the recipient, wherein the recipient is in need of a platelet transfusion, and comparing the HLA Class 1 type of the recipient to the HLA Class 1 type of the batches in a collection of FDPDs, and a comparing step is performed to select a desired batch of HLA Class 1-characterized FDPDs from the collection by comparing epitopes and/or eplets of the batches of the collection with the one or more HLA Class-1 epitopes and/or eplets in the recipient. A desired batch can then be selected based on the FDPDs having higher number of epitope and/or eplet matching between FDPDs and the HLA Class-1 antigens in the recipient as compared to the FDPDs in other batches. Methods herein can further include receiving an identity of one or more HLA Class-1 epitopes recognized by alloantibodies in the recipient, and a desired batch of FDPDs can then be selected based on eplet-based mismatch acceptability of one or more allogeneic HLA Class 1 antigens. In some embodiments, the desired batch of HLA Class 1-characterized FDPDs has at least 1 mismatch of the HLA Class 1 type of the recipient. Typically, the desired batch of HLA Class 1-characterized FDPDs is premade, before a recipient in need of a hemostatic agent is identified as such.

Epitopes are conformational arrangements of one or more continuous chains of amino acids that are targeted by antibodies. The epitopes can be private, i.e., on a single antigen, or public, shared by two or more HLA antigens. Cross-reactive Groups (CREGs) can be defined by a shared public epitope and can be grouped by serologic cross-reactivity patterns. To perform epitope-based matching, a Luminex HLA Class-1 single antigen bead (SAB) assay can be coupled with an epitope generating computer instruction code (e.g., software program), for example, HLA Matchmaker (www.hlamatchmaker.net). This software program takes into account the patient's HLA antibodies and generates epitopes that are possibly recognized by these antibodies. The software identifies immunogenic epitopes represented by amino acids located within approximately 3 to 3.5 \acute{Å} radius of a polymorphic residue in antibody accessible regions of HLA antigens. This software also uses the patient's HLA typing at higher resolution levels than the serological level. Each HLA antigen has multiple epitopes that can be recognized by specific antibodies. These epitopes are characterized using three-dimensional models and despite the high polymorphism content of HLA antigens, it has become possible to determine HLA compatibility at the structural level. Each recipient has his own repertoire of self-epitopes. HLA molecules share the same sequence comprising respective amino acid motifs which bring them a certain level of similarity. If the recipient shares the same motifs creating structural similarities with the donor's antigens or the antigens of HLA Class 1-characterized FDPDs from the collection, then the patient will not develop HLA antibodies, or may develop a very limited number of, typically non-harmful HLA antibodies against the donor. This approach of epitope matching, or structural compatibility, aims to determine the number of differences between the recipient and donor or FDPDs to assess the risk of HLA antibody development. The method focuses on epitopes recognized by HLA antibodies and the determination of epitope-based mismatch acceptability for sensitized recipients considered for compatible platelet transfusions. The method converts each HLA antigen into a string of potentially immunogenic triplets and then determines which triplets on mismatched donor HLA antigens or Class 1-characterized FDPDs from the collection are shared or not shared with the recipient's HLA antigens. In other words, a cross-reactivity pattern is to identify the acceptable HLA mismatches because the information received by the cross-reactivity pattern offers a window of opportunity of finding more suitable donors for a recipient, for example, a recipient refractory to platelet transfusion. The HLA Matchmaker-based analysis of serum reactivity patterns can determine which alleles have eplets that do not react with the recipient's antibodies and these can be considered acceptable mismatches. In some embodiments, epitope-based matching can be performed by using a software not limited to HLA Matchmaker, for screening the HLA Class 1 types of HLA-characterized FDPDs in a batch or a collection herein to determine HLA-characterized FDPDs having no or least number of epitope mismatches. A person of skill in the art can understand that depending upon the policies or restrictions in a geographical location any software that is available can be used to identify epitopes (including various structural properties).

Typically, regardless of the method of selecting that is employed, and for any method of administering herein, the desired batch is premade, and thus made before the identity is received. After selecting the desired batch, the desired batch is packaged for shipment, and labeled for delivery to a location where the HLA Class 1 compatible FDPDs in the desired batch will be delivered to the recipient. For administering to the recipient, the FDPDs from the required number of vials of the desired batch can be rehydrated to form rehydrated HLA Class 1 compatible FDPDs for the recipient, and the rehydrated FDPDs can be administered to the recipient.

After one or more batches of HLA donor-matched FDPDs are identified that match or are otherwise compatible with the HLA type of the recipient, and thus are a recipient-matched, batch of donor HLA-matched FDPDs, the one or more batches, or in some embodiments one batch, is selected, and one or more vessels containing an FDPD composition from the batch is prepared for delivery/shipment. To accomplish this, one or more vessels containing FDPD compositions of the selected batch(es) are placed in a package and/or otherwise placed in a box or other shipping container. The package is then labeled with the address of the location of a site of administration of the FDPDs to the potential recipient, or other location of a hospital, infusion center or entity that controls and/or owns a property at such location. The package containing the vessels containing FDPD compositions from the selected batch(es), is then delivered to the site of administration, or another location of the entity that controls the administration. Next, typically at the facility where the FDPD composition will be administered to the potential recipient, a liquid, in illustrative embodiments water, is added to the vessel containing the FDPDs to rehydrate the FDPDs. In illustrative embodiments, such rehydrating volume is +/−20, 10, 5, 2.5, 2, or 1% of the volume in which the platelets were lyophilized in the vessel. Such rehydrating is typically performed within 2 or 1 week, or 6, 5, 4, 3, 2, or 1 day, or 12, 8, 6, 4, 3, 2, or 1 hour, or immediately before use, Then the rehydrated FDPDs are administered to the potential recipient, now the recipient.

In some illustrative embodiments, the platelets in a pooled platelet composition, which can be used to make a collection of FDPDs as disclosed herein, can be HLA-matched for HLA Class-1 antigens. The HLA Class-1 antigens that are known in the art to be present on the surface of platelets are HLA-A, HLA-B, and HLA-C. In illustrative embodiments, the platelets in the platelet composition can be HLA-matched for HLA-A and HLA-B antigens between a plurality of donors. Typically, freeze-dried platelet derivatives of a freeze-dried platelet derivative composition as disclosed herein, obtained using HLA-matched pooled platelets are also HLA-matched, for example having the same HLA-A and HLA-B antigens if the platelet donors each expressed the same HLA-A and HLA-B antigens. Methods of treating a subject herein, in some embodiments can include freeze-dried platelet derivatives that are prepared from a platelet composition obtained from a single donor as a starting material. In illustrative embodiments, such methods of treating a subject are performed using HLA-matched, freeze-dried platelet derivatives prepared using HLA-matched, pooled platelets. Methods of treating a subject herein, in some embodiments can further include HLA-matching between the platelet derivatives, for example freeze-dried platelet derivatives of a freeze-dried platelet derivative composition administered to a subject, and the platelets of the subject. In illustrative embodiments, the HLA-matching between the freeze-dried platelet derivatives and the platelets of the subject can be done for HLA Class-1 antigens. In illustrative embodiments, HLA-matching between the freeze-dried platelet derivatives and the platelets of the subject are done for HLA-A and HLA-B antigens.

As discussed in more detail herein, in some embodiments, methods herein include steps to determine whether the subject produces anti-HLA antibodies to a defined HLA antigen, in illustrative embodiments, HLA Class-1 antigens. Compositions herein, as well as methods that include or produce such compositions, including methods for administering such compositions to subjects with anti-HLA antibodies, can include a population of platelet derivatives that have no mismatch of HLA Class-1 antigens within the population of platelet derivatives. In some embodiments, the compositions herein can include a population of platelet derivatives such that the platelet derivatives have no more than two mismatches, in illustrative embodiments, no more than one mismatch of HLA Class-1 antigens, in illustrative embodiments, HLA-A and HLA-B antigens within the population of platelet derivatives.

Compositions herein, as well as methods that include or produce such compositions, can include a population of platelet derivatives that include cross-reactive HLA antigens that are in the same cross-reactive groups (CREGs) as other platelet derivatives within the population of platelet derivatives.

Compositions herein, as well as methods that include or produce such compositions, can include a population of platelet derivatives that are HLA-matched within the population of platelet derivatives, and the HLA-matched platelet derivatives have HLA-matched eplets. A skilled artisan will understand that the degree of HLA-matched eplets is subjected to variation depending upon a specific donor-recipient pair.

Compositions herein in illustrative embodiments, include platelet derivatives that are prepared using platelets from a plurality of donors (e.g. pooled platelets). Thus, compositions herein, and methods using and for preparing the same, can include a population of platelets and/or platelet derivatives that have a biomolecule profile indicative of more than 1 platelet donor. A skilled artisan will understand that there are various molecular tests that can be used to confirm that a platelet derivative composition includes platelet derivatives from a plurality of donors. In some embodiments, the biomolecule profile indicative of more than 1 platelet donor is the presence of two or more alleles/versions/amino acid sequences of at least a first protein from at least a first gene that are significantly different than 50% in frequency within the composition. A 50% frequency would be expected, for example, if such composition was from a single donor that was heterozygous for alleles at the first gene. In certain embodiments, the first protein is not an HLA Class-1 antigen, for example, not HLA-A and/or HLA-B antigens, since in illustrative embodiments platelet derivatives in a composition herein, are from a plurality of donors who are identically HLA-matched. In illustrative embodiments, the first protein is an HLA Class1 antigen, for example, an HLA-A and/or HLA-B antigen, and the plurality of donors do not have identical HLA Class 1 antigens.

The process of HLA-typing to characterize the HLA-A and HLA-B antigens on the surface of platelets, and/or freeze-dried platelet derivatives can be done by known techniques. For example, such typing methods include serological typing to determine HLA phenotypes, and HLA typing by nucleic acid-based molecular techniques. Serological HLA typing can be based on the detection of expressed HLA antigens on the surface of platelets using defined panels of antisera. Serological HLA typing can be used to determine whether a particular allele is actually expressed on the surface of platelets. Nucleic-acid based molecular techniques for HLA typing can include RNA or DNA-based HLA typing, for example mRNA typing or mitochondrial DNA typing, since such DNA has been identified in platelets. Nucleic acid-based HLA typing can employ either nucleic acid-based (e.g. DNA-based) probes or primers, to perform, for example, PCR (e.g. qPCR), or nucleic acid sequencing, for example next-generation sequencing, or test for the presence or absence of sequence motifs. Commercial kits that utilize this technology can define the HLA alleles present in an individual to a variable level of resolution dependent on a number of factors. These include the number of probes or primers employed, the number of alleles defined for a given locus and the HLA alleles present in the individual. Nucleic acid-based HLA typing can resolve allele-level differences in HLA genes that cannot be detected by serology. Several approaches to nucleic acid (e.g. DNA) based HLA typing are used that offer a range of reported typing resolution levels from low (antigen-level) to high (allele-level). A non-limiting list of techniques for DNA-based HLA typing can include PCR with sequence specific primers (PCR-SSP), Sequence specific oligonucleotide probing (PCR-SSOP), Sanger sequencing-based typing (SBT), and Next-generation sequencing. Nucleic acid-based molecular techniques for HLA typing can include techniques that exploit the presence of RNA molecules (e.g. mRNA) on a platelet product.

In some embodiments of any aspects or embodiments herein that include a method for selecting FDPDs for a recipient or subject, include methods for selecting FDPDs for a recipient from different sets or batches of HLA-characterized FDPDs, each prepared from a single platelet donor. In illustrative embodiments, the HLA-characterized FDPDs in vial(s) or vessel(s) of such sets or batches prepared from a single platelet donor have at least one rare allele. In other embodiments, the HLA-characterized FDPDs in such sets or batches prepared from a single platelet donor do not have at least one rare allele.

In some embodiments, methods herein include subjects that are refractory to platelet administration or transfusion, the refractoriness can be because of immune-related refractoriness, non-immune-related refractoriness, or idiopathic refractoriness. Accordingly, in some embodiments, subjects, or recipients who are set to receive FDPDs as disclosed herein, in illustrative embodiments, HLA-characterized FDPDs as disclosed herein are refractory to platelet transfusion. Non-immune-related refractoriness (non-limiting list) can include refractoriness due to sepsis, infection, fever, splenomegaly, disseminated intravascular coagulation (DIC), bleeding, any medications, and hepatic sinusoidal obstruction syndrome. Immune-related refractoriness can include alloimmunization that may or may not be because of prior transplantation procedures. The alloimmunization can include the presence of anti-HLA and/or anti-HPA antibodies in the blood of the subject. The course of treatment of a subject can be based on the observation of an expected amount in a target platelet increase timeframe after administering platelets, typically, platelet transfusion. The expected amount obtained after a platelet transfusion can further be correlated with the refractoriness of the subject, for example, immune-related refractoriness, non-immune-related refractoriness, or idiopathic refractoriness, and can further decide the treatment course of the subject. For example, as per one of the treatment methods, after administering platelets, typically, 2 platelet transfusions to a subject, a platelet count increment of less than 10,000/μl after one or both platelet transfusions can be indicative of immune-related refractoriness. In case of immune-related refractoriness, the subject can be checked for panel reactive antibodies (PRA), and the subject can be typed for the HLA type. In case the PRA of the subject is elevated, immune-related refractoriness can be confirmed, and the subject can be transfused with cross-matched platelets, or antigen-restricted platelets (also known as antigen-avoidance platelets), in illustrative embodiments with HLA-matched platelets. In case the PRA of the subject is not elevated, then the possibility of HPA-mediated refractoriness or non-immune-related refractoriness can be investigated. In a different scenario, for example, after administering platelets, typically, 2 platelet transfusions to a subject, in case a platelet count increment of more than 10,000/μl after both platelet transfusions are observed, then the platelet count after 24 hours post-transfusion can be checked. If the platelet count increment after 24 hours is more than 10,000/μl, then the observation can be investigated as being inconsistent with platelet refractoriness. Alternatively, if the platelet count increment after 24 hours is less than 10,000/μl, then the observation can be consistent with non-immune-related refractoriness, and the subject can be treated accordingly. In case of using CCI as the expected amount of increase in platelet counts after platelet transfusion, one of the treatment schemes can be as follows: after administering platelets, typically, 2 platelet transfusions to a subject, if CCI is less than 5,000, then HLA/HPA antibody screening can be done of the subject, and in case the presence of antibodies against HLA and/or HPA is observed in the subject's blood, then cross-matched platelets can be administered to the subject and CCI can be then monitored. If CCI remains below 5,000 then the subject can be typed for HLA, and then HLA-matched platelets, or antigen-restricted platelets (antibody avoidance platelets) can be administered to the subject. In case the CCI still remains below 5,000 then the subject can be administered random platelets. Alternatively, during the treatment an improvement in CCI post administering cross-matched platelets, HLA-matched platelets, or antigen-restricted platelets (antibody avoidance platelets) is an indication of improved platelet count and the subject can be supported by transfusing the respective platelet types. In some embodiments, methods herein include determining whether a subject or a recipient is refractory to platelet transfusions before receiving the FDPDs herein.

Accordingly, methods herein include subjects or recipients who have undergone at least one platelet transfusion before receiving FDPDs as disclosed herein, in illustrative embodiments, HLA-characterized FDPDs. Platelet transfusion in rare congenital platelet disorders such as Bernard-Soulier syndrome, Hermansky-Pudlak syndrome, Glanzmann's thrombasthenia, thrombocytopenia with absent radii (TAR), Wiskott-Aldrich syndrome, Fanconi anaemia, amegakaryocytic thrombocytopenia can provoke the development of multi-specific HLA or platelet specific antibodies. Accordingly, in some embodiments, subjects herein would have already undergone at least 1 round of platelet transfusion before receiving the FDPDs according to the methods disclosed herein. In some embodiments, a recipient or a subject has undergone at least 1 round, or more than 1 round of platelet transfusion. For example, a recipient, or a subject has undergone 1-200, 1-175, 1-150, 1-125, 1-100, 1-75, or 1-50 rounds of platelet transfusion before receiving the FDPDs herein. In some embodiments, a recipient, or a subject has undergone at least 2, 5, 10, 25, 50, 75, 100, 125, 150, 175, 200, 225, 250, 275, or 300 rounds of platelet transfusion before receiving the FDPDs herein. In some embodiments, methods herein include determining the status of platelet transfusions of a subject or a recipient before receiving the FDPDs herein.

### Quantity of Platelet or Platelet Derivatives, and Erythrocytes

The composition comprising platelet derivatives and aqueous medium can have varying quantity of platelets and/or platelet derivatives, optionally along with erythrocytes. In some embodiments, a composition as described herein can have a platelet count of at least 10⁶ (e.g., at least 5×10⁶, 10⁷, 5×10⁷, 10⁸, 5×10⁸, 10⁹, 5×10⁹, or 10¹⁰). In some embodiments, a composition as described herein can have a platelet count of at least about 200×10³ platelets/μL (e.g., at least about 300×10³, 400×10³, 500×10³, 750×10³, 1000×10³, 1500×10³, 2000×10³, or 2500×10³ platelets/μL). In some embodiments, a composition as described herein can have a platelet count of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). In some embodiments, a composition as described herein can have a platelet count less than or equal to 1000×10⁴ platelets/μL. In some embodiments, the platelets or platelet derivatives in the composition are at least 100×10³ platelets/μL, or 200×10³ platelets/μL, or 400×10³ platelets/μL, or 1000×10³ platelets/μL, or 1250×10³ platelets/μL, or 1500×10³ platelets/μL, or 1750×10³ platelets/μL, 2000×10³ platelets/μL, or 2250×10³ platelets/μL, or 2500×10³ platelets/μL, or 2750×10³ platelets/μL, or 3000×10³ platelets/μL, 3250×10³ platelets/μL, 3500×10³ platelets/μL, 3750×10³ platelets/μL, 4000×10³ platelets/μL, or 4250×10³ platelets/μL, or 4500×10³ platelets/μL, or 4750×10³ platelets/μL, or 5000×10³ platelets/μL, or 5250×10³ platelets/μL, or 5500×10³ platelets/μL, or 5750×10³ platelets/μL, or 6000×10³ platelets/μL, or 7000×10³ platelets/μL, or 8000×10³ platelets/μL, or 9000×10³ platelets/μL, or 10,000×10³ platelets/μL, or 11,000×10³ platelets/μL, or 12,000×10³ platelets/μL, or 13,000×10³ platelets/μL, or 14,000×10³ platelets/μL, or 15,000×10³ platelets/μL, or 16,000×10³ platelets/μL, or 17,000×10³ platelets/μL, or 18,000×10³ platelets/μL, or 19,000×10³ platelets/μL, or 20,000×10³ platelets/μL. In some embodiments, the platelets or platelet derivatives in the composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, a composition as provided herein can include erythrocytes. In some embodiments, a composition as provided herein can have an erythrocyte count of less than about 10¹⁰ (e.g., less than 5×10⁹, 10⁹, 5×10⁸, 10⁸, 5×10⁷, 10⁷, 5×10⁶, or 10⁶). In some embodiments, the erythrocyte count can be less than 0.2×10⁶/μL (e.g., less than 0.1×10⁶/μL, 0.5×10⁵/μL, or 0.1×10⁵/μL). In some embodiments, the erythrocytes in the composition is in the range of 0.1×10⁵ erythrocytes/μL to 0.2×10⁶ erythrocytes/μL, or 0.5×10⁵ erythrocytes/μL to 0.1×10⁶ erythrocytes/μL.

### Reduced Plasma Protein Content and Antibodies

Transfusion-related acute lung injury (TRALI) is a condition believed to be caused by the presence of antibodies (e.g., Human Leukocyte Antigen (HLA), Human Neutrophil Antigen (HNA), or granulocyte antibodies) in a transfused blood product, which can react with antigens in a transfusion recipient. Thus, the use of plasma-based blood products from donors considered to be high-risk or who test positive for Human Leukocyte Antigen (HLA) Class I, Class II, and neutrophil-specific antibodies can cause issues in transfusion or production of human-derived platelet products (e.g., compositions comprising platelets and/or platelet derivatives (e.g., thrombosomes)) and in illustrative embodiments, are therefore omitted from a donor pool.

The use of tangential flow filtration (TFF) or multi-pass centrifugation can reduce the amount of antibody in a blood product, for example, to limits not detectable by current, FDA-approved, testing methods. In some cases, reduction of certain plasma components (e.g., HLA antibodies) can allow for this donor population to be accepted for production of blood products (e.g., compositions comprising platelets and/or platelet derivatives (e.g., thrombosomes)). In some embodiments described herein, a blood product can be a composition that includes platelets or platelet derivatives and an aqueous medium.

Thrombosomes or cryopreserved platelet production can be limited by the availability of licensed apheresis collections performed at blood donor centers around the United States. Competition for these products can be fierce, and distribution for blood product manufacturing needs is usually prioritized below the needs of patient care. Blood product manufacturing (e.g., scale-up), could be aided by apheresis collections from otherwise deferred donors. One way this could be accomplished is by reducing free antibody levels in donor plasma to meet current, FDA approved, testing thresholds by utilizing tangential flow filtration (TFF) or centrifugation and plasma removal. Centrifugation of the raw materials (e.g., donor plasma), while typically more time consuming than TFF, can have a similar effect on the raw material. In some cases, removal of the donor plasma and replacement with buffer can allow the inventors to manufacture and characterize a final product (e.g., compositions comprising platelets and/or platelet derivatives (e.g., thrombosomes)) with a reduced protein (e.g., antibody (e.g., HLA antibody or HNA antibody)) content (e.g., as measured by absorbance at 280 nm). Such a product can increase the safety for a recipient of the product by reducing the transfusion related cause for TRALI.

In some embodiments, the materials and methods provided herein can allow previously deferred donors (such as those who screen positive for HLA antibodies or whose donor history presents a risk for positive HLA) to be allowed into the donor pool of raw materials used to manufacture blood products (e.g., compositions comprising platelets and/or platelet derivatives (e.g., thrombosomes)). In some embodiments described herein, a blood product can be a composition that includes platelets and an aqueous medium. Additionally, a reduction in HLA antibodies from the raw materials (e.g., donor apheresis material (e.g., platelets or pooled platelets)) can allow for a final product (e.g., compositions comprising platelets and/or platelet derivatives (e.g., thrombosomes)) to be labeled as HLA-reduced, increasing the safety of a product for a recipient.

In some embodiments, a blood product (e.g., compositions comprising platelets and/or platelet derivatives (e.g., thrombosomes)) as provided herein can have no detectable level of HLA antibodies. In some embodiments, a blood product (e.g., compositions comprising platelets and/or platelet derivatives (e.g., thrombosomes)) as provided herein can have no detectable level of an antibody selected from the group consisting of HLA Class I antibodies, HLA Class II antibodies, and HNA antibodies. In some embodiments, a blood product (e.g., compositions comprising platelets and/or platelet derivatives (e.g., thrombosomes)) as provided herein can have no detectable level of HLA Class I antibodies. In some embodiments, a blood product (e.g., compositions comprising platelets and/or platelet derivatives (e.g., thrombosomes)) as provided herein can have no detectable level of HLA Class II antibodies. In some embodiments, a blood product (e.g., compositions comprising platelets and/or platelet derivatives (e.g., thrombosomes)) as provided herein can have no detectable level of HNA antibodies. In some embodiments, detection of antibodies can be carried out using a regulatory agency approved (e.g., FDA cleared) assay. A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software. In some embodiments described herein, a blood product can be a composition that includes platelets and an aqueous medium.

In some embodiments, a blood product (e.g., compositions comprising platelets and/or platelet derivatives (e.g., thrombosomes)) as provided herein can have a level of an antibody selected from the group consisting of HLA Class I antibodies, HLA Class II antibodies, and HNA antibodies below a reference level. In some embodiments, a blood product (e.g., compositions comprising platelets and/or platelet derivatives (e.g., thrombosomes)) as provided herein can have a level of HLA Class I antibodies below a reference level. In some embodiments, a blood product (e.g., compositions comprising platelets and/or platelet derivatives (e.g., thrombosomes)) as provided herein can have a level of HLA Class II antibodies below a reference level. In some embodiments, a blood product (e.g., a composition comprising platelets and/or platelet derivatives (e.g., thrombosomes)) as provided herein can have a level of HNA antibodies below a reference level. A reference level can be any appropriate reference level. In some embodiments described herein, a blood product can be a composition that includes platelets and an aqueous medium.

In some embodiments, a blood product (e.g., a composition comprising platelets and/or platelet derivatives (e.g., thrombosomes)) as provided herein test negative for an antibody selected from the group consisting of HLA Class I antibodies, HLA Class II antibodies, and HNA antibodies in a regulatory agency approved assay (e.g., an FDA cleared assay). In some embodiments, a blood product (e.g., a composition comprising platelets and/or platelet derivatives (e.g., thrombosomes)) as provided herein can test negative for HLA Class I antibodies in a regulatory agency approved assay (e.g., an FDA cleared assay). In some embodiments, a blood product (e.g., a composition comprising platelets and/or platelet derivatives (e.g., thrombosomes)) as provided herein can test negative for HLA Class II antibodies a in regulatory agency approved assay (e.g., an FDA cleared assay). In some embodiments, a blood product (e.g., a composition comprising platelets and/or platelet derivatives (e.g., thrombosomes)) as provided herein can test negative for HNA antibodies in a regulatory agency approved assay (e.g., an FDA cleared assay). In some embodiments described herein, a blood product can be a composition that includes platelets and an aqueous medium. A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some aspects, provided herein are compositions comprising platelets and/or platelet derivatives (e.g., thrombosomes) and an aqueous medium. In some embodiments, the aqueous medium can include a preparation agent (e.g., any of the preparation agents described herein). In some embodiments, an aqueous medium as provided herein can have a level of an antibody selected from the group consisting of HLA Class I antibodies, HLA Class II antibodies, and HNA antibodies below a reference level. In some embodiments, an aqueous medium as provided herein can have a level of HLA Class I antibodies below a reference level. In some embodiments, an aqueous medium as provided herein can have a level of HLA Class II antibodies below a reference level. In some embodiments, an aqueous medium as provided herein can have a level of HNA antibodies below a reference level. A reference level can be any appropriate reference level. In some embodiments, an aqueous medium as provided herein can test negative for an antibody selected from the group consisting of HLA Class I antibodies, HLA Class II antibodies, and HNA antibodies in a regulatory agency approved assay (e.g., an FDA cleared assay). In some embodiments, an aqueous medium as provided herein can test negative for HLA Class I antibodies in a regulatory agency approved assay (e.g., an FDA cleared assay). In some embodiments, an aqueous medium as provided herein can test negative for HLA Class II antibodies in a regulatory agency approved assay (e.g., an FDA cleared assay). In some embodiments, an aqueous medium as provided herein can test negative for HNA antibodies in a regulatory agency approved assay (e.g., an FDA cleared assay). A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some embodiments, an aqueous medium can have a reduced amount of residual plasma compared to donor apheresis plasma (e.g., single-donor apheresis plasma or pooled donor apheresis plasma) can be a percentage of residual plasma (e.g., less than or equal to about 50%, 40%, 30%, 20%, 150%, 10%, 9%, 8%, 7%, 6%, 5%, 4%, 3%, 2%, 10%, 0.9%, 0.8%, 0.7%, 0.6%, 0.50%, 0.4%, 0.30%, 0.2%, or 0.10% of residual plasma). In some embodiments, an aqueous medium can have a reduced amount of residual plasma compared to donor apheresis plasma (e.g., single-donor apheresis plasma or pooled donor apheresis plasma) can be a percentage range of residual plasma (e.g., about 5% to about 50%, about 5% to about 40%, about 5% to about 30%, about 5% to about 20%, about 5% to about 15%, about 5% to about 10%, about 10% to about 20%, about 7% to about 15%, about 7% to about 10%, about 8% to about 15%, about 8% to about 10%, about 0.1% to about 5%, about 0.1% to about 3%, about 0.1% to about 1%, about 0.5% to about 3%, about 0.5% to about 1%, or about 1% to about 3% of residual plasma). In some embodiments, an aqueous medium can have a protein concentration less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the protein concentration of donor apheresis plasma (e.g., single-donor apheresis plasma or pooled donor apheresis plasma). In some embodiments, an aqueous medium can have a protein concentration of about 5% to about 50% (e.g., about 5% to about 40%, about 5% to about 30%, about 5% to about 20%, about 5% to about 15%, about 5% to about 10%, about 10% to about 20%, about 7% to about 15%, about 7% to about 10%, about 8% to about 15%, or about 8% to about 10%) of the protein concentration of donor apheresis plasma (e.g., single-donor apheresis plasma or pooled donor apheresis plasma). In some embodiments, an aqueous medium can have a protein concentration of about 0.10% to about 50% (e.g., about 0.10% to about 30%, about 0.10% to about 1%, about 0.5% to about 3%, about 0.5% to about 1%, about 1% to about 2%, or about 1% to about 3%) of the protein concentration of donor apheresis plasma (e.g., single-donor apheresis plasma or pooled donor apheresis plasma). A protein concentration can be measured by any appropriate method. Apart from the relative protein concentration of proteins in the aqueous medium, the protein concentration in the aqueous medium can also be measured in absolute terms. Accordingly, in some embodiments, a protein concentration can be measured by absorbance at 280 nm (A280). In some embodiments, an aqueous medium can have an A280 that is less that is less than 2.0 AU (e.g., less than 1.97, 1.95, 1.93, 1.90, 1.87, 1.85, 1.83, 1.80, 1.77, 1.75, 1.73, 1.70, 1.66, 1.6, 1.5, 1.4, 1.3, 1.2, 1.1, 1.0, 0.9, 0.8, 0.7, 0.6, 0.5, 0.4, 0.3, 0.2, or 0.1 AU) with a path length of 0.5 cm. In some embodiments, the protein concentration in the aqueous medium is less than or equal to 15%, 14%, 13%, 12%, 11%, 10%, 9%, 8%, 7%, 6%, 5%, 4%, 3%, 2%, 1%, 0.1%, or 0.01%. In some illustrative embodiments, the protein concentration is less than 3% or 4%. In some embodiments, the protein concentration is in the range of 0.01-15%, 0.1-15%, 1-15%, 1-10%, 0.1-10%, 0.01-10%, 0.1-5%, 0.1-4%, 0.1-3%, 0.1-2%, 0.1-1%, 1-5%, 1-4%, 1-3%, 1-2%, 3-12%, or 5-10%. In some embodiments, an aqueous medium can have a HLA Class I antibody concentration less than about 70% (e.g., less than about 60%, 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HLA Class I antibody concentration of donor apheresis plasma (e.g., single-donor apheresis plasma or pooled donor apheresis plasma). A HLA Class I antibody concentration can be measured by any appropriate method. In some embodiments, the HLA class I antibody concentration in the aqueous medium can be quantitated in absolute terms such that the aqueous medium can have HLA Class I antibody concentration less than about 70% (e.g., less than about or less than exactly 60%, 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.10%) in the aqueous medium. In some embodiments, the HLA Class I antibody in the aqueous medium is low enough such that the composition comprising platelet derivatives and aqueous medium is negative for HLA Class I antibodies based on a regulatory agency approved test for HLA Class I antibodies.

In some embodiments, an aqueous medium can have a HLA Class II antibody concentration less than about 50% (e.g., less than about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HLA Class II antibody concentration of donor apheresis plasma (e.g., single-donor apheresis plasma or pooled donor apheresis plasma). A HLA Class II antibody concentration can be measured by any appropriate method. In some embodiments, the HLA class II antibody concentration in the aqueous medium can be quantitated in absolute terms such that the aqueous medium can have HLA Class I antibody concentration less than about 50% (e.g., less than about or less than exactly 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) in the aqueous medium. In some embodiments, the HLA Class II antibody in the aqueous medium is low enough such that the composition comprising platelet derivatives and aqueous medium is negative for HLA Class II antibodies based on a regulatory agency approved test for HLA Class II antibodies.

In some embodiments, an aqueous medium can have a HNA antibody concentration less than about 50% (e.g., less than about 40%, 30%, 20%, 15%, 10%, 9% 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HNA antibody concentration of donor apheresis plasma (e.g., single-donor apheresis plasma or pooled donor apheresis plasma). A HNA antibody concentration can be measured by any appropriate method. In some embodiments, the HNA antibody concentration in the aqueous medium can be quantitated in absolute terms such that the aqueous medium can have HNA antibody concentration less than about 50% (e.g., less than about or less than exactly 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) in the aqueous medium. In some embodiments, the HNA antibody in the aqueous medium is low enough such that the composition comprising platelet derivatives and aqueous medium is negative for HNA antibodies based on a regulatory agency approved test for HNA antibodies.

In some cases, flow cytometry can be used to evaluate compositions as provided herein. In some embodiments, an antibody selected from the group consisting of HLA Class I antibodies, HLA Class II antibodies, and HNA antibodies, as determined for a composition comprising platelets and an aqueous medium by flow cytometry using beads coated with Class I HLAs, Class II HLAs, or HNAs, respectively, is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class I antibodies, as determined for a composition comprising platelets and an aqueous medium by flow cytometry using beads coated with Class I HLAs, is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class II antibodies, as determined for a composition comprising platelets and an aqueous medium by flow cytometry using beads coated with Class II HLAs is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HNA antibodies, as determined for a composition comprising platelets and an aqueous medium by flow cytometry using beads coated with HNAs is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%).

In some embodiments, an antibody selected from the group consisting of HLA Class I antibodies, HLA Class II antibodies, and HNA antibodies, as determined for an aqueous medium by flow cytometry using beads coated with Class I HLAs, Class II HLAs, or HNAs, respectively, is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class I antibodies, as determined for an aqueous medium by flow cytometry using beads coated with Class I HLAs, is less than 10% (e.g., less than 9% 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class II antibodies, as determined for an aqueous medium by flow cytometry using beads coated with Class II HLAs is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HNA antibodies, as determined for an aqueous medium by flow cytometry using beads coated with HNAs is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%).

In some embodiments, the protein concentration is low enough to exclude the HLA Class I, HLA Class II, and HNA antibodies from the donor pheresis plasma such that the composition is negative for: a) HLA Class I antibodies based on a regulatory agency approved test for HLA Class I antibodies; b) HLA Class II antibodies based on a regulatory agency approved test for HLA Class II antibodies; and c) HNA antibodies based on a regulatory agency approved test for HNA antibodies. The platelet derivative composition of the present disclosure, and the process for obtaining the same, thus provides the flexibility to utilize the apheresis plasma from different multiple donors, and yet obtain a final product (platelet derivative composition) that is negative for the HLA Class I, HLA Class II, and HNA antibodies.

In some aspects, provided herein are platelet derivative compositions comprising platelet derivatives in the form of a solid, a composition with less than 1% water, and/or a powder. The composition in solid form, in illustrative embodiments dried form, for example with less than 1% water, can be one amongst different kinds in which the composition would be packed and commercialized. Thus, it is contemplated that the composition in the dried form would preserve the characteristics with respect to the low content, or even absence of detectable HLA Class I, HLA Class II, and HNA antibodies as described with respect to the aqueous medium in the embodiments described herein. In some embodiments, the platelet derivative composition in the form of a powder is negative for HLA Class I antibodies based on a regulatory agency approved test for HLA Class I antibodies. In some embodiments, the platelet derivative composition in the form of a powder is negative for HLA Class II antibodies based on a regulatory agency approved test for HLA Class II antibodies. In some embodiments, the platelet derivative composition in the form of a powder is negative for HNA antibodies based on a regulatory agency approved test for HNA antibodies. In some embodiments, the platelet derivative composition in the form of a powder is negative for HLA Class I antibodies, HLA Class II antibodies, and HNA antibodies based on a regulatory agency approved test for HLA Class I antibodies, HLA Class II antibodies, and HNA antibodies, respectively.

### Preparation Agent and Additional Components

In some embodiments, a composition provided herein can include one or more additional components. In some embodiments, a composition provided herein can include a preparation agent (e.g., any of the preparation agents described herein). In some embodiments, the composition can include a buffering agent, a base, a loading agent, optionally a salt, and optionally at least one organic solvent. A buffering agent can be any appropriate buffering agent. In some embodiments, a buffering agent can be HEPES (4-(2-hydroxyethyl)-1-piperazineethanesulfonic acid). A base can be any appropriate base. In some embodiments, a base can be sodium bicarbonate. A loading agent can be any appropriate loading agent. In some embodiments, a loading agent can be a monosaccharide, a polysaccharide, or a combination thereof. In some embodiments, a loading agent can be selected from the group consisting of sucrose, maltose, trehalose, glucose, mannose, and xylose. In some embodiments, a loading agent can be trehalose. In some embodiments, a polysaccharide can be polysucrose. A salt can be any appropriate salt. In some embodiments, a salt can be sodium chloride, potassium chloride, or a combination thereof. An organic solvent can be any appropriate organic solvent. In some embodiments, an organic solvent can be selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

A preparation agent can include any appropriate components. In some embodiments, the preparation agent may comprise a liquid medium. In some embodiments the preparation agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the preparation agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the preparation agent includes from about 0.5 mM to about 100 mM of the one or more salts. In some embodiments, the preparation agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) of the one or more salts. In some embodiments, the preparation agent includes about 5 mM, about 75 mM, or about 80 mM of the one or more salts. In some embodiments, the preparation agent comprises one or more salts selected from calcium salts, magnesium salts, and a combination of the two, in a concentration of about 0.5 mM to about 2 mM.

Preferably, these salts are present in the composition comprising platelets or platelet derivatives, such as freeze-dried platelets, at an amount that is about the same as is found in whole blood.

In some embodiments, the preparation agent further comprises a carrier protein. In some embodiments, the carrier protein comprises human serum albumin, bovine serum albumin, or a combination thereof. In some embodiments, the carrier protein is present in an amount of about 0.05% to about 1.0% (w/v).

The preparation agent may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the preparation agent includes one or more buffers, e.g., N-2-hydroxyethylpiperazine-N′-2-ethanesulfonic acid (HEPES), or sodium-bicarbonate (NaHCO₃). In some embodiments, the preparation agent includes from about 5 to about 100 mM of the one or more buffers. In some embodiments, the preparation agent includes from about 5 to about 50 mM (e.g., from about 5 mM to about 40 mM, from about 8 mM to about 30 mM, about 10 mM to about 25 mM) about of the one or more buffers. In some embodiments, the preparation agent includes about 10 mM, about 20 mM, about 25 mM, or about 30 mM of the one or more buffers. In some embodiments, buffers herein can have a pH in the range of 6.0 to 8.5, 6.2 to 8.5, 6.4 to 8.5, 6.0 to 7.5, or 6.0 to 7.0.

In some embodiments, the preparation agent includes one or more saccharides, such as monosaccharides and disaccharides, including sucrose, maltose, trehalose, glucose, mannose, dextrose, and xylose. In some embodiments, the saccharide is a monosaccharide. In some embodiments, the saccharide is a disaccharide. In some embodiments, the saccharide is a monosaccharide, a disaccharide, or a combination thereof. In some embodiments, the saccharide is a non-reducing disaccharide. In some embodiments, the saccharide is sucrose, maltose, trehalose, glucose (e.g., dextrose), mannose, or xylose. In some embodiments, the saccharide comprises trehalose. In some embodiments, the preparation agent comprises a starch. In some embodiments, the preparation agent includes polysucrose, a polymer of sucrose and epichlorohydrin. In some embodiments, the preparation agent includes from about 10 mM to about 1,000 mM of the one or more saccharides. In some embodiments, the preparation agent includes from about 50 to about 500 mM of the one or more saccharides. In some embodiments, one or more saccharides is present in an amount of from 10 mM 10 to 500 mM. In some embodiments, one or more saccharides is present in an amount of from 50 mM to 200 mM. In some embodiments, one or more saccharides is present in an amount from 100 mM to 150 mM. In some embodiments, the one or more saccharides are the lyophilizing agent; for example, in some embodiments, the lyophilizing agent comprises trehalose, polysucrose, or a combination thereof. In some embodiments, the preparation agent comprises trehalose in the range of 0.4-35%, or 1-35%, or 2-30%, or 1-10%, or 1-5%, or 0.5-5%. In an exemplary embodiment, the composition comprises 3.5% trehalose. In some embodiments, the preparation agent comprises polysucrose in the range of 2-8%, or 2.25-7.75%, or 2.5-7.5%, or 2.5-6.5%, wherein the composition is in a rehydrated form. In an exemplary embodiment, the composition comprises 3% polysucrose. In another exemplary embodiment, the composition comprises 6% polysucrose. Different ionic forms of polysucrose can be used in the preparation agent that would be used to lyophilize the platelet derivatives. The ionic forms of polysucrose can be exploited to increase the efficiency of the lyophilization process. The ionic forms can be optimized to accommodate higher concentrations of platelet concentrations in the solution for performing lyophilization process. In some embodiments of the composition, wherein the composition comprises polysucrose, the polysucrose is a cationic form of polysucose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. Polysucrose of different molecular weight can be used to increase the efficiency of the lyophilization process. In some embodiments of the composition, polysucrose has a molecular weight in the range of 70,000 MW to 400,000 MW. In some embodiments, polysucrose has a molecular weight in the range of 80,000 MW to 350,000 MW, or 100,000 MW to 300,00 MW. In some exemplary embodiments, polysucrose has a molecular weight in the range of 120,000 MW to 200,000 MW. In some exemplary embodiments, polysucrose has a molecular weight of 150,000 MW, or 160,000 MW, or 170,000 MW, or 180,000 MW, 190,000 MW, or 200,000 MW.

In some embodiments the composition comprising platelets or platelet derivatives, (e.g., thrombosomes), may comprise one or more of water or a saline solution. In some embodiments the composition comprising platelets or platelet derivatives, such as freeze-dried platelets, may comprise DMSO.

In some embodiments, the preparation agent comprises an organic solvent, such as an alcohol (e.g., ethanol). In such a preparation agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. In some embodiments, the organic solvent comprises ethanol, DMSO, or a combination thereof. The presence of organic solvents, such as ethanol, can be beneficial in the processing of platelets, platelet derivatives, or thrombosomes (e.g., freeze-dried platelet derivatives).

In some embodiments the preparation agent does not include an organic solvent. In some embodiments, the preparation agent comprises an organic solvent. In some embodiments the preparation agent comprises DMSO.

A preparation agent can have any appropriate pH. For example, in some embodiments, a preparation agent can have a pH of about 5.5 to about 8.0 (e.g., about 6.5 to about 6.9, or about 6.6 to about 6.8). In some embodiments, the preparation agent has a pH in the range of 5.5 to 8.0, or 6.0 to 8.0, or 6.0 to 7.5. In an exemplary embodiment, the preparation agent has a pH of 6.5. In another exemplary embodiment, the preparation agent has a pH of 7.4.

In some embodiments, one or more other components may be combined with in the platelets (e.g., as part of a preparation agent). Exemplary components may include Prostaglandin E1 or Prostacyclin and or EDTA/EGTA to prevent platelet aggregation and activation.

In some embodiments, a preparation agent can be Buffer A, as shown in Example 1 of U.S. Pat. No. 11,529,587 and Example 1 of PCT app no. PCT/US2022/079280, incorporated by reference herein in their entirety. In some embodiments, a preparation agent can comprise Buffer A, as shown in this Example 1 wherein one or more components (e.g., ethanol) is present in an amount up to three times the amount shown in this Example 1. Non-limiting examples of preparation agent compositions that may be used are shown in Tables P1-P6.

TABLE P1
Buffer

| | Concentration (mM unless |
|---|---|
| Component | otherwise specified) |
| NaCl | 75.0 |
| KCl | 4.8 |
| HEPES | 9.5 |
| NaHCO₃ | 12.0 |
| Dextrose | 3 |
| Trehalose | 100 |
| Ethanol | 1% (v/v) |

Table P1. A preparation agent that can be used

TABLE P2
Buffer

| | Concentration (mM unless |
|---|---|
| Component | otherwise specified) |
| NaCl | 75.0 |
| KCl | 4.8 |
| HEPES | 9.5 |
| NaHCO₃ | 12.0 |
| Dextrose | 3 |
| Trehalose | 100 |

Table P2. A preparation agent that can be used

TABLE P3
Buffer A1 (pH 6.5)

| | Concentration (mM unless |
|---|---|
| Component | specified otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |

Table P3. A preparation agent that can be used.

TABLE P4
Buffer B

| | Concentration (mM unless |
|---|---|
| Component | otherwise specified) |
| Buffer and Salts | Table P5 (below) |
| BSA | 0.35% |
| Dextrose | 5 |
| pH | 7.4 |

Table P4. Buffer B can used when incubating platelets, e.g., for flow cytometry. Such an incubation can be done at room temperature in the dark. Albumin is an optional component of Buffer B.

TABLE P5
Concentration of HEPES and of Salts in Buffer B

| | Concentration (mM unless |
|---|---|
| Component | otherwise specified) |
| HEPES | 25 |
| NaCl | 119 |
| KCl | 5 |
| CaCl₂ | 2 |
| MgCl₂ | 2 |
| glucose | 6 g/l |

Table P5 shows the concentrations of HEPES and salts in Buffer B. The pH can be adjusted to 7.4 with NaOH. Albumin is an optional component of Buffer B.

TABLE P6
Tyrode's HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostagalandin E1 (PGE1) | 1 μg/ml |

Table P6 is another exemplary preparation agent.

### Rehydration of the Composition Comprising Platelet Derivatives

In some aspects, the platelet derivative composition of the present disclosure is in the form of a powder. In some aspects, the process for preparing the platelet derivative composition results in the final product which is in a dry powdered form. The platelet derivative composition in its dry form comprises platelet derivatives. The platelet derivative composition in its dry form comprises platelet derivatives, and/or freeze-dried platelets. It is well-known to a skilled artisan that the platelet derivatives in the dried form shall preserve the characteristics which it is intended to observe once the platelet derivatives are rehydrated for clinical application and/or studying the characteristics such as, the presence of platelet activation markers.

In some embodiments, rehydrating the composition comprising platelets or platelet derivatives comprises adding to the platelets an aqueous liquid. In some embodiments, the aqueous liquid is water. In some embodiments, the aqueous liquid is an aqueous solution (e.g., a buffer). In some embodiments, the aqueous liquid is a saline solution. In some embodiments, the aqueous liquid is a suspension.

In some embodiments, the platelets or platelet derivatives (e.g., thrombosomes) have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets or platelet derivatives (e.g., thrombosomes), have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, rehydrating the composition comprising platelets or platelet derivatives comprises adding to the platelets or platelet derivatives sterile water (e.g., sterile water for injection) over about 10 minutes at about room temperature. In general, the rehydration volume is about equal to the volume used to fill each vial of platelet derivative composition prior to drying, for example, freeze-drying.

### Process for Preparing a Platelet Derivative Composition

In some embodiments, the platelets or pooled platelets can be initially diluted, further diluted (e.g. if initially diluted in an acidified buffer) or suspended in a preparation agent as described herein before being loaded onto a TFF unit to exchange the solution, buffer or diluted preparation agent with a preparation agent in the TFF unit. A skilled artisan would understand that before being loaded onto a TFF unit, the input composition can be initially diluted to a desirable dilution in order to carry out the TFF process in an effective manner. In some embodiments, the platelets or pooled platelets comprised in a composition can be diluted with an acidified washing buffer for example, and/or with a preparation agent as described herein before loading onto a TFF unit. In some embodiments, the platelets or pooled platelets are diluted 1:0.5, 1:1, 1:2, 1:4, 1:5, or 1:10, in a preparation agent, which in illustrative embodiments is the preparation in which the platelets will be freeze dried. In illustrative embodiments, the platelets or the pooled platelets can be diluted or suspended in a preparation agent comprising trehalose and in illustrative embodiments polysucrose before being loaded onto a TFF unit, followed by performing TFF with the preparation agent in the TFF unit. In illustrative embodiments, the platelets or the pooled platelets can be diluted or suspended in a preparation agent comprising 0.4 to 35% trehalose and 2% to 8% polysucrose, before being loaded onto a TFF unit, followed by performing TFF with the preparation agent in the TFF unit. A skilled artisan would understand that performing TFF is a continuous process of fluid exchange between the preparation agent and the platelets or the pooled platelets. The preparation agent that is used to dilute or suspend the platelets or the pooled platelets before being loaded onto a TFF unit can be the same preparation agent that is used for performing the TFF or it can be a different solution (e.g., acidified washing buffer) typically that is compatible with processing viable platelets. In some embodiments, the preparation agent used to dilute or suspend the platelets or the pooled platelets before being loaded onto a TFF unit can have the same ingredients but differ in the concentration of the ingredients than the preparation agent used for performing the TFF. A skilled artisan would understand the extent of the difference, if at all needed, based upon the dilution required to perform the TFF.

In some embodiments, the platelets or pooled platelets may be acidified to a pH of about 5.5 to about 8.0 prior to TFF or being diluted with the preparation agent. In some embodiments, the method comprises acidifying the platelets to a pH of about 6.5 to about 6.9. In some embodiments, the method comprises acidifying the platelets to a pH of about 6.6 to about 6.8. In some embodiments, the method comprises acidifying the platelets to a pH of about 6.6 to 7.5. In some embodiments, the acidifying comprises adding to the pooled platelets a solution comprising Acid Citrate Dextrose (ACD).

In some embodiments, the platelets are isolated prior to the step comprising tangential flow filtration (TFF) or being diluted with the preparation agent. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, platelets are isolated, for example in a liquid medium, prior to treating a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein. The platelets can be pooled wherein the platelets are donated by human subjects. In some other embodiments, the donor and the recipient for any aspect herein are non-human animal subjects. In some embodiments, the donor can be a canine subject. In some embodiments, the donor and the recipient for any aspect herein are equine subjects. In some embodiments, the donor and the recipient for any aspect herein are feline subjects. In illustrative embodiments the donor and the recipient for any aspect herein are human subjects.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PSCs), including embryonic stem cells (ESCs) and/or induced pluripotent stem cells (iPSCs).

Accordingly, in some embodiments, platelets or platelet derivatives (e.g., thrombosomes) are prepared prior to treating a subject as described herein. In some embodiments, the platelets or platelet derivatives (e.g., thrombosomes) are lyophilized. In some embodiments, the platelets or platelet derivatives (e.g., thrombosomes) are cryopreserved. For example, in some embodiments, the platelets or platelet derivatives can be cryopreserved in plasma and DMSO (e.g., 3-9% DMSO (e.g., 6% DMSO)). In some embodiments, the platelets or platelet derivatives are cryopreserved as described in U.S. Patent Application Publication No. 2020/0046771 A1, published on Feb. 13, 2020, incorporated herein by reference in its entirety.

In some embodiments, platelets (e.g., apheresis platelet, platelets isolated from whole blood, pooled platelets, or a combination thereof) form a suspension in a preparation agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, the method further comprises drying the platelets or platelet derivatives (e.g., thrombosomes). In some embodiments, the drying step comprises lyophilizing the platelets or platelet derivatives (e.g., thrombosomes). In some embodiments, the drying step comprises freeze-drying the platelets or platelet derivatives (e.g., thrombosomes). In some embodiments, the method further comprises rehydrating the platelets or platelet derivatives (e.g., thrombosomes) obtained from the drying step.

In some embodiments, the platelets or platelet derivatives (e.g., thrombosomes) are cold stored, cryopreserved, or lyophilized (e.g., to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table LA. Additional exemplary lyophilization methods can be found in U.S. Pat. Nos. 7,811,558, 8,486,617, and 8,097,403. An exemplary spray-drying method includes: combining nitrogen, as a drying gas, with a preparation agent according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE LA
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P | | | | | | 0 |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | Sl | 27° | C. | Hold | >8 | Hrs | 0 mT |

In some embodiments, the step of drying the platelets or platelet derivatives (e.g., thrombosomes) that are obtained as disclosed herein, such as the step of freeze-drying the platelets and/or platelet derivatives that are obtained as disclosed herein, comprises incubating the platelet and/or platelet derivatives with a lyophilizing agent (e.g., a non-reducing disaccharide). Accordingly, in some embodiments, the methods for preparing platelets and/or platelet derivatives further comprises incubating the platelets with a lyophilizing agent. In some embodiments the lyophilizing agent is a saccharide. In some embodiments the saccharide is a disaccharide, such as a non-reducing disaccharide.

In some embodiments, the platelets and/or platelet derivatives are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%. In some embodiments, polysucrose is used in the range of 2% to 8%%, or 2.25-7.75%, or 2.5-7.5%, or 2.5-6.5%. In an exemplary embodiment, the composition comprises 3% polysucrose. In another exemplary embodiment, the composition comprises 6% polysucrose. In some embodiments of the composition, wherein the composition comprises polysucrose, the polysucrose is a cationic form of polysucose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 Da to 400,000 Da. In some embodiments, polysucrose has a molecular weight in the range of 80,000 Da to 350,000 Da, or 100,000 Da to 300.00 Da. In some exemplary embodiments, polysucrose has a molecular weight in the range of 120,000 Da to 200,000 Da. In some exemplary embodiments, polysucrose has a molecular weight of 150,000 Da, or 160,000 Da, or 170,000 Da, or 180,000 Da, 190,000 Da, or 200,000 Da.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In some embodiments, the composition comprises trehalose in the range of 0.4-35%, or 1-35%, or 2-30%, or 1-10%, or 1-5%, or 0.5-5%. In an exemplary embodiment, the composition comprises 3.5% trehalose.

In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

In some cases, preparation of thrombosomes further comprises one or more of the procedures described in U.S. Pat. Nos. 8,486,617 (such as, e.g., Examples 1-5) and 8,097,403 (such as, e.g., Examples 1-3), incorporated herein by reference in their entirety. In some cases, a starting material (e.g., one or more donor platelet units) are initially pooled into a common vessel. In some embodiments, a starting material can comprise one or more donor platelet units. In some embodiments, a starting material can comprise donor plasma. The starting material may or may not be acidified with an anti-coagulation buffer (i.e. ACD-A) before centrifugation. Plasma can be aspirated off of the platelet pellet after centrifugation. Cell compatible buffer containing cryoprotectants (e.g., a loading buffer, which can be similar to or the same as a preparation agent) can be added to the platelet pellet before resuspending the cells into suspension. Platelets may or may not be diluted to a pre-determined concentration (e.g., 2200 k/ul to 2800 k/ul) with buffer if desired. Platelets in buffer may be incubated between 0 minutes and 240 minutes at an incubation temperature from 18° C. to 37° C. A lyoprotectant bulking agent (e.g., polysucrose) can be added to the platelets in buffer to achieve a final bulking agent concentration from 1% to 10% w/v (with preference at 6% w/v). The centrifuged processed platelets can then be filled into vials, lyophilized and thermally treated.

### Platelet Derivatives and Microparticles

Platelet derivatives herein have been observed to have numerous surprising properties, as disclosed in further detail herein. It will be understood, as illustrated in the Examples of U.S. Pat. No. 11,529,587 and PCT app no. PCT/US2022/079280, that although platelet derivatives in some aspects and embodiments are in a solid, such as a powder form, the properties of such platelet derivatives can be identified, confirmed, and/or measured when a composition comprising such platelet derivatives is in liquid form.

A skilled artisan would be well-versed with different techniques that are available for measuring particle sizes of platelets, platelet derivatives or FDPDs, and microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles.

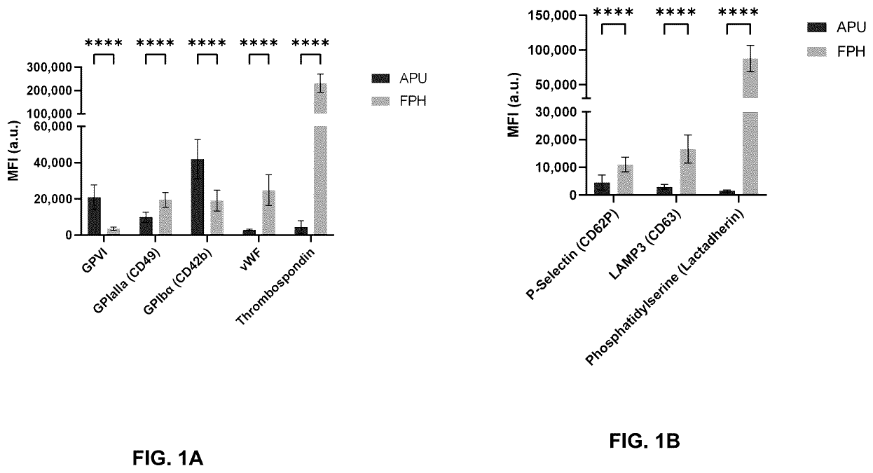

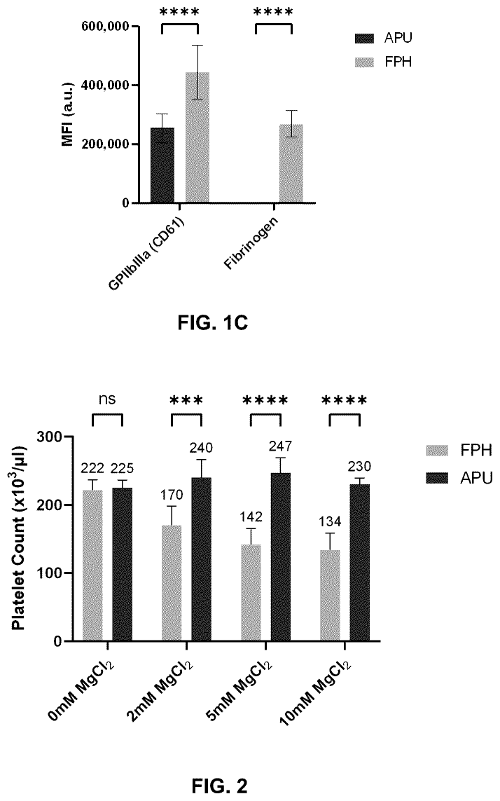

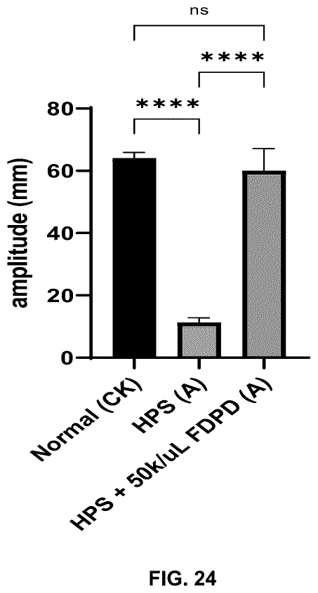

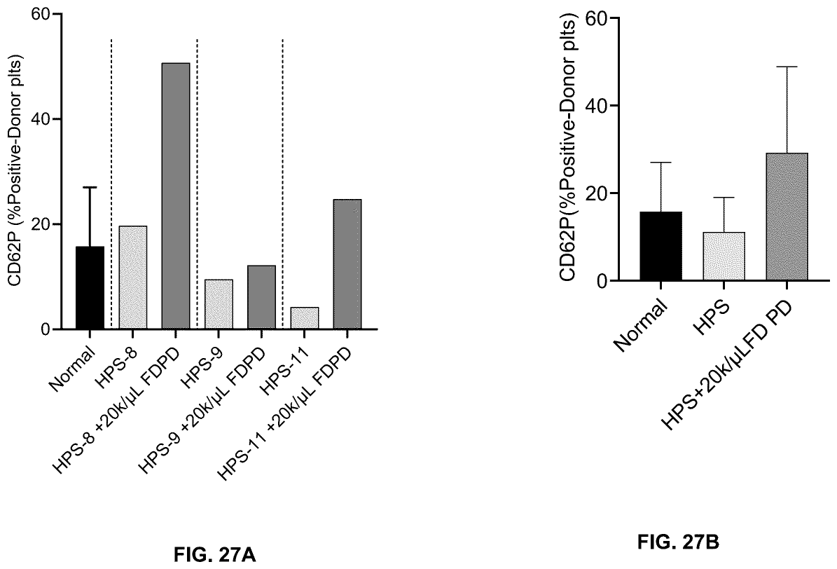

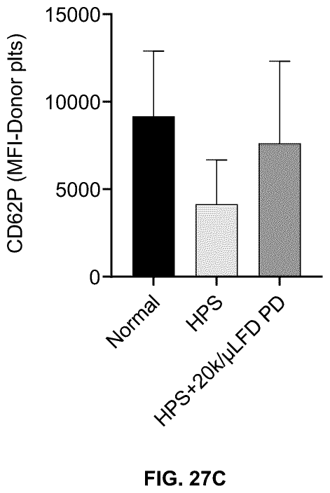

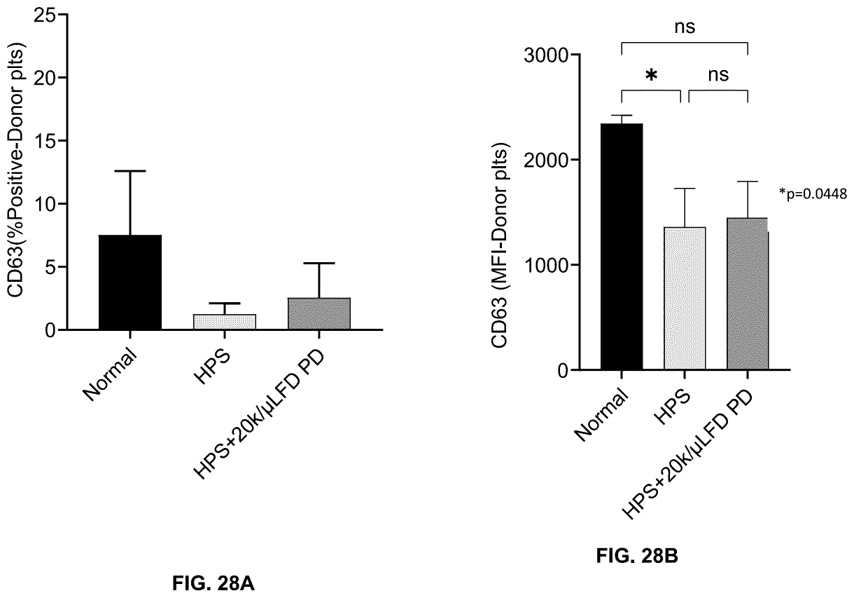

Liquid and dried compositions provided herein, in illustrative embodiments those prepared using freeze drying, and more specifically in some embodiments, prepared using methods provided herein, include particles that can be categorized broadly into populations based on at least one physical property, for example, but not limiting to, the size of the particles obtained. In some embodiments, the particles can be categorized into two populations based on size, typically in embodiments where exosomes are not present in detectable quantities, are not resolvable by the instrument analyzing particle size, and/or are not considered particles: For example, a first population comprising larger particles similar, or much more similar in size to in-dated stored platelets, which can be referred to herein as platelet derivatives, FDPDs, platelet-sized particles, a population of platelet derivatives with a size distribution centered around ˜1,000 nm radius, or ˜1,000 nm radius particles, and a second population comprising relatively smaller particles, which can be referred to herein as microparticles, a population of microparticles with a size distribution centered around ˜50 nm radius, or ˜50 nm radius particles (See e.g., FIG. 24A, FIG. 27B, or FIG. 28B, of U.S. Pat. No. 11,529,587 and of PCT app no. PCT/US2022/079280, where the main population of human in-dated stored platelets is centered at around 1,100 or 1,500 nm radius respectively, with a smaller (i.e. microparticle) peak at around 100 nm radius; and the particles in the FDPD composition (i.e. thrombosomes), which have a ˜platelet-sized major peak with a radius of approximately 1,100 nm (FIG. 24A) or 1,000 nm (FIG. 27B and FIG. 28B) (i.e. platelet derivatives) and a microparticle radius peak at approximately 50-75 nm).

A skilled artisan would further understand that the sizes determined for such populations of particles may not always be accurate enough to provide an exact cut-off value/range between these two particle size peaks. However, the difference in the sizes of the two populations can be resolved reproducibly using known methods, for example, using flow cytometry, or by using a particle/cell counter. And approximate size values or size range values can be obtained using such techniques optionally with sizing standards. In some embodiments, a composition comprising platelet derivatives or FDPDs as described herein or prepared according to methods described herein can have a population comprising platelet derivatives or FDPDs that includes between 95.1% to 99.9% of total particles in the composition, and the rest of the measurable particles, for example above 1 nm radius, can be microparticles. In some embodiments, platelet derivatives or FDPDs in such a composition can have a diameter of at least 0.4 μm (i.e., radius of at least 200 nm), and the microparticles in such a composition can have a diameter less than 0.4 μm (i.e., radius less than 200 nm). In other embodiments, platelet derivatives or FDPDs in such a composition can have a diameter of at least 0.5 μm (i.e., radius of at least 250 nm), and the microparticles in such a composition can have a diameter less than 0.5 μm (i.e., radius of less than 250 nm). In some embodiments, the platelet derivatives, or FDPDs can have a diameter of at least 0.4 μm, for example in the range of 0.5 μm to 22 μm (i.e., radius in the range of 200 nm or 250 nm to 11,000 nm), and the microparticles can have a diameter less than 0.5 μm (i.e., less than 250 nm radius), for example in the range of 0.04 μm to 0.350 μm (i.e., radius in the range of 20 nm to 175 nm). In some embodiments, the platelet derivatives, or FDPDs can have a diameter in the range of 1 μm to 18 μm (i.e., radius in the range of 500 nm to 9,000 nm), and the microparticles can have a diameter in the range of 0.06 μm to 0.2 μm (i.e., radius in the range of 30 nm to 100 nm). In some embodiments, the composition comprises platelet derivatives or FDPDs, and microparticles as the only or essentially the only particles present in the composition, optionally or typically other than exosomes, in embodiments where exosomes are not present in detectable quantities, are not resolvable by the instrument analyzing particle size, and/or are not considered particles. Of course, a composition as described herein may comprise any specific percentage number, or fraction thereof, of platelet derivatives, FDPDs or microparticles within the ranges discussed herein.

In some embodiments, the platelets or platelet derivatives (e.g., thrombosomes) have a particle size, for example a diameter, max dimension, or radius of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size, for example the diameter, max dimension, or radius, is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). From this disclosure, it will be apparent that microparticles typically have a size of less than 250 nm radius (i.e. less than 500 nm diameter). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets or platelet derivatives (e.g., thrombosomes), have a particle size of at least 0.5 μm, for example in the range of about 0.5 μm to about 25.0 μm, 20.0 μm, 15.0 μm, 12.5 μm, 10.0 μm, or 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets or platelet derivatives (e.g., thrombosomes), are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets or platelet derivatives (e.g., thrombosomes) are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some illustrative embodiments, a microparticle can be a particle having a particle size (e.g., diameter, max dimension) of less than about 0.5 μm (less than about 0.45 μm or 0.4 μm) In some cases, a microparticle can be a particle having a particle size of about 0.01 μm to about 0.5 μm (e.g., about 0.02 μm to about 0.5 μm).

Compositions comprising platelets or platelet derivatives (e.g., thrombosomes), such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, 0.5%, or 0.1%) of the total scattering intensity of all particles from about 1 nm to about 60,000 nm in radius in the composition. In some embodiments, the platelet derivative composition comprises a population of platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives have a diameter in the range of 0.5-2.5 μm, and the microparticles have a diameter less than 0.5 μm.

Platelets or platelet derivatives (e.g., thrombosomes) as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ thrombosomes/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, platelets or platelet derivatives (e.g., thrombosomes), such as those prepared by methods described herein, can have an average CD41 percent positivity of at least 55% (e.g., at least 60%, at least 65%, at least 67%, at least 70%, at least 75%, at least 80%, at least 85%, at least 90%, or at least 95%). In some embodiments, platelets or platelet derivatives herein can have an average CD41 percent positivity in the range of 70%-99%, 70%-95%, 70%-90%, 70%-86%, or 75%-86%. In some embodiments, at least 90%, 91%, 92%, 93%, 94%, 95%, 96%, 97%, 98%, 99% platelet derivatives that are positive for CD 41 have a size in the range of 0.5-2.5 μm. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., thrombosomes), such as those described herein, can have an average CD42 percent positivity of at least 65% (e.g., at least 67%, at least 70%, at least 75%, at least 80%, at least 85%, at least 90%, or at least 95%). In some embodiments, platelets or platelet derivatives can have an average CD42 percent positivity of at least 76%, 77%, 78%, or 79%. In some embodiments, platelets or platelet derivatives can have an average CD42 percent positivity in the range of 76-95%, 76-94%, 77-93%, or 78-90%. In some embodiments, at least 90%, 91%, 92%, 93%, 94%, 95%, 96%, 97%, 98%, or 99% platelet derivatives that are positive for CD 42 have a size in the range of 0.5-2.5 μm. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm.

As another example, platelets or platelet derivatives (e.g., thrombosomes), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., thrombosomes), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm. In some embodiments, at least 90%, 91%, 92%, 93%, 94%, 95%, 96%, 97%, 98%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm. In some embodiments, the presence of phosphatidyl serine in/on the platelet derivatives herein is higher than the presence of phosphatidyl serine in/on the platelets, such as, fresh platelets or apheresis platelets. For example, platelet derivatives herein exhibit at least 5 fold, 10 fold, 20 fold, 25 fold, 30 fold, 40 fold, or 50 fold higher presence of phosphatidyl serine as compared to the platelets.

Plasminogen activator inhibitor-1 (PAI-1) is a key inhibitor that regulates the thrombolytic activity of tissue-type plasminogen activator (tPA) released from vascular endothelium. Blood clots contain large amounts of PAI-1 that may originate from a granules of activated platelets. In some embodiments, platelet derivatives herein exhibit a higher presence (i.e., greater amount) of PAI-1 on/in the platelet derivatives as compared to platelets, such as fresh platelets or apheresis platelets. For example, platelet derivatives herein exhibit the presence of at least 1.5 fold, or 2 fold higher PAI-1 as compared with apheresis platelets. In some embodiments, platelet derivatives herein can have an average positivity of PAI-1 of at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%. In some embodiments, platelet derivatives herein can have an average positivity of PAI-1 in the range of 35-85%, 40-85%, 45-85%, or 50-85%.

Factor XIII (FXIII) plays an important role in normal hemostasis where it contributes to the regulation of fibrinolysis, and wound healing among other functions. The function of FXIII in hemostasis is further emphasized in its deficiency, which results in hemorrhage and slow wound healing. FXIII is present on platelets in large quantities making the quantities greater than that present in plasma. In some embodiments, platelet derivatives herein exhibit a higher presence (i.e., greater amount) of FXIII on/in the platelet derivatives as compared to platelets, such as fresh platelets or apheresis platelets. For example, platelet derivatives herein exhibit the presence of at least 5 fold, 10 fold, 25 fold, 50 fold, 100 fold, or 150 fold higher FXIII as compared to apheresis platelets. In some embodiments, platelet derivatives herein can have an average positivity of FXIII of at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

As another example, platelets or platelet derivatives (e.g., thrombosomes), such as those prepared by methods described herein, can have an average CD47 percent positivity of at least about 8% (e.g., at least about 10%, 15%, 20%, 25%, 30%, 35%, 40%, 45%, 50%, or 55%).

Glycoprotein VI (GPVI) is a platelet receptor for collagen, and the binding of collagen to GVPI activates the platelet. Receptor binding can be noticeably reduced in thrombosomes compared to fresh platelets. Without being bound by any particular theory, it is believed that the manufacturing process is blocking or destroying some copies of this receptor in thrombosomes, possibly to a reduction in collagen binding in thrombosomes relative to fresh platelets.

Platelets or platelet derivatives (e.g., thrombosomes) as described herein can have fibrinogen associated with the cell membrane. Aggregation of activated platelets is mediated by the formation of the GPIIb/IIIa complex, which can bind to fibrinogen (also called Factor 1) and form a clot. GPIIb/IIIa is a platelet fibrinogen receptor also known as CD41/CD61 complex. The GPIIb/IIIa clone PAC-1 binds to the active form of the GPIIb/IIIa. Without being bound by any particular theory, it is believed that the presence of fibrinogen on the cell membrane may be indicative of platelets or platelet derivatives (e.g., thrombosomes) capable of forming clots. Similarly, without being bound by any particular theory, it is believed that a lack of binding by anti-PAC1 antibodies to the platelets or platelet derivatives (e.g., thrombosomes), such as those prepared by methods described herein, can be indicative of fibrinogen bound to the active form of GPIIb/GPIIIa, as PAC-1 binds to the same complex. In some cases, platelets or platelets derivatives (e.g., thrombosomes), such as those prepared by methods described herein, can have a greater amount of bound fibrinogen when they retain a higher amount of residual plasma. In some embodiments of a platelet derivative composition as described herein, the platelet derivatives can have an amount of fibrinogen on their surface that is greater than that present on the surface of resting platelets, activated platelets, or lyophilized fixed platelets. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 2 folds higher mean fluorescent intensity (MFI) in the absence of an agonist as compared to the MFI of binding of anti-fibrinogen antibody to the lyophilized fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets. In some embodiments, the greater amount of fibrinogen present on the surface of the platelet derivatives as described herein as compared to that of lyophilized fixed platelets is beneficial. Without being bound by any particular theory, it is believed that the higher amount of fibrinogen on the cell membrane of the platelet derivatives (e.g., thrombosomes) as compared to that of lyophilized fixed platelets can make the platelet derivatives superior in terms of its ability to form clots as compared to lyophilized fixed platelets.

Von Willebrand factor (vWF) is a multimeric glycoprotein that plays a major role in blood coagulation. vWF serves as a bridging molecule that promotes platelet binding to sub-endothelium and other platelets, thereby promoting platelet adherence and aggregation. vWF also binds to collagens to facilitate clot formation at sites of injury. In some embodiments, the platelet derivatives as described herein have the presence of von Willebrand factor (vWF) on their surface at a level that is greater than that on the surface of resting platelets, activated platelets, or lyophilized fixed platelets. In some embodiments, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

Thrombospondin is a glycoprotein secreted from the α-granules of platelets upon activation. In the presence of divalent cations, the secreted protein binds to the surface of the activated platelets and is responsible for the endogenous lectin-like activity associated with activated platelets. In some embodiments, the platelet derivatives have the presence of thrombospondin (TSP-1) on their surface at a level that is greater than that presence on the surface of resting platelets, activated platelets, or lyophilized fixed platelets. In some embodiments, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

Platelet derivatives (e.g., FDPDs) that are included in methods, collections, and compositions herein, a) have the ability to generate thrombin in vitro, for example in the presence of tissue factor and phospholipids; b) have the ability to occlude a collagen-coated, and/or tissue factor (for example, thromboplastin)-coated microchannel, in illustrative embodiments collagen- and tissue factor-coated microchannel in vitro, in illustrative embodiments, and in the presence of divalent cations, and in further illustrative embodiments, in the absence of platelets; or c) both a) and b). Thus, in some embodiments, platelet derivatives (e.g., FDPDs) used in methods, collections, and compositions herein can be capable of generating thrombin, for example, when in the presence of a reagent containing tissue factor and phospholipids in vitro. For example, in some cases, platelets or platelet derivatives (e.g., thrombosomes) (e.g., at a concentration of about 4.8×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 pM, 0.5 pM, 1 pM, 2 pM, 5 pM or 10 pM) and optionally phospholipids. For example, in some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of a reagent containing tissue factor and (e.g., at 0.25 pM, 0.5 pM, 1 pM, 2 pM, 5 pM or 10 pM) and optionally phospholipids. In some cases, platelets or platelet derivatives (e.g., thrombosomes) (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., thrombosomes). In some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., thrombosomes).

Platelet derivatives (e.g., FDPDs) that are included in methods, collections, and compositions herein, a) have the ability to generate thrombin in vitro in the presence of tissue factor and phospholipids; b) have the ability to occlude a collagen-coated microchannel in vitro; or c) both a) and b). In some embodiments, platelet derivatives (e.g., thrombosomes) can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., thrombosomes) can have a potency of between 1.2 and 2.5 TGPU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TGPU per 10⁶ particles). TGPU can be calculated as follows: TGPU/million particles=[TPH in nM]*[Potency Coefficient in IU/(nM)]/[0.576 million particles in the well]. Similarly, the Potency Coefficient for a sample of thrombin can be calculated as follows: Potency Coefficient=Calculated Calibrator Activity (IU)/Effective Calibrator Activity (nM). In some cases, the calibrator activity can be based on a WHO international thrombin standard.

In some embodiments, platelet derivatives (e.g., FDPDs) as described herein can be capable of occluding a collagen-coated microchannel, a tissue factor-coated microchannel, or a collagen- and tissue factor-coated microchannel in vitro. For example, such occluding can be determined, for example, by using a total thrombus-formation analysis system (T-TAS®). In some embodiments, a microchannel is collagen-coated microchannel. In some embodiments, a microchannel is tissue factor-coated microchannel, for example, thromboplastin-coated microchannel. In some embodiments, a microchannel is collagen- and tissue factor-coated microchannel. In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 80) of less than 30, 25, 20, 15, or 14 minutes, or between 5 on the low end of the range, and 15, 20, or 25 on the high end, or between 10 on the low end of the range, and 15, 20, or 25 on the high end, or between 15 on the low end of the range and 20 or 25 on the high end, for example, in platelet-reduced citrated whole blood. In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood. Microchannels or capillaries having different dimensions can be used in a T-TAS system for determining the occlusion times of FDPDs under different experimental conditions as provided by numerous commercial suppliers (See e.g., Zacros, Tokyo, JP). For example, a T-TAS PL chip, AR chip, or HD chip can be used for an occlusion (e.g., T-TAS) assay, as are commercially available. Typically, an AR chip for the purposes of T-TAS assay is coated with either collagen, or a tissue-factor, such as thromboplastin, or both. Typically an HD chip for the purposes of T-TAS assay is coated with either collagen, or a tissue-factor, such as thromboplastin, or both. For example, the PL chip can have capillary dimensions of 40 μm×40 μm; or an AR chip can have capillary dimensions of 0.3 mm×80 μm; or an HD chip can have capillary dimensions of 0.3 mm×50 μm. Therefore, it is envisioned that a T-TAS assay can be performed to test the ability to occlude a collagen-coated microchannel, utilizing a microchannel or capillary with dimensions in the range of 0.02-0.5, 0.1-0.5, 0.2-0.4, 0.1-0.3, or 0.2-0.3 mm×25-200, 25-100, 50-100, 40-90, 40-80, or 50-80 μm.

Platelets or platelet derivatives (e.g., thrombosomes) as described herein can be capable of thrombin-induced trapping in the presence of thrombin. In some cases, platelets or platelet derivatives (e.g., thrombosomes) as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., thrombosomes) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin. Thrombin-induced trapping can be determined by any appropriate method, for example, light transmission aggregometry. Without being bound by any particular theory, it is believed that the thrombin-induced trapping is a result of the interaction of fibrinogen present on the surface of the platelet derivatives with thrombin.

Platelets or platelet derivatives (e.g., thrombosomes) as described herein can be capable of co-aggregating, for example, in the presence of an aggregation agonist, and fresh platelets. Non-limiting examples of aggregation agonists include, collagen, epinephrine, ristocetin, arachidonic acid, adenosine di-phosphate, and thrombin receptor associated protein (TRAP). In some cases, platelets or platelet derivatives (e.g., thrombosomes) as described herein can have a percent co-aggregation of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of an aggregation agonist, and fresh platelets. In some cases, platelets or platelet derivatives (e.g., thrombosomes) as described herein can have a percent co-aggregation of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of an aggregation agonist. Percent co-aggregation can be determined by any appropriate method, for example, light transmission aggregometry.

Platelet derivative compositions in certain illustrative embodiments herein, comprise a population of platelet derivatives having a reduced propensity to aggregate under aggregation conditions comprising an agonist but no fresh platelets, and in illustrative embodiments in the absence of divalent cations, compared to the propensity of fresh platelets and/or activated to aggregate under these conditions. Platelets or platelet derivatives (e.g., thrombosomes) as described herein in illustrative embodiments, display a reduced propensity to aggregate under aggregation conditions comprising an agonist but no fresh platelets and no divalent cations, compared to the propensity of fresh platelets and/or activated to aggregate under these conditions. It is noteworthy that aggregation of platelet derivatives is different from co-aggregation in that aggregation conditions typically do not include fresh platelets, whereas co-aggregation conditions include fresh platelets. Exemplary aggregation and co-aggregation conditions are provided in the Examples of U.S. Pat. No. 11,529,587 and of PCT App No. PCT/US2022/079280. Thus, in some embodiments, the platelet derivatives as described herein have a higher propensity to co-aggregate in the presence of fresh platelets and presence of an agonist, while having a reduced propensity to aggregate in the absence of fresh platelets, in the absence of divalent cations, and in the presence of an agonist, compared to the propensity of fresh platelets to aggregate under these conditions. In some embodiments, a platelet derivative composition comprises a population of platelet derivatives having a reduced propensity to aggregate, wherein no more than 1%, 2%, 3%, 4%, 5%, 7.5%, 10%, 12.5%, 15%, 17.5%, 20%, 22.5%, or 25% of the platelet derivatives in the population aggregate under aggregation conditions comprising an agonist but no divalent cations, no platelets, in illustrative embodiments no fresh platelets. In some embodiments, the population of platelet derivatives aggregate in the range of 0-1%, 0-2%, 0-3%, 0-4%, 0-5%, 0-7.5%, 0-10%, 2-30%, 5-25%, 10-30%, 10-25%, or 12.5-25%, and in illustrative embodiments 0-1% or 0 to about 1%, of the platelet derivatives under aggregation conditions comprising an agonist but no platelets, in illustrative embodiments no fresh platelets. In these and other illustrative embodiments the agonist is other than arachidonic acid. It will be understood that if an aggregation reaction control sample produces a background aggregation above 0% then aggregation values/ranges for FDPDs disclosed herein would be increased by the background value obtained with the control sample. Thus, if a background aggregation produced using a control sample was 1%, then the above ranges would be increased by 1% (e.g., 1-2%, 1-3%, 1-4%, 1-5%, 1-6%, 1-8.5%, and 1-11% etc.). Accordingly, in some embodiments, the values and ranges provided herein for aggregation are values above background values, for example obtained using a control sample or no sample, and thus can be referred to control-corrected, or control-adjusted aggregation values. In some embodiments, a platelet derivative composition comprises a population of platelet derivatives having a reduced propensity to aggregate, such that less than 1/5, 1/10, or 1/20 of the platelet derivatives in the population aggregate under aggregation conditions comprising an agonist but no platelets, compared to platelet rich plasma, in illustrative embodiments prepared from fresh platelets. In some embodiments, a population of platelet derivatives can aggregate in the absence of an agonist and in illustrative embodiments in the absence of thrombin, but in the presence of a divalent cation. For example, the population of platelet derivatives can aggregate in the presence of a divalent cation in the range of 5-80%, 5-75%, 5-70%, 5-65%, 5-60%, 5-50%, 5-40%, 10-80%, 15-80%, 20-80%, 25-80%, 30-80%, 35-80%, 40-80%, or 45-80%. Non-limiting examples of divalent cations can include magnesium (Mg²⁺), barium (Ba²⁺), copper (Cu²⁺), calcium (Ca²⁺), manganese (Mn²⁺), zinc (Zn²⁺), iron (Fe²⁺), nickel (Ni²⁺), and cobalt (Co²⁺). A skilled artisan can understand that any suitable salt of the divalent cations can be used for the aggregation assay, for example, a chloride salt of magnesium, barium, copper, calcium, manganese, zinc, iron, nickel, or cobalt. In some embodiments, a population of platelet derivatives can show a higher percentage of aggregation in the absence of an agonist, but in the presence of a divalent cation as compared to platelets, in illustrative embodiments, fresh platelets.

Platelets derivatives (e.g., thrombosomes) herein, in some embodiments, are dry platelet derivatives, or dry platelet derived particles. In some embodiments, such dry platelet derivatives are freeze-dried (i.e., lyophilized) platelets or platelet derivatives. One such non-limiting example of dry platelet derivatives are thrombosomes. Dry platelet derivatives are typically in the form of a platelet derivative powder. The dry platelet derivative powder when rehydrated typically forma a rehydrated platelet derivative composition comprising particles. In some embodiments, compositions comprising a population of platelet derivatives, dry platelet derivatives, platelet derivative powder, or rehydrated platelet derivatives can be characterized by the presence of CD41 on or in at least 55%, 60%, 65% or higher platelet derivatives in the population. In some embodiments, compositions comprising a population of platelet derivatives, dry platelet derivatives, platelet derivative powder, or rehydrated platelet derivatives can be characterized by the presence of CD42 on or in at least 55%, 60%, 65% or higher platelet derivatives in the population. In some embodiments, dry platelet derivative particles herein can have at least one property selected from: (a) high expression of P-selectin (CD62P), for example, at least 2 fold higher than platelets, for example, apheresis platelets, or at least 10%, 20%, 30%, 40%, 50%, 60%, 70%, or higher platelet derivative particles are positive for CD62; (b) high expression of phosphatidyl serine (PS), for example, at least 2, 3, 4, 5, 6, 7, 8, 9, 10 fold or higher than the expression on platelets, for example, apheresis platelets, or at least 25%, 30%, 40%, 50%, 60%, 70%, or higher platelet derivative particles are positive for phosphatidyl serine; (c) high expression of von Willebrand Factor (vWF), for example, at least 2, 3, 4, 5, 6, 7, 8, 9, 10 fold or higher than the expression on platelets, for example, apheresis platelets; (d) high expression of fibrinogen, for example, at least 2, 3, 4, 5, 6, 7, 8, 9, 10 fold or higher than the expression on platelets, for example, apheresis platelets; (e) high expression of thrombospondin (TSP), for example, at least 2, 3, 4, 5, 6, 7, 8, 9, 10 fold or higher than the expression on platelets, for example, apheresis platelets; (f) high expression of CD41, for example, at least 55%, 60%, 65%, 70%, 75%, 80%, 85%, 90%, or higher platelet derivative particles are positive for CD41; or (g) high expression of CD42, for example, at least 65%, 70%, 75%, 80%, 85%, 90%, 95%, or higher platelet derivative particles are positive for CD42. In some embodiments, the platelet derivative particles can have two or more properties as described herein, in illustrative embodiments, the platelet derivative particles can have all the properties as described herein.

Platelet derivatives herein, in some embodiments, are capable of generating more thrombin as compared to thrombin generated by platelets, such as fresh platelets, or apheresis platelets. For example, platelet derivatives can generate at least 2 fold, 3 fold, or more thrombin in an in vitro thrombin formation assay as compared to thrombin generated by apheresis platelets. In some embodiments, platelet derivatives can generate a maximum amount of thrombin in less time as compared to platelets, such as fresh platelets or apheresis platelets. For example, platelet derivatives herein can generate a maximum amount of thrombin in at least 10%, 20%, 30%, 40%, 50%, or 60% less time than that taken by apheresis platelets.

Compositions comprising platelets or platelet derivatives (e.g., thrombosomes) as described herein can have appropriate conditions and amounts of cellular substrates and/or metabolites, such as pH, pCO₂, PO₂, HCO₃ concentration, total carbon dioxide (TCO₂), sO₂, and lactate concentration. Lactate can be the products of glycolysis. Without being bound by any particular theory, a starting material can have high lactate concentration because it has been stored ex vivo, respirating and performing glycolysis, for a period of time (e.g., about 3 days) by the time of manufacturing. For example, in some cases, the pH can be about 5.5 to about 8.0 (e.g., about 6.0 to about 7.4, about 6.9 to about 7.5, or about 7.0 to about 7.3). As another example, the pCO₂ can be about 10 to about 20 mmHg (e.g., about 10 to about 15 mmHg, about 15 to about 20 mmHg, or about 17 to about 19 mmHg). The pO₂ can be about 140 to about 165 mmHg (e.g., about 140 to about 150 mmHg, about 150 to about 160 mmgH, or about 160 to about 165 mmHg). The HCO₃ concentration can be about 4.5 to about 6.5 mmol/L (e.g., about 5.0 to about 6.0 mmol/L). The total carbon dioxide can be about 4 to about 8 mmol/L (e.g., about 5 to about 7 mmol/L). The SO₂ can be at least about 98% (e.g., at least about 99%). The lactate concentration can be less than about 2.0 mmol/L (e.g., less than 1.5 mmol/L or 1.0 mmol/L). The lactate concentration can be about 0.4 to about 1.3 mmol/L (e.g., about 0.5 to about 0.6 mmol/L, about 0.5 to about 1.0 mmol/L, or about 0.8 to about 1.3 mmol/L).

### Membrane Integrity of the Platelet Derivatives

Platelet derivatives in certain illustrative aspects and embodiments herein are surrounded by a compromised plasma membrane. In these illustrative aspects and embodiments, the platelet derivatives lack an integrated membrane around them. Instead, the membrane has pores on them that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The increased permeability can be identified by targeting IgG antibodies against a stable intracellular antigen. One non-limiting type of stable intracellular antigen is R tubulin. The compromised membrane of the platelet derivatives can also be determined by flow cytometry studies.

Platelet or platelet derivatives (e.g., thrombosomes) as described herein can retain some metabolic activity, for example, as evidenced by lactate dehydrogenase (LDH) activity. In some cases, platelets or platelet derivatives (e.g., thrombosomes) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., thrombosomes) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

### Pathogen Reduction

The reduction of pathogens is generally desirable in blood products. Without being bound by any particular theory, it is believed that some methods of pathogen reduction can cause the formation of microparticles in the treated blood product. One method of pathogen reduction involves the use of a photosensitive nucleic acid-intercalating compound to alter the nucleic acids of pathogens upon illumination with an appropriate wavelength. The INTERCEPT® system (made by Cerus) uses amotosalen, a nucleic acid intercalating compound that forms cross-links in nucleic acid upon illumination with UVA.

### Starting Material Comprising Platelets or a Platelet Composition

A final blood product (e.g., platelets, cryopreserved platelets, freeze-dried platelets (e.g., thrombosomes)) as described herein can be prepared by any appropriate method. A final blood product (e.g., platelets, cryopreserved platelets, freeze-dried platelets (e.g., thrombosomes)) as described herein can be prepared by a method as disclosed herein. In some embodiments described herein, a final blood product can be a composition that includes platelets and an aqueous medium. In some embodiments, a final blood product can be the result of freeze-drying a composition that includes platelets and an aqueous medium, as described herein. In some embodiments, a final blood product can be prepared using tangential flow filtration (TFF) of a starting material (e.g., an unprocessed blood product (e.g., donor apheresis material (e.g., pooled donor apheresis material)), or a partially processed blood product (e.g., a blood product that has undergone filtration)). In some embodiments, a final blood product can be prepared using centrifugation of a starting material (e.g., an unprocessed blood product (e.g., donor apheresis material (e.g., pooled donor apheresis material)), or a partially processed blood product (e.g., a blood product that has undergone filtration)). It will be appreciated that while the methods described herein are generally described in the context of a starting material being apheresis material, other materials, such as platelets cultured in vitro, or whole blood, may be used. In some cases, platelets may be isolated from whole blood (e.g. pooled whole blood).

A starting material can be any appropriate starting material. In some embodiments, a starting material can have a protein concentration of about 60 to about 80 mg/mL. In some embodiments, a protein concentration can be based on the protein concentration in the plasma of whole blood. In some embodiments, a protein concentration can be based on the protein concentration of donor apheresis plasma. In some embodiments, a starting material can be donor blood product (e.g., whole blood or fractionated blood). In some embodiments, the starting material can be pooled donor blood product (e.g., pooled whole blood or pooled fractionated blood). In some embodiments, a starting material can include donor apheresis plasma. In some embodiments, a starting material can be derived from donor apheresis plasma. As used herein, “donor apheresis plasma” can refer to the plasma component of apheresis material, whether or not the material contains platelets or other blood cells.

In some embodiments, a starting material can be donor apheresis material (e.g., donor platelets or a pool of donor platelets). In some embodiments, a starting material is positive for one or more of: HLA Class I antibodies, HLA Class II antibodies, and HNA antibodies based on a regulatory agency-approved assay (e.g., an FDA cleared assay). In some embodiments, starting material can test positive for HLA Class I antibodies in a regulatory agency approved assay (e.g., an FDA cleared assay). In some embodiments, a starting material can test positive for HLA Class II antibodies in a regulatory agency approved assay (e.g., an FDA cleared assay). In some embodiments, starting material can test positive for HNA antibodies in a regulatory agency approved assay (e.g., an FDA cleared assay). A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some embodiments, a starting material can undergo a pathogen reduction step, for example, a nucleic acid intercalating compound that forms cross-links in nucleic acid upon illumination with UVA.

In some embodiments, a starting material (e.g., one or more units of donor platelets) can be initially pooled into a common vessel. The starting material may or may not be initially diluted with an acidified washing buffer (e.g., a control buffer). Without being bound by any particular theory, it is believed that washing with an acidified washing buffer can reduce platelet activation during processing. In some cases, a starting material can undergo two general processing pathways; either washed with control buffer (e.g. using TFF) until a desired residual component is reached (e.g., a percentage of residual donor plasma) before being concentrated to a final concentration; or the starting material can be concentrated to a final concentration before being washed with control buffer (e.g., using TFF) until a desired residual component is reached (e.g., a percentage of residual donor plasma). TFF processed material can then be filled into vials, lyophilized and thermally treated.

Different steps in processing of a starting material, and TFF

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×10³ to about 4000×10³ platelets/μL (e.g., about 1000×10³ to about 2000×10³, about 2000×10³ to about 3000×10³, or about 4000×10³ platelets/μL). In some embodiments, a concentration step can be carried out using the TFF apparatus.

The concentration of platelets or platelet derivatives (e.g., thrombosomes) can be determined by any appropriate method. For example, a counter can be used to quantitate concentration of blood cells in suspension using impedance (e.g., a Beckman Coulter AcT 10 or an AcT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200 kDa, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain the platelet derivatives and allow the microparticles to pass through the membrane. The pore size in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×10³ to 20,000×10³. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 150%, 10%, 9%, 8%, 7%, 6%, 5%, 4%, 3%, 2%, 10%, 0.9%, 0.8%, 0.7%, 0.6%, 0.50%, 0.4%, 0.30%, 0.2%, or 0.10% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). As another example, an endpoint can be a platelet concentration of about 1000×10³ to about 2500 platelets/μL (e.g., about 1000×10³ to about 2000×10³, about 1500×10³ to about 2300×10³, or about 1700×10³ to about 2300×10³ platelets/μL). In some embodiments, an endpoint can be a concentration of platelets in the TFF-treated composition are at least 100×10³ platelets/μL, 200×10³ platelets/μL, 400×10³ platelets/μL, 1000×10³ platelets/μL, 1250×10³ platelets/μL, 1500×10³ platelets/μL, 1750×10³ platelets/μL, 2000×10³ platelets/μL, 2250×10³ platelets/μL, 2500×10³ platelets/μL, 2750×10³ platelets/μL, 3000×10³ platelets/μL, 3250×10³ platelets/μL, 3500×10³ platelets/μL, 3750×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, 20,000×10³ platelets/μL. In some embodiments, the platelets or platelet derivatives in the TFF-treated composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, freeze-dried platelets (e.g., thrombosomes)). However, in some cases, a TFF-treated composition can be considered to be a final blood product.

In some embodiments, a blood product can be prepared using centrifugation of a blood product (e.g., an unprocessed blood product (e.g., donor apheresis material (e.g., pooled donor apheresis material)), or a partially processed blood product (e.g., a blood product that has undergone TFF)). In some embodiments, a blood product can be prepared without centrifugation of a blood product (e.g., an unprocessed blood product (e.g., donor apheresis material), or a partially processed blood product (e.g., a blood product that has undergone TFF)). Centrifugation can include any appropriate steps. In some embodiments, centrifugation can include a slow acceleration, a slow deceleration, or a combination thereof. In some embodiments, centrifugation can include centrifugation at about 1400×g to about 1550×g (e.g., about 1400 to about 1450×g, about 1450 to about 1500×g, or 1500 to about 1550×g, about 1400×g, about 1410×g, about 1430×g, about 1450×g, about 1470×g, about 1490×g, about 1500×g, about 1510×g, about 1530×g, or about 1550×g). In some embodiments, the duration of centrifugation can be about 10 min to about 30 min (e.g., about 10 to about 20 min, about 20 to about 30 min, about 10 min, about 20 min, or about 30 min).

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

In some embodiments, a composition as described herein can be analyzed at multiple points during processing. In some embodiments, donor apheresis plasma can be analyzed for antibody content (e.g., HLA or HNA antibody content). In some embodiments, donor apheresis plasma can be analyzed for protein concentration (e.g., by absorbance at 280 nm). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

The protein concentration of a blood product can be measured by any appropriate method. In some embodiments, the protein concentration of a blood product can be measured using absorbance at 280 nm.

The antibody content (e.g., HLA or HNA antibody content) of a blood product can be measured by any appropriate method.

In some embodiments, a FLOWPRA™ Screening or a LABScreen Multi test kits from One Lambda, Thermo Fisher Scientific can be used as a method of HLA detection. Raw materials can be tested prior to the TFF or centrifugation processes to determine a baseline level of class I and II antibodies for Human Leukocyte Antigen (HLA) and Human Neutrophil Antigens (HNA). Testing can be repeated after processing by centrifugation or TFF to measure the removal of HLA and HNA. Additional testing points can be performed throughout the TFF procedure to maintain in-process control. Post-lyophilization and annealing, random samples can be selected from a batch and qualitative HLA/HNA antibody testing can be performed to ensure reduction and compliance with current FDA testing and acceptance requirements.

In some embodiments, the antibody content (e.g., HLA or HNA antibody content) of two blood products can be compared by determining the percentage of beads positive for a marker (e.g., HLA or HNA coated beads bound to HLA or HNA antibodies, respectively). Any appropriate comparative method can be used. In some embodiments, the antibody content of two blood products can be compared using a method as described herein. In some embodiments, such a method can be carried out as follows. An aliquot of plasma (e.g., about 1 mL) platelet-poor plasma can be obtained. In some embodiments, an aliquot of filtered (e.g., using a 0.2 μm filter) platelet-poor plasma (PPP) (e.g., about 1 mL) can be obtained. Beads coated with Class I HLA and/or beads coated with Class II HLA can be added to the plasma (e.g., about 5 μL of each type of bead to about 20 μL of PPP) to form a mixture of PPP and beads. The mixture of PPP and beads can be vortexed. The mixture of PPP and beads can be incubated to form an incubated mixture. Any appropriate incubation conditions can be used. For example, in some embodiments, incubation can occur for a time (e.g., for about 30 minutes) at a temperature (e.g., at room temperature) with other conditions (e.g., in the dark) to form an incubated mixture. In some embodiments, incubation can include agitation (e.g., gentle rocking). The beads in the incubated mixture can be washed using any appropriate conditions. In some embodiments, the beads in the incubated mixture can be washed with a wash buffer. Washed beads can be separated from the incubated mixture by any appropriate method. In some embodiments, the washed beads can be separated by centrifugation (e.g., at 9,000×g for 2 minutes) to obtain pelleted beads. In some embodiments, the washing step can be repeated. The beads can be resuspended to form a bead solution. An antibody (e.g., an antibody that will bind to the assayed antibody content (e.g., HLA or HNA antibody content)) conjugated to a detectable moiety can be added to the bead solution (e.g., an αIgG conjugated to a fluorescent reporter, such as FITC). The antibody can be incubated with the bead solution under any appropriate conditions. In some embodiments, the antibody can be incubated for a time (e.g., for about 30 minutes) at a temperature (e.g., at room temperature) with other conditions (e.g., in the dark) to form labeled beads. Labeled beads can be washed to remove unbound antibody conjugated to a detectable moiety. The labeled beads can be washed using any appropriate conditions. In some embodiments, the labeled beads can be washed with a wash buffer. Washed labeled beads can be separated by any appropriate method. In some embodiments, the washed labeled beads can be separated by centrifugation (e.g., at 9,000 g for 2 minutes) to obtain pelleted labeled beads. In some embodiments, the washing step can be repeated. Labeled beads can be detected by any appropriate method. In some embodiments, labeled beads can be detected by flow cytometry. In some embodiments, detection can include measurement of the percentage of beads that are positive for the detectable moiety as compared to a negative control. In some embodiments, a negative control can be prepared as above, using a PPP sample that is known to be negative for antibodies (e.g. HLA Class I, HLA Class II, or HNA antibodies).

In some embodiments, a blood product (e.g., platelets, cryopreserved platelets, freeze-dried platelets (e.g., thrombosomes)) can be analyzed at multiple points during processing. In some embodiments, a starting material (e.g., donor apheresis material) can be analyzed to determine the percent of positive beads (e.g., HLA or HNA coated beads). In some embodiments, a starting material (e.g., donor apheresis material) can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

In some embodiments, a blood product (e.g., platelets, cryopreserved platelets, freeze-dried platelets (e.g., thrombosomes)) can be analyzed at multiple points during processing. In some embodiments, donor apheresis plasma can be analyzed to determine the percent of positive beads (e.g., HLA or HNA coated beads). In some embodiments, donor apheresis plasma can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%5%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis plasma. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

A percentage of positive beads can be determined using any appropriate method. In some embodiments, positive beads can be determined compared to a negative control sample. A negative control sample can be any appropriate negative control sample. In some embodiments, a negative control sample can be used to determine positivity gating such that less than a certain percentage (e.g., between about 0.01% and about 1% (e.g., about 0.01% to about 0.05%, about 0.05% to about 0.1%, about 0.1% to about 0.5%, about 0.5% to about 1%, about 0.01%, about 0.05%, about 0.1%, about 0.5%, or about 1%)) of the negative control sample is present within the positivity gate. In some embodiments, a negative control sample can be a buffer (e.g., PBS). In some embodiments, a negative control sample can be a synthetic plasma composition. In some embodiments, a negative control sample can be a blood product known to be negative for the assayed antibodies (e.g., HLA or HNA antibodies).

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration.

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation.

In some embodiments of any of the methods described herein, the amount of an antibody (e.g., a HLA antibody (e.g., a HLA Class I antibody or a HLA Class II antibody) or a HNA antibody) in a composition (e.g., a blood product) can be reduced to below a reference level. A reference level can be any appropriate reference level. In some embodiments of any of the methods described herein, the percentage of beads positive an antibody (e.g., a HLA antibody (e.g., a HLA Class I antibody or a HLA Class II antibody) or a HNA antibody) in a composition (e.g., a blood product) can be reduced as compared to the blood product before undergoing the methods described herein. A percentage of beads positive for an antibody can be reduced by any appropriate amount. In some embodiments, a percentage of beads positive for an antibody can be reduced by at least 5% (e.g., reduced by at least 10%, 20%, 30%, 40%, 50%, 60%, 70%, 75%, 80%, 85%, 90%, 95%, 96%, 97%, 98%, 99%, or more) compared to the blood product before undergoing any of the methods described herein.

In some embodiments, a composition as described herein can undergo any appropriate additional processing steps. In some embodiments, a composition as described herein can be freeze-dried. In some embodiments, freeze-dried platelets can be thermally treated (e.g., at about 80° C. for about 24 hours).

For example, in some embodiments, a composition can be cryopreserved or freeze-dried. In some embodiments, a first composition (e.g., a composition comprising platelets and an aqueous medium as described herein) can be treated with a mixture. In some embodiments, a mixture can include a lyophilizing agent, including a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form a second composition comprising platelets. In some embodiments, a loading agent can be a saccharide. In some embodiments, a saccharide can be a monosaccharide. In some embodiments, a saccharide can be sucrose, maltose, trehalose, glucose (e.g., dextrose), mannose, or xylose. In some embodiments, the loading agent can be polysucrose.

In some embodiments, a first composition or a second composition can be dried. In some embodiments, a first composition or a second composition can be dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a third composition. In some embodiments, a cryoprotectant can be polysucrose.

In some embodiments, a first composition or a second composition can be freeze-dried. In some embodiments, a first composition or a second composition can be freeze-dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a fourth composition. In some embodiments freeze-drying can occur at a temperature of about −40° C. to about 5° C. In some embodiments, freeze-drying can occur over a gradient (e.g., about −40° C. to about 5° C.). In some embodiments, a secondary drying step can be carried out (e.g., at about 20° C. to about 40° C.).

Also provided herein are blood products (e.g., platelets, cryopreserved platelets, freeze-dried platelets (e.g., thrombosomes)) produced by any of the methods described herein.

In some embodiments, the percentage of beads positive for an antibody selected from the group consisting of HLA Class I antibodies, HLA Class II antibodies, and HNA antibodies, as determined for a composition as described herein by flow cytometry using beads coated with Class I HLAs, Class II HLAs, or HNAs, respectively, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class I antibodies, as determined for a composition as described herein by flow cytometry using beads coated with Class I HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class II antibodies, as determined for a composition as described herein by flow cytometry using beads coated with Class II HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HNA antibodies, as determined for a composition as described herein by flow cytometry using beads coated with HNAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

Within the process provided herein for making the compositions provided herein, optional addition of a lyophilizing agent can be the last step prior to drying. However, in some embodiments, the lyophilizing agent can be added at the same time or before other components of the composition, such as a salt, a buffer, optionally a cryoprotectant, or other components. In some embodiments, the lyophilizing agent is added to a preparation agent, thoroughly mixed to form a drying solution, dispensed into a drying vessel (e.g., a glass or plastic serum vial, a lyophilization bag), and subjected to conditions that allow for drying of a TFF-treated composition to form a dried composition.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., thrombosomes) for at least 30 minutes at a temperature higher than 30° C.; heating the dried platelets or platelet derivatives (e.g., thrombosomes) for at least 10 hours at a temperature higher than 50° C.; heating the dried platelets or platelet derivatives (e.g., thrombosomes) for at least 18 hours at a temperature higher than 75° C.; and heating the dried platelets or platelet derivatives (e.g., thrombosomes) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., thrombosomes).

In some cases, compositions comprising platelets or platelet derivatives (e.g., thrombosomes) can be rehydrated with water (e.g., sterile water for injection) over about 10 minutes at about room temperature. In general, the rehydration volume is about equal to the volume used to fill each vial of thrombosomes prior to drying.

In some embodiments, the platelets or platelet derivatives (e.g., thrombosomes) prepared as disclosed herein have a storage stability that is at least about equal to that of the platelets prior to the preparation.

In some embodiments, the method further comprises cryopreserving the platelets or platelet derivatives prior to administering the platelets or platelet derivatives (e.g., with a preparation agent, e.g., a preparation agent described herein).

In some embodiments, the method further comprises drying a composition comprising platelets or platelet derivatives, (e.g., with a preparation agent e.g., a preparation agent described herein) prior to administering the platelets or platelet derivatives (e.g., thrombosomes). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with a preparation agent e.g., a preparation agent described herein) prior to administering the platelets or platelet derivatives (e.g., thrombosomes) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the thrombosomes prior to administering the platelets, platelet derivatives, or thrombosomes (e.g., with a preparation agent, e.g., a preparation agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., thrombosomes) (e.g., with a preparation agent e.g., a preparation agent described herein) prior to administering the platelets or platelet derivatives (e.g., thrombosomes). For example, in some embodiments, the method further comprises drying (e.g., freeze-drying) a composition comprising platelets or platelet derivatives (e.g., with a preparation agent e.g., a preparation agent described herein) (e.g., to form thrombosomes) prior to administering the platelets or platelet derivatives (e.g., thrombosomes). In some embodiments, the method may further comprise rehydrating the composition obtained from the drying step.

In some embodiments, provided herein is a method for preparing a composition comprising platelets or platelet derivatives (e.g., thrombosomes). The method can include diluting a starting material comprising platelets with an approximately equal weight (±10%) of a preparation agent (e.g., Buffer A, as provided in Example 1 of U.S. Pat. No. 11,529,587 and Example 1 of PCT app no. PCT/US2022/079280), concentrating the platelets to about 2250×10³ cells/μL (+250×10³) and then washed with 2-4 diavolumes (DV) (e.g., about 2 diavolumes) of the preparation agent to form a TFF-treated composition. The residual plasma percentage can be less than about 15% relative plasma (as determined by plasma protein content). Following washing, if the concentration of the cells in the TFF-treated composition is not about 2000×10³ cells/μL (+300×10³), the cells can be diluted with the preparation agent or can be concentrated to fall within this range. The method can further include lyophilizing the TFF-treated composition and subsequently treating the lyophilized composition comprising platelets or platelet derivatives (e.g., thrombosomes) at about 80° C. for about 24 hours. In some embodiments, the method can further include a pathogen reduction step, for example, before diluting the starting material.

### Plurality of Containers Containing Platelet Derivative Composition

The platelet derivative composition as described herein can be contained in containers/vials, which further can be packed into a plurality of containers for shipping to a customer, which can be part of a commercialization process to fulfill an order for such platelet derivative composition. The containers/vessels, in certain embodiments, are 5 ml vials, 10 ml vials, 20 ml vials, 25 ml vs, 30 ml vials, 40 ml vials, 50 ml vials, 60 ml vials, 75 ml vials, 100 ml vials, 125 ml vials, 150 ml vials, 200 ml vials, or 250 ml vials. The vial(s) can be a cryovial, or a cryotube especially in illustrative embodiments where the TFF-treated composition that includes platelets is lyophilized to obtain the platelet derivative composition in the form of a powder, which further can be baked or not baked after it is lyophilized. In some embodiments, the volume of the containers in a plurality of containers (e.g. vials or tubes), which for example can be all from one lot, or from more than one lot (e.g. 2, 3, 4, 5, 6, 7, 8, 9 or 10 lots), can vary from one or more than one size between 10-100 ml. Typically, the volume of the vial/container in embodiments where the platelet derivative is a freeze-dried solid/powder, is 1× the volume of, or 1.10, 1.25, 1.5, 2, 2.5, 3, 4 or 5 times the volume of a composition that was filled in the vial before lyophilization, and/or the volume in which the powder in the vials will be rehydrated, which is an illustrative embodiment. Thus, the maximum volume of such vials can be the same or more than the volume of the composition that was filled inside prior to lyophilization or the volume in which the platelet derivative composition in the form of a powder can be rehydrated. For example, in one non-limiting embodiment, a vial with a maximum capacity of 100 ml, can be used to fill 10 ml of a TFF-treated composition that includes platelets for lyophilization. In certain embodiments, the capacity of a vial in which a TFF-treated composition that includes platelets is lyophilized, is 1-2.5 times and in other embodiments, 1-2 times, 1-3 times, 1-4 times, 1-5 times, and in certain illustrative embodiments, 1.1 to 2 times or 1.25 to 2 times the volume of a TFF-treated composition that is lyophilized therein.

The TFF-treated platelet composition, in illustrative embodiments, FDPDs or HLA-characterized FDPDs, FPH or HLA-characterized FPH before lyophilization, or in some embodiments, the platelet derivative composition obtained after the lyophilization step, with or without post-lyophilization heat treatment (baking), can be filled into a plurality of vessels or other powder and liquid-holding containers, such as vials, in a sterile manner. In some embodiments, the containers/vessels can vary in volume from 5-100 ml, 10-90 ml, 25-75 ml, or 5-40 ml. In some embodiments, the volume of containers/vessels can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers/vessels can be above 100 ml, for example, 125 ml, 150 ml, 175 ml, or 200 ml. The platelet derivative composition as described herein can be filled in vials of different volumes as per the commercialization requirements. A plurality (or collection) of containers/vessels having the platelet derivative composition as per any of the embodiments herein, obtained by lyophilizing the composition that includes platelets during one process (e.g. TFF or other process) for preparing a platelet derivative, can be referred to as a “batch” or a “lot”. In some embodiments, a batch/lot can have 10-500 vials, 25-450 vials, 50-350 vials, 100-300 vials, or 150-250 vials. In some embodiments, a batch/lot can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the number of vials per batch/lot can be increased to more than 500 as per the requirements, for example, 600, 700, 800, 900, or 1000 vials. In some embodiments, the number of vials can be 10-1000, 50-1000, 100-900, 200-800, 100-500, 100-400, 150-700, or 150-500 vials. The containers in a batch/lot can have a volume in the range of 5-100 ml, for example, such that a lot has several containers with the same volume or containers with different volumes. For example, 200 vials/containers in a batch/lot can have a volume of 10 ml each, 100 vials/containers in the same or a separate batch/lot can have a volume of 20 ml each, 100 vials/containers in the same or another batch/lot can have a volume of 30 ml each, or 300 vials/containers in the same or a different batch/lot can have a volume of 10 ml each. The number of containers (e.g. vials) in which a platelet derivative composition as per one of the embodiments or aspects described herein can be packed in a batch/lot can vary with manufacturing requirements, the requirements of downstream processes, for example clinical processes, and the amount of starting material comprising platelet composition.

The quantity of platelet derivatives that is present in a batch/lot can vary based on the units of starting material comprising platelets that is used to produce the platelet derivatives. Certain methods provided herein, such as the TFF methods provided herein, allow more platelet units to be used to make platelet derivatives with the characteristics provided herein than prior methods. This is the result, for example, of the ability to reduce the level of certain components in a platelet composition starting material, such as HLA antibodies, HNA antibodies, and/or microparticles, to very low levels, as provided herein. Accordingly, the starting material comprising platelets, the corresponding composition (e.g. TFF-treated composition) that is lyophilized in illustrative embodiments, and the resulting platelet derivative composition powder, can vary, such that for example, in some embodiments, the starting material, the TFF-treated composition, and/or the resulting platelet derivative composition powder can include 10-500 units of platelets or platelet derivatives (e.g. 0.5 to 2.5 μm in diameter), with one unit being 3×10¹¹ platelets or platelet derivatives. In some embodiments, the starting platelet material, the composition to be lyophilized, and/or the platelet derivative (e.g. 0.5 to 2.5 μm in diameter) composition can include, for example, 20-500 units, 30-400 units, 40-350 units, or 50-200 units of platelets or platelet derivatives, respectively. In some embodiments, the platelet units in the starting platelet composition can be a pooled platelet product from multiple donors as described herein, or multiple batches of processed platelet compositions, such as TFF-treated compositions comprising platelets, can be pooled before lyophilization. In some embodiments, there can be 1×10⁹ to 1×10¹⁶ platelets in a starting platelet composition for processing, in a platelet composition that is lyophilized, and/or of platelet derivatives in a platelet derivative composition that is produced after lyophilization, per batch/lot. In some embodiments, the platelet-containing starting composition, the platelet composition that is lyophilized, and/or the platelet derivatives that are produced, typically after lyophilization per batch/lot can vary from 1×10¹⁰ to 1×10¹¹, 1×10¹¹ to 1×10¹⁵, 1×10¹² to 1×10¹⁶, 1×10¹³ to 1×10¹⁵ or 1×10¹³ to 1×10¹⁴.

In certain illustrative embodiments, platelet derivative compositions that are present in a liquid, or in illustrative embodiments, a solid form such as a dried powder in the plurality of containers (e.g. vials), in illustrative embodiments of a 1 or more lots, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are 0.5 to 2.5 um in diameter. Such platelet derivatives and platelet derivative compositions comprising the same, can have additional characteristics disclosed herein for such derivatives and compositions.

Processes provided herein for producing platelet derivative compositions, provide better lot to lot consistency than prior processes. For example, TFF methods provided herein provide improved lot to lot variability with respect to the components of compositions that include platelet derivatives prepared therein, in illustrative embodiments, compositions that include freeze-dried platelet derivates. Such freeze-dried platelet derivatives can be one of, or the main active ingredient(s). In some embodiments, a plurality of containers provided herein comprise the platelet derivative composition from at least 2 different lots in separate containers. In some embodiments, the amount of plasma protein in the powder of any two containers chosen from different lots, differs by less than 50%, 40%, 30%, 25%, or 20%, and in illustrative embodiments less than 10%, 5%, 2%, 1%, or 0.5%. The TFF process is highly controllable and can be stopped at a certain A280 for example, from 2.0 AU to 0.01 AU, or when it reaches 15% to 0.01% protein concentration in the composition that is to be lyophilized (e.g. TFF-treated composition), therefore, the plasma protein content can be very consistent not only within the containers/vials of a lot, but even between lots as well. Since different lots of platelet derivative compositions provided herein are typically prepared from platelets from different subjects or different combinations of subjects (e.g. pooled platelets from 2, 3, 4, 5, 6, 7, 8, 9, 10, 15, 20, 25, 30, 40, 50, 60, 70, 75, 80, or 100 subjects), different lots in illustrative embodiments differ in amino acid sequence of at least one (e.g., 1, 2, 3, 4, 5, 6, 7, 8, 9, 10, 1-100, or 1-10) of the proteins in, on, and/or associated with platelet derivatives of the compositions therein between the lots. In illustrative embodiments, these one or more amino acid differences occur at one or more (e.g., 1, 2, 3, 4, 5, 6, 7, 8, 9, 10, 1-100, or 1-10) of the site(s) of SNPs, in illustrative embodiments non-synonymous SNPs. In certain embodiments, such SNPs or non-synonymous SNPs have a minor allele frequency of less than or equal to 5%. In some embodiments, such pooled platelets are provided by processes provided herein, for example because HLA or HNA antibody levels can be reduced to very low or non-existent levels. Thus, not only can platelets be pooled from more subjects, before processing to form platelet derivatives, but those subjects can be males or females. As a result, platelet derivatives, in illustrative embodiments, FDPDs, of compositions herein, for example liquid or dried compositions, in some embodiments have different amino acid sequences for at least 1 or a plurality of FDPD proteins. Furthermore, as a result, in certain embodiments, within a lot or between lots, greater than 10%, 20%, 25%, 30%, or 40%, and in illustrative embodiments greater than 50%, 60%, 70%, 75%, 80%, 90%, or 95% of amino acids encoded by SNPs, in illustrative embodiments encoded by non-synonymous SNPs in one or more proteins that are bound to or otherwise associated with or part of a platelet derivative, are present for SNPs, for example with a minor allele frequency of greater than 5%, in certain embodiments including in proteins that result from expression of coding sequences comprising SNPs, in illustrative embodiments non-synonymous SNPs on a mammalian X and Y chromosome.

In some embodiments, the amount of microparticles that are less than 0.5 μm in the powder of any two containers chosen from different lots, differs in amount by less than 10%, 5%, 2%, or 1%. Since, for example, a TFF process disclosed herein is very controllable, the concentration of microparticles to be obtained in the platelet derivative composition can be optimized, for example, by performing scattering intensity studies at different time points. Once the desired level is achieved, the TFF-treated composition can be lyophilized and packed in the vials with or without the baking step.

In some embodiments, the percentage by weight of platelet derivative in the powder of any two containers chosen from different lots, differs by less than 10%, 5%, 2%, or 1%. The TFF process can be optimized to achieve a pre-determined level of platelet derivatives in the TFF-treated composition. Such a TFF-treated composition when lyophilized gives a platelet composition in the form of a powder having a certain weight percentage of platelet derivatives. Since, the TFF process is controllable, in some embodiments, there can be a minimum or a negligible variation in the weight percentages of the platelet derivatives in any two containers chosen from different lots.

In some embodiments, at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

As per one of the embodiments, a platelet derivative composition as described herein can be prepared from multiple donors of a single species, for example, mammals, such as for example canine, equine, porcine and in illustrative embodiments humans that are genetically different, in order to obtain a platelet derivative composition to prepare allogenic platelet derivatives, an allogenic platelet derivative product, and/or a composition comprising allogenic platelet derivatives. Such a platelet derivative composition can be filled in vials and a plurality of such vials can be packaged in containers, for example boxes for commercialization as described herein, to obtain a commercial product that is a composition comprising allogeneic platelet derivatives, in illustrative embodiments allogeneic freeze-dried platelet derivatives. The allogenic platelet derivatives as described herein, in some embodiments, can be a U.S. FDA-approved product comprising an allogenic platelet derivative composition. In some embodiments, a platelet derivative composition as described herein can be a European EMA-approved product comprising an allogenic platelet derivative composition. In some other embodiments, a platelet derivative composition as described herein can be a China FDA-approved product comprising an allogenic platelet derivative composition.

In some embodiments, platelets are pooled from a plurality of donors before they are used as starting material for a process for producing a platelet derivative as provided herein. Such platelets pooled from a plurality of donors can be also referred herein to as pooled platelets. In some embodiments, the donors are more than 5, such as more than 10, such as more than 20, such as more than 50, such as up to about 100 donors. In some embodiments, the donors are from 5 to 100, such as from 10 to 50, such as from 20 to 40, such as from 25 to 35. Pooled platelets can be used to make any of the platelet derivative compositions as described herein. The platelets can be pooled wherein the platelets are donated by mammalian (e.g. bovine, feline, porcine, canine, and in illustrative embodiments, human) subjects. In some embodiments, the gender of the subjects can be male or female. In some embodiments, the donor can vary from any number of male to any number of female subjects, for example, from a total of 100 donors, any number can be female donors, ranging from 0-100, 5-95, 10-90, 20-80, 30-70, or 40-60, and the rest can be male donors. In some other embodiments, the donor can be a non-human animal. In some embodiments, the donor can be a canine, equine, porcine, bovine, or feline subject.

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

### Platelet Derivatives Comprising an Imaging Agent

In some embodiments, provided herein is a platelet derivative composition, wherein the platelet derivatives can include an imaging agent. In some embodiments, platelets can include/be loaded with an imaging agent, for example before being dried or after being dried. In some embodiments, platelet derivatives that include an imaging agent, such as imaging agent-loaded platelet derivatives can retain the ability or properties of platelet derivatives that are not loaded with imaging agents. Loading of platelet derivatives with an agent typically facilitates imaging of the platelet derivatives in vivo. Thus, a step of detecting an imaging agent, for example an MRI agent, in a subject can be included in any method herein that includes administering or otherwise delivering platelet derivatives to the subject. The imaging agent that can be used to load the platelet derivatives as described herein can include a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 123I, ¹³¹I or ⁷⁷Br. For example, a positron-emitting radioactive non-metal can include, but is not limited to ¹¹C, ¹³N, ¹⁵O, ¹⁷F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s (dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y, ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, an MRI agent loaded into platelets can be associated with gold or other equivalent metal particles (such as nanoparticles). For example, a metal particle system can include, but is not limited to gold nanoparticles (e.g., Nanogold™).

In some embodiments, an MRI agent loaded into platelets that is modified with an imaging agent is imaged using an imaging unit. The imaging unit can be configured to image the MRI agent loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen, Z., et. al., Advance of Molecular Imaging Technology and Targeted Imaging Agent in Imaging and Therapy, Biomed Res Int., 819324, doi: 10.1155/2014/819324 (2014) have described various imaging techniques and which is incorporated by reference herein in its entirety. In some embodiments, an imaging agent is an MRI agent. In other embodiments, the MRI agent can be gadolinium-based compounds. In some embodiments, platelet derivatives that include an MRI agent, such as, MRI agent-loaded platelet derivatives can retain the ability or properties of platelet derivatives that are not loaded with MRI agents.

In some embodiments, an imaging agent as provided herein is loaded on platelets or platelet derivatives using a cell penetrating peptide (CPP). In some embodiments, an imaging agent can be conjugated to a CPP and further can be incubated with platelets or platelet derivatives to form imaging agent-loaded platelets or platelet derivatives. In some embodiments, an imaging agent can be an MRI agent to form MRI agent-loaded platelets or platelet derivatives. In some embodiments, a CPP can be any of the CPP well known in the art that can cross the cytoplasmic membrane efficiently. In some embodiments, CPP can be any peptide having 10 to 30 amino acids and are capable of crossing the cytoplasmic membrane. In some embodiments, CPP can be any of the peptides that are described in Kersemans et al 2008 (Kersemans, Veerle et al. “Cell penetrating peptides for in vivo molecular imaging applications.” Current pharmaceutical design vol. 14, 24 (2008): 2415-47.). In some embodiments, CPP can be a protein-derived CPP, a synthetic CPP, or a mixed CPP. In some embodiments, a protein-derived CPP are derived from naturally occurring protein such as, but not limiting to, TAT protein, and penetratin. In some embodiments, a synthetic CPP such as, but not limiting to polyarginines can be a CPP that is developed by known techniques, such as, phage display method. In some embodiments, a mixed CPP can be a CPP which are a combination of naturally occurring (protein derived) CPP, and synthetic CPP, such as transportan CPP, a combination of the N-terminal fragment of the neuropeptide gelanin and the membrane-interacting wasp venom peptide, mastoparan. In some embodiments, a protein-derived CPPs is selected from the group consisting of penetratin, TAT peptide (49-57 amino acids), TAT peptide (48-60 amino acids), calcitonin-derived CPP, nuclear localization sequences, new polybasic CPPs, N-terminal repetitive domain of maize gamma-zein, peptides from gp41 fusion sequence, preS2-TLM, signal-sequence hydrophobic region (SSHR), pVEC, Vpr, VP22, Human integrin b3 signal sequence, gp41 fusion sequence, Caiman crocodylus Ig(v) light chain, hCT derived peptide, Kaposi FGF signal sequences, CPP from pestivirus envelope glycoprotein, CPP derived from the prion protein, Yeast PRP6 (129-144), Phi21 N (12-29), Delta N (1-22), FHV coat (35-49), BMV Gag (7-25), HTLV-II Rex (4-16), HIV-1 Rev (9-20), RSG-1.2, Lambda-N (48-62), SV40 NLS, Bipartite, Nucleoplasmin (155-170), NLS, Herpesvirus, 8 k8 protein (124-135), Buforin-II (20-36), Magainin, PDX-1-PTD, crotamine, pIsl, SynB1, Fushi-tarazu (254-313), and Engrailed (454-513). In certain illustrative embodiments, a protein-derived CPP is penetratin. In certain illustrative embodiments, a protein-derived CPP is TAT peptide (49-57 amino acids). In certain illustrative embodiments, a protein-derived CPP is TAT peptide (48-60 amino acids). In certain illustrative embodiments, a protein-derived CPP can be any peptide described in the publication Kersemans et al 2008.

In some embodiments, a synthetic and/or mixed CPP is selected from the group consisting of transportan, polyarginine CPPs, poly-d-arginine, KLAL peptide/model amphipathic peptide (MAP), KALA model amphipathic peptide, modeled Tat peptide, Loligomer, b-sheet-forming peptide, retro-inverso forms of established CPPs, W/R penetratin, MPG, Pep-1, Signal-sequence-based peptides (I), Signal-sequence-based peptides (II), Carbamate 9, PTD-4, PTD-5, RSV-A9, CTP-512, and U2AF. In certain illustrative embodiments, a synthetic and/or mixed CPP can be any peptide described in the publication Kersemans et al 2008.

### EXEMPLARY EMBODIMENTS

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In one aspect, provided herein is a composition comprising an effective dose of platelet derivatives for use for controlling bleeding in a subject, wherein said composition is administered to the subject:
i) as a continuous infusion procedure; or
ii) at a frequency of at least one dose every 6 hours, 5 hours, 4 hours, 3 hours, 2 hours, or more frequently, in illustrative embodiments, every 1 hour, 45 minutes, 30 minutes, 15 minutes, or more frequently, wherein the administration comprises administering a total of 2 to 10, 3 to 10 doses, 4 to 10 doses, or 5 to 10 doses in a 24-hour period,
until bleeding of the subject is reduced as compared to the bleeding before the administration. In illustrative embodiments, the platelet derivatives:
a) have the ability to generate thrombin in vitro in the presence of tissue factor and phospholipids;
b) have the ability to occlude a collagen-coated microchannel in vitro; or
c) both a) and b). In some embodiments, a dose of platelet derivatives is in the range of 1.0×10⁵ to 1.0×10¹²/kg, 1.0×10⁷ to 1.0×10¹²/kg, 1.0×10⁸ to 1.0×10¹¹/kg or 5.0×10⁸ to 1.0×10¹⁰/kg of the subject. In some embodiments, at least 55%, 60%, 65%, in illustrative embodiments, at least 70%, 75%, or 80% of the platelet derivatives are positive for CD41. In some embodiments, at least 55%, 60%, 65%, and in illustrative embodiments, at least 70% of the platelet derivatives are positive for CD42. In some embodiments, at least 55%, 60%, 65%, in illustrative embodiments, at least 70%, or 75% of the platelet derivatives are positive for CD62. In some embodiments, platelet derivatives have a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation. In some embodiments, a population of platelet derivatives can aggregate in the absence of an agonist and in illustrative embodiments in the absence of thrombin, but in the presence of a divalent cation. In some embodiments, platelet derivatives herein are capable of generating more thrombin as compared to thrombin generated by platelets, such as fresh platelets, or apheresis platelets.

In one aspect, provided herein is a composition comprising an effective dose of platelet derivatives for use for controlling bleeding in a subject,
wherein said composition is administered to the subject multiple times by administering 2 to 20 doses, 3 to 20 doses, 4 to 20 doses, or 5 to 20 doses, 2 to 10 doses, 3 to 10 doses, 4 to 10 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, until bleeding of the subject is reduced as compared to the bleeding before the administering, and
wherein the platelet derivatives have the ability to generate thrombin in vitro in the presence of tissue factor and phospholipids. In some embodiments, a dose of platelet derivatives is in the range of 1.0×10⁵ to 1.0×10¹²/kg, 1.0×10⁷ to 1.0×10¹²/kg, 1.0×10⁸ to 1.0×10¹¹/kg or 5.0×10⁸ to 1.0×10¹⁰/kg of the subject. In some embodiments, at least 55%, 60%, 65%, in illustrative embodiments, at least 70%, 75%, or 80% of the platelet derivatives are positive for CD41. In some embodiments, at least 55%, 60%, 65%, and in illustrative embodiments, at least 70% of the platelet derivatives are positive for CD42. In some embodiments, at least 55%, 60%, 65%, in illustrative embodiments, at least 70%, or 75% of the platelet derivatives are positive for CD62. In some embodiments, platelet derivatives have a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation. In some embodiments, a population of platelet derivatives can aggregate in the absence of an agonist and in illustrative embodiments in the absence of thrombin, but in the presence of a divalent cation. In some embodiments, platelet derivatives herein are capable of generating more thrombin as compared to thrombin generated by platelets, such as fresh platelets, or apheresis platelets.

In one aspect, provided herein is a method for administering a platelet derivative composition to a subject for reducing bleeding of the subject, said method comprising: administering an effective dose of platelet derivatives in the platelet derivative composition to the subject, in illustrative embodiments, the subject is a human subject with a platelet dysfunction or a platelet disorder, for example, Hermansky-Pudlak syndrome (HPS),
i) as a continuous infusion procedure; or
ii) at a frequency of at least one dose every 6 hours, 5 hours, 4 hours, 3 hours, 2 hours, or more frequently, in illustrative embodiments, every 1 hour, 45 minutes, 30 minutes, 15 minutes, or more frequently, wherein the administration comprises administering a total of 2 to 10, 3 to 10 doses, 4 to 10 doses, or 5 to 10 doses in a 24-hour period,
until bleeding of the subject is reduced as compared to the bleeding before the administration, and
in illustrative embodiments, wherein the platelet derivatives have a reduced propensity to aggregate under aggregation conditions comprising an agonist but no fresh platelets, and in the absence of divalent cations, compared to the propensity of fresh platelets to aggregate under the conditions.

In one aspect, provided herein is a method for administering a platelet derivative composition to a subject for reducing bleeding of the subject, said method comprising:
administering an effective dose of platelet derivatives in the platelet derivative composition to the subject, in illustrative embodiments, the subject is a human subject with a platelet dysfunction or a platelet disorder, for example, Hermansky-Pudlak syndrome (HPS),
wherein the administering is performed by administering to the subject multiple times 2 to 10 doses, 3 to 10 doses, 4 to 10 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, until bleeding of the subject is reduced as compared to the bleeding before the administering,
wherein the platelet derivatives have the ability to generate thrombin in vitro in the presence of tissue factor and phospholipids, and
wherein the platelet derivatives have a reduced propensity to aggregate under aggregation conditions comprising an agonist but no fresh platelets, and in the absence of divalent cations, compared to the propensity of fresh platelets to aggregate under the conditions. In some embodiments, a dose of platelet derivatives is in the range of 1.0×10⁵ to 1.0×10¹²/kg, 1.0×10⁷ to 1.0×10¹²/kg, 1.0×10⁸ to 1.0×10¹¹/kg or 5.0×10⁸ to 1.0×10¹⁰/kg of the subject. In some embodiments, the administering increases the levels of at least one platelet biomarker selected from CD62P, PAC-1, and CD63 for endogenous platelets of the subject (recipient) as compared to before the administering. In illustrative embodiments, levels of both CD62P and PAC-1 are increased in the subject after the administering. In some embodiments, the method further comprises before the administering, rehydrating the freeze-dried platelet derivatives to form a rehydrated platelet derivative composition, and wherein the administering is administering an effective dose of the rehydrated platelets from the rehydrated platelet derivative composition to the subject.

In one aspect, provided herein is a method for administering a composition comprising platelet derivatives to a subject in need thereof, the method comprising:
administering the composition to the subject as a continuous infusion procedure, or
administering the composition at a frequency, wherein the administering is performed multiple times at a dose in the range of 1.0×10⁵ to 1.0×10¹⁰/kg, 1.0×10⁸ to 1.0×10¹²/kg, or 5.0×10⁸ to 1.0×10¹¹/kg by administering at least a single dose of the composition to the subject at a frequency of every 2 minutes to 15 minutes starting from a first dose, until bleeding of the subject is reduced or stopped,
wherein the platelet derivatives,
a) have the ability to generate thrombin in vitro in the presence of tissue factor and phospholipids;
b) have the ability to occlude a collagen-coated microchannel in vitro, in the presence of divalent cations and in the absence of platelets; or
c) both a) and b), and in illustrative embodiments, less than 5% of platelet derivatives in the composition are microparticles having a diameter of less than 0.5 μm.

In one aspect, provided herein is a composition comprising an effective dose of platelet derivatives for use for controlling bleeding in a subject, wherein said composition is administered:
as a continuous infusion procedure, or
to the subject multiple times at a dose in the range of 1.0×10⁵ to 1.0×10¹⁰/kg, 1.0×10⁸ to 1.0×10¹²/kg, or 5.0×10⁸ to 1.0×10¹¹/kg by administering at least a single dose of the composition, in illustrative embodiments multiple times, to the subject at a frequency of every 2 minutes to 15 minutes starting from a first dose, until bleeding of the subject is reduced or stopped,
wherein the platelet derivatives,
a) have the ability to generate thrombin in vitro in the presence of tissue factor and phospholipids;
b) have the ability to occlude a collagen-coated microchannel in vitro, in the presence of divalent cations and in the absence of platelets; or
c) both a) and b), and in illustrative embodiments, less than 5% of platelet derivatives in the composition are microparticles having a diameter of less than 0.5 μm. In some embodiments, at least 55%, 60%, 65%, in illustrative embodiments, at least 70%, 75%, or 80% of the platelet derivatives are positive for CD41. In some embodiments, at least 55%, 60%, 65%, and in illustrative embodiments, at least 70% of the platelet derivatives are positive for CD42. In some embodiments, at least 55%, 60%, 65%, in illustrative embodiments, at least 70%, or 75% of the platelet derivatives are positive for CD62. In some embodiments, platelet derivatives have a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation. In some embodiments, a population of platelet derivatives can aggregate in the absence of an agonist and in illustrative embodiments in the absence of thrombin, but in the presence of a divalent cation. In some embodiments, platelet derivatives herein are capable of generating more thrombin as compared to thrombin generated by platelets, such as fresh platelets, or apheresis platelets.

In one aspect, provided herein is a method for administering a platelet derivative composition to a subject for reducing or stopping bleeding of the subject, said method comprising:
administering an effective dose of platelet derivatives in the platelet derivative composition to the subject,
wherein the administering is performed multiple times at a dose in the range of 1.0×10⁵ to 1.0×10¹⁰/kg, 1.0×10⁸ to 1.0×10¹²/kg, or 5.0×10⁸ to 1.0×10¹¹/kg by administering 1 to 10 doses of the platelet derivatives at a frequency of every 15 minutes, or more frequently,
until the bleeding of the subject is reduced as compared to the bleeding before the administering, or until the bleeding of the subject is stopped, and
wherein the platelet derivatives:
a) have the ability to generate thrombin in vitro in the presence of tissue factor and phospholipids;
b) have the ability to occlude a collagen-coated microchannel in vitro in the presence of divalent cations and in the absence of platelets; or
c) both a) and b).

In one aspect, provided herein is a composition comprising an effective dose of platelet derivatives for use for reducing or stopping bleeding of the subject,
wherein the composition is administered to the subject multiple times at a dose in the range of 1.0×10⁵ to 1.0×10¹⁰/kg, 1.0×10⁸ to 1.0×10¹²/kg, or 5.0×10⁸ to 1.0×10¹¹/kg by administering 1 to 10 doses of the platelet derivatives at a frequency of every 15 minutes, or more frequently,
until the bleeding of the subject is reduced as compared to the bleeding before the administering, or until the bleeding of the subject is stopped, and
wherein the platelet derivatives:
a) have the ability to generate thrombin in vitro in the presence of tissue factor and phospholipids;
b) have the ability to occlude a collagen-coated microchannel in vitro in the presence of divalent cations and in the absence of platelets; or
c) both a) and b).

In one aspect, provided herein is a method for administering a platelet derivative composition to a subject for reducing bleeding of the subject, said method comprising:
administering an effective dose of platelet derivatives in the platelet derivative composition to the subject,
wherein the administering is performed multiple times by administering, in illustrative embodiments a dose of at least 1×10⁸/kg, 5×10⁸/kg, 1×10⁹/kg, 1.2×10⁹/kg, 1.4×10⁹/kg, or 1.6×10⁹/kg, a total of 3 to 50 doses within 1 hour starting from a first dose, until the bleeding is reduced, and
wherein the platelet derivatives:
a) have the ability to generate thrombin in vitro in the presence of tissue factor and phospholipids;
b) have the ability to occlude a collagen-coated microchannel in vitro in the presence of divalent cations and in the absence of platelets; or
c) both a) and b). In some embodiments, at least 55%, 60%, 65%, in illustrative embodiments, at least 70%, 75%, or 80% of the platelet derivatives are positive for CD41. In some embodiments, at least 55%, 60%, 65%, and in illustrative embodiments, at least 70% of the platelet derivatives are positive for CD42. In some embodiments, at least 55%, 60%, 65%, in illustrative embodiments, at least 70%, or 75% of the platelet derivatives are positive for CD62. In some embodiments, platelet derivatives have a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation. In some embodiments, a population of platelet derivatives can aggregate in the absence of an agonist and in illustrative embodiments in the absence of thrombin, but in the presence of a divalent cation. In some embodiments, platelet derivatives herein are capable of generating more thrombin as compared to thrombin generated by platelets, such as fresh platelets, or apheresis platelets.

In one aspect, provided herein is a composition comprising an effective dose of platelet derivatives for reducing bleeding of the subject, wherein the composition is administered by administering multiple times, in illustrative embodiments a dose of at least 1×10⁸/kg, 5×10⁸/kg, 1×10⁹/kg, 1.2×10⁹/kg, 1.4×10⁹/kg, or 1.6×10⁹/kg, a total of 3 to 50 doses within 6 hours, 5 hours, 4 hours, 3 hours, 2 hours, 1 hour, in illustrative embodiments, within 45, 30, or 15 minutes starting from a first dose, until the bleeding is reduced, and
wherein the platelet derivatives:
a) have the ability to generate thrombin in vitro in the presence of tissue factor and phospholipids;
b) have the ability to occlude a collagen-coated microchannel in vitro in the presence of divalent cations and in the absence of platelets; or
c) both a) and b).

In one aspect, provided herein is a method for administering a platelet derivative composition to a subject for reducing the bleeding of the subject, said method comprising:
administering an effective dose of platelet derivatives in the platelet derivative composition to the subject,
wherein the administering comprises administering a total of 3 to 20, 3 to 15, 3 to 12, 3 to 9, 4 to 20, 4 to 15, 4 to 12, 4 to 8, 5 to 20, 5 to 15, or 5 to 10 doses within 1 hour starting from the first dose, in illustrative embodiments until the bleeding is reduced, and
wherein the platelet derivatives:
a) have the ability to generate thrombin in vitro in the presence of tissue factor and phospholipids;
b) have the ability to occlude a collagen-coated microchannel in vitro in the presence of divalent cations and in the absence of platelets; or
c) both a) and b).

In one aspect provided herein is a method for administering freeze-dried platelet derivatives (FDPDs) to a subject having Hermansky Pudlak Syndrome (HPS), comprising administering an effective dose of the freeze-dried platelet derivatives in a platelet derivative composition to the subject. The FDPDs can have numerous characteristics provided herein, that make them well suited to restore hemostatic functions in the subject. In some embodiments, the FDPDs are from a pool of donors, and are HLA Class 1-characterized FDPDs, which in certain illustrative embodiments are HLA Class 1-matched FDPDs.

In another aspect, provided herein is a method for administering freeze-dried platelet derivatives to a subject having Hermansky Pudlak Syndrome (HPS), comprising:
administering an effective dose of the freeze-dried platelet derivatives in a platelet derivative composition to the subject,
wherein the platelet derivative composition comprises a population of freeze-dried platelet derivatives (FDPDs) comprising CD 41-positive platelet derivatives, wherein less than 5% of the CD 41-positive platelet derivatives are microparticles,
and wherein the FDPDs
   a) have the ability to generate thrombin in vitro in the presence of tissue factor and phospholipids;
   b) have the ability to occlude a collagen-coated microchannel in vitro; or
   c) both a) and b).

In one aspect, provided herein is a composition comprising platelets or platelet derivatives and an aqueous medium, wherein the aqueous medium has a protein concentration less than or equal to 50% of the protein concentration of donor apheresis plasma.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours. In some embodiments, multiple doses are delivered or administered in methods herein, at the same frequency, and in illustrative embodiments, they are delivered at different frequencies, or some at the same and some at different frequencies. In illustrative embodiments, such frequency is based on an effective of the prior dose(s) on bleeding. In some embodiments, said composition comprises a population of platelet derivatives having a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation, and wherein the platelet derivatives are capable of generating thrombin, for example in vitro in the presence of tissue factor and phospholipids, and in certain embodiments have a potency of at least 0.5, 1.0, and in non-limiting illustrative embodiments 1.5 thrombin generation potency units (TGPU) per 10⁶ platelet derivatives, or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising a population of platelet derivatives having a reduced propensity to aggregate, wherein no more than 25%, 10%, 5%, 4%, 3%, or in illustrative embodiments, no more than 2% no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation; and having one or more, two or more, or all of the following characteristics of a super-activated platelet selected from: a. the presence of thrombospondin (TSP) on their surface at a level that is greater than on the surface of resting platelets; b. the presence of von Willebrand factor (vWF) on their surface at a level that is greater than on the surface of resting platelets; c. an inability to increase expression of a platelet activation marker in the presence of an agonist as compared to the expression of the platelet activation marker in the absence of an agonist.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising platelet derivatives, wherein less than 15%, and in certain non-limiting illustrative embodiments less than 5% of the CD 41-positive platelet derivatives are microparticles, in non-limiting illustrative embodiments having a diameter of less than 1 μm, and in certain non-limiting illustrative embodiments less than 0.5 μm, and wherein the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition, comprising a population of platelet derivatives comprising CD 41-positive platelet derivatives, wherein the population comprises platelet derivatives having a reduced propensity to aggregate such that no more than 25%, 10%, 5%, 4%, 3%, or in illustrative embodiments, no more than 2% no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation, wherein the platelet derivatives have an inability to increase expression of a platelet activation marker in the presence of an agonist as compared to the expression of the platelet activation marker in the absence of the agonist, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets; and wherein less than 15%, and in certain non-limiting illustrative embodiments less than 5% of the CD 41-positive platelet derivatives are microparticles having a diameter of less than 1 μm, and in certain non-limiting illustrative embodiments less than 0.5 μm.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising a population of platelet derivatives having a reduced propensity to aggregate, such that no more than 25%, 10%, 5%, 4%, 3%, or in illustrative embodiments, no more than 2% no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising a population of platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm, and comprising platelet derivatives having one or more of, two or more of, three or more of, and in illustrative embodiments all of the following: a reduced propensity to aggregate, in certain embodiments such that no more than 25%, 10%, 5%, 4%, 3%, and in illustrative embodiments no more than 2% of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising trehalose in the range of 20-35% by weight, polysucrose in the range of 45-60% by weight, and platelet derivatives in the range of 0.5-20% by weight, wherein the platelet derivatives to microparticles have a numerical ratio of at least 95:1 in the platelet derivative composition, and wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition comprising trehalose in the range of 20-35% by weight, polysucrose in the range of 45-60% by weight, and platelet derivatives in the range of 0.5-20% by weight, wherein the platelet derivative composition comprises a population of platelet derivatives having a reduced propensity to aggregate such that no more than 25%, 10%, 5%, 4%, 3%, or in illustrative embodiments, no more than 2%, of the platelet derivatives in the population aggregate under aggregation conditions comprising an agonist but no platelets, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a plurality of containers each containing a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, wherein the platelet derivative composition in each container comprises a population of platelet derivatives having a reduced propensity to aggregate such that no more than 25%, 10%, 5%, 4%, 3%, or in illustrative embodiments, no more than 2% of the platelet derivatives in the population aggregate under aggregation conditions comprising an agonist but no platelets, wherein the platelet derivative compositions in each container are capable of generating thrombin. In illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, wherein the platelet derivatives have an inability to increase expression of a platelet activation marker in the presence of an agonist as compared to the expression of the platelet activation marker in the absence of the agonist, wherein the plurality of containers comprise the platelet derivative composition from at least 2 different lots in separate containers, and wherein one or more of: the amount of plasma protein in the powder of any two containers chosen from different lots, differs by less than 10%, 5%, 2%, or 1%, and the amount of microparticles that are less than 1 μm, and in certain non-limiting illustrative embodiments less than 0.5 μm in the powder of any two containers chosen from different lots, differs by less than 10%, 5%, 2%, or 1%.

In one aspect, provided herein is a plurality of containers each filled with a platelet derivative composition in the form of a powder, a composition comprising platelet derivatives for use for controlling bleeding in a subject, or for a method for controlling bleeding in a subject using a composition comprising platelet derivatives, wherein said use or method comprises administering at least a single dose of platelet derivatives multiple times at a frequency of every 30 minutes, 45 minutes, 1 hour, 2 hours, 3 hours, 4 hours, 5 hours, 6 hours, or more frequently, and in illustrative embodiments, comprising a total of 2-20, 2-15, 2-12, 2-10, 2-8, 3-20, 3-15, 3-12, 3-10, 3-8, 4-20, 4-15, 4-12, 4-10, 4-8, 5-20, 5-15, 5-12, 5-10, or 5-8 doses within 1 hour, 2, 3, 4, 5, 6, 7, 8, 9, 10, 11, 12, or 24 hours, said composition, wherein the platelet derivative composition comprises trehalose in the range of 20-35% by weight; polysucrose in the range of 45-60% by weight; and platelet derivatives in the range of 0.5-20% by weight, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, and a population of platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm.

In one aspect, provided herein is a process for preparing a composition comprising platelets or platelet derivatives, FPDPs, or FPH used in in any of the methods herein, and an aqueous medium, the process comprising: tangential flow filtration (TFF) of a starting material comprising platelets, a diluted starting material comprising platelets, a concentrated platelet composition, or a combination thereof, thereby preparing a composition comprising platelets or platelet derivatives and aqueous medium, wherein the aqueous medium has a protein concentration less than or equal to 50% of the protein concentration of donor apheresis plasma.

In one aspect, provided herein is a process for preparing freeze-dried platelets, comprising: a) preparing a composition comprising platelets and an aqueous medium using the process comprising: tangential flow filtration (TFF) of a starting material comprising platelets, a diluted starting material comprising platelets, a concentrated platelet composition, or a combination thereof, thereby preparing a composition comprising platelets or platelet derivatives and aqueous medium, wherein the aqueous medium has a protein concentration less than or equal to 50% of the protein concentration of donor apheresis plasma; and b) freeze-drying the composition comprising platelets and the aqueous medium.

In one aspect, provided herein is a process for preparing a composition comprising platelets or platelet derivatives and an aqueous medium, the process comprising: diluting a starting material comprising platelets to form a diluted starting material; concentrating the diluted starting material such that the platelets have a concentration of about 2250×10³ cells/μL (±250×10³) to form a concentrated platelet composition; and washing the concentrated platelet composition with at least 2 diavolumes (DV) of a preparation agent to form a TFF-treated composition.

In one aspect, provided herein is a process for preparing a platelet derivative composition, comprising performing tangential flow filtration (TFF) of a platelet composition with a preparation agent, in a preparation agent, or in a solution that optionally can have components of the preparation agent or can be a different solution, wherein the TFF is performed to at least partially exchange the solution with the preparation agent having a pH in the range of 5.5 to 8.0 and comprising 0.4 to 35% trehalose and 2% to 8% polysucrose, wherein said TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl, in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivatives in the platelet derivative composition are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, and a reduced propensity to aggregate, wherein no more than 25%, 10%, 5%, 4%, 3%, or in illustrative embodiments, no more than 2%, of the platelet derivatives in the population aggregate under aggregation conditions comprising an agonist but no platelets. The platelets can be diluted 1:0.5, 1:1, 1:2, 1:5, or 1:10 in a solution or in the preparation agent before performing the TFF.

In one aspect, provided herein is a process for preparing a platelet derivative composition, comprising performing tangential flow filtration (TFF) of a platelet composition with a preparation agent, in a preparation agent, or in a solution that optionally can have components of the preparation agent or can be a different solution, wherein the TFF is performed to at least partially exchange the solution with the preparation agent having a pH in the range of 5.5 to 8.0 and comprising 0.4 to 35% trehalose and 2% to 8% polysucrose, wherein the TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl, in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm, freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried platelet composition to form the platelet derivative composition, wherein the platelet derivative composition is: a) negative for HLA Class I antibodies based on a regulatory agency approved test for HLA Class I antibodies; b) negative for HLA Class II antibodies based on a regulatory agency approved test for HLA Class II antibodies; and c) negative for HNA antibodies based on a regulatory agency approved test for HNA antibodies. The platelets can be diluted 1:0.5, 1:1, 1:2, 1:5, or 1:10 in a solution or in the preparation agent before performing the TFF.

In one aspect, provided herein is a process for preparing a platelet derivative composition, comprising performing tangential flow filtration (TFF) of a platelet composition with a preparation agent, in a preparation agent, or in a solution that optionally can have components of the preparation agent or can be a different solution, wherein the TFF is performed to at least partially exchange the solution with the preparation agent having a pH in the range of 5.5 to 8.0 and comprising 0.4 to 35% trehalose and 2% to 8% polysucrose, wherein said TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivative composition comprising a population of platelet derivatives having a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets and having one or both of: the presence of thrombospondin (TSP) on their surface at a level that is greater than on the surface of resting platelets; and the presence of von Willebrand factor (vWF) on their surface at a level that is greater than on the surface of resting platelets. The platelets can be diluted 1:0.5, 1:1, 1:2, 1:5, or 1:10 in a solution or in the preparation agent before performing the TFF.

In one aspect, provided herein is a process for preparing a process for preparing a platelet derivative composition, comprising performing tangential flow filtration (TFF) of a platelet composition with a preparation agent, in a preparation agent, or in a solution that optionally can have components of the preparation agent or can be a different solution, wherein the TFF is performed to at least partially exchange the solution with the preparation agent having a pH in the range of 5.5 to 8.0 and comprising 0.4 to 35% trehalose and 2% to 8% polysucrose, wherein said TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivatives in the platelet derivative composition display one or more of, two or more of, three or more of, four or more of, or all of the following properties: a reduced propensity to aggregate, wherein no more than 25%, 10%, 5%, 4%, 3%, or in illustrative embodiments, no more than 2%, of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and are capable of generating thrombin, such that in illustrative embodiments, the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives. The platelets can be diluted 1:0.5, 1:1, 1:2, 1:5, or 1:10 in a solution or in the preparation agent before performing the TFF.

In one aspect, provided herein is a method for treating a subject, a method for controlling bleeding in a subject, said method comprising administering to the subject, a therapeutically effective amount of platelet derivatives, FDPDs, or FPH in a composition comprising platelet derivatives, FDPDs, or FPH of any aspect or embodiment herein or the platelet derivative composition prepared by a process of any method of preparing or making provided herein.

In one aspect, provided herein is a platelet derivative composition comprising platelet derivatives of any aspect provided herein, or a platelet derivative composition prepared by the process of any method for preparing or making provided herein, for use as a medicament in treating a subject.

In one aspect, provided herein is a platelet derivative composition comprising platelet derivatives of any aspect provided herein, or a platelet derivative composition prepared by the process of any method for preparing or making provided herein, wherein the platelet derivatives comprise an imaging agent.

In one aspect, provided herein is a method of delivering an imaging agent to a subject, comprising: (a) rehydrating platelet derivative composition comprising platelet derivatives of any aspect provided herein, or a platelet derivative composition prepared by the process of any method for preparing or making provided herein to form rehydrated platelet derivative composition; (b) contacting the rehydrated platelet derivative composition with an imaging agent, to form imaging agent loaded-platelet derivatives; and (c) administrating an effective dose of the imaging agent-loaded platelet derivatives to the subject.

In one aspect, provided herein is a platelet derivative composition for use in the treatment, or for controlling bleeding in a subject, in illustrative embodiments having an indication selected from the group consisting of intracranial hemorrhage (ICH), traumatic brain Injury (TBI), and Hermansky Pudlak Syndrome (HPS), in illustrative embodiments, for controlling bleeding, wherein the treatment comprises rehydrating platelet derivatives in the platelet derivative composition to form a rehydrated platelet derivative composition and administering an effective dose of the platelet derivatives in the rehydrated platelet derivative composition to the subject, in illustrative embodiments, the administering comprises administering at least a single dose of at least 1×10⁸/kg multiple times at a frequency of every 30 minutes, 45 minutes, 1 hour, 2 hours, 3 hours, 4 hours, 5 hours, 6 hours, or more frequently, or administering at least a single dose of at least 1×10⁸/kg multiple times by administering 2 to 20 doses, 2 to 15 doses, 2 to 12 doses, 2 to 10 doses, 2 to 8 doses, 3 to 20 doses, 3 to 15 doses, 3 to 12 doses, 3 to 10 doses, 4 to 20 doses, 4 to 15 doses, 4 to 12 doses, 4 to 10 doses, 4 to 8 doses, 5 to 20 doses, 5 to 15 doses, 5 to 12 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, wherein the platelet derivative composition comprises a population of platelet derivatives: comprising CD 41-positive platelet derivatives, wherein less than 10%, 8%, in illustrative embodiments, less than 5% of the CD 41-positive platelet derivatives are microparticles; having a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets; and having a potency of at least 0.5, 1.0, and in illustrative embodiments, 1.5 thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a platelet derivative composition for use in the treatment of a subject having an indication of immune thrombocytopenia (ITC), or Bernard Soulier syndrome, in illustrative embodiments, for controlling bleeding, wherein the treatment comprises rehydrating platelet derivatives in the platelet derivative composition to form a rehydrated platelet derivative composition, and administering an effective dose of the platelet derivatives in the rehydrated platelet derivative composition to the subject, in illustrative embodiments, the administering comprises administering at least a single dose of at least 1×10⁸/kg multiple times at a frequency of every 30 minutes, 45 minutes, 1 hour, 2 hours, 3 hours, 4 hours, 5 hours, 6 hours, or more frequently, or administering at least a single dose of at least 1×10⁸/kg multiple times by administering 2 to 20 doses, 2 to 15 doses, 2 to 12 doses, 2 to 10 doses, 2 to 8 doses, 3 to 20 doses, 3 to 15 doses, 3 to 12 doses, 3 to 10 doses, 4 to 20 doses, 4 to 15 doses, 4 to 12 doses, 4 to 10 doses, 4 to 8 doses, 5 to 20 doses, 5 to 15 doses, 5 to 12 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, wherein the platelet derivative composition comprises a population of platelet derivatives: comprising CD 41-positive platelet derivatives, wherein less than 10%, 8%, in illustrative embodiments, less than 5% of the CD 41-positive platelet derivatives are microparticles; having a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets; and having a potency of at least 0.5, 1.0, and in illustrative embodiments, 1.5 thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a platelet derivative composition for use in the treatment of a subject, or for controlling bleeding in a subject having an indication selected from the group consisting of Von Willebrand disease, immune thrombocytopenia, intracranial hemorrhage (ICH), traumatic brain Injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof, wherein the treatment comprises rehydrating platelet derivatives in the platelet derivative composition to form a rehydrated platelet derivative composition and administering an effective dose of the platelet derivatives in the rehydrated platelet derivative composition to the subject, in illustrative embodiments, administering comprises administering at least a single dose of at least 1×10⁸/kg multiple times at a frequency of every 30 minutes, 45 minutes, 1 hour, 2 hours, 3 hours, 4 hours, 5 hours, 6 hours, or more frequently, or administering at least a single dose of at least 1×10⁸/kg multiple times by administering 2 to 20 doses, 2 to 15 doses, 2 to 12 doses, 2 to 10 doses, 2 to 8 doses, 3 to 20 doses, 3 to 15 doses, 3 to 12 doses, 3 to 10 doses, 4 to 20 doses, 4 to 15 doses, 4 to 12 doses, 4 to 10 doses, 4 to 8 doses, 5 to 20 doses, 5 to 15 doses, 5 to 12 doses, or 5 to 10 doses of the platelet derivatives at a frequency of every 6 hours, 5 hours, 4 hours, 3 hours, 2 hours, or more frequently, in illustrative embodiments, every 1 hour, 45 minutes, 30 minutes, 15 minutes, or more frequently, and wherein the platelet derivative composition comprises a population of platelet derivatives: comprising CD 41-positive platelet derivatives, wherein less than 10%, 8%, in illustrative embodiments, less than 5% of the CD 41-positive platelet derivatives are microparticles; having a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets; and having a potency of at least 0.5, 1.0, and in illustrative embodiments 1.5, thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a plurality of containers each containing a platelet derivative composition in the form of a powder, wherein the platelet derivative composition comprises a population of platelet derivatives comprising CD 41-positive platelet derivatives, wherein less than 10%, 8%, in illustrative embodiments, less than 5% of the CD 41-positive platelet derivatives are microparticles having a diameter of less than 0.3 μm, 0.4 μm, 0.5 μm, 0.6 μm, 0.7 μm, 0.8 μm, 0.9 μm, or 1 μm having a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets, and having a potency of at least 0.5, 1.0, and in illustrative embodiments, 1.5 thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

In one aspect, provided herein is a process for preparing a platelet derivative composition comprising a population of platelet derivatives, the process comprising performing tangential flow filtration (TFF) of a platelet composition in a solution to at least partially exchange the solution with a preparation agent having a pH in the range of 5.5 to 8.0 and comprising 0.4 to 35% trehalose and 2% to 8% polysucrose, wherein said TFF is performed using a 0.3 to 1 micron filter, thereby preparing a TFF-treated composition comprising 100×10³-20,000×10³ platelets/μL, 1000×10³-20,000×10³ platelets/μL, 1000×10³-10,000×10³ platelets/μL, 500×10³-5,000×10³ platelets/μL, 1000×10³-5,000×10³ platelets/μL, 2000×10³-8,000×10³ platelets/μL, or 15,000×10³-20,000×10³ platelets/μL, in illustrative embodiments, 10,000×10³ to 20,000×10³ platelets/μl in an aqueous medium having less than or equal to 5%, 10%, or 15% plasma protein, and having less than 10%, 8%, in illustrative embodiments, less than 5.0% microparticles having a radius less than 0.1 μm, 0.2 μm, 0.25 μm, or 0.5 μm, by scattering intensity; and freeze drying the TFF-treated composition comprising platelets in the aqueous medium to form a freeze-dried composition comprising platelet derivatives to form the platelet derivative composition, wherein the platelet derivatives in the platelet derivative composition display a potency of at least 0.5, 1.0, and in illustrative embodiments, 1.5 thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets, and a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets.

In one aspect, provided herein is a platelet derivative composition in the form of a powder, comprising a population of platelet derivatives having a compromised plasma membrane and a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets, wherein at least 30%, 40%, 50%, 60%, 70%, or 80% of the platelet derivatives are CD 41-positive platelet derivatives, wherein less than 1%, 2%, 3%, 4%, or 5% of the CD 41-positive platelet derivatives are microparticles having a diameter or radius of less than 0.3 μm, 0.4 μm, 0.5 μm, 0.7 μm, or 1 μm and wherein the platelet derivatives have a potency of at least 0.5, 1.0, and in illustrative embodiments, 1.5 thrombin generation potency units (TGPU) per 10⁶ platelet derivatives or having an ability for generating more thrombin as compared to thrombin generated by platelets, such as fresh platelets, or apheresis platelets, in illustrative embodiments, platelet derivatives can generate at least 2 fold, 3 fold, or more thrombin in an in vitro thrombin formation assay as compared to thrombin generated by apheresis platelets.

To reiterate, any embodiments herein in this section and in this specification and associated claims, can be combined and/or used in any of the aspects herein and in combination with any of the other embodiments herein. Furthermore, a “powder” recited in any aspect or embodiment can alternatively be a solid, or a composition comprising less than 1% water content in such aspect or embodiment.

In some embodiments of any of the aspects or embodiments herein that include compositions comprising platelet derivatives for use for controlling bleeding in a subject, or include methods for controlling bleeding in a subject comprise administering at least one dose of platelet derivatives every 1 hour, or more frequently, in illustrative embodiments, the administration comprises administering a total of 2 to 30 doses, 3 to 30 doses, 4 to 30 doses, 5 to 30 doses, 5 to 25 doses, 5 to 20 doses, 5 to 15 doses, 5 to 10 doses in a 24-hour period, until bleeding of the subject is reduced as compared to the bleeding before the administration. In some embodiments of any of the aspects or embodiments herein that include compositions comprising platelet derivatives for use for controlling bleeding in a subject, or include methods for controlling bleeding in a subject, wherein said composition is administered to the subject multiple times by administering 2 to 10 doses of the platelet derivatives, in illustrative embodiments at a frequency of every 1 hour, or more frequently, until bleeding of the subject is reduced as compared to the bleeding before the administering, and wherein the platelet derivatives have the ability to generate thrombin in vitro in the presence of tissue factor and phospholipids. In some embodiments, the administering is performed until bleeding of the subject is stopped. In some embodiments, the administering is performed at a frequency of every 30 minutes, or more frequently. In some embodiments, the administering is performed at a frequency of every 15 minutes, or more frequently. In some embodiments, the administering is performed as the continuous infusion. In some embodiments, the administering is performed at a frequency of every 45 minutes, or more frequently. In some embodiments, the administering is performed by administering 3 to 10 doses at the frequency. In some embodiments, the administering is performed until bleeding of the subject is reduced that is observable by visual inspection. In some embodiments, the administering is performed until bleeding of the subject is reduced such that it is no longer considered life threatening. In some embodiments, the administering is performed until bleeding of the subject is reduced such that the bleeding is considered minor. In some embodiments, the administering is performed until it is determined that bleeding of the subject is stopped by a visual inspection. In some embodiments, the administering is performed until bleeding of the subject is stopped at a primary site. In some embodiments, the administering is performed until bleeding of the subject is stopped at any other site than a primary site of bleeding. In some embodiments, the administering is done by a topical administration of the composition. In some embodiments, the administering is done by a parenteral administration of the composition. In illustrative embodiments, the parenteral administration is done by intravenous administration. In some embodiments, the administering includes administering one or multiple doses of platelet derivatives provided herein, by each of a parenteral and a topical route of administration. In some embodiments, the administering further comprises a topical administration of a second composition, and wherein the second composition comprises platelet derivatives. In some embodiments, the second composition is the same as the composition administered by the parenteral route. In other embodiments, the second composition is different from the composition administered by the parenteral route. In some embodiments, the administering comprises the parenteral administration and the topical administration in an alternate manner. In some embodiments, a single dose or an effective dose of the platelet derivatives is in the range of 1.0×10⁷ to 1.1×10¹⁴/kg, 1.0×10⁸ to 1.1×10¹⁴/kg, or 1.5×10⁹ to 1.1×10¹⁴/kg of the subject. In some embodiments, a total of 2 to 20, 2 to 12, 4 to 10, 5 to 8, 5 to 20, or 5 to 15 doses are administered to the subject. In some embodiments, the subject is refractory to platelet transfusion. In some embodiments, the subject has HLA alloantibodies, or Human Platelet Antigen (HPA) alloantibodies. In some embodiments, the subject has a PRA score of greater than 15%, for example, between 15% and 70%. In some embodiments, the subject has a PRA score of greater than 70%. In some embodiments, the administering leads to an improvement in clot formation in the subject as compared to the subject after being administered apheresis platelets, but before the administering of the platelet derivatives. In some embodiments, the subject has Hermansky Pudlak Syndrome (HPS), and in illustrative embodiments, the administering leads to an improvement in clot formation in the subject as compared to the subject after being administered apheresis platelets, but before the administering of the platelet derivatives, and in further illustrative embodiments, the subject has HLA Class 1 alloantibodies, and in other illustrative embodiments, the administering increases the levels of at least one, two, or all of platelet biomarkers selected from CD62P, PAC-1, and CD63 for endogenous platelets in the subject as compared to the subject before the administering. In some embodiments, the administering restores the levels of at least one platelet biomarker selected from CD62P, PAC-1, and CD63 to within 25% of levels of endogenous platelets in the subject as compared to normal levels, wherein the similar levels are in between the range of 20% lower to 20% higher levels as compared to normal levels. In some embodiments, the subject has been treated with antiplatelet agent. In some embodiments, the subject is being treated with an antiplatelet agent. In some embodiments, the subject has been treated with an anticoagulant. In some embodiments, the subject is being treated with an anticoagulant. In some embodiments, less than 25%, 20%, 15%, 10%, in illustrative embodiments, less than 5%, or 3% of the platelet derivatives in the platelet derivative composition are microparticles, in illustrative embodiments CD-41 microparticles having less than 0.5 μm diameter, in illustrative embodiments, measured by scattering intensity. In some embodiments, the platelet derivatives have one, two, three, or all of the following properties: have compromised membranes, at least 55%, 60%, 65%, in illustrative embodiments, at least 70%, 75%, or 80% of the platelet derivatives are positive for CD41, at least 55%, 60%, 65%, and in illustrative embodiments, at least 70% of the platelet derivatives are positive for CD42, at least 55%, 60%, 65%, in illustrative embodiments, at least 70%, or 75% of the platelet derivatives are positive for CD62, at least 70%, in illustrative embodiments, at least 75%, 80%, 85%, or 90% of the platelet derivatives have diameter in the range of 0.5-2.5 μm, the platelet derivatives have the ability to generate thrombin in vitro in the presence of tissue factor and phospholipids, or the platelet derivatives have the ability to occlude a collagen-coated, and tissue factor coated microchannel in vitro. In some embodiments, platelet derivatives have a reduced propensity to aggregate, such that no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation. In some embodiments, platelet derivatives have one or more, two or more, or all of the following characteristics of a super-activated platelet selected from: a. the presence of thrombospondin (TSP) on their surface at a level that is greater than on the surface of resting platelets; b. the presence of von Willebrand factor (vWF) on their surface at a level that is greater than on the surface of resting platelets; c. an inability to increase expression of a platelet activation marker in the presence of an agonist as compared to the expression of the platelet activation marker in the absence of an agonist. In some embodiments, a population of platelet derivatives can aggregate in the absence of an agonist and in illustrative embodiments in the absence of thrombin, but in the presence of a divalent cation. For example, the population of platelet derivatives can aggregate in the presence of a divalent cation in the range of 5-80%, 5-75%, 5-70%, 5-65%, 5-60%, 5-50%, 5-40%, 10-80%, 15-80%, 20-80%, 25-80%, 30-80%, 35-80%, 40-80%, or 45-80%. In some embodiments, platelet derivatives herein have less than 10%, 5%, 4%, 3%, 2%, 1%, in illustrative embodiments, no crosslinking of platelet membranes via proteins and/or lipids present on the membranes.

In certain illustrative embodiments of a composition, or in some compositions used in or formed by a process, the platelet derivatives in a composition, as a non-limited example a powder, and/or formed by a process disclosed herein, are surrounded by a compromised plasma membrane, are positive for CD 41, and/or are 0.5 to 25.0 μm, 20.0 μm, 15.0 μm, 12.5 μm, 10.0 μm, or 2.5 μm in radius or diameter. In some embodiments, the composition comprises platelet derivatives such that at least 95% platelet derivatives positive for CD 41 have a radius or diameter in the range of 0.5 to 25.0 μm, 20.0 μm, 15.0 μm, 12.5 μm, 10.0 μm, or 2.5 μm. Such radius or diameter can be measured, for example by flow cytometry technique as known to a skilled artisan in the art.

In some embodiments of any of the aspects and embodiments herein that include platelet derivatives in a hydrated or rehydrated form, the protein concentration, or plasma protein concentration, is in the range of 0.01%-50%, 5%-50%, 5%-30%, 5-15%, 8%-10%, 7%-10%, or 3-7% of the protein concentration of donor apheresis plasma. In some embodiments of a composition or in some compositions used in or formed by a process herein, the protein concentration, or plasma protein concentration is less than or equal to 30%, 25%, 20%, 15%, 10%, 5%, 4%, 3%, 2%, or 1% of the protein concentration of donor apheresis plasma. In some embodiments of a composition or a process herein, the protein concentration, or plasma protein concentration is less than or equal to 15%, 14%, 13%, 12%, 11%, 10%, 9%, 8%, 7%, 6%, 5%, 4%, 3%, 2%, 1%, 0.1%, or 0.01%. In some exemplary embodiments, the protein concentration, or plasma protein concentration is less than 3% or 4%. In some embodiments, the protein concentration, or plasma protein concentration is between 0.01% and 20%, 0.01% and 15%, 0.01% and 10%, 0.010% and 5%, 0.10% and 20%, 0.10% and 15%, 0.10% and 10%, 0.10% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments of a composition or a process herein, the protein concentration is in the range of 0.01-15%, 0.1-15%, 1-15%, 1-10%, 0.01-10%, 3-12%, or 5-10%. In some embodiments, the absorbance at 280 nm is less than or equal to 2.0 AU, or 1.90 AU, or 1.80 AU, or 1.7 AU, or 1.66 AU, or 1.6 AU when measured using a path length of 0.5 cm.

In some embodiments of any of the aspects and embodiments herein that include platelet derivatives in a powdered form, the protein concentration is in the range of 0.01-15%, 0.1-15%, 1-15%, 1-10%, 0.01-10%, 3-12%, or 5-10%. In some embodiments, the protein concentration is less than or equal to 25%, 20%, 15%, 10%, 7.5%, 5%, 2.5%, 1%, or 0.1%.

In some embodiments of any of the aspects and embodiments herein that include a process for preparing a platelet derivative composition, the process comprises performing TFF of a platelet composition in a solution to at least partially exchange the solution with a preparation agent having a pH in the range of 5.5 to 8.0 and comprising trehalose and polysucrose, wherein said TFF is performed using a 0.3 to 1 micron filter, thereby preparing a TFF-treated composition.

In some embodiments of any of the aspects and embodiments herein that include a composition or in some compositions used in or formed by a process, or compositions for use for controlling bleeding of a subject that includes a population of platelet derivatives in a hydrated or rehydrated form, the composition comprises less than 10%, 7.5%, 7%, 6%, 5%, 4.5%, 4%, 3.5%, 3%, 2.5%, 2%, 1.5%, 1.0%, 0.75%, 0.5%, 0.25%, or 0.1% (by scattering intensity) microparticles. In some embodiments, the composition comprises microparticles (by scattering intensity) in the range of 0.01%-10%, 0.01%-7.5%, 0.01%-5%, 0.1%-10%, 0.1%-5%, 0.1%-4.9%, 0.5%-4.5%, 1%-10%, 1%-5%, 0.01%-4%, −0.1%-4%, 1%-4%, 1.5%-3%, 0.1%-3%, or 1%-3%. In some embodiments, the microparticles have a diameter less than 1 μm. In illustrative embodiments, the microparticles have a radius or diameter less than 0.5 μm. In some embodiments, the microparticles have a radius or diameter in the range of 0.01-0.5 μm, 0.1-0.5 μm, or 0.1-0.49 μm, 0.1-0.47 μm, or 0.1-0.45 μm, or 0.1-0.4 μm, or 0.2-0.49 μm, or 0.25-0.49 μm, or 0.3-0.47 μm. In some embodiments, the radius or diameter of the microparticles is measured using flow cytometry.

In some embodiments of any of the aspects and embodiments herein that include a platelet derivative composition in a powdered form, or compositions for use for controlling bleeding of a subject, the platelet derivative composition comprises a population of platelet derivatives comprising CD41-positive platelet derivatives, wherein less than 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-41-positive platelet derivatives are microparticles. In some embodiments, the platelet derivative composition comprises a population of platelet derivatives comprising CD42-positive platelet derivatives, wherein less than 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-42-positive platelet derivatives are microparticles. In some embodiments, the platelet derivative composition comprises a population of platelet derivatives comprising CD61-positive platelet derivatives, wherein less than 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-62-positive platelet derivatives are microparticles. In some illustrative embodiments, the microparticles are having a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. In some embodiments, the diameter of the microparticles is determined by flow cytometry.

In some embodiments of any of the aspects and embodiments herein that include a composition or in some compositions used in or formed by a process, or compositions for use for controlling bleeding of a subject, the platelet derivatives have a radius or diameter greater than 0.25 μm, greater than 0.3 μm, greater than 0.4 μm, or in illustrative embodiments, greater than 0.5 μm. In some embodiments, the platelet derivatives have a radius or diameter greater than 0.75 μm. In some embodiments, the platelet derivatives have a radius or diameter in the range of 0.25-4 μm, 0.27-3.5 μm, 0.3-3.25 μm, 0.35-3.50 μm, or 0.4-3 μm. In illustrative embodiments, the platelet derivatives have a radius or diameter of at least 0.5 μm, for example in the range of 0.5 μm on the low end of the range to 25.0 μm, 20.0 μm, 15.0 μm, 12.5 μm, 10.0 μm, 5.0 μm or 2.5 μm on the high end of the range. In some embodiments, the diameter of the platelet derivatives is measured using flow cytometry.

In some embodiments of any of the aspects and embodiments herein that include a composition or in some compositions used in or formed by a process, or compositions for use for controlling bleeding of a subject, the platelet derivatives are CD-41 positive. In some embodiments, at least 40%, 45%, 50%, 55%, 60%, 65%, 70%, or 75% of the platelet derivatives are CD-41 positive. In some embodiments, the platelet derivatives in the range of 35-97%, 40-97%, 50-97%, 60-97%, 40-95%, 45-90%, 50-95%, 60-90%, or 75-95% are positive for CD-41. In some embodiments, at least 50% of the platelet derivatives are CD 41-positive platelet derivatives, wherein less than 5% of the CD 41-positive platelet derivatives are microparticles having a diameter of less than 0.5 μm, and wherein the platelet derivatives have a potency of at least 1.5 thrombin generation potency units (TGPU) per 10⁶ platelet derivatives.

In some embodiments of any of the aspects and embodiments herein that include a platelet derivative composition in a powdered form, or compositions for use for controlling bleeding of a subject, the platelet derivatives in the platelet derivative composition have a weight percentage of at least 0.5%, 0.75%, 1%, 1.5%, 2%, 2.5% 3%, 3.5%, 4%, 4.5%, 5%, 5.5%, 6%, 6.5%, 7%, 7.5%, 8%, 8.5%, 9%, 9.5%, or 10%. In some embodiments, the platelet derivatives in the platelet derivative composition have a weight percentage in the range of 0.5 to 25%, 0.5% to 20%, 1% to 20%, 2.5% to 20%, 5% to 20%, 5% to 10%, 2.5% to 20%, 2.5% to 15%, 2.5% to 10%, or 2.5% to 7.5%.

In some embodiments of the aspects and embodiments herein that include a platelet derivative composition in a powdered form, or compositions for use for controlling bleeding of a subject, a platelet derivative composition is devoid of plasma protein. In some embodiments, the plasma protein is in the range of 0.01-15%, 0.1-15%, 1-10%, 2-15%, 3-9%, 1-5%, 1-3%, 0.1-3%, 0.5-2%, or 0.25-2%.

In some embodiments of the aspects and embodiments herein that include a platelet derivative composition in a powdered form, or compositions for use for controlling bleeding of a subject, a platelet derivative composition comprises a buffering agent in the range of 0.5-3%, 0.75-2.75%, 1-2.5%, or 1.5-2.5%. In some embodiments, the buffering agent is HEPES.

In some embodiments of any of the aspects and embodiments herein that include a composition or in some compositions used in or formed by a process, or compositions for use for controlling bleeding of a subject, the platelet derivatives have an inability to increase expression of a platelet activation marker in the presence of an agonist as compared to the expression of the platelet activation marker in the absence of the agonist. In some embodiments, the platelet activation marker is selected from the group consisting of Annexin V, and CD 62. In some embodiments, the platelet activation marker is Annexin V. In some embodiments, the platelet activation marker is CD 62. In some embodiments, the agonist is selected from the group consisting of collagen, epinephrine, ristocetin, arachidonic acid, adenosine di-phosphate, and thrombin receptor associated protein (TRAP).

In some embodiments of any of the aspects and embodiments herein that include a composition, or in some compositions used in or formed by a process, or compositions for use for controlling bleeding of a subject, the aqueous medium has a concentration of human leukocyte antigen (HLA) Class I antibodies that is less than 30%, 10%, 5%, 3%, or 1% of the human leukocyte antigen (HLA) Class I antibody concentration in donor apheresis plasma. In some embodiments, the aqueous medium has a concentration of human leukocyte antigen (HLA) Class II antibodies that is less than 30%, 10%, 5%, 3%, or 1% of the human leukocyte antigen (HLA) Class II antibody concentration in donor apheresis plasma. In some embodiments, the composition is negative for HLA Class I antibodies based on a regulatory agency approved test. In some embodiments, the composition is negative for HLA Class II antibodies based on a regulatory agency approved test. In some embodiments of the composition, a percentage of beads positive for HLA Class I antibodies, as determined for the composition by flow cytometry using beads coated with Class I HLAs, is less than 5%, 3%, or 1%. In some embodiments of the composition, a percentage of beads positive for HLA Class II antibodies, as determined for the composition by flow cytometry using beads coated with Class II HLAs is less than 5%, 3%, or 1%.

In some embodiments of any of the aspects and embodiments herein that include a composition, or in some compositions used in or formed by a process, or compositions for use for controlling bleeding of a subject, the aqueous medium has a concentration of human neutrophil antigen (HNA) antibodies that is less than 30%, 10%, 5%, 3%, or 1% of the HNA antibody concentration in donor apheresis plasma. In some embodiments, the composition is negative for HNA antibodies based on a regulatory agency approved test. In some embodiments of the composition, a percentage of beads positive for HNA antibodies, as determined for the composition by flow cytometry using beads coated with HNAs is less than 5%, 3%, or 1%.

In some embodiments of any of the aspects and embodiments herein that include a composition, or in some compositions used in or formed by a process, or compositions for use for controlling bleeding of a subject, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments of the composition, a percentage of beads positive for HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments, the composition is negative for the antibodies selected from the group consisting of HLA Class I antibodies, HLA Class II antibodies, and HNA antibodies based on a regulatory agency approved test for the respective antibodies. In some embodiments, the composition is negative for HLA Class I antibodies, HLA Class II antibodies, and HNA antibodies based on a regulatory agency approved test for the respective antibodies.

In some embodiments of any of the aspects and embodiments herein that include a composition, or in some compositions used in or formed by a process, or compositions for use for controlling bleeding of a subject that includes a population of platelet derivatives in a hydrated or rehydrated form, the platelets or platelet derivatives in a composition are at least 100×10³ platelets/μL, or 200×10³ platelets/μL, or 400×10³ platelets/μL, or 1000×10³ platelets/μL, or 1250×10³ platelets/μL, or 1500×10³ platelets/μL, or 1750×10³ platelets/μL, 2000×10³ platelets/μL, or 2250×10³ platelets/μL, or 2500×10³ platelets/μL, or 2750×10³ platelets/μL, or 3000×10³ platelets/μL, 3250×10³ platelets/μL, 3500×10³ platelets/μL, 3750×10³ platelets/μL, 4000×10³ platelets/μL, or 4250×10³ platelets/μL, or 4500×10³ platelets/μL, or 4750×10³ platelets/μL, or 5000×10³ platelets/μL, or 5250×10³ platelets/μL, or 5500×10³ platelets/μL, or 5750×10³ platelets/μL, or 6000×10³ platelets/μL, or 7000×10³ platelets/μL, or 8000×10³ platelets/μL, or 9000×10³ platelets/μL, or 10,000×10³ platelets/μL, or 11,000×10³ platelets/μL, or 12,000×10³ platelets/μL, or 13,000×10³ platelets/μL, or 14,000×10³ platelets/μL, or 15,000×10³ platelets/μL, or 16,000×10³ platelets/μL, or 17,000×10³ platelets/μL, or 18,000×10³ platelets/μL, or 19,000×10³ platelets/μL, or 20,000×10³ platelets/μL. In some embodiments of the composition, the platelets or platelet derivatives in the composition is in the range of 100×10³-20,000×10³ platelets/μL, 1000×10³-20,000×10³ platelets/μL, 1000×10³-10,000×10³ platelets/μL, 500×10³-5,000×10³ platelets/μL, 1000×10³-5,000×10³ platelets/μL, 2000×10³-8,000×10³ platelets/μL, 10,000×10³-20,000×10³ platelets/μL, 15,000×10³-20,000×10³ platelets/μL, 5000×10³ to 20,000×10³ platelets/μl, 6000×10³ to 18,000×10³ platelets/μl or 6000×10³ to 15,000×10³ platelets/μl.

In some embodiments, the above concentrations are at any point in a process herein, such as in the volume that is freeze dried. In some embodiments, the above concentrations are for platelet-derivatives herein. It is contemplated that the platelet derivative composition in the form of a powder has to be rehydrated with a solution to determine the platelet-derivative concentration, typically in the intended volume for rehydration of a powder, e.g. freeze-dried, composition, which in illustrative embodiments is a recommended volume of a container containing the powder and/or a same volume as the composition was in before it was dried to form the powder. In some embodiments, the solution for rehydrating can be water. In some embodiments, the solution for rehydrating can be a well-known buffer. In some embodiments, the amount of solution for rehydrating the platelet derivative composition is equal to the amount of buffer or preparation agent present at the step of freeze-drying. In some embodiments, the platelet concentration is in

In some embodiments of any of the aspects and embodiments herein that include a composition, or in some compositions used in or formed by a process, the composition(s) comprises a population of platelet derivatives having a reduced propensity to aggregate. In certain embodiments, no more than 25%, 22.5%, 20%, 17.5%, 12.5%, 10%, 7.5%, 5%, 4%, 3%, 2%, or 1% of the platelet derivatives in the population aggregate under aggregation conditions. In an illustrative embodiment no more than 10% of the platelet derivatives in the population aggregate under aggregation conditions. Illustrative embodiments of exemplary aggregation conditions are provided herein. For example, in illustrative embodiments such aggregation conditions comprise an agonist but no platelets are present in the aggregation conditions. In some embodiments, the agonist is selected from the group consisting of collagen, epinephrine, ristocetin, arachidonic acid, adenosine di-phosphate, and thrombin receptor associated protein (TRAP). In some embodiments, the population of platelet derivatives aggregate in the range of 2-30%, 5-25%, 10-30%, 10-25%, 12.5-25%, 2-10%, 2-8%, 2-7.5%, 2-5%, 2-4%, 0-1%, 0-2%, 0-3%, 0-4%, 0-5%, 0-7.5%, or 0-10%, or in illustrative embodiments 0 to about 1% of the platelet derivatives under aggregation conditions comprising an agonist but no platelets. It can be contemplated that aggregation conditions involve rehydrating the platelet derivative composition in an appropriate amount of water or an appropriate buffer.

In some embodiments of any of the aspects and embodiments herein that include a composition, or in some compositions used in or formed by a process, comprises erythrocytes in an amount lesser than 0.2×10⁶ erythrocytes/μL, or 0.1×10⁶ erythrocytes/μL, or 0.5×10⁵ erythrocytes/μL, or 0.1×10⁵ erythrocytes/μL. In some embodiments, the erythrocytes in the composition is in the range of 0.1×10⁵ erythrocytes/μL to 0.2×10⁶ erythrocytes/μL, or 0.5×10⁵ erythrocytes/μL to 0.1×10⁶ erythrocytes/μL.

In some embodiments of any of the aspects and embodiments herein that include a composition, or in some compositions used in or formed by a process, the aqueous medium further comprises a buffering agent, a base, a loading agent, optionally a salt, and optionally at least one organic solvent. In some embodiments, the buffering agent is HEPES (4-(2-hydroxyethyl)-1-piperazineethanesulfonic acid). In some embodiments, the base is sodium bicarbonate. In some embodiments, the loading agent is a monosaccharide, a polysaccharide, or a combination thereof. In some embodiments, the monosaccharide is selected from the group consisting of sucrose, maltose, trehalose, glucose, mannose, and xylose. In some embodiments, the monosaccharide is trehalose. In some embodiments, the polysaccharide is polysucrose. In some embodiments, the salt is sodium chloride, potassium chloride, or a combination thereof. In some embodiments, the organic solvent is selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

In some embodiments of any of the aspects and embodiments herein that include a composition, or in some compositions used in or formed by a process, or compositions for use for controlling bleeding of a subject that includes a population of platelet derivatives in a hydrated or rehydrated form, comprises trehalose in the range of 0.4-35%, or 1-35%, or 2-30%, 1-20%, or 1-10%, or 1-5%, or 0.5-5%. In an exemplary embodiment, the composition comprises 3.5% trehalose.

In some embodiments of any of the aspects and embodiments herein that include a composition, or in some compositions used in or formed by a process, or compositions for use for controlling bleeding of a subject that includes a platelet composition in a powdered form, comprises trehalose having a weight percentage in the range of 10-60%, 15-55%, 20-60%, 20-50%, 25-60%, 25-50%, 10-50%, 20-40%, 20-35%, or 1-20%. In some embodiments, the weight percentage of trehalose can vary on the weight percentage of other components in the composition like, polysucrose, platelet derivatives, plasma protein, and buffering agents.

In some embodiments of any of the aspects and embodiments herein that include a composition, or in some compositions used in or formed by a process, or compositions for use for controlling bleeding of a subject that includes a population of platelet derivatives in a hydrated or rehydrated form, comprises polysucrose in the range of 2-8%, 2.25-7.75%, 2.5-7.5%, or 2.5-6.5%. In an exemplary embodiment, the composition comprises 3% polysucrose. In another exemplary embodiment, the composition comprises 6% polysucrose.

In some embodiments of any of the aspects and embodiments herein that include a composition, or in some compositions used in or formed by a process that includes a platelet composition in a powdered form, or compositions for use for controlling bleeding of a subject, comprises polysucrose having a weight percentage in the range of 20-80%, 25-75%, 30-70%, 35-65%, 30-80%, or 45-60%. In some embodiments, the weight percentage of trehalose can vary on the weight percentage of other components in the composition like, trehalose, platelet derivatives, plasma protein, and buffering agents.

In some embodiments of any of the aspects and embodiments herein that include a composition, or in some compositions used in or formed by a process, or compositions for use for controlling bleeding of a subject that includes a platelet composition in a powdered form, comprises trehalose and polysucrose having a combined weight percentage in the range of 30-95%, 35-95%, 40-90%, 40-90%, 45-90%, or 60-95%.

In some embodiments of any of the aspects and embodiments herein that include a composition or in some compositions used in or formed by a process herein, or compositions for use for controlling bleeding of a subject, comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 MW to 400,000 MW, 100,000 MW to 400,000 MW, 200,00 MW to 400,000 MW, 80,000 MW to 350,000 MW, 100,000 MW to 300,00 MW, 100,000 MW to 200,000 MW, 120,000 MW to 200,000 MW. In some exemplary embodiments, polysucrose has a molecular weight of 150,000 MW, 160,000 MW, 170,000 MW, 180,000 MW, 190,000 MW, or 200,000 MW.

In some embodiments of any of the aspects and embodiments herein that include a composition or in some compositions used in or formed by a process herein, or compositions for use for controlling bleeding of a subject, comprises platelet derivatives that are positive for at least one platelet activation marker selected from the group consisting of Annexin V, and CD 62. In some embodiments, the platelet derivatives are positive for at least one platelet marker selected from the group consisting of CD 41, CD 42, and CD 61. In some embodiments, the platelet derivatives are positive for CD 47. In some embodiments, the platelet derivatives are positive for Annexin V. In some embodiments, the platelet derivatives are positive for Annexin V. In some embodiments, at least 25%, 50%, or 75% of the platelet derivatives in the platelet derivative composition are Annexin V positive. In some embodiments, the platelet derivatives are positive for CD 41. In some embodiments, at least 30%, 35%, 40%, 45%, 50%, 55%, 60%, or 65% of the platelet derivatives in the platelet derivative composition are CD41 positive. In some embodiments, at least 90%, 91%, 92%, 93%, 94%, 95% 96%, 97%, 98%, or 99% platelet derivatives that are positive for CD 41 have a size in the range of 0.5-2.5 μm. In some exemplary embodiments, at least 95% platelet derivatives that are positive for CD 41 have a size in the range of 0.5-2.5 μm. In some embodiments, the platelet derivatives are positive for CD 42. In some embodiments, at least 65%, 80%, or 90% of the platelet derivatives in the platelet derivative composition are CD42 positive. In some embodiments, the platelet derivatives are positive for CD 47. In some embodiments, at least 8%, 10%, 15%, or 20% of the platelet derivatives in the platelet derivative composition are CD47 positive. In some embodiments, the platelet derivatives are positive for CD 62. In some embodiments, at least 10%, 50%, 65%, 80%, or 90% of the platelet derivatives in the platelet derivative composition are CD62 positive. In some embodiments, the platelet derivatives in the platelet derivative composition are positive for CD41, CD62, and Annexin V. In some embodiments, the platelet derivatives in the platelet derivative composition are at least 50% platelet derivatives are positive for CD41, at least 70% platelet derivatives are positive for CD62, and at least 70% platelet derivatives are positive for Annexin V.

In some embodiments of any of the aspects and embodiments herein that include a composition or in some compositions used in or formed by a process herein, or compositions for use for controlling bleeding of a subject, the platelet derivatives have fibrinogen associated with their cell membrane. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets.

In some embodiments of any of the aspects and embodiments herein that include a composition or in some compositions used in or formed by a process herein, or compositions for use for controlling bleeding of a subject that includes a population of platelet derivatives in a hydrated or rehydrated form, the platelet derivatives in the platelet derivative composition retain at least 10%, or 15%, or 20% of the lactate dehydrogenase activity of donor apheresis platelets. In some embodiments, the aqueous medium has a lactate concentration of less than 2.0 mmol/L, or 1.5 mmol/L. In some embodiments, the lactate concentration is in the range of 0.4 to 1.3 mmol/L, or 0.5 to 1.0 mmol/L.

In some embodiments of any of the aspects and embodiments herein that include a composition or in some compositions used in or formed by a process, or compositions for use for controlling bleeding of a subject herein that includes a population of platelet derivatives in a hydrated or rehydrated form, the platelet derivatives, when at a concentration of about 4.8×10³ particles/μL generate a thrombin peak height (TPH) of at least 25 nM, at least 50 nM, at least 75 nM, or at least 100 nM when in the presence of a reagent containing tissue factor and phospholipids. In some embodiments, the platelet derivatives, when at a concentration of about 4.8×10³ particles/μL generate a thrombin peak height (TPH) in the range of 25-100 nM, 30-80 nM, or 25-75 nM.

In some embodiments of any of the aspects and embodiments herein that include a composition or in some compositions used in or formed by a process, or compositions for use for controlling bleeding of a subject, the platelet derivatives, have a potency of at least 1.25, at least 1.5, at least 1.75, at least 2.0, at least 2.25, at least 2.5 thrombin generation potency units (TGPU) per 10⁶ particles. In some embodiments, the platelet derivatives have a potency in the range of 1.2 to 2.5, 1.2 to 2.0, 1.3 to 1.5, 1.5 to 2.25, 2 to 2.5, or 2.25 to 2.5 TGPU per 10⁶ particles.

In some embodiments of any of the aspects and embodiments herein that include a composition or in some compositions used in or formed by a process, or compositions for use for controlling bleeding of a subject, the platelet derivatives have the presence of thrombospondin (TSP-1) on their surface at a level that is at least 10%. 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives have the presence of thrombospondin (TSP-1) on their surface at a level that is at least 50%, 60%, 70%, 80%, 90%, or 100% higher than on the surface of activated platelets, or lyophilized fixed platelets. In some embodiments, the platelet derivatives have the presence of thrombospondin (TSP-1) on their surface at a level that is more than 100% higher than on the surface of activated platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

In some embodiments of any of the aspects and embodiments herein that include a composition or in some compositions used in or formed by a process, or compositions for use for controlling bleeding of a subject, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of von Willebrand factor (vWF) on their surface at a level that is more than 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

In some embodiments of any of the aspects and embodiments herein that include a composition or in some compositions used in or formed by a process, or compositions for use for controlling bleeding of a subject, the platelet derivatives lack an integrated membrane as compared to platelets. In some embodiments, the platelet derivatives are surrounded by a compromised plasma membrane. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives can retain 35%-75%, 40-70%, 45-65%, or 35-50% lactate dehydrogenase as compared to fresh platelets, or cold stored platelets, or cryopreserved platelets. In some embodiments, the platelet derivatives exhibit an increased permeability to antibodies. In some embodiments, the antibodies can be IgG antibodies.

In some embodiments of any of the aspects and embodiments herein that include a composition or in some compositions used in or formed by a process, or compositions for use for controlling bleeding of a subject, the platelet derivatives, when at a concentration of at least about 70×10³ particles/μL, produce an occlusion time of less than 14 minutes, or less than 12 minutes in a total thrombus-formation analysis system (T-TAS) assay. In some embodiments, the occlusion time is in the range of 1 to 13 minutes, 1 to 11 minutes, 1 to 10 minutes, or 1 to 7 minutes.

In some embodiments of any of the aspects and embodiments herein that include a composition or in some compositions used in or formed by a process, or compositions for use for controlling bleeding of a subject, the platelet derivatives in the platelet derivative composition comprise thrombosomes. In some embodiments, the platelet derivatives comprise freeze-dried platelets. In some embodiments, the platelet derivatives comprise thermally-treated freeze-dried platelets.

In some embodiments of any of the aspects and embodiments herein that include a process, comprises tangential flow filtration (TFF), centrifugation of a starting material comprising platelet composition, or a combination thereof. In some embodiments of the process, the starting material comprising platelet composition is: a) positive for HLA Class I antibodies based on a regulatory agency approved test; or b) positive for HLA Class II antibodies based on a regulatory agency approved test; or c) positive for HNA antibodies based on a regulatory agency approved test; or two or more of a), b), and c). In some embodiments, the starting material comprising platelet composition has a protein concentration in the range of 60 to 80 mg/mL, or 65 to 75 mg/mL. In some embodiments, the starting material comprising platelet composition comprises donor blood product. In some embodiments, the donor blood product is pooled donor blood product. In some embodiments, the starting material comprising platelet composition comprises donor apheresis material.

In some embodiments of any of the aspects and embodiments herein that include a process, that does not comprise centrifugation of the starting material comprising platelets or platelet composition, the diluted starting material comprising platelets or platelet composition, the concentrated platelet composition, the TFF-treated composition, or the combination thereof. In some embodiments, the process does not comprise centrifugation of a composition comprising platelets or platelet derivatives.

In some embodiments of any of the aspects and embodiments herein that include a process, the TFF comprises concentrating. In some embodiments, the TFF comprises diafiltering. In some embodiments, the diafiltering comprises diafiltering with at least two diavolumes. In some embodiments, the diafiltering is done with at least three diavolumes, or four diavolumes, or five diavolumes, or six diavolumes. In some embodiments, the diafiltering is done with diavolumes in the range of two to ten. In some embodiments, the TFF comprises buffer exchange.

In some embodiments of any of the aspects and embodiments herein that include a process, diluting comprises diluting with an approximately equal weight (±10%) of the preparation agent.

In some embodiments of the process of any of the aspects and embodiments herein that include a process, further comprises a pathogen reduction step. In some embodiments, the pathogen reduction step occurs before the diluting of the starting material. In some embodiments, the pathogen reduction step precedes TFF.

In some embodiments of any of the aspects and embodiments herein that include a process, wherein following washing if the concentration of the platelets or cells in the TFF-treated composition is not 2000×10³ cells/μL (±300×10³), 3000×10³ cells/μL (±300×10³), 4000×10³ cells/μL (±300×10³), 5000×10³ cells/μL (±300×10³), 6000×10³ cells/μL (±300×10³), 7000×10³ cells/μL (±300×10³), 8000×10³ cells/μL (±300×10³), 10000×10³ cells/μL (±300×10³), 12000×10³ cells/μL (±300×10³), 14000×10³ cells/μL (±300×10³), 16000×10³ cells/μL (±300×10³), 18000×10³ cells/μL (±300×10³), or 20000×10³ cells/μL (±300×10³), diluting the preparation agent or concentrating the platelets or the cells to fall within this range.

In some embodiments of any of the aspects and embodiments herein that include a process, further comprises lyophilizing or freeze-drying the TFF-treated composition to form a lyophilized composition. In some embodiments, a process further comprises treating the lyophilized composition at a temperature in the range of 60-90° C., or 65-85° C., or 70-90° C. for a time period in the range of 1-36 hours, or 5-30 hours, or 10-25 hours.

In some embodiments of any of the aspects and embodiments herein that include a process, the TFF is carried out using a membrane with a pore size in the range of 0.2 μm to 1 μm. In some embodiments the TFF is carried out using a membrane with pore size in the range of 0.3 μm to 1 μm, or 0.4 μm to 1 μm, or 0.4 μm to 0.8 μm, or 0.4 μm to 0.7 μm. In illustrative embodiments, the TFF is carried out using a membrane with a pore size of 0.45 μm, or 0.65 μm.

In some embodiments of any of the aspects and embodiments herein that include a process, the TFF is carried out until the absorbance at 280 nm of the aqueous medium is less than or equal to 50%, or 30%, or 10%, or 5%, or 3%, or 1% of the absorbance at 280 nm of the starting material comprising platelet composition, using a path length of 0.5 cm. In some embodiments, the TFF is carried out until the protein concentration or plasma protein concentration in the aqueous medium is less than or equal to 20%, 15%, 12.5%, 10%, 7.5%, 5%, 2.5%, or 1%. In some embodiments, the TFF is carried out until the protein concentration or plasma protein concentration is in between 0.01% and 20%, 0.01% and 15%, 0.01% and 10%, 0.01% and 5%, 0.1% and 20%, 0.1% and 15%, 0.1% and 10%, 0.1% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments, the TFF is carried out until the absorbance at 280 nm of the aqueous medium is less than or equal to 2.0 AU, or 1.90 AU, or 1.80 AU, or 1.70 AU, or 1.66 AU, or 1.60 AU, using a path length of 0.5 cm. In some embodiments, the TFF is carried out until the platelet concentration is at least 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL.

In some embodiments of any of the aspects and embodiments herein that include a process, the TFF-treated composition comprises at least 1000×10³ platelets/μL, 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL. In some embodiments, the TFF-treated composition comprises 1000×10³ platelets/μL to 20,000×10³ platelets/μL, 10,000×10³ platelets/μL to 20,000×10³ platelets/μL, 5000×10³ platelets/μL to 20,000×10³ platelets/μL, or 5000×10³ platelets/μL to 10,000×10³ platelets/μL.

In some embodiments of any of the aspects and embodiments herein that include a process, the TFF comprises diafiltering with a preparation agent comprising a buffering agent, a base, a loading agent, optionally a salt, and optionally at least one organic solvent. In some embodiments of the process, the TFF comprises buffer exchange into a preparation agent comprising a buffering agent, a base, a loading agent, optionally a salt, and optionally at least one organic solvent. In some embodiments, the buffering agent is HEPES (4-(2-hydroxyethyl)-1-piperazineethanesulfonic acid). In some embodiments, the base is sodium bicarbonate. In some embodiments, the loading agent is a monosaccharide, a polysaccharide, or a combination thereof. In some embodiments, the monosaccharide is selected from the group consisting of sucrose, maltose, trehalose, glucose, mannose, xylose, and combinations thereof. In some embodiments, the monosaccharide is trehalose. In some embodiments, the polysaccharide is polysucrose. In some embodiments, the salt is sodium chloride, potassium chloride, or a combination thereof. In some embodiments, the organic solvent is selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

In some embodiments of any of the aspects and embodiments herein that include a process, the preparation agent has a pH in the range of 5.5 to 8.0, or 6.0 to 8.0, or 6.0 to 7.5. In an illustrative embodiment, the preparation agent has a pH of 6.5. In another illustrative embodiment, the preparation agent has a pH of 7.4.

In some embodiments of any of the aspects and embodiments herein that include a process, that does not comprise a step for fixing the platelets, or platelet derivatives. In some embodiments, the process does not comprise fixing the platelets, or platelet derivatives using a fixative agent known in the art for fixing the platelets or platelet derivatives. In some embodiments, the process does not comprise contacting the platelets, or platelet derivatives with at least one fixative agent. In some embodiments, the fixative agent is an aldehyde. In some embodiments, the fixative agent is an alcohol. In illustrative embodiments, the fixative agent is selected from the group consisting of formaldehyde, paraformaldehyde, glutaraldehyde, and isopropanol.

In some embodiments of any of the aspects and embodiments herein that include a process, further comprises lyophilizing the composition comprising platelets or platelet derivatives.

In some embodiments of any of the aspects and embodiments herein that include a process, further comprises cryopreserving the composition comprising platelets or platelet derivatives.

In some embodiments of any of the aspects and embodiments herein that include a process, further comprises thermally treating the composition comprising platelets or platelet derivatives.

In some of the embodiments of any of the aspects and embodiments herein that include a process, the TFF is performed at a temperature in the range of 20° C. to 37° C., or 25° C. to 37° C., or 20° C. to 35° C., or 25° C. to 35° C.

In some embodiments of any of the aspects and embodiments herein that include a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is reduced by at least 50%, or by at least 75%, or by at least 90%, or by at least 95%, as compared to a similar composition not prepared by a process comprising tangential flow filtration of a blood product composition, centrifugation of a blood product composition, or a combination thereof.

In some embodiments of any of the aspects and embodiments herein that include a composition, or in some compositions used in or formed by a process, the platelet derivatives are derived from human platelets and are positive for at least one marker selected from the group consisting of CD 41, CD 42, and CD 61. In some embodiments, the platelet derivatives are derived from human platelets that are positive for CD 41. In some embodiments, embodiments, the platelet derivatives are derived from human platelets that are positive for CD 42. In some embodiments, embodiments, the platelet derivatives are derived from human platelets that are positive for CD 61. In some illustrative embodiments, the platelet derivatives are derived from human platelets that are positive for CD 41, CD 42, and CD 61.

In some embodiments of any of the aspects and embodiments herein that include a composition, or in some compositions used in or formed by a process, the platelet derivatives are derived from a non-human animal. In some embodiments, the non-human animal is selected from the group consisting of canines, equines, and felines. In some exemplary embodiments, the platelet derivatives are derived from canines.

In some embodiments of any of the aspects and embodiments herein that include a composition, or in some compositions used in or formed by a process that includes a population of platelet derivatives a powdered form, the platelet derivative composition comprises no more than 0.1%, 0.2%, 0.3%, 0.4%, 0.5%, 0.6%, 0.7%, 0.8% r 0.9%, 1%, 1.5%, 2%, 2.5%, 3%, 3.5%, 4.0%, 4.5%, or 4.9% residual moisture. In some embodiments, wherein the platelet derivative composition is in a powdered form, the platelet derivative composition comprises residual moisture in the range of 0.1-2%, 0.2-1.5%, 0.5-1.5%, 0.75-1.25%, 2-3%, 2.5-4.9%, 3-4.5%, 1.5-3%, or 1-2% residual moisture. In some illustrative embodiments, the platelet derivative composition comprises no more than 0.5% residual moisture.

In some embodiments of any of the aspects and embodiments herein that include a composition, or in some compositions used in or formed by a process that includes a plurality of containers each filled with a platelet derivative composition in the form of a powder, the platelet derivative composition in at least one of the plurality of containers comprises or is associated with a first protein from a first gene that has a different amino acid sequence than found in all the versions of the first protein from the first gene in the platelet derivative composition in one or more other containers of the plurality.

In some embodiments of any of the aspects and embodiments herein that include a composition, or in some compositions used in or formed by a process that includes a plurality of containers each filled with a platelet derivative composition in the form of a powder, the at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequence for at least one protein of a collection of protein gene products from a corresponding collection of encoding genes. In illustrative embodiments all, of the lots have a different amino acid sequence for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one protein of a collection of protein gene products from a corresponding collection of encoding genes. In some embodiments, the different amino acid sequences differ at one or more residues corresponding to amino acid residues encoded by a non-synonymous single nucleotide polymorphism (SNP). In some embodiments, the platelet derivative composition is in a container, and wherein the container is filled with at least one inert gas.

In some embodiments of any of the aspects and embodiments herein that include a composition, or in some compositions used in or formed by a process that includes a plurality of containers each filled with a platelet derivative composition in the form of a powder, the amount of plasma protein in the powder of any two containers chosen from different lots, differs by less than 50%, 40%, 30%, 20%, 1%, 50%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the amount of microparticles that are less than 0.5 μm in the powder of any two containers chosen from different lots, differs by less than 50%, 40%, 30%, 20%, 10%, 5%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one, two, three, four, or five protein of a collection of protein gene products from a corresponding collection of encoding genes. In some embodiments, the different amino acid sequences differ at one or more residues corresponding to amino acid residues encoded by a non-synonymous single nucleotide polymorphism (SNP).

In some embodiments of any of the aspects and embodiments herein that includes a plurality of containers each filled with a platelet derivative composition in the form of a powder, the containers can vary in volume from 5-100 ml, 10-90 ml, 25-75 ml, or 5-40 ml. In some embodiments, the volume of containers can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers can be above 100 ml, for example, 125 ml, 150 ml, 175 ml, or 200 ml. In some illustrative embodiments, the volume of vials is 30 ml. In some other illustrative embodiments, the volume of vials is 10 ml. In some embodiments, the plurality of containers can have 10-500 vials, 25-450 vials, 50-350 vials, 100-300 vials, or 150-250 vials. In some embodiments, the plurality of containers can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the plurality of containers can be increased to more than 500 as per the requirements, for example, 600, 700, 800, 900, or 1000 vials. In some embodiments, the number of vials can be 10-1000, 50-1000, 100-900, 200-800, or 150-700, or 150-500 vials. The number of vials in which a platelet derivative composition as per one of the embodiments, or aspects described herein can be filled and/or present can vary with the manufacturing requirements and the amount of starting material comprising platelets.

In some embodiments of any of the aspects and embodiments herein that includes a plurality of containers each filled with a platelet derivative composition in the form of a powder, the amount of platelet derivatives when the plurality of containers is taken as a whole can be 1×10⁹ to 1×10¹⁶, 1×10¹⁰ to 1×10¹¹, 1×10 to 1×10¹, 1×10¹² to 1×10¹⁶, or 1×10¹³ to 1×10¹⁵.

In some embodiments of any of the aspects and embodiments herein that include a composition, or in some compositions used in or formed by a process, the platelet derivatives are allogenic platelet derivatives. In some embodiments, the platelet derivatives are allogenic platelet derivative product. In some embodiments, a platelet derivative composition as per any of the embodiments or aspects herein, is a composition comprising allogenic platelet derivatives. In some embodiments, a platelet derivative composition as described herein is a U.S. FDA-approved product comprising allogenic platelet derivative composition. In some embodiments, a platelet derivative composition as described herein is a European EMA-approved product comprising allogenic platelet derivative composition. In some embodiments, a platelet derivative composition as described herein is a China FDA-approved product comprising an allogenic platelet derivative composition.

In some embodiments of the aspects and embodiments herein that include a composition, or in some compositions used in or formed by a process, or a process for preparing a platelet derivative composition, the platelets in the starting material can be donated from a human subject. In some embodiments, the human subject can be a male, or a female. In some embodiments, the platelets can be a pooled product from a number of male and female donors. In some embodiments, from a total of 100 donors, any number can be female donors, ranging from 0-100, 5-95, 10-90, 20-80, 30-70, or 40-60, and the rest can be male donors.

In some embodiments of the aspects and embodiments herein that include a composition, or in some compositions used in or formed by a process, or a process for preparing a platelet derivative composition, a starting material can comprise 10-500 units of platelets. In some embodiments, the starting material can comprise 20-500 units, 30-400 units, 40-350 units, or 50-200 units. In some embodiments, the units can be a pooled platelet product from multiple donors as described herein.

In some embodiments of any of the aspects and embodiments herein that include a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, the composition is passed through a filter of 18 μm before administering to the subject.

In some embodiments, the platelet derivative composition of any of the aspects or embodiments herein is provided for use in the treatment of a disorder selected from the group consisting of alopecia areata, Von Willebrand Disease, hemophilia, thrombasthenia, thrombocytopenia, thrombocytopenic purpura, trauma, or a combination thereof.

In some embodiments, the platelet derivatives as described herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject. In some embodiments, the platelet derivative composition of any of the aspects or embodiments herein is provided for use in wound healing in a subject.

In some embodiments, the platelet derivative composition of any of the aspects or embodiments herein can be used in treating a coagulopathy in a subject that has been administered or is being administered an antiplatelet agent. In some embodiments, the platelet derivative composition of any of the aspects or embodiments herein is provided for use an anti-platelet reversal agent. In some embodiments, the platelet derivative composition of any of the aspects or embodiments herein can be used in treating a coagulopathy in a subject that has been administered or is being administered an anticoagulant agent. In some embodiments, the platelet derivative composition of any of the aspects or embodiments herein is provided for use as an anti-coagulant reversal agent.

In some embodiments, there is provided a method of treating a subject, the method comprising administering to the subject in need thereof, an effective amount of any of the platelet derivative compositions disclosed herein, or the platelet derivative composition prepared by any of the methods disclosed herein, wherein the subject has been treated or is being treated with an antiplatelet agent and/or an anti-coagulant, and wherein the method for treating is a) a method for restoring normal hemostasis in the subject, or for controlling bleeding in the subject; b) for treating a coagulopathy in the subject; or c) for preparing the subject for surgery. In some embodiments, the method of treating is the method of restoring normal hemostasis in the subject. In some embodiments, the method of treating is the method for treating the coagulopathy in the subject. In some embodiments, the method of treating is the method for preparing the subject for surgery. In some embodiments of any of the methods for treating aspects, or method for controlling bleeding in the subject herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, a supplement, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, sarpogrelate, and a combination thereof.

In some embodiments of any of the methods for treating aspects, or for controlling bleeding in the subject herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof. In some embodiments of any of the methods for treating aspects herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof. In some embodiments of any of the methods for treating aspects herein, the administering comprises administering topically, parenterally, intravenously, intramuscularly, intrathecally, subcutaneously or intraperitoneally. In some embodiments of any of the methods for treating aspects herein, where the composition is in the form of a powder, the method further includes, before the administering, rehydrating the composition. In some embodiments provided herein is use of any of the platelet derivative compositions herein, in the manufacture of a kit for performing any of the methods of treating provided herein.

In some embodiments, there is provided a composition comprising platelets or platelet derivatives prepared by any of the process described in any of the aspects or embodiments herein. In some embodiments, there is provided a composition comprising platelets or platelet derivatives and an aqueous medium prepared by any of the process described in any of the aspects or embodiments herein. In some of the embodiments, there is provided a composition comprising freeze-dried platelets prepared by any of the process described in any of the aspects or embodiments herein. In some of the embodiments, there is provided a composition comprising thrombosomes prepared by any of the aspects or embodiments herein. In some of the embodiments of any of the aspects and embodiments herein that include a composition, or in some compositions used in or formed by a process, a composition prepared by a process comprising tangential flow filtration (TFF) of a starting material comprising platelets, centrifugation of a starting material comprising platelets, or a combination thereof. In some embodiments, the centrifugation comprises centrifugation at 1400×g to 1550×g, or 1450×g to 1500×g. In some embodiments, the composition is prepared by a process that does not comprise centrifugation.

In some embodiments of the aspects and embodiments herein that include a platelet derivative composition, or in some compositions used in or formed by a process, or a process for preparing a platelet derivative composition, or a method for treating a subject, or a platelet derivative composition comprising platelet derivatives for use as a medicament in treating a subject, a therapeutically effective dose of platelet derivatives is based on units of thrombin generation activity administered per kilogram of body weight of the subject. In further embodiments of these embodiments the effective dose is not based on the number of platelet derivatives delivered to the subject. In some embodiments of any aspect or embodiment herein the effective dose is based on both A) units of thrombin generation activity administered per kilogram of body weight of the subject; and B) the number of platelet derivatives administered to the subject. In some embodiments of any aspect or embodiment herein the effective dose is based on the weight of the subject.

In some embodiments of any aspect or embodiment herein the subject is suffering from a condition, or a disease selected from the group including only thrombocytopenia, hematologic malignancy, bone marrow aplasia, myeloproliferative disorders, myelodysplastic syndromes, and platelet refractoriness. In some embodiments, the subject is suffering from thrombocytopenia. In some embodiments, the subject is suffering from hematologic malignancy. In some embodiments, the subject is suffering from bone marrow aplasia. In some embodiments, the subject is suffering from myeloproliferative disorders. In some embodiments, the subject is suffering from myelodysplastic syndromes. In some embodiments, the subject is suffering from platelet refractoriness. In some embodiments, the subject is suffering from two or more of the disease or condition selected from the group consisting of thrombocytopenia, hematologic malignancy, bone marrow aplasia, myeloproliferative disorders, myelodysplastic syndromes, and platelet refractoriness.

In some embodiments of any aspect or embodiment herein a dose, a single dose, or therapeutically effective dose or amount of the platelet derivatives in a platelet derivative composition is in the range of 1.0×10⁷ to 1.0×10¹²/kg of the subject.

In some embodiments of any aspect or embodiment herein a therapeutically effective dose or amount of the platelet derivatives is an amount that has a potency in the range of 250 to 5000 TGPU per kg of the subject.

In some embodiments of any aspect or embodiment herein, a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition.

In some embodiments of any aspect or embodiment herein, a method of treatment or a composition for use as a medicament as described herein, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject.

In some embodiments of any aspect or embodiment herein, a method of treatment or a composition for use as a medicament as described herein, a method or a medicament leads to changes, or in some embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In some embodiments, the method or the medicament leads to changes, or in some embodiments, does not lead to changes, in one or more markers of endothelial cell injury in the subject at 72 hours after administering the platelet derivative composition. In some embodiments, the one or more markers of endothelial cell injury is selected from the group consisting of Syndecan-1, hyaluronan, thrombomodulin, vascular endothelial growth factor (VEGF), interleukin 6, and sVE cadherin. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments of any aspect or embodiment herein, a method of treatment or a composition for use as a medicament as described herein, a method or a medicament leads to acceptable measures of coagulation in the subject at 12 hours to 35, 24 hours to 32 days, 24 hours to 30 days, or 48 hours to 28 days after administering the platelet derivative composition. In some embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments of any aspect or embodiment herein, a method of treatment or a composition for use as a medicament as described herein, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments of any aspect or embodiment herein, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments of any aspect or embodiment herein, a method of treatment or a composition for use as a medicament as described herein, administering is performed in a maximum of 2, 3, 4, 5, 6, 7, 8, 9, or 10 doses in a 72-hour period of treatment. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding.

In some embodiments of any aspect or embodiment herein the subject at the time of administering has two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

In some embodiments of any aspect or embodiment herein the administering confers an improved survival at 10, 15, 20, 25, 30, 35, 40, 45, or 50 days after administering the platelet derivatives. In some embodiments of any aspect or embodiment herein the administering leads to a decrease in administration of secondary blood products, platelets, or platelet derivatives to the subject for the first 5, 6, 7, 8, 9, or 10 days after the administering of the platelet derivatives.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, the subject has an indication selected from the group consisting of Von Willebrand disease, immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, acute lymphoblastic lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof. In some embodiments, an indication is Von Willebrand disease. In some embodiments, an indication is Immune thrombocytopenia. In some embodiments, an indication is Chemotherapy induced thrombocytopenia (CIT). In some embodiments, an indication is Fetal and neonatal alloimmune thrombocytopenia.

In some embodiments of any aspect or embodiments herein that include an indication, the indication is selected from the group consisting of Von Willebrand disease, Immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, or combinations thereof.

In some embodiments of any aspect or embodiments herein that include an indication, the indication is selected from the group consisting of Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof.

In some embodiments of any aspect or embodiments herein that include an indication, the indication is selected from the group consisting of Fetal and neonatal alloimmune thrombocytopenia, intracranial hemorrhage (ICH), traumatic brain injury (TBI), Von Willebrand disease, Immune thrombocytopenia, and the indication is not treatable by administering unmodified platelets. In some embodiments, the indication is Von Willebrand disease, and wherein the indication is not treatable by administering unmodified platelets. In some embodiments, the indication is Immune thrombocytopenia, and wherein the indication is not treatable by administering unmodified platelets. In some embodiments, the indication is Fetal and neonatal alloimmune thrombocytopenia, wherein the indication is not treatable by administering unmodified platelets. In some embodiments, the indication is intracranial hemorrhage (ICH), and wherein the indication is not treatable by administering unmodified platelets. In some embodiments, the indication is traumatic brain injury (TBI), and wherein the indication is not treatable by administering unmodified platelets.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, the subject has an indication that, typically cannot be treated with unmodified platelet preparation but can be treated with a platelet derivative composition as described herein. In some embodiments, such type of indication is Von Willebrand disease, Immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, or combinations thereof.

In some aspects, provided herein is a method of treating immune thrombocytopenia in a subject, the method comprises administering an effective dose of freeze-dried platelet derivatives, in a platelet derivative composition, to the subject. In some aspects, provided herein is a method of treating intracranial hemorrhage (ICH) in a subject, the method comprises administering an effective dose of freeze-dried platelet derivatives, in a platelet derivative composition, to the subject. In some aspects, provided herein is a method of treating traumatic brain injury (TBI) in a subject, the method comprises administering an effective dose of freeze-dried platelet derivatives, in a platelet derivative composition, to the subject.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, platelet derivatives can have an effective dose or a therapeutically effective dose in the range of 1.0×10⁷ to 1.0×10¹¹ particles/kg of the subject. In some embodiments, platelet derivatives can have an effective dose or a therapeutically effective dose in the range 250 and 5000 TGPU per kg of the subject.

In some embodiments, provided herein is a platelet derivative composition comprising platelet derivatives of any aspect provided herein, or a platelet derivative composition prepared by the process of any method for preparing or making provided herein, or compositions for use for controlling bleeding of a subject, wherein the platelet derivatives comprise an imaging agent, to form imaging agent-loaded platelet derivatives. In some embodiments, the platelet derivatives in imaging agent-loaded platelet derivatives are freeze-dried platelet derivatives (FDPDs) that retain one, two, three, or more properties of FDPDs that are not loaded as described herein. In some embodiments, the imaging agent can be an MRI agent, such as gadolinium. In some embodiments, platelet derivatives comprise an MRI agent, termed as MRI agent-loaded platelet derivatives. In some embodiments, MRI agent-loaded platelet derivatives are freeze-dried platelet derivatives (FDPDs) that retain one, two, three, or more properties of FDPDs that are not loaded as described herein.

In some embodiments, provided herein is imaging agent-loaded platelets, cryopreserved platelets, or platelet derivatives. In some embodiments, provided herein is MRI agent-loaded platelets, cryopreserved platelets, or platelet derivatives. In some embodiments, imaging agent-loaded or MRI agent-loaded platelets, cryopreserved platelets, or platelet derivatives comprise a cell penetrating peptide (CPP). A CPP is coupled to an imaging agent or an MRI agent that facilitates the imaging agent or MRI agent to load onto platelets, cryopreserved platelets, or platelet derivatives. In illustrative embodiments, a CPP is a TAT peptide.

In some embodiments, provided herein is a method of delivering an imaging agent to a subject. In some embodiments, platelet derivatives or platelets as described herein are loaded with an imaging agent to form imaging agent-loaded platelet derivatives or platelets, such imaging agent-loaded platelet derivatives or platelets are administered to a subject. In some embodiments, platelet derivatives or platelets as described herein are loaded with an MRI agent to form MRI agent-loaded platelet derivatives or platelets, such MRI agent-loaded platelet derivatives or platelets are administered to a subject.

Also provided herein are compositions produced by any of the methods described herein. In some embodiments, any of the compositions provided herein can be made by the methods described herein. Specific embodiments disclosed herein may be further limited in the claims using “consisting of” or “consisting essentially of” language.

The following non-limiting examples are provided purely by way of illustration of exemplary embodiments, and in no way limit the scope and spirit of the present disclosure.

### EXAMPLES

### Example 1: Flow Cytometry Surface Marker Characterization of Freeze-Dried Platelet-Derived Hemostat Versus Apheresis Platelet Units

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

APU and FPH counts were determined by diluting APU and FPH samples 1:500 in phosphate-buffered saline (PBS) and then acquiring events on the Quanteon flow cytometer (Agilent, agilent.com). Counts were determined using a bivariant logarithmic size density plot of forward scatter (FSC-Hvs (SSC-H) in a known volume of sample. Based on counts, FPH and APU were normalized to 100 k cells/ul in HEPES modified Tyrode's buffer with albumin (HMTA) before staining. 1 million total cells (10 μL) of either FPH or APU were stained and incubated for 20 minutes in final sample volumes of 30 uL containing the antibody of interest and HMTA to fill the remaining volume. Staining was performed on FPH and day 4 aged APU across 10 samples measured in duplicate for each group.

All antibodies volumes/concentrations used in this characterization work was first obtained through antibody titration testing using isotype controls on FPH and APU in similarly prepared sample staining volumes. Final concentrations used was as follows: 17.7 μg/mL of vWF antibody (Novus Biologicals, novusbio.com), 267 μg/mL at 1:3 mix of bovine lactadherin-FITC labeled (Prolytix, goprolytix.com) and bovine lactadherin (Prolytix, goprolytix.com), 16.7 μg/mL APC anti-human CD63 antibody (Biolegend, biolegend.com), 0.45 μg/mL PE mouse anti-human CD62P (BD Biosciences, bdbiosciences.com), 6 μL of FITC mouse anti-human CD42b (BD Biosciences, bdbiosciences.com), 2 μg/mL of PE mouse anti-human platelet GPVI (BD Biosciences, bdbiosciences.com), 6 μL PE mouse anti-human CD49b (BD Biosciences, bdbiosciences.com), 8.3 μg/mL of PE mouse anti-human CD61 (BD Biosciences, bdbiosciences.com), 33.3 μg/mL of Alexa Fluor 546 anti-thrombospondin 1 antibody (Santa Cruz, scbt.com), 5 μL FITC anti-fibrinogen antibody (Abcam, abcam.com) prediluted 1:7 in HMTA, 5 μL anti-fibrinogen antibody, receptor-induced binding site (RIBS) clone 9F9 (BioCytex, biocytex.com), 0.71 μg/mL anti-CD41 antibody, GPIIbIIIa complex dependent clone P2 (Beckman Coulter, beckmancoulter.com), and 12.5 μg/mL anti-CD41/CD61 antibody, active conformation dependent clone PAC-1 (Biolegend, biolegend.com). All samples were then diluted 3:50 (v/v) in PBS and mean fluorescence intensity (MFI) was acquired on the Quanteon Flow Cytometer (Agilent, agilent.com). FIG. 1A-C show the mean fluorescence intensity for the different surface markers for FPH and APU. For the two collagen receptors, GpIaIIa and GpVI, FPH had 2-fold higher and 6-fold reduced expression in comparison to APU, respectively. The higher active conformation GpIaIIa expression on FPH suggests that GpIaIIa may mediate FPH collagen adhesion. Thrombospondin is involved in platelet adhesion to vessel walls and in platelet aggregation, there is a 66-fold increase of thrombospondin expression in FPH relative to APU. FPH show about a 60 percent reduction in CD42b but has a higher amount of VWF expression. Therefore, there is a potential for vWF to mediate initial collagen interactions. Expression of CD61, CD62P and CD63 is higher in FPH relative to APU. Phosphatidylserine expression for FPH is increased by 58-fold relative to APU. FPH is phenotypically similar to activated platelets. The increased CD62P and CD63 expression together with increased surface bound fibrinogen, thrombospondin, and vWF gives FPH a phenotype similar to collagen and thrombin-activated (COAT) platelets.

### Example 2: FPH and APU Stimulated by Different Doses of MgCl₂ in Octaplas and HMTA

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

MgCl₂ (1 M) was prediluted 1:4 in HEPES modified Tyrode's buffer with albumin (HMTA) to create a 250 mM stock solution. A master mix of FPH or APU was prepared by combining cells dosed at 250 k/μL with either Octoplas® (Octophrama, octopharma.com) or HMTA to arrive at enough volume to fill 225 μl of sample into 8 cuvettes, plus extra buffer volume if necessary. 225 μl of master mix was transferred to cuvettes as specified along with a magnetic stir bar in each cuvette. HMTA was added to the cuvettes to bring up the total volume in the cuvette to 250 μl, leaving enough volume to add 0, 2, 5, and 10 mM of MgCl₂ from the 250 mM prediluted stock solution respectively. Each concentration of MgCl₂ to be tested was prepared in duplicate. Cuvettes were transferred to the pre-warmed wells of a PAP-8 Platelet Aggregometer at 37° C. and allowed to incubate for 2 minutes to come up to temperature. Cuvettes were then transferred to the testing wells and ran immediately. When prompted by the instrument, the appropriate volumes of prediluted MgCl₂ were injected into the corresponding cuvettes as quickly as possible. Samples were run on PAP-8 Platelet Aggregometer (Bio/Data, biodatacorp.com) for 15 minutes. At completion of the run, stir bars were removed from each cuvette before the counts were collected on the AcT Diff Hematology Analyzer.

Percent aggregation was calculated as the percent fold change of MgCl₂ treated samples as compared to the 0 mM MgCl₂ or untreated sample. FPH partially aggregates when divalent cation (magnesium) is added, whereas APU do not (FIG. 2). FPH counts decreased with increasing magnesium concentrations indicating an increase in aggregation with increasing concentrations of MgCl₂ as shown in FIG. 2. APU do not aggregate in response to divalent cations (magnesium) because they do not have GpIIbIIIa (CD41/CD61) in the active conformation.

### Example 3: FPH Flow Surface Marker Characterization with and without 10 mM MgCl₂

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

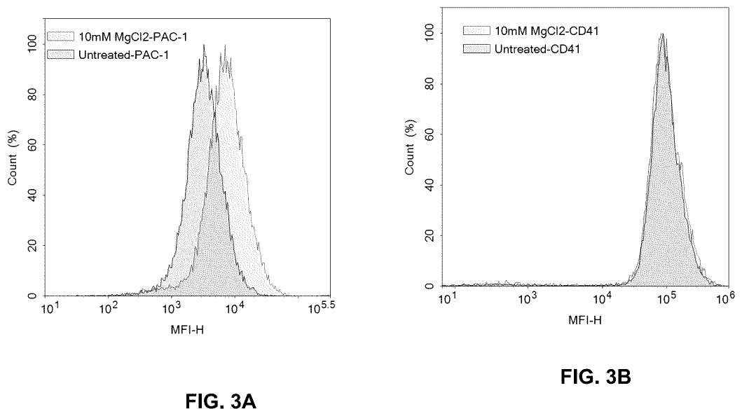

APU and FPH counts were determined by diluting samples 1:500 in phosphate-buffered saline (PBS) and then acquiring events on the Quanteon flow cytometer (Agilent, agilent.com). Counts were determined using a bivariant logarithmic size density plot of forward scatter (FSC-H) vs (SSC-H) in a known volume of sample. Based on counts, FPH were normalized to 100 k cells/ul in either HMTA alone or HMTA+10 mM MgCl₂ preparations. 1 million total cells (10 uL) of either FPH with or without 10 mM MgCl₂ were stained and incubated for 20 minutes in final sample volumes of 30 μL containing anti-CD41/CD61(PAC-1) antibody and HMTA to fill the remaining volume. The amount of antibodies used was first determined by antibody titration testing using isotype controls on FPH and APU in similarly prepared sample staining volumes. The final concentration used was 12.5 μg/mL of CD41/CD61(PAC-1) antibody (Biolegend, biolegend.com) and 0.71 μg/mL anti-CD41 antibody, GPIIbIIIa complex dependent clone P2 (Beckman Coulter, beckmancoulter.com), with additional 2 mM MgCl₂. FIG. 3A and FIG. 3B show that divalent cations allow GpIIbIIIa complexes to form active, high affinity conformation as demonstrated by increased PAC-1 binding with MgCl₂ without increased CD41 antibody binding. The induction of unoccupied GpIIbIIa complexes into the high affinity conformation by divalent cations is required to allow FPH to bind adjacent particles surface bound fibrinogen and aggregate.

### Example 4: FPH and APU Aggregation Dose Response in HMTA with Tirofiban

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

MgCl₂ (1 M) was prediluted 1:4 in by combining 25 ul MgCl₂ with 75 ul HEPES modified Tyrode's buffer with albumin (HMTA) to create a 250 mM stock solution. tirofiban (3.1 mg/mL suspended in DMSO) was prediluted 1:100 by combining 2 μl Tirofiban with 198 μl HMTA to create a 31 μg/mL stock solution. DMSO (1 mg/mL) was prediluted 1:100 by combining 21 tirofiban with 198 μl HMTA to create a 10 μg/mL stock solution. A master mix of FPH or APU was prepared by combining cells dosed at 250 k/μL with HMTA to arrive at enough volume to fill 225 μl of sample into 8 cuvettes, plus extra buffer volume if necessary. 225 μl of master mix was transferred to cuvettes as specified along with a magnetic stir bar in each cuvette. HMTA was added to the cuvettes to bring up the total volume in the cuvette to 250 μl, leaving enough volume to add Tirofiban, DMSO, and/or MgCl₂ from their prediluted stock solutions depending on the sample groups. The groups tested were as follows. (+) indicates the presence while (−) indicates the absence of the specified reagent:
0.32 mM DMSO−/5 mM Mg−/1 mM Tiro− (negative control)
0.32 mM DMSO+/5 mM Mg−/1 mM Tiro− (negative control)
0.32 mM DMSO−/5 mM Mg+/1 mM Tiro− (positive control)
0.32 mM DMSO+/5 mM Mg+/1 mM Tiro− (positive control)
0.32 mM DMSO+/5 mM Mg−/1 mM Tiro− (#of samples=2)
0.32 mM DMSO+/5 mM Mg−/1 mM Tiro− (#of samples=2)

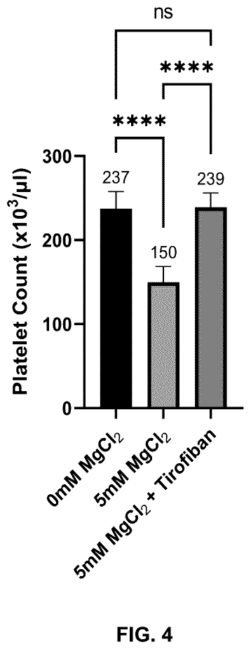

DMSO was prepared in control samples to demonstrate that its use to rehydrate Tirofiban was not influencing the aggregation behavior of FPH. Cuvettes were transferred to the pre-warmed wells of a PAP-8 Platelet Aggregometer at 37° C. and allowed to incubate for 2 minutes to come up to temperature. Cuvettes were then transferred to the testing wells and ran immediately. When prompted by the instrument, the appropriate volumes of prediluted tirofiban, DMSO, and/or MgCl₂ were injected into the corresponding cuvettes as quickly as possible. Samples were run on PAP-8 Platelet Aggregometer for 15 minutes. At completion of the run, stir bars were removed from each cuvette before the counts were collected on the AcT Diff Hematology Analyzer. Percent aggregation was calculated as the percent fold change of MgCl₂ treated samples as compared to the untreated control (0.32 mM DMSO−/5 mM Mg−/1 mM Tiro−). FIG. 4 shows that FPH treated with tirofiban did not aggregate in response to MgCl₂. The divalent cation induced aggregation of FPH is abrogated by the addition of tirofiban. The induction of unoccupied GpIIbIIa complexes into the high affinity conformation by divalent cations is required to allow FPH to bind adjacent particles surface bound fibrinogen and aggregate.

### Example 5: MgCl₂ Aggregation Dose Response Following Plasmin Treatment of FPH

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

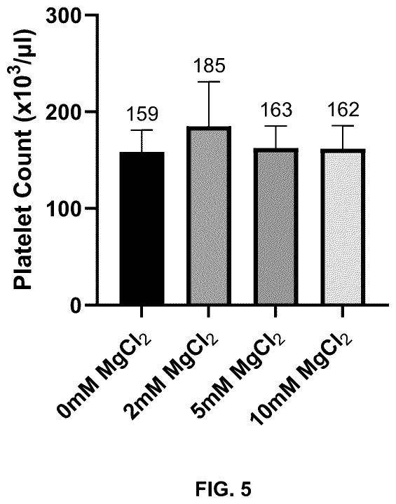

MgCl₂ (1 M) was prediluted 1:4 in HEPES modified Tyrode's buffer with albumin (HMTA) to create a 250 mM stock solution. A master mix of FPH prepared by combining cells dosed at 250 k/μL with HMTA and 100 mM plasmin to arrive at enough volume to fill 225 μl of sample into 4 cuvettes, plus extra buffer volume if necessary. A separate master mix of FPH was prepared by combining cells dosed at 250 k/μL with HMTA and 100 mM plasmin to arrive at enough volume to fill 225 μl of sample into 4 cuvettes, plus extra buffer volume if necessary. Both master mixes were incubated at room temperature with agitation for 1 hour. 225 μl of the plasmin treated FPH was transferred to all cuvettes that were to be placed in the odd wells of the PAP-8 Platelet Aggregometer and 225 μl of the other untreated master mix was transferred to all the cuvettes to be placed in the even wells. Magnetic stir bars were placed in all the cuvettes. HMTA was added to all cuvettes to bring up the total volume in the cuvette to 250 μl, leaving enough volume to add 0, 2, 5, and 10 mM of MgCl₂ from the 250 mM prediluted stock solution respectively. Each concentration of MgCl₂ to be tested was prepared in duplicate. Cuvettes were transferred to the pre-warmed wells of a PAP-8 Platelet Aggregometer at 37° C. and allowed to incubate for 2 minutes to come up to temperature. Cuvettes were then transferred to the testing wells and ran immediately. When prompted by the instrument, the appropriate volumes of prediluted MgCl₂ were injected into the corresponding cuvettes as quickly as possible. Samples were run on PAP-8 Platelet Aggregometer for 15 minutes. At completion of the run, stir bars were removed from each cuvette before the counts were collected on the AcT Diff Hematology Analyzer. Percent aggregation was calculated as the percent fold change of MgCl₂ treated samples as compared to the 0 mM MgCl₂ or untreated sample. Plasmin causes the breakdown of fibrinogen. As shown in FIG. 5, there is no dose dependent response to the MgCl₂ due to the plasmin addition since fibrinogen is required for the aggregation.

### Example 6: Mixed Aggregation of FPH with Washed Platelet Units with and without EDTA in the Presence of Different Agonists

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization. Whole blood was collected from healthy donors in vacutainer ACD blood collection tubes (Becton Dickinson, bdbiosciences.com) centrifugated at 180×g for 20 minutes to collect platelet rich plasma (PRP).

The aggregation of washed FPH with washed platelets was screened in the presence of different agonists with and without EDTA (Sigma Aldrich, sigmaaldrich.com) using the PAP-8 Platelet Aggregometer. To obtain washed FPH and washed PRP, 6 μl of 1M citric acid was added to 1 ml of PRP and 1 ml of FPH, then centrifuged at 1000×g for 10 minutes and resuspended in HMTA. This was repeated 5 times. Washed FPH and washed platelets (WPS) count was 300,000/μl using the Beckman Coulter AcT Diff 2 Hematology Analyzer in duplicate to reach 7 ml and 5 ml, respectively. In one of the duplicates, EDTA was added to achieve a 5 mM concentration. Then 3 ml FPH at 300,000/μl was added to 1 ml washed platelets at 300,000/μl to reach 4 ml. These samples were prepared immediately prior to running on the PAP-8E Platelet Aggregometer. The different agonist were prediluted in HMTA according: arachidonic acid (Helena Biosciences, Helena-biosciences.com) 1:10 in HMTA, TRAP-6 (Tocris, tocris.com) by adding 7.5 μl to 292.5 μl HMTA.

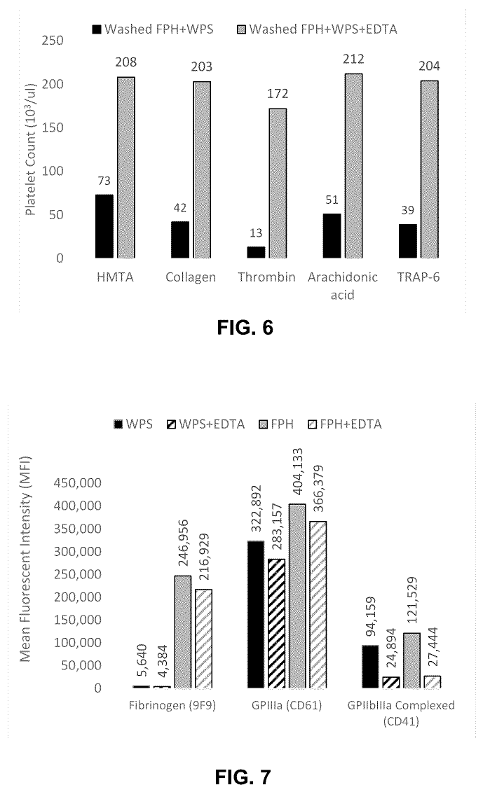

The PAP-8E Platelet Aggregometer was turned on and samples were incubated with stirring for 10 minutes at 37° C. for all mixed aggregation experiments. The samples with HMTA was used as a control for agonists used in aggregation. The following agonists were used to induce aggregation in samples: 10 μg/ml collagen (Helena Biosciences, Helena-biosciences.com), 10U/ml thrombin (Helena Biosciences, Helena-biosciences.com), 10 μg/ml arachidonic acid, and 10 μM TRAP-6. 225 μl of the samples were added to cuvettes by reverse pipetting and inserted in the channels. Cuvettes were transferred from the warming wells to the testing wells. The test was started and 25 μl of each agonist was added to the appropriate cuvettes when the timer for the channel reached 8:00 minutes. HMTA was added in place of the agonist to the first channel in each module. Once measurements were completed for each agonist, the samples were transferred to a microcentrifuge tube and AcT count was taken. FIG. 6 shows the washed FPH mixed with washed platelets (WPS) in the presence of the control HMTA resulted in more than a 50% reduction in platelet count, representing substantial sample aggregation. The addition of the agonists (collagen, thrombin, arachidonic acid, or TRAP) further decreased platelet counts, suggesting that the sample aggregation was not primarily agonist driven. The impact of the GpIIbIIIa inhibitor, EDTA, prevents aggregation between the washed FPH and washed platelets as shown in the higher platelet counts in comparison to the untreated mixed samples (FIG. 6). Further there was no substantial change in the platelet counts in the EDTA treated samples with the addition of the different agonists. Fluorescent microscopy visually confirmed the presence of large aggregates when washed FPH and washed platelets were mixed in untreated samples. But in samples treated with EDTA, no aggregation was visually observed in either washed platelet and washed FPH alone or mixed together.

### Example 7: Surface Marker Detection of Washed FPH and Washed Platelets with and without EDTA

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization. Whole blood was collected from healthy donors in vacutainer ACD blood collection tubes (Becton Dickinson, bdbiosciences.com) centrifugated at 180×g for 20 minutes to collect platelet rich plasma (PRP).

Flow cytometry measurements of surface markers were determined for platelets and freeze-dried platelet hemostats (FPH) with and without EDTA treatment. To obtain washed FPH and washed PRP, 6 μl of 1M citric acid was added to 1 ml of PRP and 1 ml of FPH, then centrifuged at 1000×g for 10 minutes and resuspended in HMTA. Washed FPH and washed platelets (WPS) were diluted to 100,000/μl in the appropriate amount of HMTA by AcT count to reach 1 ml each in duplicate. EDTA (Sigma Aldrich, sigmaaldrich.com) was added to one of the duplicates to achieve a 5 mM concentration. Dissociation of GPIIbIIIa by EDTA was confirmed on the Quanteon Flow Cytometer (Agilent, agilent.com) for FPH and WPS after incubation with a RIBS antibody for fibrinogen, clone 9F9 (BioCytex, biocytex.com), an anti-CD61 antibody (BD Biosciences, bdbiosciences.com), a conformationally dependent anti-CD41 antibody (Beckman Coulter, beckmancoulter.com), and an anti-vWF antibody (Novus Biologicals, novusbio.com). Stain volumes were as follows: Unstained: 10 μl of sample and 20 μl HMTA, 9F9: 10 μl of sample, 5 μl antibody, and 15 μl HMTA, CD41: 5 μl of sample and 12 μl antibody, and CD61: 10 μl of sample, 10 μl antibody, and 10 μl HMTA. Stained samples were incubated for 20 minutes for 9F9, CD61, and vWF and 5 minutes for CD41. 470 μl of the samples were added to 9F9 and CD61 stains and 233 μl of the samples were added to CD41 stains. 100 μl of each sample was transferred to wells in a 96-well plate. All samples were measured using the Quanteon flow cytometer. FIG. 7 shows the surface marker detection for washed platelets and washed FPH with and without EDTA. CD61 (GpIIIa) signal decreased slightly with the addition of EDTA to both washed FPH and washed platelets. The drop in the GpIIbIIIa complex is more than half with the addition of EDTA to both washed FPH and washed platelets. This indicates the disassociation of the active conformation of GpIIb/IIIa with the addition of EDTA. Bound fibrinogen decreases slightly in washed FPH with the addition of EDTA, suggesting irreversibly bound fibrinogen on the washed FPH surface,

### Example 8: Mixed Aggregation of FPH with Washed Platelet with and without RGDS in the Presence of Different Collagen

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization. Whole blood was collected from healthy donors in vacutainer ACD blood collection tubes (Becton Dickinson, bdbiosciences.com) centrifugated at 180×g for 20 minutes to collect platelet rich plasma (PRP).

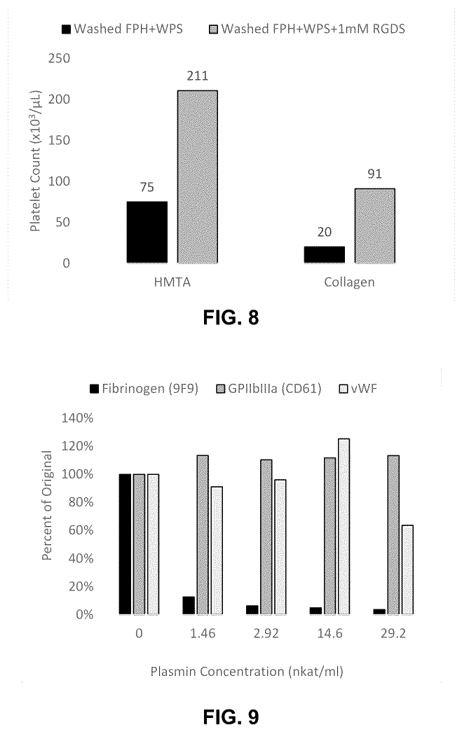

The aggregation of washed FPH with washed platelets was screened in the presence of collagen agonist with and without Arg-Gly-Asp-Ser (RGDS, Cayman Chemical, caymanchem.com) using the PAP-8 Platelet Aggregometer. To obtain washed FPH and washed PRP, 6 μl of 1M citric acid was added to 1 ml of PRP and 1 ml of FPH, then centrifuged at 1000×g for 10 minutes and resuspended in HMTA. Washed FPH and washed platelets (WPS) were diluted to 300,000/μl in the appropriate amount of HMTA by AcT count to reach 1 ml each in duplicate. For one of the replicates of each, RGDS was added to achieve a 1 mM concentration. Washed FPH was mixed with washed platelets (WPS) with and without RGDG by adding 0.5 ml washed FPH 300,000/μl to 0.5 ml washed platelets 300,000/μl to reach 1 ml. The mixture was prepared immediately prior to running on the PAP-8E Platelet Aggregometer. PAP-8E was turned on and samples were incubated with stirring for 10 minutes at 37° C. and HMTA was used as a control for collagen agonist used in aggregation. Stir bars were added to cuvettes and 225 μl of sample was added to cuvettes by reverse pipetting and inserted into channels. Cuvettes were then transferred from warming wells to the testing wells. Run was started for each channel with 10 seconds in between. 25 μl of collagen (Helena Biosciences, helenabiosciences.com) was added to appropriate cuvettes when the timer for the channel reached 8:00 minutes. HMTA was added in place of the agonist to the first channel as a control. After measurement was completed for collagen agonist, each sample was transferred to a microcentrifuge tube and AcT count was taken. FIG. 8 shows the mixed aggregation response to collagen agonist with and without RGDS. The GpIIbIIa receptor antagonist RGDS peptide also inhibited aggregation as shown by the more that 2-fold lower platelet counts in untreated mixed sample in comparison to the treated mixed sample (FIG. 8).

### Example 9: Surface Marker Detection of Washed FPH in Different Concentrations of Plasmin

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

Flow cytometry measurements of surface markers were determined for (FPH) with increasing concentrations of plasmin (Innovation Research, innov-research.com). To obtain washed FPH 6 μl of 1M citric acid was added to 1 ml of FPH, then centrifuged at 1000×g for 10 minutes and resuspended in HMTA. Washed FPH and washed platelets (WPS) were diluted to 300,000/μl in the appropriate amount of HMTA by AcT count to reach 1 ml each. This was repeated 5 times. Plasmin was added to each replicate for a concentration of 0, 1.46, 2.92, 14.6, and 29.2 nkat/ml and incubated at room temperature for 1 hour. Anti-vWF antibody was prediluted to 1:10 by combining 5 μL antibody with 45 μl of HTMA. FPH samples were diluted to 100,000/μl in the appropriate amount of HMTA by AcT count to reach 200 μl each. The stained samples were prepared accordingly: (1) Unstained: 10 μl of FPH sample and 20 μl HMTA, (2) 9F9 (Biocytex, biocytex.com): 10 μl of sample, 5 μl antibody, and 15 μl HMTA, (3) anti-CD61 (BD Biosciences, bdbiosciences.com): 10p of sample, 10 μl antibody, and 10p HMTA, (4) anti-vWF: 10 μl of sample, 9 μl antibody, and 11 μl HMTA. Each sample was incubated for 20 minutes and 470 μl of PBS was added to the stained samples. Each sample was then transferred to wells in a 96-well plate. Measurements were taken using the Quanteon flow cytometer using the protocol below. FIG. 9 shows the surface marker detection with increasing doses of plasmin. As expected plasmin degrades fibrinogen on the surface of FPH, the degradation increases with increasing concentration of plasmin (FIG. 9).

### Example 10: Mixed Aggregation of Washed FPH with Washed Platelet with and without the Presence of Plasmin (3E)

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization. Whole blood was collected from healthy donors in vacutainer ACD blood collection tubes (Becton Dickinson, bdbiosciences.com) centrifugated at 180×g for 20 minutes to collect platelet rich plasma (PRP).

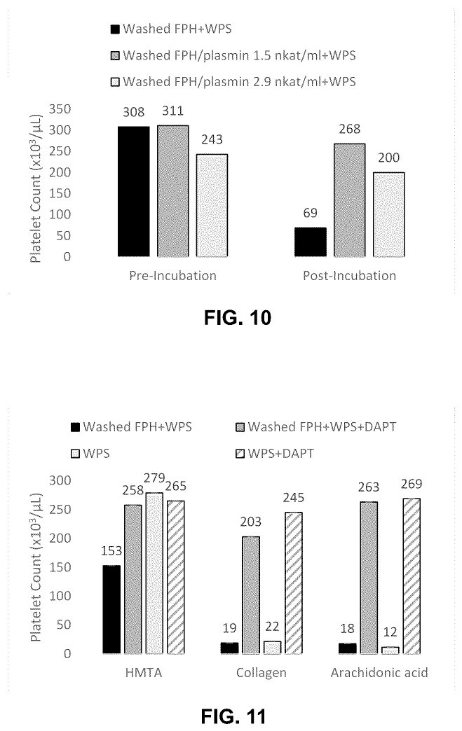

The aggregation of washed FPH with washed platelets was screened with and without plasmin using the PAP-8 Platelet Aggregometer. To obtain washed FPH and washed PRP, 6 μl of 1M citric acid was added to 1 ml of PRP and 1 ml of FPH, then centrifuged at 1000×g for 10 minutes and resuspended in HMTA. Washed FPH and washed platelets (WPS) were diluted to 100,000/μl in the appropriate amount of HMTA by AcT count to reach 2 ml. 0.5 ml of washed platelets (WPS) at 100,000/μl was added to 0.5 ml washed FPH 100,000/μl to reach 1 ml. This was repeated for optimal concentration of plasmin added based on flow results. To initiate spontaneous aggregation between the washed platelets and washed FPH the following steps were performed for the mixed samples. Stir bars were added to cuvettes, 225 μl of sample was added to the cuvettes by reverse pipetting and inserted into channels. The cuvettes were transferred from the warming wells to the testing wells. The run was started for each channel with 10 seconds in between. 25 μl of HMTA was added to all cuvettes once the timer for the channel reached 8:00 minutes. After measurements were completed, each sample was transferred to a microcentrifuge tube and AcT counts were taken. FIG. 10 shows platelet counts before and after incubation of washed FPH with plasmin mixed with washed platelets. FIG. 10 shows that plasmin inhibits the aggregation of washed FPH with washed platelets based on the increased platelet count after plasmin addition.

### Example 11: Mixed Aggregation of Washed FPH with Washed Platelet with and without DAPT in the Presence of Different Agonists

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization. Whole blood was collected from healthy donors in vacutainer ACD blood collection tubes (Becton Dickinson, bdbiosciences.com) centrifugated at 180×g for 20 minutes to collect platelet rich plasma (PRP).

The aggregation of washed FPH with washed platelets was screened with and without dual antiplatelet therapy (DAPT) using the PAP-8 Platelet Aggregometer. To obtain washed FPH and washed PRP, 6 μl of 1M citric acid was added to 1 ml of PRP and 1 ml of FPH, then centrifuged at 1000×g for 10 minutes and resuspended in HMTA. Washed FPH and washed platelets (WPS) were diluted to 300,000/μl in the appropriate amount of HMTA by AcT count to reach 3 ml each in duplicate. Aspirin (Cayman Chemical, caymanchem.com) and ticagrelor (Sigma Aldrich, sigmaaldrich.com) was added to one of the duplicates of washed platelets to achieve a concentration of a 100 μM and 1 μg/ml, respectively. 1 ml of washed FPH at 300,000/μl was added to 1 ml of washed platelets at 300,000/μl to reach 2 ml for both with and without DAPT (aspirin and ticagrelor) samples. The samples were immediately prepared prior to running on the PAP-8E Platelet Aggregometer. Next arachidonic acid (Helena Biosciences, helena-biosciences.com) was prediluted to 1:10 in HMTA. For each agonist (collagen and arachidonic acid) the following steps were performed in singlicate for each test sample group. 225 μl of sample was added to cuvettes by reverse pipetting and inserted into channels. The cuvettes were transferred from the warming wells to the testing wells. The run was started for each channel with 10 seconds in between. 25 μl of each agonist (collagen and arachidonic acid) was added to all cuvettes once the timer for the channel reached 8:00 minutes. HMTA was added in place of the agonist to the first channel in each module. After measurements were completed, each sample was transferred to a microcentrifuge tube and AcT counts were taken. FIG. 11 shows the effect of different agonists in the presence and absence of aspirin and ticagrelor (DAPT). Platelet counts for washed platelets (WPS) treated with DAPT were higher following agonist addition than untreated washed for both collagen and arachidonic acid, confirming that DAPT inhibition of washed platelets (FIG. 11). The inhibition of the interaction between FPH and platelets by DAPT indicates the ability of FPH to activate fresh platelets and induce aggregation. Though this interaction is mechanistically intriguing, FPH is expected to promote fresh platelet activation in vivo primarily through robust thrombin generation, even in the presence of DAPT.

### Example 12: Thrombin Generation Assay for Freeze-Dried Platelet-Derived Hemostats (FPH) and Apheresis Platelet Units (APU)

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

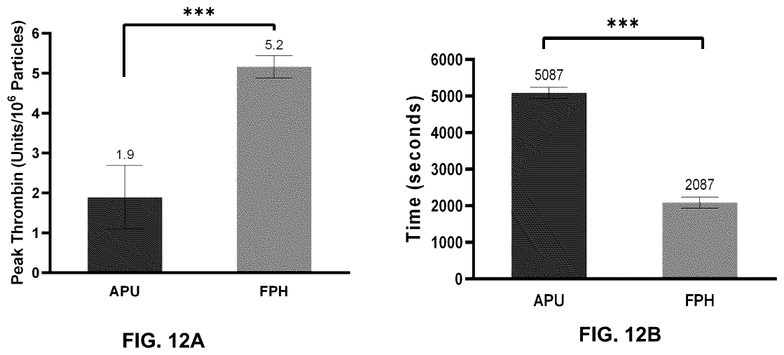

60 mM GPRP (BACHEM, bachem.com), thrombin (Sigma Aldrich, sigmaaldrich.com) and Octoplas® (Octophrama, octopharma.com) were thawed according to manufacturer's guidelines. CLARIOstar^{Plus} was used to perform the Thrombin generation assay (TGA). A master mix of Octaplas, PRP reagent (Diagnostica Stago, Cat. No. 86196), and GPRP was prepared in a ratio of 14:3:1, respectively. Flu substrate and Fluo-buffer were combined according to manufacterer's guidelines to create FluCa (Diagnostica Stago, stago.com. Master mix and FluCa solution were placed in the 37° C. air incubator with agitation for 45±5 minutes. The following dilutions of FPH and APU were prepared in duplicates in TGA dilution buffer: 1:32, 1:64, 1:128, and 1:256. 10 μl of each sample was dispensed into selected wells of a 96-well plate (Corning, corning.com) by reverse pipetting. TGA Dilution Buffer for thrombin was used in the thrombin standard ladder. CLARIOstar^{plus} injectors 1 and 2 were primed with master mix and FluCa solution with 250 μl, then the injector was placed in the active position. The CLARIOstar^{plus} TGA program was loaded with parameters for TGA assay with injectors set to add 90 μl of master mix and 40 μl of FluCa solution to each well. Plate was inserted to be read. This protocol was repeated for 5 different lots of FPH and 5 units of APUs. FIG. 12A and FIG. 12B show the peak thrombin and time to maximum thrombin generation for FPH and APUs. FPH generates more than twice as much peak thrombin as APU per particle. Further the time to maximum was also twice as fast as APU. FPH has substantially higher phosphatidylserine (PS) expression than resting platelets and can better catalyze thrombin generation.

### Example 13: FPH Thrombin Generation Response to PS Blocking by Lactadherin

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

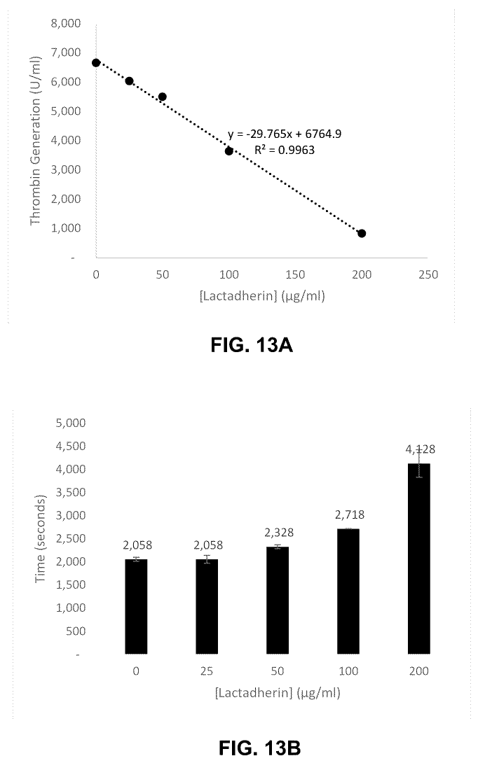

Lactadherin (Haematologic Technologies, haemtech.com) was thawed per manufacturer instructions. FPHs were incubated with lactadherin at room temperature for 30 minutes at a concentration of 0, 25, 50, 100, and 200 μg/ml. 60 mM GPRP (BACHEM, bachem.com), thrombin (Sigma Aldrich, sigmaaldrich.com) and Octoplas (Octophrama, octopharma.com) were thawed according to manufacturer's guidelines. CLARIOstarPlus was used to perform TGA. A master mix of Octaplas, PRP reagent (Diagnostica Stago, Cat. No. 86196), and GPRP was prepared in a ratio of 14:3:1, respectively. Flu substrate and Fluo-buffer were combined according to manufacturer's guidelines to create FluCa (Diagnostica Stago, stago.com). Master mix and the FluCa solution were placed in in the 37° C. air incubator with agitation for 45±5 minutes. The following dilutions of FPH were prepared in duplicates in TGA dilution buffer for each sample: 1:32, 1:64, 1:128, and 1:256. 10 μl of each sample was dispensed into selected wells of a 96-well plate (Corning, corning.com) by reverse pipetting. TGA dilution buffer for thrombin was used in the thrombin standard ladder. CLARIOstarPlus injectors 1 and 2 were primed with master mix and FluCa solution with 250 μl, then the injector was placed in the active position. The CLARIOstarPlus TGA program was loaded with parameters for TGA assay with injectors set to add 90 μl of master mix and 40 μl of FluCa solution to each well. Plate was inserted to be read. FIGS. 13A and 13B confirms that phosphatidylserine is necessary for the generation of thrombin based on the decreasing thrombin generation (FIG. 13A) and increasing maximum peak time (FIG. 13B) with dose dependent lactadherin for FPH.

### Example 14: FPH Thrombin Generation Response to Factor V Blocking

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

Monoclonal anti-Factor V antibody (Sigma Aldrich, sigmaaldrich.com) was diluted in HMTA to 0, 700, 1400, and 2800 μg/ml. 60 mM GPRP (BACHEM, bachem.com), thrombin (Sigma Aldrich, sigmaaldrich.com) and Octoplas® (Octophrama, octopharma.com) were thawed according to manufacturer's guidelines. CLARIOstarPlus was used to perform TGA. Different master mixes of Octaplas, PRP reagent (Diagnostica Stago, stago.com), GPRP, and each anti-FV antibody concentration were prepared in a ratio of 68:15:5:2, respectively. Additionally, a master mix of Octaplas, PRP reagent, GPRP, and 4 mg/ml anti-FV antibody was prepared in a ratio of 67.2:15:5:2.8, respectively. Flu substrate and Fluo-buffer were combined according to manufacturer's guidelines to create FluCa (Diagnostica Stago, stago.com). Master mix and FluCa solution were placed in the 37° C. air incubator with agitation for 45±5 minutes. The following dilutions of FPH were prepared in duplicates in TGA dilution buffer: 1:32, 1:64, 1:128, and 1:256. 10 μl of each sample was dispensed into selected wells of a 96-well plate (Corning, corning.com) by reverse pipetting. TGA dilution buffer for thrombin was used in the thrombin standard ladder. CLARIOstarPlus injectors 1 and 2 were primed with master mix and FluCa solution with 250 μl, then the injector was placed in the active position. The CLARIOstarPlus TGA program was loaded with parameters for TGA assay with injectors set to add 90 μl of master mix and 40 μl of FluCa solution to each well. Plate was inserted to be read.

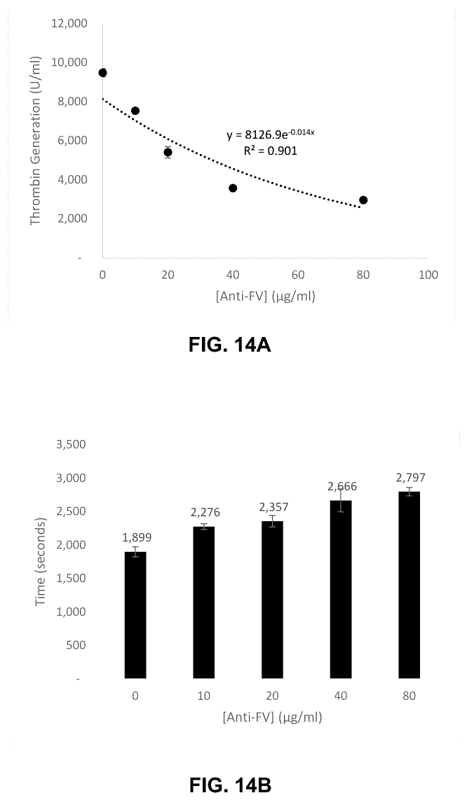

FIGS. 14A and 14B confirms that FV is necessary for the generation of thrombin based on the decreasing thrombin generation (FIG. 14A) and increasing maximum peak time (FIG. 14B) with dose dependent blocking of Factor V.

### Example 15: FPH Thrombin Generation Response to Factor X Blocking by Rivaroxaban

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

Rivaroxaban (Cayman Chemical, caymanchem.com) was diluted in TGA dilution buffer to 0 μg/ml, μg/ml, 3.5 μg/ml, 350 ng/ml, and 70 ng/ml. 60 mM GPRP (BACHEM, bachem.com), thrombin (Sigma Aldrich, sigmaaldrich.com) and Octoplas (Octophrama, octopharma.com) were thawed according to manufacturer's guidelines. CLARIOstarPlus was used to perform TGA. A master mix of Octaplas, PRP reagent (Diagnostica Stago, Cat. No. 86196), and GPRP was prepared in a ratio of 68:15:5, respectively. Flu substrate and Fluo-buffer were combined according to manufacturer's guidelines to create FluCa (Diagnostica Stago, stago.com). Master mix and FluCa solution were placed in the 37° C. air incubator with agitation for 45±5 minutes. The following dilutions of FPH were prepared in duplicates in TGA dilution buffer: 1:32, 1:64, 1:128, and 1:256. 10 μl of each sample was dispensed into selected wells of a 96-well plate (Corning, corning.com) by reverse pipetting. TGA dilution buffer for thrombin was used in the thrombin standard ladder. 2 μl of the rivaroxaban dilutions were dispensed into each duplicate FPH dilution set. CLARIOstarPlus injectors 1 and 2 were primed with master mix and FluCa solution with 250 μl, then the injector was placed in the active position. The CLARIOstarPlus TGA program was loaded with parameters for TGA assay with injectors set to add 90 μl of master mix and 40 μl of FluCa solution to each well. Plate was inserted to be read.

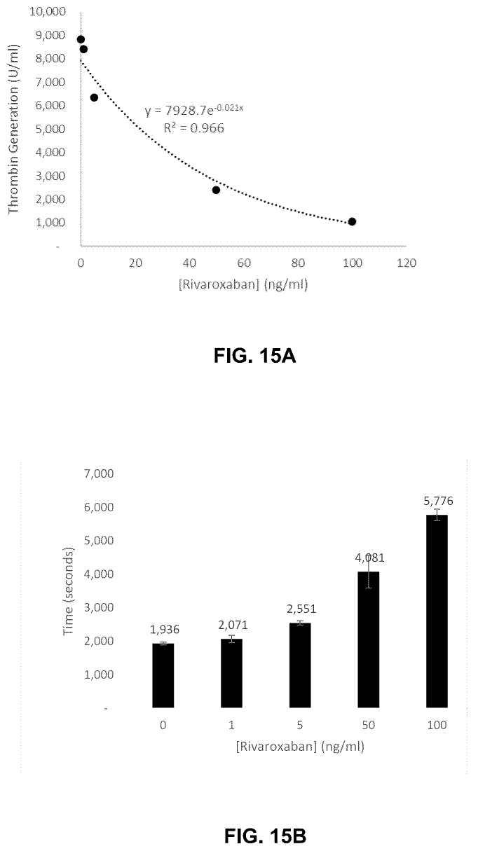

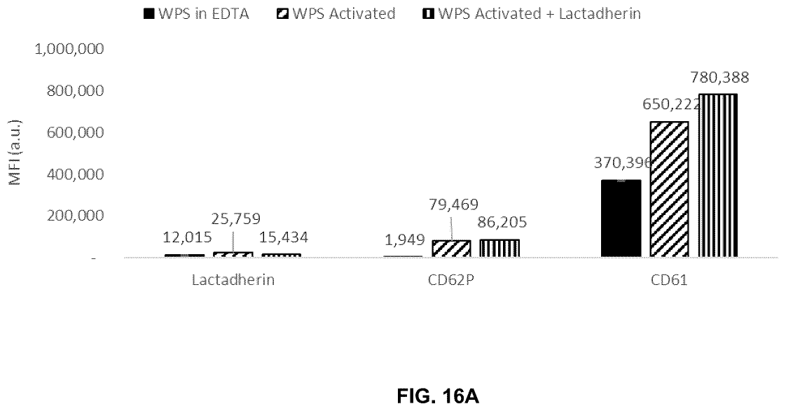

FIGS. 15A and 15B confirms that Factor X is necessary for the generation of thrombin based on the decreasing thrombin generation (FIG. 15A) and increasing maximum peak time (FIG. 15B) with dose dependent blocking of Factor X by Rivaroxaban.

### Example 16: Coagulation Factor Binding FPH and Washed Platelets

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. Whole blood was collected from healthy donors in vacutainer ACD blood collection tubes (Becton Dickinson, bdbiosciences.com) centrifugated at 180×g for 20 minutes to collect platelet rich plasma (PRP).

6 μl of 1M Citric acid was added to three tubes each containing 1 ml of PRP each and centrifuged at 1000×g for 10 minutes. Two pellets were resuspended with 5 mM Ca²⁺/Mg²+, 5 mM GPRP (BACHEM, bachem.com), and 20 μM TRAP (Tocris, tocris.com) in HMTA and the other pellet with 5 mM EDTA (Sigma Aldrich, sigmaaldrich.com) in HMTA. The platelets units were now washed platelet units (WPS). Counts were taken on the Beckman Coulter AcT Diff 2 Hematology Analyzer. One of the resuspensions in 5 mM Ca²⁺/Mg²⁺, 5 mM GPRP, and 20 μM TRAP was incubated with 200 μg/ml of unlabeled lactadherin (Haematologic Technologies, heamtech.com) for 30 minutes at room temperature.

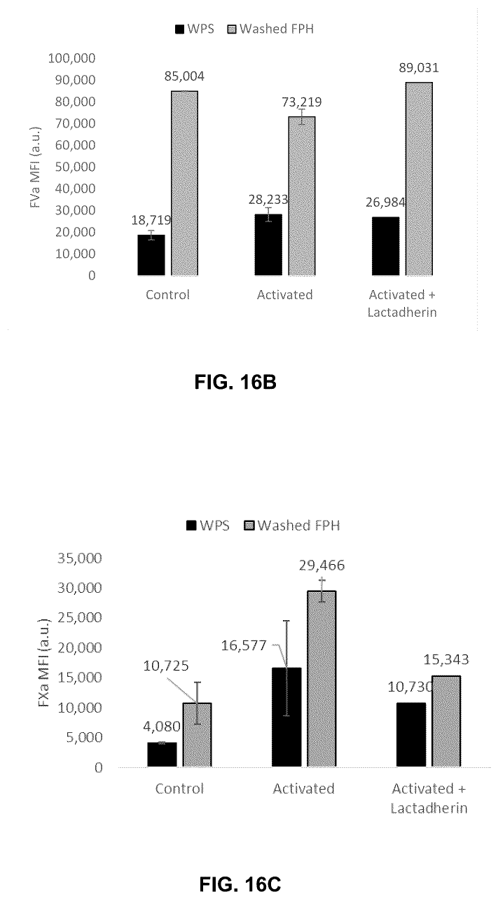

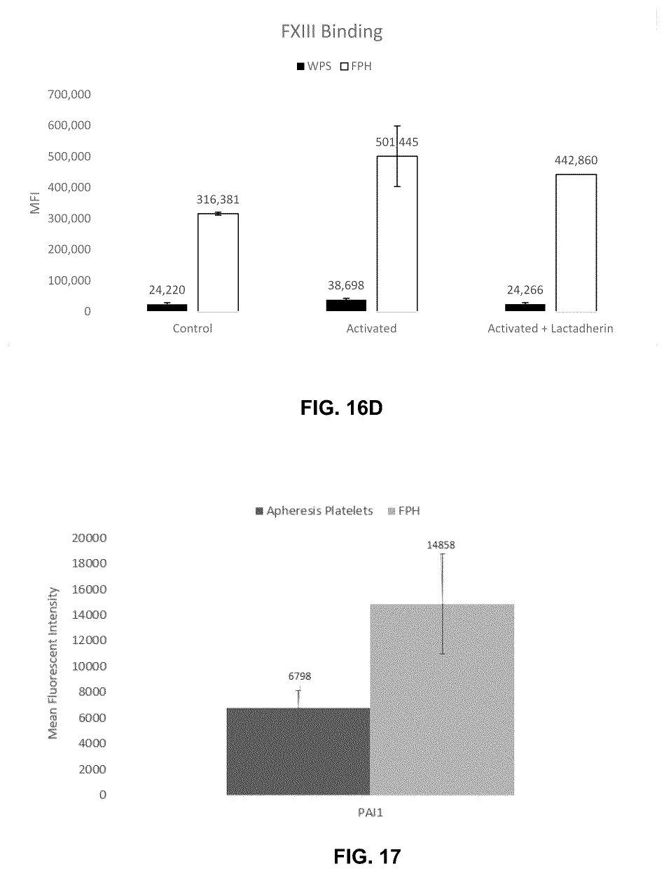

Prior to continuing the experiment, WPS were tested for platelet activation by flow cytometry with lactadherin, anti-CD62P antibody (BD Biosciences, bdbiosciences.com), and anti-CD61 antibody (BD Biosciences, bdbiosciences.com). All washed platelet samples were adjusted to 100,000/μl, diluted with the solution used for each resuspension. For all washed platelets samples, the following stains were prepared in duplicate: Unstained: 10 μL cells and 20 μL HMTA, Lactadherin: 5 μL cells, 20 μL lactadherin, and 5 μL HMTA, CD62P: 10 μL cells, 9 μL antibody, and 11 μL HMTA, and CD61: 10 μl cells, 5 μL antibody, and 15 μL HMTA. All samples were incubated away from open light for 20 minutes. After incubation, 500 μL of PBS were added to each sample. All samples were measured on the Quanteon flow cytometer. FIG. 16A shows that activated washed platelets have increased CD62 and PS expression confirming that activation of washed platelets. Further increased lactadherin binding in activated washed platelets was reduced by pre-incubating with unlabeled lactadherin, confirming PS expression in those samples.

FPH was rehydrated with 5 mM EDTA and allowed to sit for 10 minutes. The sample was sheared with a P1000 pipette and transferred 1 ml to an Eppendorf sample tube for further use. 6 μl of 1M CA was added to three tubes each containing 1 ml of FDPDs each and centrifuged at 1000×g for 10 minutes. Two pellets were resuspended with 5 mM Ca²⁺/Mg²+, 5 mM GPRP, and 20 μM TRAP in HMTA and the other pellet with 5 mM EDTA in HMTA. The FPH were now washed FPH. Counts were taken of each on the Beckman Coulter AcT Diff 2 Hematology Analyzer. One of the resuspensions in 5 mM Ca²⁺/Mg²⁺, 5 mM GPRP, and 20 μM TRAP was incubated with 200 μg/ml of unlabeled lactadherin for 30 minutes at room temperature. Based off the AcT counts, a sample was prepared of each FPH sample at a concentration of 50,000/μL. Based off the AcT counts, a sample was prepared of each WPS sample at a concentration of 50,000/μL. For each conjugated factor, each sample was stained using the protocols below for 90 minutes in the dark: Unstained: 10 μL cells and 10 μL HMTA, 1000 nM FX (Creative Biomart Cat #F10-355HA): 10 μL cells, 4 μL 5000 nM FX, and 6 μL HMTA, and 500 nM (FV (Creative Biomart Cat #F5-1176HF) and FXIII (Creative Biomart Cat #F13-53HA): 10 μL cells and 10 μL 1000 nM coagulation factor. 2 μl of sample was added to 98 μl of PBS in a well on a 96 well U-bottom plate, mixing thoroughly by pipetting for each tube. All samples were measured on the Quanteon flow cytometer. FIG. 16B shows the coagulation factor binding of Factor Va for washed FPH and washed platelets. Coagulation factor binging was higher for Factor Va in FPH than for WPS for control samples, activated samples, and activated samples plus lactadherin. Factor Va did not appear to differ in FPH across the different conditions, while activation increased Factor Va binding in WPS. FPH's ability to bind to Factor Va did not appear to be affected by lactadherin blocking of PS.

FIG. 16C shows the coagulation factor binding of Factor Xa for washed FPH and washed platelets. Factor Xa binding was higher for FPH than for WPS in the control, was substantially increased by activation, and reduced by lactadherin treatment. FPH's impact on thrombin generation is highly dependent on available PS and the assembly of Factor Va and Factor Xa into the prothrombinase complex. FPH can directly bind coagulation factors Va and Xa in higher quantities than platelets. Thus, FPH promotes the assembly of the prothrombinase complex on exposed PS to increase thrombin production.

FIG. 16D shows the expression of bound plasma Factor XIII in FPH and washed platelets. FPH has substantially greater demonstrated binding of FXIII indicating a potential explanation of stronger clot persistence of FPH in comparison to washed platelets.

### Example 17: PAI-1 Expression on FPH and APU

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

APU and FPH counts were determined by diluting APU and FPH samples 1:500 in phosphate-buffered saline (PBS) and acquiring events on the Quanteon flow cytometer (Agilent, agilent.com). Counts were determined using a bivariant logarithmic size density plot of forward scatter (FSC-Hvs (SSC-H) in a known volume of sample. Based on counts, FPH and APU were normalized to 100 k cells/ul in HEPES modified Tyrode's buffer with albumin (HMTA) before staining. 1 million total cells (10 uL) of either FPH or APU were stained and incubated for 20 minutes in final sample volumes of 30 uL containing the PAI-I antibody and HMTA to fill the remaining volume. Staining was performed on FPH and day 4 aged APU across 10 samples measured in duplicate for each group. The antibody concentration used was first obtained through antibody titration testing using isotype controls on FPH and APU in similarly prepared sample staining volumes. Final concentrations used was 33.3 μg/mL of anti-PAI1 antibody (Santa Cruz Biotechnology, scbt.com). All samples were then diluted 3:50 (v/v) in PBS and mean fluorescence intensity (MFI) was acquired on the Quanteon Flow Cytometer (Agilent, agilent.com). FIG. 17 shows PAI-1 expression of FPH and APU. PAI-1 is involved in fibrinolysis and encourages fibrin deposition at sites of injury and promotes hemostasis. FIG. 17 shows FPH expresses nearly twice as much PAI-1 as compared to APU, confirming that FPH support procoagulant response by preventing fibrinolysis.

### Example 18: Clot Lysis Analysis of FPH and APU

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

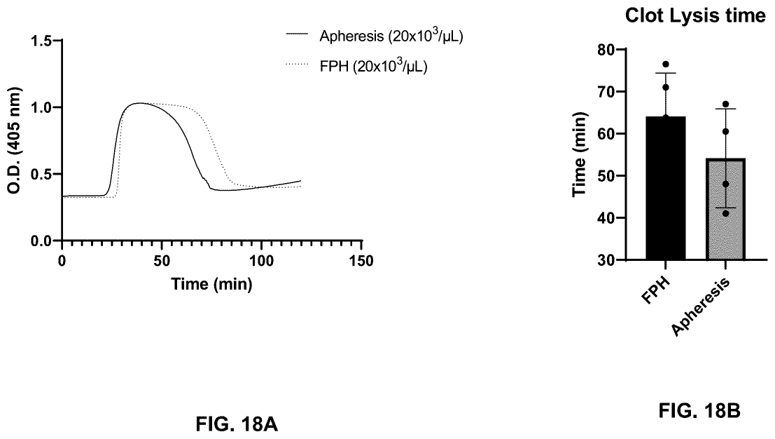

Samples were created by diluting FPH or in-date apheresis platelets to 20,000/uL in Octaplas containing 100 ng/mL tissue plasminogen activator (tPA). 100 uL of sample was added per well of a 96-well U-bottom plate by reverse pipetting. Each sample was tested in duplicate. 20 uL of PBS containing 42 mM CaCl₂ was added to each well, for a final concentration of 7 mM CaCl₂ in each well. The plate was immediately transferred to a prewarmed plate reader (Tecan) and the absorbance/Optical density (O.D.) read at 405 nm every 30 seconds for two hours. The clot lysis time was calculated as the time between 50% max O.D from clot formation to clot lysis. FIG. 18A shows the optical density versus time of the clot for both APU and FPH. FIG. 18B shows the clot lysis time for APU and FPH. FIGS. 18A and 18B depict a delayed clot lysis time for FPH relative to APU. This is in part due to the surface expressed PAI-1 as shown in example 17 and the bound plasma factor XIII as shown in example 16.

### Example 19: T-TAS® of FPH and Apheresis Platelet Unit Data

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

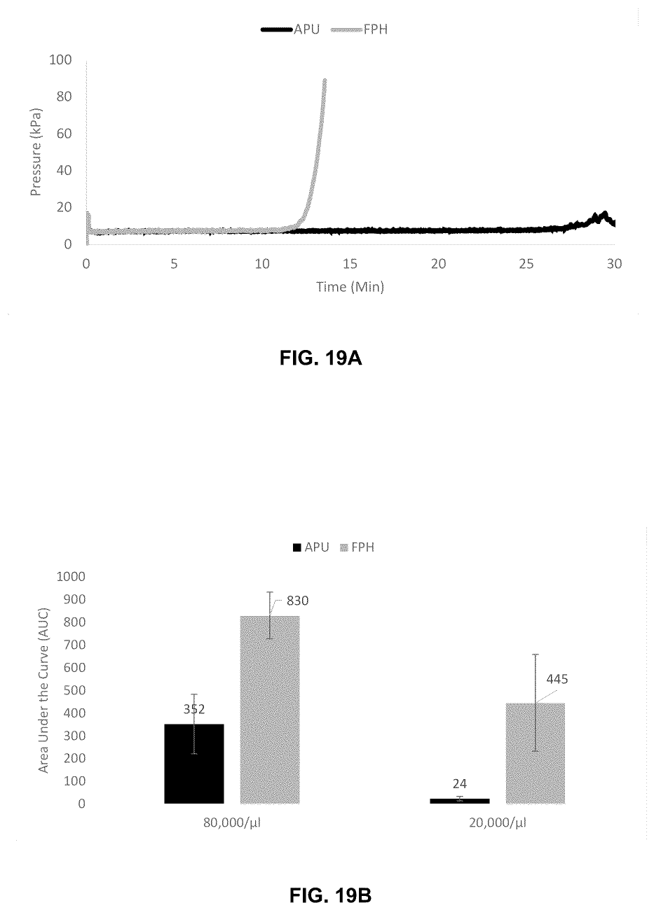

FPH and APU were first diluted 1:10 in triplicate and acquired for count on the AcT diff hematology analyzer. Cells were adjusted to 2×10⁴ cells/μl or 8×10⁴ cells/μl by diluting in Octaplas. Samples (480 ul) were normalized to the above counts and then transferred to microcentrifuge tubes containing 20 μl of CaCTI (CaCl₂ and corn trypsin inhibitor). The CaCTI-treated samples were transferred to sample reservoirs on a T-TAS® (Zacros®, zacros.con.co.jp). The sample reservoirs were then inserted into the AR microfluidic chips and acquired on the T-TAS. Data was collected and averaged for FPH and day 4 aged APU across 10 samples measured in singlicate for each group. FIG. 19A shows the T-TAS signal curves for FPH and APU at 8×10⁴ cells/μl. FIG. 19B shows the area under the curve (AUC) measurements for APU and FPH at 8×10⁴ cells/μl and 2×10⁴ cells/μl. FIGS. 18A and 18B confirm FPH's ability to create thrombus as tested by the T-TAS. The area under the curve for FPH was significantly higher for both concentrations of the channel (FIG. 18B). In fact at 2×10⁴ cells/μl concentration, APU often did not occlude the channel, while FPH consistently occluded the channel.

### Example 20: T-TAS® of FPH with Increasing Dose of Lactadherin

A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

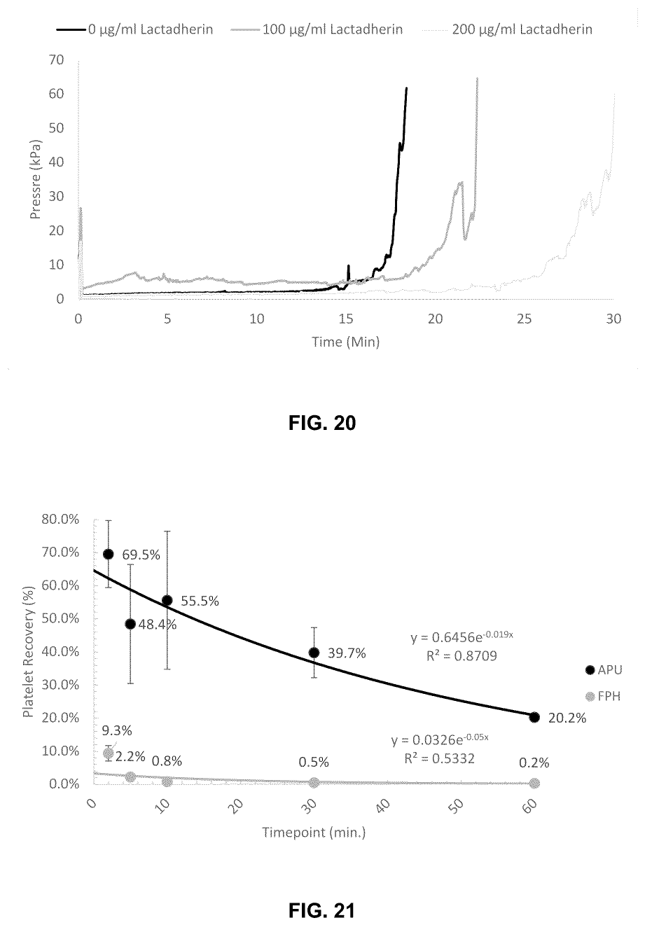

FPH were first diluted 1:10 in triplicate and acquired for count on the AcT diff hematology analyzer. Cells were adjusted to 8×10⁴ cells/μl by diluting in Octaplas and in preparations of either 0, 100, or 200 μg/mL of lactadherin and incubated for 30 minutes at room temperature before acquisition on T-TAS® (Zacros®, zacros.con.co.jp). Samples (480 μl) were normalized to the above counts and then transferred to microcentrifuge tubes containing 20 ul of CaCTI (CaCl₂ and corn trypsin inhibitor). The CaCTI-treated samples were transferred to sample reservoirs on a T-TAS®. The sample reservoirs were then inserted into the AR microfluidic chips and acquired on the T-TAS. Data was collected and averaged for FPH across 10 samples measured in singlicate for each group. FIG. 20 shows the T-TAS signal curves for FPH tested at 8×10⁴ cells/μl with 0, 100, and 200 μg/mL of lactadherin pretreatment. The highest lactadherin concentration approximately doubled the time to thrombus formation from the untreated FPH. This confirms the necessity of retaining the negatively charged PS surface on FPH to generate thrombin.

### Example 21: Circulation Persistence of FPH Versus APU in NOD-SCID Mouse Model

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

A non-obese diabetic/severe combined immunodeficiency (NOD-SCID) mouse model of human platelet circulation persistence was used for this experiment. Female mice were weighed and placed under a warming lamp before injection. Then, 200 μL of CFDA-SE-labeled platelet suspension of FPH or APU was injected into the lateral tail vein. Blood was collected via submandibular vein puncture at 2-, 5-, 10-, 30-, and 60-minutes following infusion. About 20 μL of blood was collected into clean 0.65 ml tubes and fixed with 20 μL ThromboFix (Beckman Coulter, beckmancoulter.com) at different time intervals. Whole blood samples were diluted in PBS in a 1:251 ratio to obtain an appropriate event rate and acquired on the Quanteon flow cytometer. The sample dilution factor was entered into the software and the absolute platelet count was determined for particles falling within the FITC+ (CFDA-SE positive) gate. Circulation persistence was calculated as the FITC+ platelet count at each time point divided by the theoretical maximum platelet count. The theoretical count was calculated using the known infusion volume, product platelet count, and an estimated mouse blood volume.

Theoretical count=Platelet Product Count×Injection Volume/(Mouse Weight×80 μ/g)

FPH had an initial recovery of 10% of infused platelets at the 2-minute timepoint, which was a 7-fold lower than the APU recovery of 70% (FIG. 21). By the 10-minute sampling point, less than 1% of FPH remained in circulation while 56% of APU were detected in circulation. (FIG. 21). FPH has substantially shorter circulation half-life than APU in the NOD-SCID mouse model.

### Example 22: Hemostasis Analysis of FPH and APU in NOD-SCID Tail Bleed

Single donor apheresis platelet units (APU) were purchased from LifeShare Blood Center. A thrombosome composition with hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

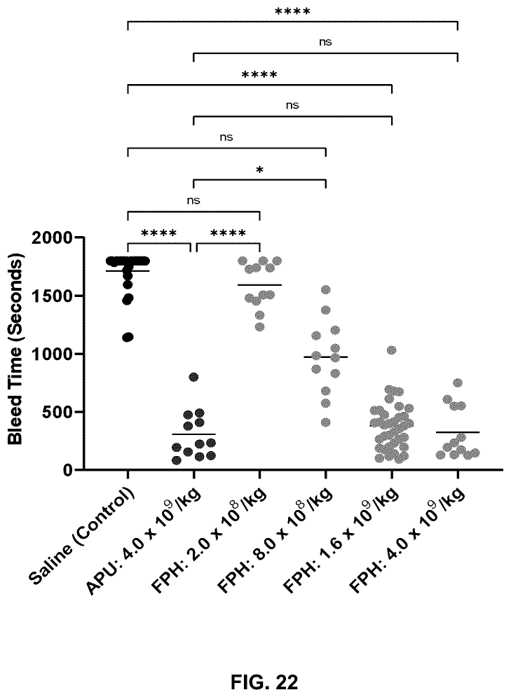

A non-obese diabetic/severe combined immunodeficiency (NOD-SCID) mouse model was used for the hemostasis analysis. NOD-SCID female mice (n=28) were anesthetized, and the femoral artery was exposed and infused with 80 μL of the either saline, APU, or FPH. Mice were infused with saline, APU at 4.0×10⁹/kg, or FPH at 2.0×10⁸/kg, 8.0×10⁸/kg, 1.6×10⁹/kg, or 4.0×10⁹/kg. Immediately after infusion (within 20 seconds), the tail was cut to a 2 mm diameter. The tail was immersed in warm saline and the time to cessation of bleeding was visually observed. FIG. 22 shows the time for cessation of bleeding for the saline, APU, and different dose of FPH. Mice treated with saline control bled for an average of 1712 seconds. The FPH dose of 1.6×10⁹/kg also restored hemostasis rapidly with a mean bleeding time of 379 seconds, which was not statistically significantly different from the 4.0×10⁹/kg APU mean bleeding time of 307 seconds (p=0.31). The intermediate dose of 8.0×10/kg restored hemostasis with an average bleed time of 971 seconds. All doses of FPH reduced mean bleeding time in comparison to saline control. Based on dose dependent hemostatic response top FPH, it shows strong potential as a hemostatic agent as a first in case hemostatic treatment.

### Example 23. Freeze-Dried Platelet-Derived Hemostat (FPH) Improve Thrombin Generation in Hermansky Pudlak Syndrome (HPS) Patients

Analysis of thrombin generation by Platelet Rich Plasma (PRP) obtained from blood of patients with HPS and further treated with varying doses of FPHs, was performed. Human FPHs were prepared according to the method as described in Example 1 of U.S. Pat. No. 11,529,587 B2, incorporated herein by reference in its entirety. The FPHs were rehydrated with sterile water equivalent to the fill volume prior to lyophilization.

Whole blood from patients with HPS (n=4) and healthy donors (n=21) were collected in 3.2% sodium citrate tubes (BD #363083). Platelet rich plasma (PRP) was generated by centrifugation of whole blood for 10 minutes at a Relative Centrifugal Force (RCF) of 180×g and removal of the top PRP layer. The remainder of the tube was centrifuged for 10 minutes at a RCF of 1800×g and the top platelet poor plasma (PPP) layer was removed. The PRP was adjusted to 200 k platelets/μL with PPP (k=10³).

Thrombin calibrator reagent (Stago #86192) was prepared according to manufacturer's guidelines (Stago, available on the internet at stago.com). 20 μL of the thrombin calibrator reagent was added to each calibration well, and 20 μL of PBS was added to each thrombin generation well. PRP was aliquoted into microcentrifuge tubes and treated with sFPHs at 5, 20, and 50 k/μL of FPHs. Samples were diluted 1:10 in OCTOPLAS® and a multi-channel pipette was used to add 80 μL of the diluted sample to each of the assay wells of an assay plate (calibration and thrombin generator wells). Three wells per sample were used for the thrombin calibrator and three wells were used for thrombin generation wells. Octaplas® plasma is a solvent/detergent treated, pooled human plasma available from Octapharma USA, Inc.

The assay plate was inserted into Thrombinoscope (Stago) connected to a computer with CAT software running. The assay plate was incubated at 37° C. for 10 min. During plate incubation Fluo-substrate and Fluo-buffer (Stago #86197) were combined according to manufacturer's guidelines to obtain FluCa buffer. ADP (Chronolog #384) and PGE1 (Cayman #13010) were added to FluCa buffer. ADP was added to FluCa buffer to obtain a final concentration of 1p M of ADP in assay wells and PGE1 was added to FluCa buffer to obtain a final concentration of 20 nM of PGE1 in the assay wells. After plate incubation, 20 μL FluCa solution was injected into each well. Thrombin generation was read for 180 min at 40 second intervals. Data analysis was performed in GraphPad Prism (GraphPad Software, San Diego, CA, graphpad.com).

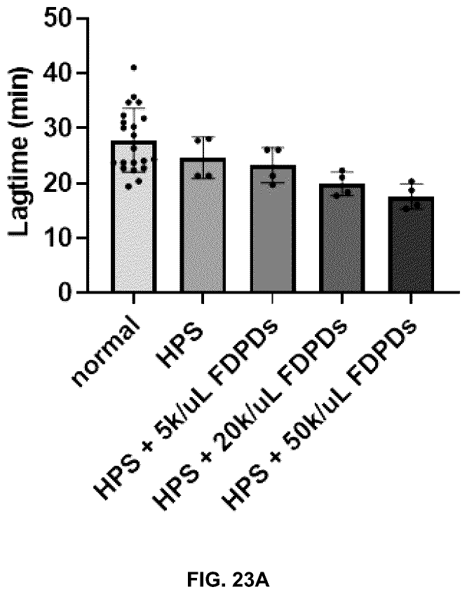

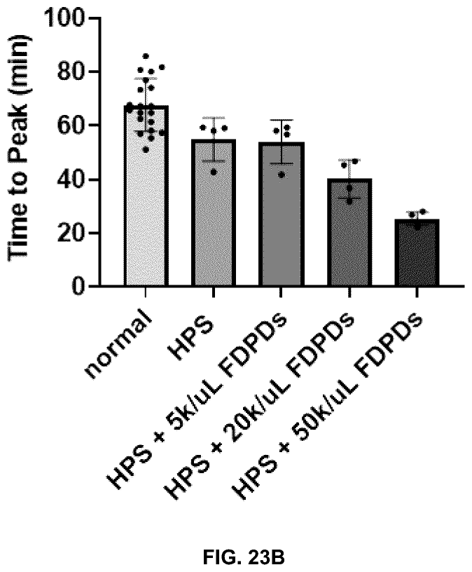

Results of this experiment as shown in FIG. 23A and FIG. 23B. FIG. 23A and FIG. 23B show a reduced lag time and reduced time to peak of thrombin production with the addition of FPHs to the PRP obtained from HPS patient blood, respectively. Thus, these ex vivo results support that FPHs can be effective in improving hemostasis in HPS patients.

### Example 24. FPHs Improve Clot Formation of HPS Patients as Shown in Thromboelastography (TEG) Method

Analysis of blood coagulation factors (via clot formation) from blood obtained from normal patients, HPS patients, and HPS patients after ex vivo addition of 50 k/μL (k=10³) of FPHs, was performed. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

TEG assays monitor clot formation. Clot formation parameters are measured including time to clot initiation (r time), the rate of clot formation (k time), the angle of the clot, the maximum amplitude “MA” or “size of the clot”, and the percent platelet activation normalized to the citrated kaolin sample. The instrument measures the resistance to movement in a pin submerged in the sample as the cup of sample moves.

Whole blood was retrieved from HPS patients and healthy patients via sodium citrate vacutainers (BD ref #363083) and lithium heparin vacutainers (BD ref #367884) and used within two hours to perform the experiment. Citrate tubes were kept on a rocker and heparin tubes were kept upright and mixed by inversion prior to using them. Whole blood for HPS patients was tested untreated and treated by ex vivo addition of 50 k/μL of FPHs. The TEG® PlateletMapping® Assay (Haemonetics cat #07-014, Boston, MA, haemonetics.com) was run according to manufacturer's instructions on TEG 5000 instrument. This required 2000 μL of citrated blood and 2,160 μL of heparin blood. For the samples treated with FPHs, the test article was added to a microcentrifuge tube, blood to which FPHs had been added ex vivo, was added to the tube and slowly mixed with a pipette, and then the run was immediately carried out according to the manufacturer's instructions. The results were returned automatically by the TEG software. Statistical analysis was carried out in GraphPad Prism (GraphPad Software, San Diego, CA, graphpad.com) using one-way ANOVA with Tukey's post-hoc test.

FIG. 24 shows the maximum amplitude of the clot of normal patient, HPS patient, and HPS patient blood treated with 50 k/μL of FPHs. FIG. 24 confirms that HPS patients have reduced maximum amplitude of the clot and shows that the addition of 50 k/μL of FPHs to HPS patient blood restores clot amplitude to near normal amplitude. The TEG® PlateletMapping® Assay clearly indicates that the additional of FPHs to HPS blood ex vivo improves coagulation via clot formation amplitude improvement.

### Example 25. FPHs Further Improve Clot Formation of HPS Patients in Comparison to Apheresis Platelets as Shown in Collagen Modified Thromboelastography (TEG) Method

Analysis of blood coagulation factors (via clot formation) from blood obtained from normal patients (n=5), HPS patients (n=3), and HPS patients after ex vivo addition of 50 k/μL (k=10³) (n=3) of FPHs or 50 k/μL of apheresis platelets (n=1), was performed. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization. TEG assays monitors clot formation. Clot formation parameters are measured including time to clot initiation (r time), the rate of clot formation (k time), the angle of the clot, the maximum amplitude “MA” or “size of the clot”, and the percent platelet activation normalized to the citrated kaolin sample. The instrument measures the resistance to movement in a pin submerged in the sample as the cup of sample moves.

Whole blood was retrieved from HPS patients and healthy patients via sodium citrate vacutainers (BD ref #363083) and lithium heparin vacutainers (BD ref #367884) and used within two hours to perform the experiment. Citrate tubes were kept on a rocker and heparin tubes were kept upright and mixed by inversion prior to using them. Whole blood for HPS patients was tested untreated and treated with 50 k/μL of FPHs or 50 k/μL of apheresis platelets.

FIG. 3A, of U.S. Pat. No. 11,529,587 and of PCT app no. PCT/US2022/079280, incorporated by reference herein in its entirety, shows the maximum amplitude of the clot of blood from normal patients, HPS patients, HPS patients treated with 50 k/μL of FPHs, and an HPS patient treated with 50 k/μL of apheresis platelets. FIG. 25B shows the rate of clot formation (k time) for blood from normal patients, HPS patients, HPS patients treated with 50 k/μL of FPHs, and an HPS patient treated with 50 k/μL of apheresis platelets. The results illustrated in FIG. 25A confirm that HPS patients have reduced maximum amplitude of the clot and surprisingly show that the addition of 50 k/μL of FPHs to HPS patient blood restores clot amplitude better than the same dose of apheresis platelets. Similarly, the results shown in FIG. 25B surprisingly show that 50 k/μL of FPHs reduced the k time better than the same dose of apheresis platelets in this ex vivo assay. These results using the collagen modified TEG PlateletMapping Assay clearly indicate that the additional of FPHs to HPS blood improves coagulation via clot formation amplitude and reduced rate of clot formation.

### Example 26. FPHs Contribute to an Increase of Endogenous PAC-1 Expression for HPS Patient Blood

The results in Examples 26 to 29 demonstrate that FPHs contribute to an increase in levels of platelet activation biomarkers for endogenous platelets in HPS patient blood. Analysis of the first procaspase activating compound (PAC-1) expression from blood obtained from normal patients, HPS patients, and HPS patients after ex vivo addition of 20 k/μL (k=10³) of FPHs was performed. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

Whole blood was retrieved from HPS patients (n=3) and normal healthy patients (n=6) via sodium citrate vacutainers (BD ref #363083). Normal whole blood was left untreated and used as a control for reagents and instruments used in the assay. 1 mL of the HPS whole blood from the sodium citrate tube was transferred to two tubes. The first tube of HPS blood was left untreated and the second tube of HPS blood was treated by the addition of 20 k/μL of FPHs. The tubes were inverted slowly three times and used immediately for PAC-1 staining.

For PAC-1 staining, 50 μL of each sample was stained in a well of a 96 well U-bottom plate with the following conditions: (1) 70 μL of HEPES-modified Tyrode's Albumin buffer (HMTA), 20 μL of muse-antihuman CD42b-FTIC (BD Biosciences Cat #555472), and 5 μL of mouse anti-human PAC-1AF647 (Biolegend 3628206), (2) 75 μL HMTA, 20 μL of mouse IgG1k-FITC (BD Cat #555748), and (3) 93.5 μL of HMTA, 1.5 μL of mouse IgM-AF647 (Biolegend Cat #401618).

To compensate for any color bleeding or any spillover of the different stains, single color controls were created. Single color controls are essential for any multicolor experiment. The single color controls reveal the level of spillover of the fluorophore into the other detectors. The spill over is mathematically removed, ensuring only specific signals are used in the final analysis.

Single color controls were created by combining 10 μL of each blood sample and adding 5 μL of the mixture into the wells with the following conditions: (1) 5 μL of PAC-1-AF647 in 90 μL of HMTA, (2) 20 μL CD42b-FITC in 75 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium and compensation was automatically calculated by the instrument and applied to all samples. To study only the endogenous HPS population, the contribution of FPHs to the CD42b positive or CD41 positive was removed by gating on the negative population for FPHs specific marker.

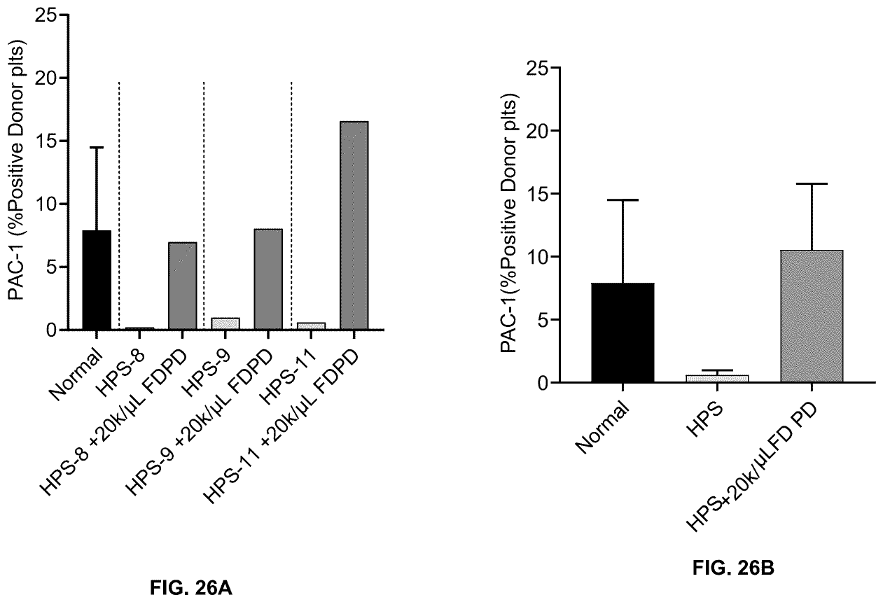

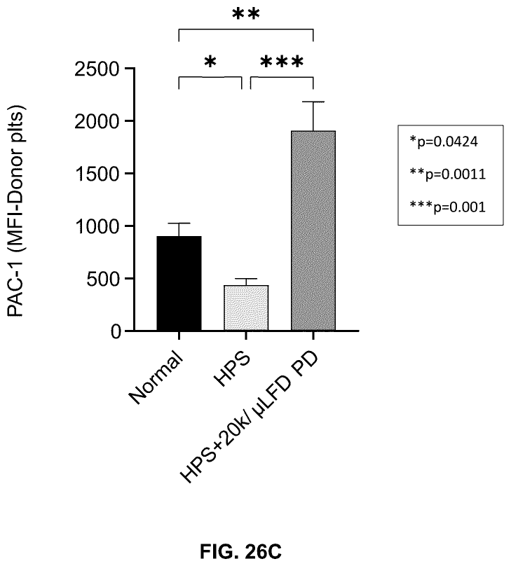

FIG. 26A and FIG. 26B show PAC-1 percent positivity of normal patients, HPS untreated patients, and HPS patients treated with 20 k/μL dose of FPHs. FIG. 26A shows individual HPS patients separated by dashed vertical lines in comparison to normal and untreated HPS donor blood, FIG. 26B shows the same data as the mean and standard error of the mean of the HPS patients' data. Both FIG. 26A and FIG. 26B show that the addition of 20 k/μL of FPHs to HPS blood is able to restore the percentage of endogenous platelets that are PAC-1 back to normal levels. FIG. 26C is the MFI representation of the same data as FIG. 26B and demonstrates the ability of exposure of HPS patient blood to FPHs to increase the levels of PAC-1 in the HPS patient blood.

### Example 27. FPHs Contribute to an Increase of Endogenous CD62P Expression for HPS Patient Blood

Analysis of CD62P expression from blood obtained from normal patients, HPS patients, and HPS patients after ex vivo addition of 20 k/μL (k=10³) of FPHs was performed. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

Whole blood was retrieved from HPS patients (n=3) and normal healthy patients (n=6) via sodium citrate vacutainers (BD ref #363083). Normal whole blood was left untreated and used as a control for reagents and instruments used in the assay. 1 mL of the HPS whole blood from the sodium citrate tube was transferred to two tubes. The first tube of HPS blood was left untreated and the second tube of HPS blood was treated by the addition of 20 k/μL of FPHs. The tubes were inverted slowly three times and incubated on the rocker for an additional 10 minutes. After 10 minutes on the rocker, 50 μL of each blood sample was combined with 50 μL of Thrombofix (Beckman Coulter Cat #6607130). The blood samples were left with the fixation solution for at least 1 hour followed by staining for CD62P (P-selectin).

For CD62P staining 5 μL of each sample was stained in a well of a 96 well U-bottom plate with the following conditions: (1) 70 μL of HMTA, 5 μL CD41-PE (Beckman Coulter Cat #1M1416U, and 20 μL CD62P-PECy5 (BD Biosciences Cat #551142), (2) 75 μL HMTA buffer containing 20 μL of mouse IgG1k-PECy5 (BD Biosciences Cat #555750), and (3) 90 μL of HMTA buffer containing 5 μL of mouse IgG1-PE (Beckman Coulter IM0670U).

Single color controls were created by combining 10 μL of each blood sample and adding 5 μL of the mixture into the wells with the following conditions: (1) 20 μL CD62P-PECy5 in 75 μL of HMTA, (2) 5 μL CD41-PE in 90 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium and compensation was automatically calculated by the instrument and applied to all samples. To study only the endogenous HPS population, the contribution of FPHs to the CD42b positive or CD41 positive was removed by gating on the negative population for FPHs specific marker.

FIG. 27A and FIG. 27B show CD62P-percent positivity when analyzing blood of normal patients, HPS patients without addition of FPHs, and HPS patients in which 20 k/μL of FPHs had been added before performing the analysis. FIG. 27A shows each individual HPS patients separated by dashed vertical lines in comparison to normal and untreated HPS donor blood, FIG. 27B shows the same data as mean and standard deviation error of the mean of the HPS patients' data. Both FIG. 27A and FIG. 27B show that the addition of 20 k/μL of FPHs to HPS blood restores the percentage of positivity of endogenous platelets that are CD62P back to normal levels. FIG. 27C is the MFI representation of the same data as FIG. 27B and demonstrate the ability of exposure of HPS patient blood to FPHs to increase the levels of CD62P in the HPS patient blood.

### Example 28. FPHs Contribute to an Increase of Endogenous CD63 Expression for HPS Patient Blood

Analysis of CD63 expression from blood obtained from normal patients, HPS patients, and HPS patients after ex vivo addition of 20 k/μL (k=10³) of FPHs was performed. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

Whole blood was retrieved from HPS patients (n=3) and normal healthy patients (n=4) via sodium citrate vacutainers (BD ref #363083). Normal whole blood was left untreated and used as a control for reagents and instruments used in the assay. 1 mL of the HPS whole blood from the sodium citrate tube was transferred to two tubes, the first tube of HPS blood was left untreated and the second tube of HPS blood was treated with 20 k/μL of FPHs. The tubes were inverted slowly three times and incubated on the rocker for an additional 10 minutes. After 10 minutes on the rocker, 50 μL of each blood sample was combined with 50 μL of Thrombofix (Beckman Coulter Cat #6607130). The blood samples were left with the fixation solution for at least 1 hour followed by staining for CD63.

For CD63 staining 5 μL of each sample was stained in a well of a 96 well U-bottom plate with the following conditions: (1) 85 μL of HMTA, 5 μL of mouse anti-human CD41-PE (Beckman Coulter Cat #1M1416U) and 5 μL of mouse anti-humanCD63-APC (Biolegend Cat #353008), (2) 90 μL HMTA, 5 μL of mouse IgG1-PE (Beckman Coulter IM0670U), and (3) 90 μL of HMTA, 5 μL of mouse IgG1-APC (Biolegend Cat #400122).

Single color controls were created by combining 10 μL of each blood sample and adding 5 μL of the mixture into the wells with the following conditions: (1) 5 μL CD63-APC in 90 μL of HMTA, (2) 5 μL CD41-PE in 90 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium, and compensation was automatically calculated by the instrument and applied to all samples. To study only the endogenous population of platelets in the HPS blood treated with FPHs, the contribution of FPHs to the CD42b positive or CD41 positive was removed by gating on the negative population for FPHs specific marker.

FIG. 28A shows cumulative data for CD63-percent positivity of normal patients, HPS patients, and HPS patients after ex vivo addition of 20 k/μL of FPHs. FIG. 28A shows that the addition of 20 k/μL of FPHs to HPS blood increases the number of cells that are positive for CD63 in comparison to the untreated HPS blood, bringing the percent positive cell numbers in the FPH-treated sample closer to the normal sample. FIG. 28B is the MFI representation of the same data as FIG. 28A.

### Example 29. Crossmatching Assay to Detect Level of IgG Via Flow Cytometry

Platelet antibodies in plasma or serum controls were detected by incubating samples with FPHs to perform a crossmatching assay. Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

HPS citrated plasma was retrieved from HPS whole blood. 60 μL of 30 k/μL FPHs were added to 60 μL of HPS plasma and incubated for 20 minutes at room temperature in a shaker. A positive serum and negative human serum were included and treated as the HPS plasma. After incubation cells were recovered by centrifugation at RCF of 1,100×g for 10 minutes. The supernatant was removed, and the pellet was washed twice with a (FWS) flow wash buffer (1×PBS and 5% Heat-inactivated normal goat serum). The cell pellet was resuspended in FWS at a concentration of 100K cells/μL. The staining of the samples was done by incubating with PE-labeled anti-human IgG (GOAT, F(ab′)₂ fragment) (Jackson Immuneresearch Laboratories, Inc, West Grove PA, jacksonimmuno.com) at room temperature for 20 minutes away from light. After the staining incubation, 500 μL of 1×PBS was added to all samples.

Analysis was performed by transferring 100 μL of the samples to a 96 well plate using a NovoCyte flow cytometer (Agilent, Santa Clara, CA, agilent.com). Thirty thousand events in the relevant gate surrounding FPH population were collected. Fluorescent intensity from the gated population was used to calculate IgG levels.

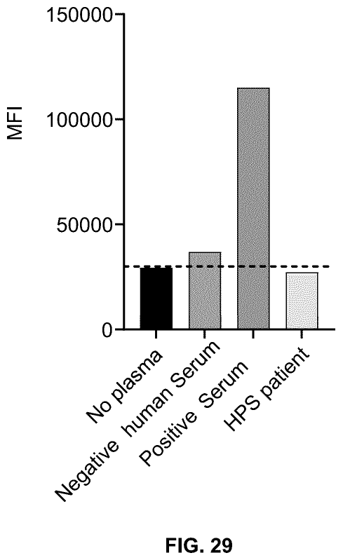

As shown in FIG. 29, anti-platelet IgG antibodies were detected in the positive serum. However, anti-platelet IgG antibodies were not detected in the HPS patient with ex vivo addition of FPHs. Therefore, HPS patient with ex vivo addition of FPHs does not lead to production of IgG antibodies against the FPHs.

### Example 30. Addition of FPHs to HPS Patient Blood Shows Dose Dependent Improvement of the Occlusion Time in PRP

Analysis of occlusion time with the addition of FPHs to HPS PRP patient sample was measured on the Total Thrombin formation Analysis System (T-TAS® 01) using HD chips (collagen and tissue factor stimulant with higher shear speed). Human FPHs were prepared according to the method as described in EXAMPLE 1 of U.S. Pat. No. 11,529,587 B2, incorporated herein by reference in its entirety. The FPHs were rehydrated with sterile water equivalent to the fill volume prior to lyophilization.

Analysis samples were prepared from HPS whole blood collected in acid citrate dextrose (ACD) collection tubes (BD vacutainers Ref #364606). Platelet rich plasma (PRP) was prepared by centrifugation of the ACD whole blood at a RFC of 180×g for 10 minutes and removal of the top PRP layer. The remainder of the tube was centrifugated for 10 minutes at a RCF of 1800×g and the top platelet poor plasma (PPP) layer was removed. The PRP was adjust to 75,000/μL with PPP.

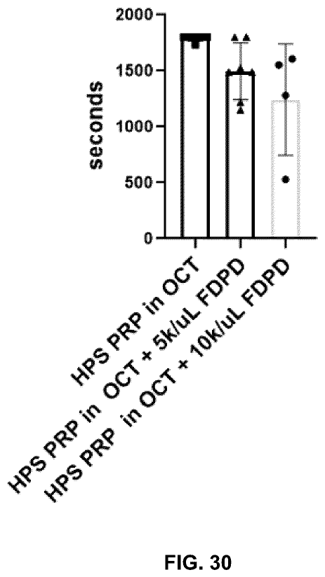

T-TAS® 01 (Zacros, zacros.co.jp) instrument was loaded with the HD chip and prepared for use according to the manufacturer's instructions. Three samples were run on the T-TAS® 01 machine, (1) HPS PRP and Octaplas®, (2) HPS PRP, Octaplas®, and 5 k/μL of FPHs, and (3) HPS PRP, Octaplas®, and 10 k/μL of FPHs. Octaplas® plasma is a solvent/detergent treated, pooled human plasma available from Octapharma USA, Inc. For each sample 480 μL of the prepared sample in the Octaplas® plasma was placed into a microcentrifuge tube with 20 μL of provided calcium CTI (CaCTI) reagent (Zacros, zacros.co.jp). The mixture was gently pipetted, loaded into T-TAS® 01 reservoir, and run on machine. FIG. 30 shows that addition of FPHs to HPS PRP improves the time to occlude within the HD channel in comparison to untreated HPS PRP. Furthermore, a high dose of FPHs is shown to further reduce the occlusion time.

### Example 31: FPH Detection in Whole Blood, In Vitro

The ability to detect unlabeled freeze-dried platelet-derived hemostats (FPH) in whole blood was determined in vitro. FPH and fresh platelets retain many of the same surface markers, making the discrimination between the two populations difficult. Surface characterization of FPH revealed that a large quantity of fibrinogen is bound to the surface compared to fresh platelets. The difference in fibrinogen provides a potential surface marker that allows for discrimination of FPH in the presence of other cells. In addition to fibrinogen as a prominent surface marker, FPH has previously been demonstrated to have a proprietary identification mechanism. This proprietary identification mechanism allows for detection of FPH in vitro in whole blood in concert with 9F9 antibody detection of fibrinogen in whole blood. An additional layer of discrimination in the detection of FPH in whole blood was added by staining FPH with 9F9 antibody as well as an anti-CD41 antibody conjugated to APC.

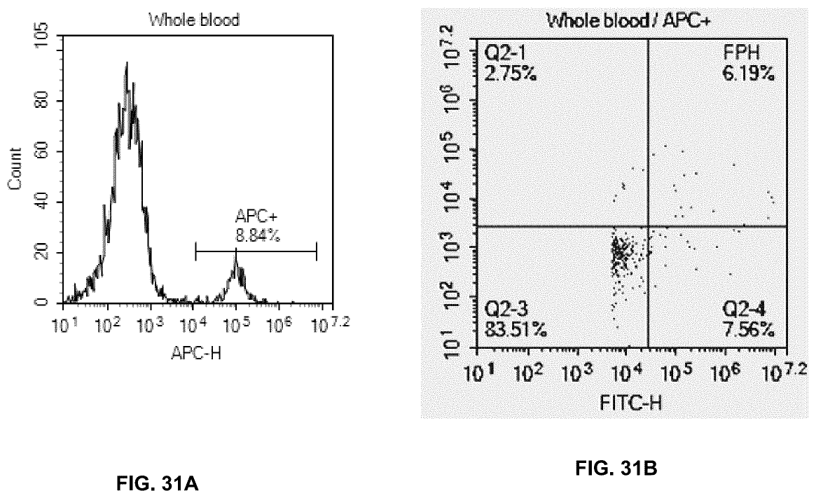

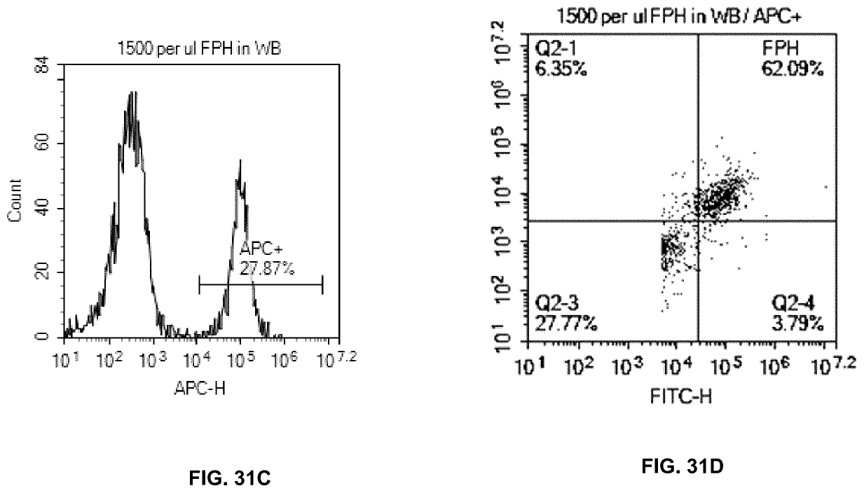

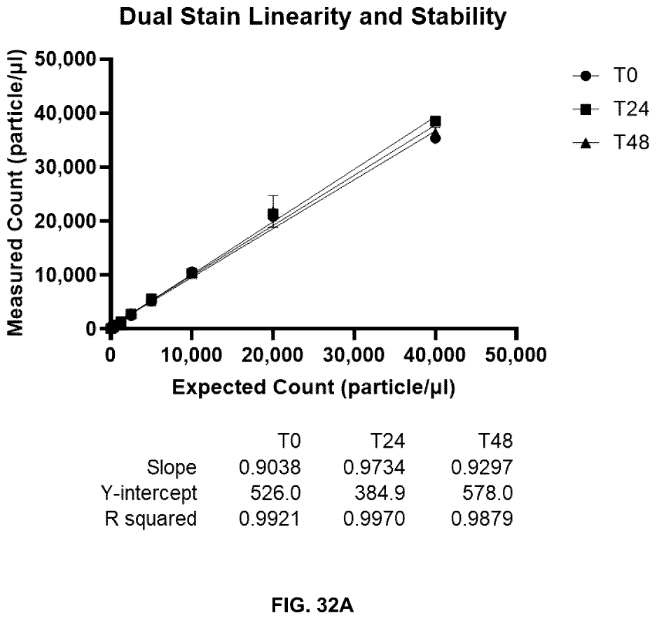

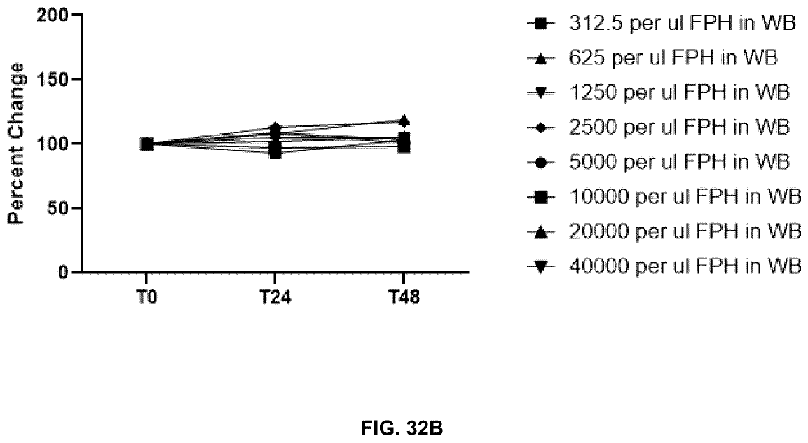

FPH at different concentrations was diluted in whole human blood collected in ACD tubes or in PBS buffer. Samples were then either fixed with equal volume of Thrombofix or immediately acquired on a NovaCyte Quanteon flow cytometer (Agilent, agilent.com). To fix samples, Thrombofix was added in a 1:1 dilution and incubated at room temperature for 1 hour. The samples were tested immediately after fixing, represented as TO in FIG. 32A and FIG. 32B, and at 24, and 48 hours after fixation, represented as T24 and T48 in FIG. 32A and FIG. 32B. Then 9F9 FITC antibody (BioCytex, biocytex.com) and anti-CD41-APC (Biolegend, biolegend.com) were added to each sample and incubated for 10 minutes at room temperature in the dark. FPH was labeled with RIBS anti-fibrinogen antibody (clone 9F9). The 9F9 antibody is specific to fibrinogen bound to the GpIIbIIIa on activated platelets, thus allowing specific detection in whole blood of only fibrinogen bound to FPH. Samples were then diluted at 1:500, then 1 ml of PBS and 1 μl sample was added to FACS tubes. For each tube, the samples were mixed thoroughly by pipetting and then vortexing at speed “g”. Samples were then transferred to NovaCyte Quanteon flow cytometer for collecting 20,000 events with FITC (B530) channel for 9F9, the proprietary identification mechanism, and APC (R660) channel for anti-CD41. Analysis was conducted by applying a gate for APC-CD41 positivity, then sub gating with bivariate plot for FITC 9F9 and the proprietary identification mechanism to determine FPH population. FIG. 31A, and FIG. 31B are the histogram and scattergram of the sample, respectively, without FPH, FIG. 31C, and FIG. 31D are the histogram and scattergram of the sample with FPH and gated for the three events (FITC 9F9, APC CD41+, proprietary identification mechanism). The linearity and stability of detection of FPH was tested for the combined methodology using the 9F9 antibody, CD41 antibody, and proprietary identification mechanism. FIG. 32A shows that the counts for FPH are linear across the entire range of concentrations of FPH tested. FIG. 32B shows that counts for FPH are stable across different time points after fixation (0, 24, 48 hours). Therefore, the methods for detecting FPH as demonstrated herein are able to detect FPH in various concentrations, and across different time points after fixation.

### Example 32: Delayed Fixation Analysis of FPH in Whole Blood

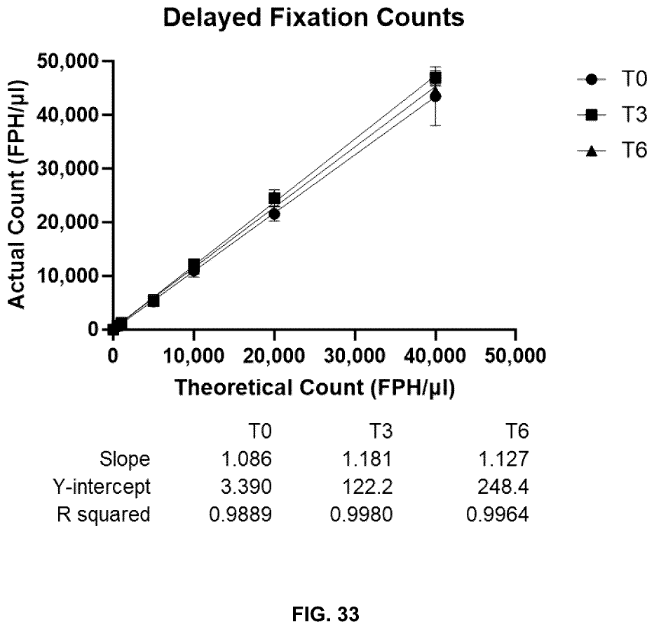

A time course of delayed fixation was performed to test the limits of how quickly blood needs to be fixed to maintain accurate FPH counts. FPH was diluted in different concentrations in whole blood collected in EDTA tubes, then fixed with equal volume of Thrombofix at 0, 3, and 6 hours post addition of FPH to whole blood. Samples were stained 24 hours post addition of FPH to whole blood with 9F9 (BioCytex, biocytex.com) and anti-CD41 (Biolegend, biolegend.com) antibodies. Samples were then collected on the NovaCyte Quanteon flow cytometer (Agilent, agilent.com) with channels B530, R667 and the proprietary identification mechanism. FIG. 33 shows the measured counts based on the gating scheme depicted in Example 31 versus the theoretical counts in whole blood sampled at the different concentrations of FPH added. Lines of best fit was applied to the data sets. FIG. 33 shows that counts remain accurate with delayed fixation. These results support the detection of FPH in whole blood in likely clinical scenarios where blood will not be able to be fixed immediately following collection from a patient.

### Example 33: Circulation Persistence of FPH in Non-Human Primates (NHP-Cynomolgus Macaques)

A study to evaluate the circulation persistence was conducted in 3 cynomolgus macaques at Biomere. Baseline blood draws were performed a week prior to the study initiation to test the feasibility of the method as well as establish background measurements in the NHPs. The animals received a single intravenous (IV) injection (over 2 minutes) of FPH, 3.18×10⁸ cells/kg on Day 1 followed by 0.5 mL of saline to flush the dose from the IV catheter. Doses were administered through the saphenous vein, cephalic vein or tail vein with a Terumo SURFLASH™ 22G or 24G×¾ temporary IV catheter. Injection site, dosing times, and dose volumes were recorded in the study file. The end time of dose administration was used to determine target times for blood sample collection time points. Vital signs, including blood pressure, heart rate, and respiration rate, were taken prior to dosing on Day 1 as well as following all blood collections.

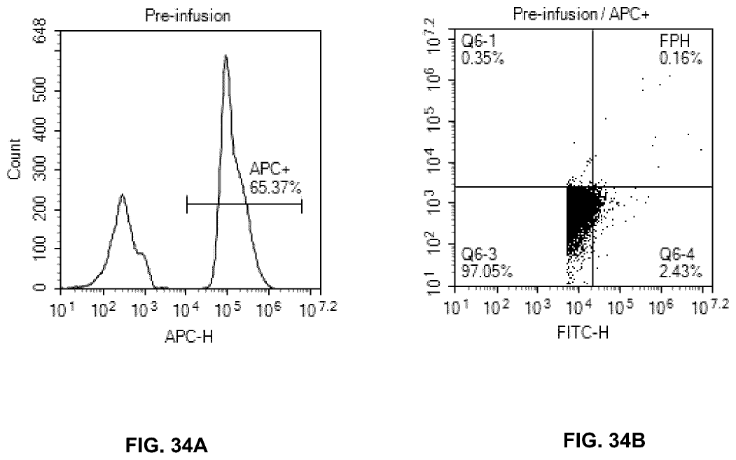

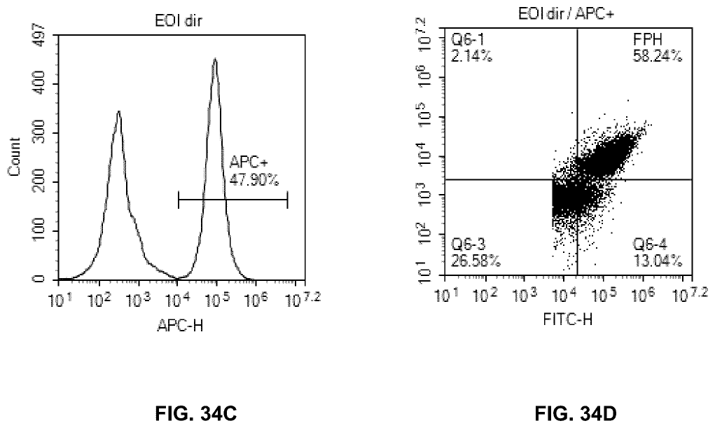

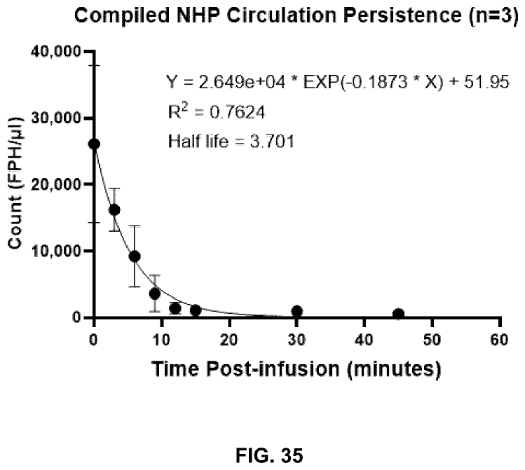

The sample collection schedule on Day 1 was 5-15 minutes prior to infusion, end of infusion (EOI), 3, 6, 9, 12, 15, 30, and 45 minutes post-infusion. All blood samples were collected via catheter, except an additional blood sample via direct stick was collected at EOI and 3 minutes post-infusion. Blood samples were fixed by adding 1:1 Thrombofix and shipped to a Cellphire facility at room temperature for analysis. Fixed samples collected in the study were stained in technical duplicates and analyzed by flow cytometry for quantification of FPH using the thrice gating method described in Example 31. FIG. 34A, and FIG. 34B show the histogram and scattergram representations pre-infusion (pre FPH infusion). FIG. 34C, and FIG. 34D show the histogram and scattergram at end of infusion (EOI of FPH). FIG. 35 shows the measured FPH counts in all the samples from the NHP study using the method of Example 31. Exponential decay lines of best fit were applied to the data sets. Rapid clearance of FPH is shown upon infusion. An initial product recovery of 39% was observed at the end of infusion and the half-life of FPH was calculated as about 3.7 minutes in this study. Table A provides the quantities of FPH determined in this NHP circulation persistence pilot study. Samples at all timepoints in each NHP were measured for FPH counts by the method of Example 31 and acquisition on the flow cytometer. The mean, minimum, maximum, and standard deviation of the samples across NHPs for each timepoint were calculated.

TABLE A

| | | Pre- | EOI | EOI | 3 min | 3 min | 6 | 9 | 12 | 15 | 30 | 45 |
|---|---|---|---|---|---|---|---|---|---|---|---|---|
| | Baseline | infusion | (catheter) | (direct) | (catheter) | (direct) | min | min | min | min | min | min |
| Mean | 320 | 120 | 16,956 | 32,263 | 19,167 | 15,254 | 9,219 | 3,631 | 1,403 | 1,098 | 980 | 594 |
| Minimum | 17 | 50 | 9,029 | 15,732 | 18,160 | 11,313 | 4,026 | 1,350 | 720 | 449 | 187 | 155 |
| Maximum | 666 | 264 | 24,186 | 46,732 | 20,174 | 19,748 | 16,404 | 7,207 | 2,614 | 1,892 | 1,879 | 1,441 |
| Std. Deviation | 282 | 82 | 6,249 | 10,713 | 1,424 | 3,052 | 4,564 | 2,736 | 866 | 736 | 752 | 523 |

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A method for administering a platelet derivative composition to a subject for reducing bleeding of the subject, said method comprising:
administering an effective dose of platelet derivatives in the platelet derivative composition to the subject, wherein the subject is a human subject with Hermansky-Pudlak syndrome (HPS),
wherein the administering is performed:
i) as a continuous infusion procedure; or
ii) at a frequency of at least one dose every 1 hour, or more frequently, wherein the administration comprises administering a total of 2 to 10 doses in a 24-hour period,
until bleeding of the subject is reduced as compared to the bleeding before the administration, and
wherein the platelet derivatives have the ability to generate thrombin in vitro in the presence of tissue factor and phospholipids, and
wherein the platelet derivatives have a reduced propensity to aggregate under aggregation conditions comprising an agonist but no fresh platelets, and in the absence of divalent cations, compared to the propensity of fresh platelets to aggregate under the conditions.

2. The method of claim 1, wherein the administering is performed until bleeding of the subject is stopped.

3. The method of claim 1, wherein the administering is performed at a frequency of every 30 minutes, or more frequently.

4. The method of claim 1, wherein the administering is performed at a frequency of every 1 hour, or more frequently, at variable time intervals, and at a dose of platelet derivatives in the range of 1.0×10⁷ to 1.1×10¹⁴/kg of the subject, and wherein the administering increases the levels of at least one platelet biomarker selected from CD62P, PAC-1, and CD63 for endogenous platelets of the subject as compared to before the administering.

5. A method for administering a platelet derivative composition to a subject for reducing bleeding of the subject, said method comprising:
administering an effective dose of platelet derivatives in the platelet derivative composition to the subject, wherein the subject is a human subject with Hermansky-Pudlak syndrome (HPS),
wherein the administering is performed by administering to the subject multiple times by administering 2 to 10 doses of the platelet derivatives at a frequency of every 1 hour, or more frequently, until bleeding of the subject is reduced as compared to the bleeding before the administering, and
wherein the platelet derivatives have the ability to generate thrombin in vitro in the presence of tissue factor and phospholipids, and
wherein the platelet derivatives have a reduced propensity to aggregate under aggregation conditions comprising an agonist but no fresh platelets, and in the absence of divalent cations, compared to the propensity of fresh platelets to aggregate under the conditions.

6. The method of claim 5, wherein the administering is performed until bleeding of the subject is stopped.

7. The method of claim 5, wherein the administering is performed at a frequency of every 30 minutes, or more frequently.

8. The method of claim 5, wherein the administering is performed by administering 3 to 10 doses at the frequency.

9. The method of claim 5, wherein the administering is performed by administering a total of 4 to 15 doses.

10. The method of claim 5, wherein the administering is performed at the frequency of every 15 minutes to 1 hour.

11. The method of claim 5, wherein the administering is performed by a parenteral route at variable time intervals, wherein the effective dose of platelet derivatives is in the range of 1.0×10⁷ to 1.1×10¹⁴/kg of the subject, and wherein the administering increases the levels of at least one platelet biomarker selected from CD62P, PAC-1, and CD63 for endogenous platelets of the subject as compared to before the administering.

12. The method of claim 2, wherein the administering is by intravenous administration, wherein the administration is performed at the frequency of every 1 hour, or more frequently, at a dose of platelet derivatives in the range of 1.0×10⁷ to 1.1×10¹⁴/kg of the subject.

13. The method of claim 4, wherein the administering is by intravenous administration, and wherein the administering leads to an improvement in clot formation in the subject as compared to the subject after being administered apheresis platelets, but before the administering of the platelet derivatives.

14. The method of claim 11, wherein the administering leads to an improvement in clot formation in the subject as compared to the subject after being administered apheresis platelets, but before the administering of the platelet derivatives.

15. The method of claim 11, wherein the administering further comprises administering a second composition comprising platelet derivatives by a topical route at the site of bleeding, and wherein the second composition is the same composition being administered by the parenteral route.

16. The method of claim 6, wherein the administering is performed intravenously at the frequency of every 15 minutes to 1 hour, and wherein a dose of platelet derivatives is in the range of 1.0×10⁷ to 1.1×10¹⁴/kg of the subject.

17. The method of claim 1, wherein less than 5% of the platelet derivatives in the platelet derivative composition are microparticles having less than 0.5 μm diameter.

18. The method of claim 17, wherein at least 65% of the platelet derivatives are positive for CD41, at least 65% of the platelet derivatives are positive for CD62, and at least 65% of the platelet derivatives are positive for CD42, wherein the platelet derivatives exhibit an increased thrombin generation in an in vitro assay as compared to platelets, and wherein:
(a) the presence of thrombospondin (TSP) on their surface is at a level that is greater than on the surface of resting platelets;
(b) the presence of von Willebrand factor (vWF) on their surface at a level that is greater than on the surface of resting platelets; or
(c) both (a) and (b).

19. The method of claim 5, wherein at least 65% of the platelet derivatives are positive for CD41, at least 65% of the platelet derivatives are positive for CD62, and at least 65% of the platelet derivatives are positive for CD42, wherein less than 5% of the platelet derivatives in the platelet derivative composition are microparticles having less than 0.5 μm diameter.

20. The method of claim 19, wherein the platelet derivatives exhibit an increased thrombin generation in an in vitro assay as compared to platelets, and wherein:
(a) the presence of thrombospondin (TSP) on their surface is at a level that is greater than on the surface of resting platelets;
(b) the presence of von Willebrand factor (vWF) on their surface at a level that is greater than on the surface of resting platelets; or
(c) both (a) and (b).
